SEQUENCE LISTING (14 sequences)

SEQ ID NO: 1 - protein, 451 aa, Artificial Sequence
  Heavy chain amino acid sequence of the anti-CD20 monoclonal antibody rituximab.
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Light chain amino acid sequence of the anti-CD20 monoclonal antibody rituximab.
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial Sequence
  Heavy chain amino acid sequence of the anti-CD20 monoclonal antibody obinutuzumab.
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 219 aa, Artificial Sequence
  Light chain amino acid sequence of the anti-CD20 monoclonal antibody obinutuzumab.
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 122 aa, Artificial Sequence
  Variable heavy chain amino acid sequence of the anti-CD20 monoclonal antibody ofatumumab.
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 6 - protein, 107 aa, Artificial Sequence
  Variable light chain amino acid sequence of the anti-CD20 monoclonal antibody ofatumumab.
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 7 - protein, 222 aa, Artificial Sequence
  Fab fragment of heavy chain amino acid sequence of  the anti-CD20 monoclonal antibody ofatumumab.
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 8 - protein, 211 aa, Artificial Sequence
  Fab fragment of light chain amino acid sequence of the anti-CD20 monoclonal antibody ofatumumab.
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 9 - protein, 451 aa, Artificial Sequence
  Heavy chain amino acid sequence of the anti-CD20 monoclonal antibody veltuzumab.
QVQLQQSGAEVKKPGSSVKVSCKASGYTFTSYNMHWVKQAPGQGLEWIGAIYPGMGDTSYNQKFKGKATLTADESTNTAYMELSSLRSEDTAFYYCARSTYYGGDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  Light chain amino acid sequence of the anti-CD20 monoclonal antibody veltuzumab.
DIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 447 aa, Artificial Sequence
  Heavy chain amino acid sequence of the anti-CD20 monoclonal antibody tositumomab.
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 210 aa, Artificial Sequence
  Light chain amino acid sequence of the anti-CD20 monoclonal antibody tositumomab.
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 13 - protein, 443 aa, Artificial Sequence
  Heavy chain amino acid sequence of the anti-CD20 monoclonal antibody ibritumomab.
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 14 - protein, 209 aa, Artificial Sequence
  Light chain amino acid sequence of the anti-CD20 monoclonal antibody ibritumomab.
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

(19) United States
(12) Patent Application Publication
(10) Pub. No.: US 2017/0035881 A1
(43) Pub. Date: Feb. 9, 2017

(54) Therapeutic Combinations of an IRAK4 Inhibitor and a BTK Inhibitor

(71) Applicant: Acerta Pharma B.V., Oss (NL)

(72) Inventors: Brian Lannutti, Solana Beach, CA (US); Wayne Rothbaum, Delray Beach, FL (US); Tjeerd Barf, Ravenstein (NL); Allard Kaptein, Zaltbommel (NL)

(21) Appl. No.: 15/297,998

(22) Filed: Oct. 19, 2016

Related U.S. Application Data
(60) Provisional application No. 62/243,319, filed on Oct. 19, 2015.

(51) Int. Cl. A61K 39/395; A61K 31/519; A61K 31/4439; A61K 31/4184; A61K 31/5377; A61K 31/4985; A61K 31/454

(52) U.S. Cl. CPC A61K 39/3955; A61K 31/4985; A61K 31/519; A61K 31/454; A61K 31/4184; A61K 31/5377; A61K 31/4439; A61K 2039/505

(57) ABSTRACT

Therapeutic combinations of an interleukin-1 receptor-associated kinase 4 (IRAK4) inhibitor and a Bruton's tyrosine kinase (BTK) inhibitor are described. In some embodiments, the invention provides pharmaceutical compositions comprising combinations of an IRAK4 inhibitor and a BTK inhibitor, and methods of using the pharmaceutical compositions for treating a disease, in particular a cancer.

### FIELD OF THE INVENTION

Therapeutic combinations of a Bruton's tyrosine kinase (BTK) inhibitor, and an interleukin-1 receptor-associated kinase 4 (IRAK4) inhibitor, and uses of the therapeutic combinations are disclosed herein. In particular, a combination of a BTK inhibitor and an IRAK4 inhibitor and compositions and uses thereof are disclosed.

### BACKGROUND OF THE INVENTION

Bruton's Tyrosine Kinase (BTK) is a Tec family non-receptor protein kinase expressed in B cells and myeloid cells. The function of BTK in signaling pathways activated by the engagement of the B cell receptor (BCR) and FCER1 on mast cells is well established. Functional mutations in BTK in humans result in a primary immunodeficiency disease characterized by a defect in B cell development with a block between pro- and pre-B cell stages. The result is an almost complete absence of B lymphocytes, causing a pronounced reduction of serum immunoglobulin of all classes. These findings support a key role for BTK in the regulation of the production of auto-antibodies in autoimmune diseases.

Other diseases with an important role for dysfunctional B cells are B cell malignancies. The reported role for BTK in the regulation of proliferation and apoptosis of B cells indicates the potential for BTK inhibitors in the treatment of B cell lymphomas. BTK inhibitors have thus been developed as potential therapies, as described in D'Cruz and Uckun, OncoTargets and Therapy 2013, 6, 161-176.

Interleukin-1 receptor-associated kinase 4 (IRAK4) is a member of the IRAK family of intracellular serine-threonine kinases, which consists of IRAK1, IRAK2, IRAK3 (or IRAKM), and IRAK4. Li, et al., Proc. Nat'l. Acad. Sci. USA, 2002, 99, 5567. IRAK4 signals downstream of the pathogen sensing toll-like receptors (TLRs), except for TLR3, and the innate/adaptive immune signaling IL-1 family (the IL-1, IL-18, and IL-33 receptors). Chaudhary, et al., J. Med. Chem. 2015, 58, 96-110. Upon binding to the IL-1 receptors or the TLRs, these receptors recruit the adaptor protein myeloid differentiation primary response gene 88 (MyD88) through the conserved Toll-IL-R (TIR) domain. MyD88 then utilizes the death domain (DD) homotypic interaction to recruit IRAK4. Lin, et al., Nature 2010, 465, 885. IRAK4 activation leads to the recruitment and phosphorylation of IRAK1 or IRAK2, which then leads to MAP kinase/IKK activation and proinflammatory cytokine production. The relationship of IRAK4 to key immune signaling receptors has led to interest in its inhibition for potential treatments for cancer as well as autoimmune and inflammatory diseases. Activating MyD88 mutations such as L265P in activated diffuse large B-cell lymphoma (DLBCL) and Waldenström's macroglobulinemia (WM) have established a role for IRAK family signaling in these and other cancers. Rhyasen and Starczynowski, Brit. J Cancer 2015, 112, 232-37.

In many solid tumors, the supportive microenvironment (which may make up the majority of the tumor mass) is a dynamic force that enables tumor survival. The tumor microenvironment is generally defined as a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment. Addressing the tumor cells themselves with e.g. chemotherapy has also proven to be insufficient to overcome the protective effects of the microenvironment. New approaches are thus urgently needed for more effective treatment of solid tumors that take into account the role of the microenvironment.

The CD20 antigen, also called human B-lymphocyte-restricted differentiation antigen Bp35, or B1), is found on the surface of normal “pre-B” and mature B lymphocytes, including malignant B lymphocytes. Nadler, et al., J. Clin. Invest. 1981, 67, 134-40; Stashenko, et al., J. Immunol. 1980, 139, 3260-85. The CD20 antigen is a glycosylated integral membrane protein with a molecular weight of approximately 35 kD. Tedder, et al., Proc. Natl. Acad. Sci. USA, 1988, 85, 208-12. CD20 is also expressed on most B cell non-Hodgkin's lymphoma cells, but is not found on hematopoietic stem cells, pro-B cells, normal plasma cells, or other normal tissues. Anti-CD20 antibodies are currently used as therapies for many B cell hematological malignancies, including indolent non-Hodgkin's lymphoma (NHL), aggressive NHL, and chronic lymphocytic leukemia (CLL)/small lymphocytic leukemia (SLL). Lim, et. al., Haematologica 2010, 95, 135-43; Beers, et. al., Sem. Hematol. 2010, 47, 107-14; Klein, et al., mAbs 2013, 5, 22-33. However, there is an urgent need to provide for more efficiacious therapies in many B cell hematological malignancies.

The present invention provides the unexpected finding that the combination of an IRAK4 inhibitor and a BTK inhibitor is synergistically effective in the treatment of any of several types of cancers such as leukemia, lymphoma, and solid tumor cancers, as well as inflammatory, immune, and autoimmune disorders. The present invention also provides the unexpected finding that a combination of an IRAK4 inhibitor, and a BTK inhibitor is synergistically effective in the treatment of any of several types of cancers such as leukemia, lymphoma, and solid tumor cancers, as well as inflammatory, immune, and autoimmune disorders. The present invention further provides the unexpected finding that the combination of an anti-CD20 antibody with a BTK inhibitor and an IRAK4 inhibitor, or a combination thereof, is synergistically effective in the treatment of any of several types of cancers such as leukemia, lymphoma, and solid tumor cancers, as well as inflammatory, immune, and autoimmune disorders.

### SUMMARY OF THE INVENTION

In an embodiment, the invention provides a method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof. In an embodiment, the IRAK4 inhibitor is administered to the mammal before administration of the BTK inhibitor. In an embodiment, the IRAK4 inhibitor is administered to the mammal simultaneously with the administration of the BTK inhibitor. In an embodiment, the IRAK4 inhibitor is administered to the mammal after administration of the BTK inhibitor.

In an embodiment, the invention provides a method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a BTK inhibitor or a pharmaceutically acceptable salt, solvate, hydrate, cocrystal, or prodrug thereof, wherein the BTK inhibitor is selected from the group consisting of:

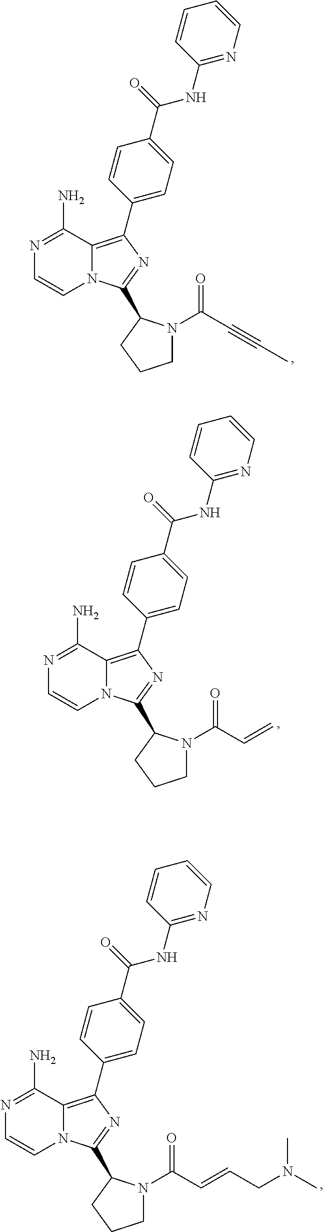 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","]

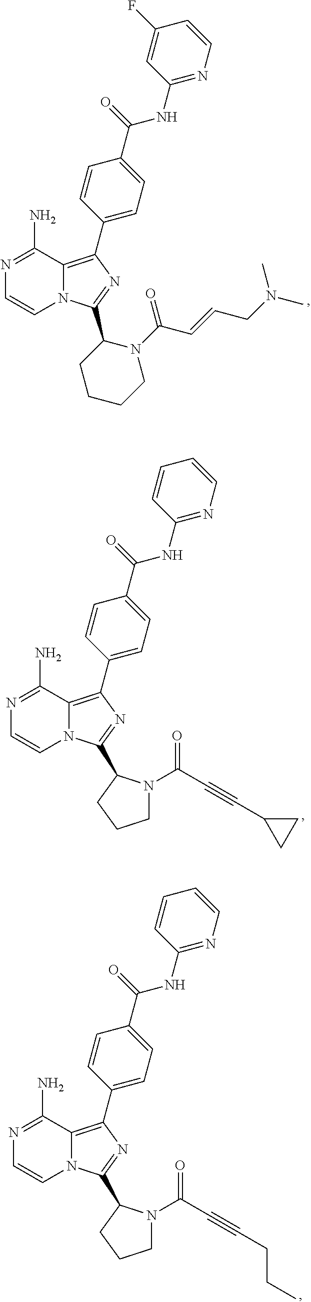 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]

and pharmaceutically-acceptable salts, cocrystals, hydrates, solvates, and prodrugs thereof.

In an embodiment, the invention provides a method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof, wherein the BTK inhibitor is selected from the group consisting of:

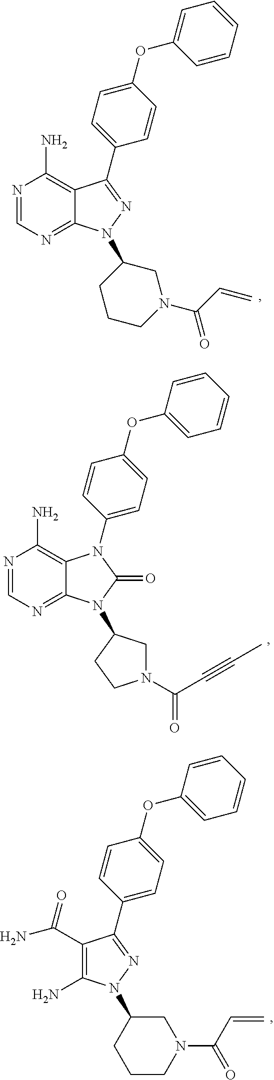

and pharmaceutically-acceptable salts, cocrystals, hydrates, solvates, and prodrugs thereof.

In an embodiment, the invention provides a method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a BTK inhibitor or a pharmaceutically acceptable salt, solvate, hydrate, cocrystal, or prodrug thereof, wherein the IRAK4 inhibitor is selected from the group consisting of:

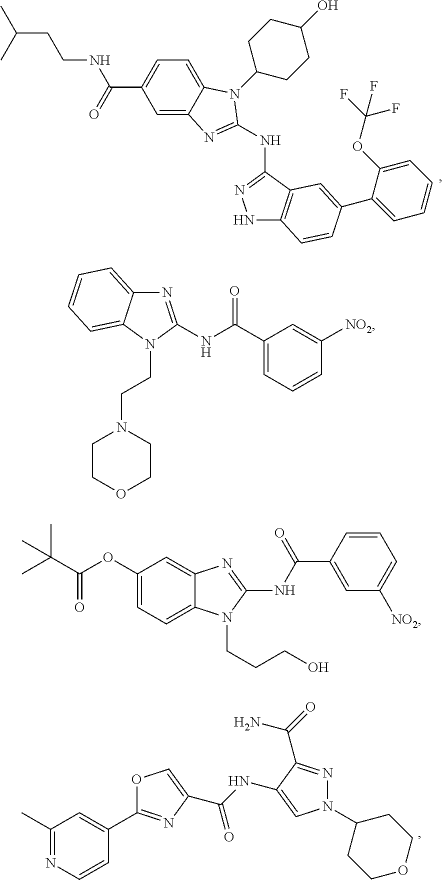

and pharmaceutically-acceptable salts, cocrystals, hydrates, solvates, or prodrugs thereof.

In an embodiment, the invention provides a method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a BTK inhibitor or a pharmaceutically acceptable salt, solvate, hydrate, cocrystal, or prodrug thereof, further comprising the step of administering a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof.

In an embodiment, the invention provides a method of treating a hyperproliferative disease, wherein the hyperproliferative disease is a cancer, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof, wherein the cancer is a B cell hematological malignancy, and wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, and myelofibrosis. In an embodiment, the cancer is a solid tumor cancer, wherein the solid tumor cancer is selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer.

In an embodiment, the invention provides a method of treating a cancer in a human comprising the step of co-administering (1) a therapeutically effective amount of an IRAK4 inhibitor or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein the therapeutically effective amount is effective to inhibit signaling between the tumor cells of the cancer and at least one tumor microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In an embodiment, the cancer is a solid tumor cancer selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer. In an embodiment, the BTK inhibitor is selected from the group consisting of:

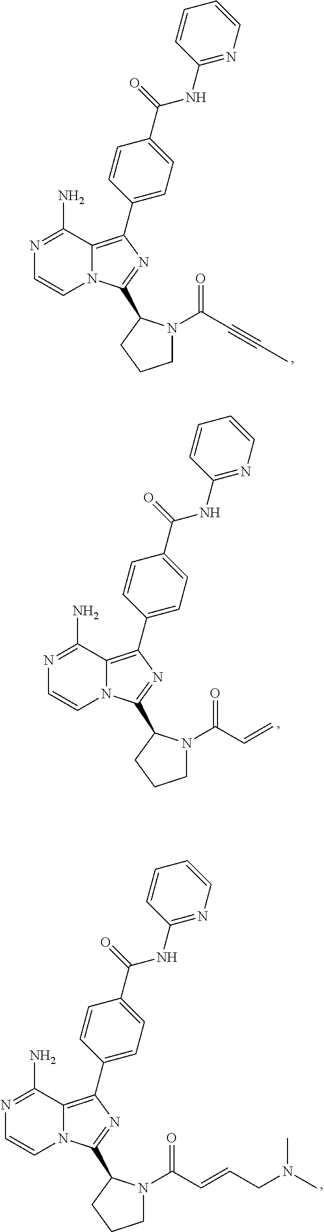 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","]

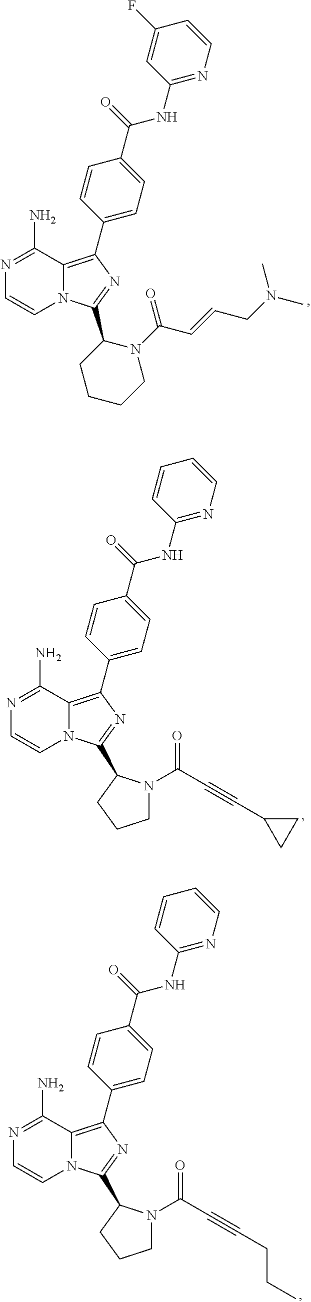 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]

and pharmaceutically-acceptable salts, cocrystals, hydrates, solvates, or prodrugs thereof. In an embodiment, the IRAK4 inhibitor is selected from the group consisting of:

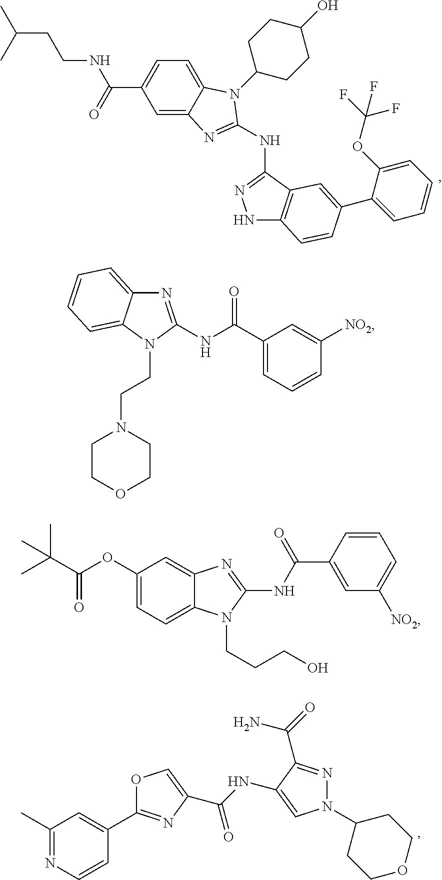

and pharmaceutically-acceptable salts, cocrystals, hydrates, solvates, and prodrugs thereof.

In an embodiment, the invention provides a method of treating a cancer in a human intolerant to a bleeding event comprising the step of administering (1) a therapeutically effective amount of an IRAK4 inhibitor or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein the BTK inhibitor is selected from the group consisting of:

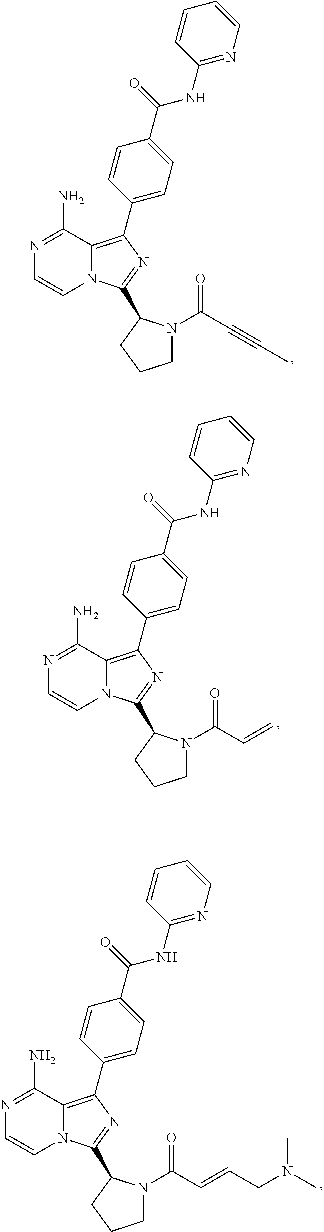 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","]

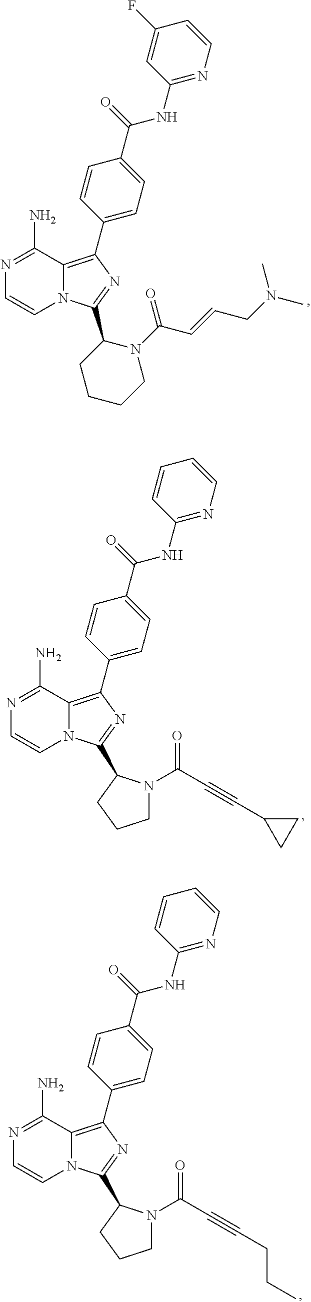 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]

and pharmaceutically-acceptable salts, cocrystals, hydrates, solvates, or prodrugs thereof. In an embodiment, the bleeding event is selected from the group consisting of subdural hematoma, gastrointestinal bleeding, hematuria, post-procedural hemorrhage, bruising, petechiae, and combinations thereof. In an embodiment, the IRAK4 inhibitor is selected from the group consisting of:

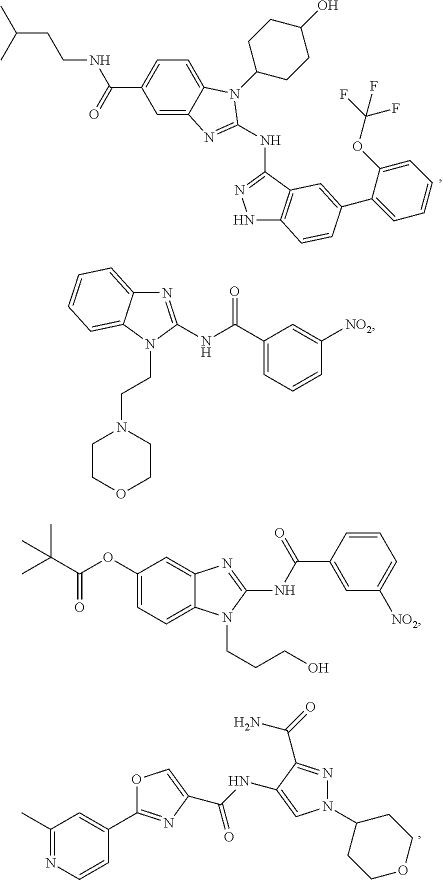

and pharmaceutically-acceptable salts, cocrystals, hydrates, solvates, and prodrugs thereof.

In an embodiment, the invention provides a method of treating a cancer in a human intolerant to a bleeding event comprising the step of administering (1) a therapeutically effective amount of an IRAK4 inhibitor or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, further comprising the step of administering a therapeutically effective amount of an anticoagulant or antiplatelet active pharmaceutical ingredient. In an embodiment, the anticoagulant or antiplatelet active pharmaceutical ingredient is selected from the group consisting of acenocoumarol, anagrelide, anagrelide hydrochloride, abciximab, aloxiprin, antithrombin, apixaban, argatroban, aspirin, aspirin with extended-release dipyridamole, beraprost, betrixaban, bivalirudin, carbasalate calcium, cilostazol, clopidogrel, clopidogrel bisulfate, cloricromen, dabigatran etexilate, darexaban, dalteparin, dalteparin sodium, defibrotide, dicumarol, diphenadione, dipyridamole, ditazole, desirudin, edoxaban, enoxaparin, enoxaparin sodium, eptifibatide, fondaparinux, fondaparinux sodium, heparin, heparin sodium, heparin calcium, idraparinux, idraparinux sodium, iloprost, indobufen, lepirudin, low molecular weight heparin, melagatran, nadroparin, otamixaban, parnaparin, phenindione, phenprocoumon, prasugrel, picotamide, prostacyclin, ramatroban, reviparin, rivaroxaban, sulodexide, terutroban, terutroban sodium, ticagrelor, ticlopidine, ticlopidine hydrochloride, tinzaparin, tinzaparin sodium, tirofiban, tirofiban hydrochloride, treprostinil, treprostinil sodium, triflusal, vorapaxar, warfarin, warfarin sodium, ximelagatran, salts thereof, solvates thereof, hydrates thereof, and combinations thereof. In an embodiment, the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hogkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, Burkitt's lymphoma, and myelofibrosis.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor or a pharmaceutically acceptable salt, solvate, hydrate, cocrystal, or prodrug thereof, for use in the treatment of cancer. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof, for use in the treatment of cancer; and (3) a therapeutically effective amount of an anti-CD20. In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a therapeutically effective amount of an anti-CD20. In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a therapeutically effective amount of an anti-CD20. In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a method of treating leukemia, lymphoma or a solid tumor cancer in a subject, comprising co-administering to a mammal in need thereof any of the foregoing compositions.

In some embodiments, the invention provides a method of treating leukemia, lymphoma or a solid tumor cancer in a subject, comprising co-administering to a mammal in need thereof a therapeutically effective amount of an IRAK4 inhibitor and a BTK inhibitor.

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and an anti-CD20 antibody.

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and albumin-bound paclitaxel.

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and bendustamine.

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and a combination of cyclophosphamide, doxorubicin, vincristine, and prednisone (CHOP).

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and a combination of rituximab, cyclophosphamide, doxorubicin, vincristine, and prednisone (R-CHOP).

In some embodiments, the invention provides a method of treating leukemia, lymphoma or a solid tumor cancer in a subject, comprising co-administering to a mammal in need thereof a therapeutically effective amount of an IRAK4 inhibitor, a BTK inhibitor, and a combination of fludarabine, cyclophosphamide, and rituximab (FCR).

### BRIEF DESCRIPTION OF THE DRAWINGS

The foregoing summary, as well as the following detailed description of the invention, will be better understood when read in conjunction with the appended drawings.

FIG. 1 illustrates in vivo potency of Formula (2) (labeled “BTK inhibitor”) and Formula (10) (ibrutinib). Mice were gavaged at increasing drug concentration and sacrificed at one time point (3 hours post-dose). BCR is stimulated with IgM and the expression of activation markers CD69 and CD86 are monitored by flow cytometry to determine EC₅₀values. The results show that Formula (2) is more potent at inhibiting expression of activation makers than Formula (10) (ibrutinib).

FIG. 2 illustrates in vitro potency in whole blood of Formula (2), Formula (10) (ibrutinib) and Formula (17) (CC-292) in inhibition of signals through the B cell receptor.

FIG. 3 illustrates EGF receptor phosphorylation in vitro for Formula (2) and Formula (10) (ibrutinib).

FIG. 4 illustrates the effects of vehicle on flux at two timepoints in the ID8 syngeneic orthotropic ovarian cancer model.

FIG. 5 illustrates the effects of the BTK inhibitor of Formula (2) on flux at two timepoints, for comparison with FIG. 4, in the ID8 syngeneic orthotropic ovarian cancer model.

FIG. 6 illustrates tumor response to treatment with the BTK inhibitor of Formula (2) correlates with a significant reduction in immunosuppressive tumor associated lymphocytes in tumor-bearing mice, in comparison to a control (vehicle).

FIG. 7 illustrates that treatment with the BTK inhibitor of Formula (2) impairs ID8 ovarian cancer growth in the syngeneic murine model in comparison to a control (vehicle).

FIG. 8 illustrates that treatment with the BTK inhibitor of Formula (2) induces a tumor response that correlates with a significant reduction in total B cells in tumor-bearing mice.

FIG. 9 illustrates that treatment with the BTK inhibitor of Formula (2) induces a tumor response that correlates with a significant reduction in B regulatory cells (Bregs) in tumor-bearing mice.

FIG. 10 illustrates that treatment with the BTK inhibitor of Formula (2) induces a tumor response that correlates with a significant reduction in immunosuppressive tumor associated Tregs.

FIG. 11 illustrates that treatment with the BTK inhibitor of Formula (2) induces a tumor response that correlates with an increase in CD8⁺ T cells.

FIG. 12 illustrates the effects of treatment with the active pharmaceutical ingredient of Formula (2) on tumor volumes in the KPC pancreatic cancer model.

FIG. 13 illustrates the results of analysis of tumor tissues showing that immunosuppressive TAMs (CD11b⁺Ly6ClowF4/80⁺Csflr⁺) were significantly reduced with Formula (2) treatment in the KPC pancreatic cancer model.

FIG. 14 illustrates the results of analysis of tumor tissues showing that immunosuppressive MDSCs (Gr1⁺Ly6CHi) were significantly reduced with Formula (2) treatment in the KPC pancreatic cancer model.

FIG. 15 illustrates the results of analysis of tumor tissues showing that immunosuppressive Tregs (CD4⁺CD25⁺FoxP3⁺) were significantly reduced with Formula (2) treatment in the KPC pancreatic cancer model.

FIG. 16 illustrates that the decrease in immunosuppressive TAMs, MDSCs, and Tregs in the KPC pancreatic cancer model correlated with a significant increase in CD8⁺ cells.

FIG. 17 illustrates the dosing schema used with the KrasLA2 non-small cell lung cancer (NSCLC) model.

FIG. 18 illustrates tumor volume variation from baseline as assessed by microcomputerized tomography (microCT) in the KrasL2 NSCLC model.

FIG. 19 illustrates TAMs in the KrasL2 NSCLC model, and indicates that Formula (2) induces a tumor response that correlates with a significant reduction in immunosuppressive tumor associated TAMs.

FIG. 20 illustrates MDSCs in the KrasL2 NSCLC model, and indicates that Formula (2) induces a tumor response that correlates with a significant reduction in immunosuppressive tumor associated MDSCs.

FIG. 21 illustrates Tregs in the KrasL2 NSCLC model, and indicates that Formula (2) induces a tumor response that correlates with a significant reduction in immunosuppressive tumor associated Tregs.

FIG. 22 illustrates CD8⁺ T cells in the KrasL2 NSCLC model.

FIG. 23 illustrates BTK inhibitory effects on MDSCs.

FIG. 24 shows in vitro analysis of antibody-dependent NK cell-mediated interferon-γ (IFN-γ) release with BTK inhibitors. To evaluate NK cell function, purified NK cells were isolated from healthy peripheral blood mononuclear cells and cultured with 0.1 or 1 μM of Formula (10) (ibrutinib) or 1 μM of Formula (2) for 4 hours together with rituximab-coated (10 μg/mL) lymphoma cells, DHL4, or trastuzumab-coated (10 μg/mL) HER2+ breast cancer cells, HER18, and supernatant was harvested and analyzed by enzyme-linked immunosorbent assay for IFN-γ. All in vitro experiments were performed in triplicate. Labels are defined as follows: *p=0.018, **p=0.002, ***p=0.001.

FIG. 25 shows in vitro analysis of antibody-dependent NK cell-mediated degranulation with BTK inhibitors. To evaluate NK cell function, purified NK cells were isolated from healthy peripheral blood mononuclear cells and cultured with 0.1 or 1 LM of Formula (10) (ibrutinib) or 1 μM of Formula (2) for 4 hours together with rituximab-coated (10 μg/mL) lymphoma cells, DHL4, or trastuzumab-coated (10 μg/mL) HER2⁺ breast cancer cells, HER18, and NK cells isolated and analyzed for degranulation by flow cytometry for CD107a⁺ mobilization. All in vitro experiments were performed in triplicate. Labels are defined as follows: *p=0.01, **p=0.002, ***p=0.003, ****p=0.0005.

FIG. 26 shows that Formula (10) (ibrutinib) antagonizes antibody-dependent NK cell-mediated cytotoxicity using the Raji cell line. NK cell cytotoxicity as percent lysis of tumor cells was analyzed in chromium release assays with purified NK cells incubated with chromium-labeled Raji for 4 hours at variable rituximab concentrations at a constant effector:target ratio of 25:1 and Formula (10) (ibrutinib) (1 μM), Formula (2) (1 μM), or other interleukin-2 inducible tyrosine kinase (ITK) sparing BTK inhibitors CGI-1746, inhibA (1 μM) and BGB-3111 (“inhibB,” 1 μM). All in vitro experiments were performed in triplicate. Labels are defined as follows: *p=0.001.

FIG. 27 shows that Formula (10) (ibrutinib) antagonizes antibody-dependent NK cell-mediated cytotoxicity in primary CLL cells. NK cell cytotoxicity as percent lysis of tumor cells was analyzed in chromium release assays with purified NK cells incubated with chromium-labeled Raji for 4 hours at variable rituximab concentrations at a constant effector:target ratio of 25:1 and Formula (10) (ibrutinib) (1 μM), Formula (2) (1 μM), or other ITK sparing BTK inhibitors CGI-1746, inhibA (1 μM) and BGB-3111 (“inhibB,” 1 μM). All in vitro experiments were performed in triplicate. Labels are defined as follows: *p=0.001.

FIG. 28 shows a summary of the results given in FIG. 27 at the highest concentration of rituximab (“Ab”) (10 μg/mL).

FIG. 29 shows NK cell degranulation results for combinations of obinutuzumab with Formula (2) and Formula (10). The percentage of CD56⁺/CD107a⁺NK cells observed in whole blood after pretreatment for 1 hour with the BTK inhibitors and stimulatation with MEC-1 cells opsonised with obinutuzumab at 1 μg/mL for 4 hours (n=3) is shown.

FIG. 30 shows the effects of BTK inhibition on generalized NK cell mediated cytotoxicity.

FIG. 31 shows that Formula (2) has no adverse effect on T helper 17 (Th17) cells, which are a subset of T helper cells that produce interleukin 17 (IL-17), while Formula (10) (ibrutinib) strongly inhibits Th17 cells.

FIG. 32 shows that Formula (2) has no effect on regulatory T cell (Treg) development, while Formula (10) (ibrutinib) strongly increases Treg development.

FIG. 33 shows that Formula (2) has no effect on CD8⁺ T cell viability, development, while Formula (10) (ibrutinib) strongly affects CD8⁺ T cell viability at higher doses.

FIG. 34 illustrates the results of the cytotoxicity assay for CD8⁺ T cell function. Formula (10) (ibrutinib) affects CD8⁺ T cell function as measured by % cytotoxicity, while Formula (2) has no effect on CD8⁺ T cell function as measured by % cytotoxicity relative to vehicle.

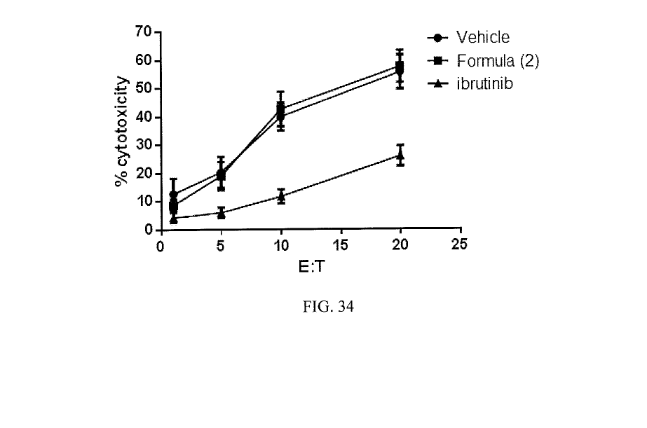

FIG. 35 illustrates the results of IFN-γ level measurements for CD8⁺ T cell function. Formula (10) (ibrutinib) affects CD8⁺ T cell function as measured by IFN-γ level, while Formula (2) has no effect on CD8⁺ T cell function as measured by IFN-γ level relative to vehicle.

FIG. 36 illustrates the results of the clinical study of Formula (2) (labeled “BTK inhibitor”) in CLL, which are shown in comparison to the results reported for Formula (10) (ibrutinib) in FIG. 1A of Byrd, et al., N. Engl. J. Med. 2013, 369, 32-42. The results show that the BTK inhibitor of Formula (2) causes a much smaller relative increase and much faster decrease in absolute lymphocyte count (ALC) relative to the BTK inhibitor of Formula (10) (ibrutinib). The sum of the product of greatest diameters (SPD) also decreases more rapidly during treatment with the BTK inhibitor than with the BTK inhibitor of Formula (10) (ibrutinib).

FIG. 37 shows SPD of enlarged lymph nodes in CLL patients as a function of dose (cohort) of the BTK inhibitor of Formula (2).

FIG. 38 shows a comparison of progression-free survival (PFS) in CLL patients treated with the BTK inhibitor of Formula (10) (ibrutinib) or the BTK inhibitor of Formula (2). The ibrutinib data is taken from Byrd, et al., N. Engl. J. Med. 2013, 369, 32-42. CLL patients treated with Formula (2) for at least 8 days are included.

FIG. 39 shows a comparison of number of patients at risk in CLL patients treated with the BTK inhibitor of Formula (10) (ibrutinib) or the BTK inhibitor of Formula (2). CLL patients treated with Formula (2) for at least 8 days are included.

FIG. 40 shows a comparison of progression-free survival (PFS) in CLL patients exhibiting the 17p deletion and treated with the BTK inhibitor of Formula (10) (ibrutinib) or the BTK inhibitor of Formula (2). The ibrutinib data is taken from Byrd, et al., N. Engl. J. Med. 2013, 369, 32-42.

FIG. 41 shows a comparison of number of patients at risk in CLL patients exhibiting the 17p deletion and treated with the BTK inhibitor of Formula (10) (ibrutinib) or the BTK inhibitor of Formula (2). The ibrutinib data is taken from Byrd, et al., N. Engl. J. Med. 2013, 369, 32-42. CLL patients treated with Formula (2) for at least 8 days are included.

FIG. 42 shows improved BTK target occupancy of Formula (2) at lower dosage versus Formula (10) (ibrutinib) in relapsed/refractory CLL patients.

FIG. 43 shows the % change in myeloid-derived suppressor cell (MDSC) (monocytic) level over 28 days versus % ALC change at Cycle 1, day 28 (C1D28) with trendlines.

FIG. 44 shows the % change in MDSC (monocytic) level over 28 days versus % ALC change at Cycle 2, day 28 (C2D28) with trendlines.

FIG. 45 shows the % change in natural killer (NK) cell level over 28 days versus % ALC change at Cycle 1, day 28 (C2D28) with trendlines.

FIG. 46 shows the % change in NK cell level over 28 days versus % ALC change at Cycle 2, day 28 (C2D28) with trendlines.

FIG. 47 compares the % change in MDSC (monocytic) level and % change in NK cell level over 28 days versus % ALC change with the % change in level of CD4⁺ T cells, CD8⁺ T cells, CD4⁺/CD8⁺ T cell ratio, NK-T cells, PD-1⁺CD4⁺ T cells, and PD-1⁺CD8⁺ T cells, also versus % ALC change, at Cycle 1 day 28 (C1D28). Trendlines are shown for % change in MDSC (monocytic) level and % change in NK cell level.

FIG. 48 compares the % change in MDSC (monocytic) level and % change in NK cell level over 28 days versus % ALC change with the % change in level of CD4⁺ T cells, CD8⁺ T cells, CD4⁺/CD8⁺ T cell ratio, NK-T cells, PD-1⁺CD4⁺ T cells, and PD-1⁺CD8⁺ T cells, also versus % ALC change, at Cycle 2 day 28 (C2D28). Trendlines are shown for % change in MDSC (monocytic) level and % change in NK cell level.

FIG. 49 shows updated the results of the clinical study of Formula (2) (labeled “BTK inhibitor”) in CLL, which are shown in comparison to the results reported for ibrutinib in FIG. 1A of Byrd, et al., N. Engl. J. Med. 2013, 369, 32-42. The results show that the BTK inhibitor of Formula (2) causes a much smaller relative increase and much faster decrease in absolute lymphocyte count (ALC) relative to the BTK inhibitor of Formula (10) (ibrutinib). The sum of the product of greatest diameters (SPD) also decreases more rapidly during treatment with the BTK inhibitor than with the BTK inhibitor of Formula (10) (ibrutinib).

FIG. 50 shows improved BTK target occupancy of Formula (2) at lower dosage versus ibrutinib in relapsed/refractory CLL patients, and includes BID dosing results.

FIG. 51 illustrates PFS for patients with 17p deletion.

FIG. 52 illustrates PFS across relapsed/refractory patients with 17p deletion and with 11q deletion and no 17p deletion.

FIG. 53 illustrates PFS for patients with 11q deletion and no 17p deletion.

FIG. 54 illustrates updated SPD results from the clinical study of Formula (2) in relapsed/refractory CLL patients.

FIG. 55 illustrates that treatment of CLL patients with Formula (2) resulted in increased apoptosis.

FIG. 56 illustrates a decrease in CXCL12 levels observed in patients treated with Formula (2).

FIG. 57 illustrates a decrease in CCL2 levels observed in patients treated with Formula (2).

FIG. 58 illustrates representative photomicrographs and comparison of maximal thrombus size in laser injured arterioles of VWF HA1 mutant mice infused with human platelets in the absence or presence of various BTK inhibitors. Representative photomicrographs are given as a comparison of maximal thrombus size in laser-injured arterioles (1 μM concentrations shown).

FIG. 59 illustrates a quantitative comparison obtained by in vivo analysis of early thrombus dynamics in a humanized mouse laser injury model using three BTK inhibitors at a concentration 1 M.

FIG. 60 illustrates the effect of the tested BTK inhibitors on thrombus formation. The conditions used were N=4, 3 mice per drug; anti-clotting active pharmaceutical ingredients <2000 μM². In studies with Formula (10) (ibrutinib), 48% MCL bleeding events were observed with 560 mg QD and 63% CLL bleeding events were observed with 420 mg QD, where bleeding event is defined as subdural hematoma, ecchymoses, gastrointestinal (GI) bleeding, or hematuria.

FIG. 61 illustrates the effect of the concentration of the tested BTK inhibitors on thrombus formation.

FIG. 62 illustrates the results of GPVI platelet aggregation studies of Formula (2) (IC50=1.15 μM) and Formula (10) (ibrutinib, IC50=0.13 μM).

FIG. 63 illustrates the results of GPVI platelet aggregation studies of Formula (2) and Formula (10) (ibrutinib).

FIG. 64 illustrates the dose-effect curves obtained for the tested RI-1 cell line (Activated B Cell-Diffuse Large B Cell Lymphoma (ABC-DLBCL) cell line obtained from ATCC) using combined dosing of the BTK inhibitor of Formula (2) (“BTK1”) and the IRAK4 inhibitor of Formula (46) (“AS”). The y-axis (“Effect”) is given in units of Fa (fraction affected) and the x-axis (“Dose”) is given in linear units of μM.

FIG. 65 illustrates the synergy observed in the RI-1 cell line when the BTK inhibitor of Formula (2) and the IRAK4 inhibitor of Formula (46) (“AS”) are combined.

FIG. 66 illustrates the dose-effect curves obtained for the tested RI-1 cell line using combined dosing of the BTK inhibitor of ibrutinib (Formula (10)) (“IBR”) and the IRAK4 inhibitor of Formula (46) (“AS”). The y-axis (“Effect”) is given in units of Fa (fraction affected) and the x-axis (“Dose”) is given in linear units of μM.

FIG. 67 illustrates the synergy observed in the RI-1 cell line when the BTK inhibitor of ibrutinib (Formula (10)) and the IRAK4 inhibitor of Formula (46) (“AS”) are combined.

FIG. 68 illustrates the dose-effect curves obtained for the tested RI-1 cell line using combined dosing of the BTK inhibitor of Formula (21) (“ONO” or “ONO-4059”) and the IRAK4 inhibitor of Formula (46) (“AS”). The y-axis (“Effect”) is given in units of Fa (fraction affected) and the x-axis (“Dose”) is given in linear units of μM.

FIG. 69 illustrates the synergy observed in the RI-1 cell line when the BTK inhibitor of Formula (21) and the IRAK4 inhibitor of Formula (46) (“AS”) are combined.

FIG. 70 illustrates the dose-effect curves obtained for the tested TMD-8 cell line (Activated B Cell-Diffuse Large B Cell Lymphoma (ABC-DLBCL) cell line obtained from Tokyo Medical and Dental University) using combined dosing of the BTK inhibitor of Formula (2) (“BTKI”) and the IRAK4 inhibitor of Formula (46) (“AS”). The y-axis (“Effect”) is given in units of Fa (fraction affected) and the x-axis (“Dose”) is given in linear units of μM.

FIG. 71 illustrates the synergy observed in the TMD-8 cell line when the BTK inhibitor of Formula (2) and the IRAK4 inhibitor of Formula (46) (“AS”) are combined.

FIG. 72 illustrates the dose-effect curves obtained for the TMD-8 cell line using combined dosing of the BTK inhibitor of ibrutinib (Formula (10)) (“IBR”) and the IRAK4 inhibitor of Formula (46) (“AS”). The y-axis (“Effect”) is given in units of Fa (fraction affected) and the x-axis (“Dose”) is given in linear units of μM.

FIG. 73 illustrates the synergy observed in the TMD-8 cell line when the BTK inhibitor of ibrutinib (Formula (10)) and the IRAK4 inhibitor of Formula (46) (“AS”) are combined.

FIG. 74 illustrates the dose-effect curves obtained for the TMD-8 cell line using combined dosing of the BTK inhibitor of Formula (21) (“ONO” or “ONO-4059”) and the IRAK4 inhibitor of Formula (46) (“AS”). The y-axis (“Effect”) is given in units of Fa (fraction affected) and the x-axis (“Dose”) is given in linear units of μM.

FIG. 75 illustrates the synergy observed in the TMD-8 cell line when the BTK inhibitor of Formula (21) and the IRAK4 inhibitor of Formula (46) (“AS”) are combined.

FIG. 76 illustrates the dose-effect curves obtained for the tested Mino cell line (mantle cell lymphoma cell line obtained from ATCC) using combined dosing of the BTK inhibitor of Formula (2) (“BTK1”) and the IRAK4 inhibitor of Formula (46) (“AS”). The y-axis (“Effect”) is given in units of Fa (fraction affected) and the x-axis (“Dose”) is given in linear units of μM.

FIG. 77 illustrates the synergy observed in the Mino cell line when the BTK inhibitor of Formula (2) and the IRAK4 inhibitor of Formula (46) (“AS”) are combined.

FIG. 78 illustrates the dose-effect curves obtained for the Mino cell line using combined dosing of the BTK inhibitor of ibrutinib (Formula (10)) (“IBR”) and the IRAK4 inhibitor of Formula (46) (“AS”). The y-axis (“Effect”) is given in units of Fa (fraction affected) and the x-axis (“Dose”) is given in linear units of μM.

FIG. 79 illustrates the synergy observed in the Mino cell line when the BTK inhibitor of ibrutinib (Formula (10)) and the IRAK4 inhibitor of Formula (46) (“AS”) are combined.

FIG. 80 illustrates the dose-effect curves obtained for the Mino cell line using combined dosing of the BTK inhibitor of Formula (21) (“ONO” or “ONO-4059”) and the IRAK4 inhibitor of Formula (46) (“AS”). The y-axis (“Effect”) is given in units of Fa (fraction affected) and the x-axis (“Dose”) is given in linear units of μM.

FIG. 81 illustrates the synergy observed in the Mino cell line when the BTK inhibitor of Formula (21) and the IRAK4 inhibitor of Formula (46) (“AS”) are combined.

FIG. 82 illustrates the dose-effect curves obtained for the tested RI-1 cell line using combined dosing of the BTK inhibitor of Formula (2) (“BTK1”) and the IRAK1/4 inhibitor of Formula (45) (“IR1.4”). The y-axis (“Effect”) is given in units of Fa (fraction affected) and the x-axis (“Dose”) is given in linear units of μM.

FIG. 83 illustrates the synergy observed in the RI-1 cell line when the BTK inhibitor of Formula (2) and the IRAK1/4 inhibitor of Formula (45) (“IR1.4”) are combined.

FIG. 84 illustrates the dose-effect curves obtained for the tested RI-1 cell line using combined dosing of the BTK inhibitor of ibrutinib (Formula (10)) (“IBR”) and the IRAK1/4 inhibitor of Formula (45) (“IR1.4”). The y-axis (“Effect”) is given in units of Fa (fraction affected) and the x-axis (“Dose”) is given in linear units of μM.

FIG. 85 illustrates the synergy observed in the RI-1 cell line when the BTK inhibitor of ibrutinib (Formula (10)) and the IRAK1/4 inhibitor of Formula (45) (“IR1.4”) are combined.

FIG. 86 illustrates the dose-effect curves obtained for the tested RI-1 cell line using combined dosing of the BTK inhibitor of Formula (21) (“ONO” or “ONO-4059”) and the IRAK1/4 inhibitor of Formula (45) (“IR1.4”). The y-axis (“Effect”) is given in units of Fa (fraction affected) and the x-axis (“Dose”) is given in linear units of μM.

FIG. 87 illustrates the synergy observed in the RI-1 cell line when the BTK inhibitor of Formula (21) and the IRAK1/4 inhibitor of Formula (45) (“IR1.4”) are combined.

FIG. 88 illustrates the dose-effect curves obtained for the tested Mino cell line (mantle cell lymphoma cell line obtained from ATCC) using combined dosing of the BTK inhibitor of Formula (2) (“BTK1”) and the IRAK1/4 inhibitor of Formula (45) (“IR1.4”). The y-axis (“Effect”) is given in units of Fa (fraction affected) and the x-axis (“Dose”) is given in linear units of M.

FIG. 89 illustrates the synergy observed in the Mino cell line when the BTK inhibitor of Formula (2) and the IRAK1/4 inhibitor of Formula (45) (“IR1.4”) are combined.

FIG. 90 illustrates the dose-effect curves obtained for the Mino cell line using combined dosing of the BTK inhibitor of ibrutinib (Formula (10)) (“IBR”) and the IRAK1/4 inhibitor of Formula (45) (“IR1.4”). The y-axis (“Effect”) is given in units of Fa (fraction affected) and the x-axis (“Dose”) is given in linear units of μM.

FIG. 91 illustrates the synergy observed in the Mino cell line when the BTK inhibitor of ibrutinib (Formula (10)) and the IRAK1/4 inhibitor of Formula (45) (“IR1.4”) are combined.

FIG. 92 illustrates the dose-effect curves obtained for the Mino cell line using combined dosing of the BTK inhibitor of Formula (21) (“ONO” or “ONO-4059”) and the IRAK1/4 inhibitor of Formula (45) (“IR1.4”). The y-axis (“Effect”) is given in units of Fa (fraction affected) and the x-axis (“Dose”) is given in linear units of μM.

FIG. 93 illustrates the synergy observed in the Mino cell line when the BTK inhibitor of Formula (21) and the IRAK1/4 inhibitor of Formula (45) (“IR1.4”) are combined.

FIG. 94 illustrates the dose-effect curves obtained for the tested SU-DHL-6 cell line (follicular lymphoma cell line obtained from ATCC) using combined dosing of the BTK inhibitor of Formula (2) (“BTK1”) and the IRAK1/4 inhibitor of Formula (45) (“IR1.4”). The y-axis (“Effect”) is given in units of Fa (fraction affected) and the x-axis (“Dose”) is given in linear units of M.

FIG. 95 illustrates the synergy observed in the SU-DHL-6 cell line when the BTK inhibitor of Formula (2) and the IRAK1/4 inhibitor of Formula (45) (“IR1.4”) are combined.

FIG. 96 illustrates the dose-effect curves obtained for the SU-DHL-6 cell line using combined dosing of the BTK inhibitor of ibrutinib (Formula (10)) (“IBR”) and the IRAK1/4 inhibitor of Formula (45) (“IR1.4”). The y-axis (“Effect”) is given in units of Fa (fraction affected) and the x-axis (“Dose”) is given in linear units of μM.

FIG. 97 illustrates the synergy observed in the SU-DHL-6 cell line when the BTK inhibitor of ibrutinib (Formula (10)) and the IRAK1/4 inhibitor of Formula (45) (“IR1.4”) are combined.

FIG. 98 illustrates the dose-effect curves obtained for the SU-DHL-6 cell line using combined dosing of the BTK inhibitor of Formula (21) (“ONO” or “ONO-4059”) and the IRAK1/4 inhibitor of Formula (45) (“IR1.4”). The y-axis (“Effect”) is given in units of Fa (fraction affected) and the x-axis (“Dose”) is given in linear units of μM.

FIG. 99 illustrates the synergy observed in the SU-DHL-6 cell line when the BTK inhibitor of Formula (21) and the IRAK1/4 inhibitor of Formula (45) (“IR1.4”) are combined.

### BRIEF DESCRIPTION OF THE SEQUENCE LISTING

SEQ ID NO:1 is the heavy chain amino acid sequence of the anti-CD20 monoclonal antibody rituximab.

SEQ ID NO:2 is the light chain amino acid sequence of the anti-CD20 monoclonal antibody rituximab.

SEQ ID NO:3 is the heavy chain amino acid sequence of the anti-CD20 monoclonal antibody obinutuzumab.

SEQ ID NO:4 is the light chain amino acid sequence of the anti-CD20 monoclonal antibody obinutuzumab.

SEQ ID NO:5 is the variable heavy chain amino acid sequence of the anti-CD20 monoclonal antibody ofatumumab.

SEQ ID NO:6 is the variable light chain amino acid sequence of the anti-CD20 monoclonal antibody ofatumumab.

SEQ ID NO:7 is the Fab fragment heavy chain amino acid sequence of the anti-CD20 monoclonal antibody ofatumumab.

SEQ ID NO:8 is the Fab fragment light chain amino acid sequence of the anti-CD20 monoclonal antibody ofatumumab.

SEQ ID NO:9 is the heavy chain amino acid sequence of the anti-CD20 monoclonal antibody veltuzumab.

SEQ ID NO:10 is the light chain amino acid sequence of the anti-CD20 monoclonal antibody veltuzumab.

SEQ ID NO:11 is the heavy chain amino acid sequence of the anti-CD20 monoclonal antibody tositumomab.

SEQ ID NO:12 is the light chain amino acid sequence of the anti-CD20 monoclonal antibody tositumomab.

SEQ ID NO:13 is the heavy chain amino acid sequence of the anti-CD20 monoclonal antibody ibritumomab.

SEQ ID NO:14 is the light chain amino acid sequence of the anti-CD20 monoclonal antibody ibritumomab.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, at least one IRAK4 inhibitor and at least one BTK inhibitor) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

As used herein, the term “warhead” or “warhead group” refers to a functional group present on a compound of the present invention wherein that functional group is capable of covalently binding to an amino acid residue present in the binding pocket of the target protein (such as cysteine, lysine, histidine, or other residues capable of being covalently modified), thereby irreversibly inhibiting the protein.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, heptyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers. The present chemical entities, pharmaceutical compositions and methods are meant to include all possible tautomers, including mixtures thereof.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Co-Administration of Compounds

An aspect of the invention is a composition, such as a pharmaceutical composition, comprising a combination of a BTK inhibitor, and/an IRAK4 inhibitor.

Another aspect is a kit containing any a BTK inhibitor and an IRAK4 inhibitor, wherein each of the inhibitors is formulated into a separate pharmaceutical composition, and wherein said separate pharmaceutical compositions are formulated for co-administration.

Another aspect of the invention is a method of treating a disease or condition in a subject, in particular a hyperproliferative disorder such as leukemia, lymphoma or a solid tumor cancer in a subject, comprising co-administering to the subject in need thereof a therapeutically effective amount of a combination of a BTK inhibitor, and an IRAK4 inhibitor. In an embodiment, the foregoing method exhibits synergistic effects that may result in greater efficacy, less side effects, the use of less active pharmaceutical ingredient to achieve a given clinical result, or other synergistic effects. A combination of a BTK inhibitor and an IRAK4 inhibitor is a preferred embodiment. The pharmaceutical composition comprising the combination, and the kit, are both for use in treating such disease or condition.

In a preferred embodiment, the solid tumor cancer is selected from the group consisting of breast, lung, colorectal, thyroid, bone sarcoma, and stomach cancers.

In a preferred embodiment, the leukemia is selected from the group consisting of acute myelogenous leukemia (AML), chronic myelogenous leukemia (CML), acute lymphoblastic leukemia (ALL), B cell chronic lymphocytic leukemia (B-CLL), and chronic lymphoid leukemia (CLL).

In a preferred embodiment, the lymphoma is selected from the group consisting of Burkitt's lymphoma, mantle cell lymphoma, follicular lymphoma, indolent B-cell non-Hodgkin's lymphoma, histiocytic lymphoma, activated B-cell like diffuse large B cell lymphoma (DLBCL-ABC), germinal center B-cell like diffuse large B cell lymphoma (DLBCL-GCB), and diffuse large B cell lymphoma (DLBCL).

In an embodiment, the BTK inhibitor is in the form of a pharmaceutically acceptable salt, solvate, hydrate, complex, derivative, prodrug (such as an ester or phosphate ester), or cocrystal.

In an embodiment, the IRAK4 inhibitor is in the form of a pharmaceutically acceptable salt, solvate, hydrate, complex, derivative, prodrug (such as an ester or phosphate ester), or cocrystal.

In an embodiment, the IRAK4 inhibitor is administered to the subject before administration of the BTK inhibitor.

In an embodiment, the IRAK4 inhibitor is administered concurrently with the administration of the BTK inhibitor.

In an embodiment, the IRAK4 inhibitor is administered to the subject after administration of the BTK inhibitor.

In a preferred embodiment, the subject is a mammal, such as a human. In an embodiment, the subject is a human. In an embodiment, the subject is a companion animal. In an embodiment, the subject is a canine, feline, or equine.

### BTK Inhibitors

The BTK inhibitor may be any BTK inhibitor known in the art. In particular, it is one of the BTK inhibitors described in more detail in the following paragraphs.

In an embodiment, the BTK inhibitor is a compound of Formula (1):

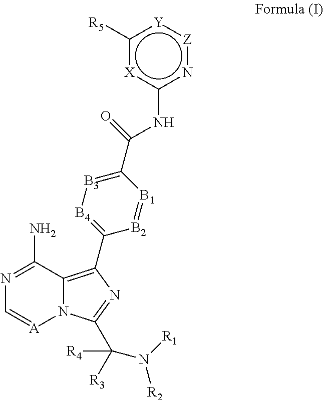

or a pharmaceutically acceptable salt, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
B₂ is N or C(R₈);
B₃ is N or C(R₉);
B₄ is N or C(R₁₀);
R₁ is R₁₁C(═O), R₁₂S(═O), R₁₃S(═O)₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₄)alkyl, (C₁₋₃)alkoxy, (C₃₋₆)cycloalkyl, any alkyl group of which is optionally substituted with one or more halogen; or R₅ is (C₆₋₁₀)aryl or (C₂₋₆)heterocycloalkyl;
R₆ is H or (C₁₋₃)alkyl; or
R₅ and R₆ together may form a (C₃₋₇)cycloalkenyl or (C₂₋₆)heterocycloalkenyl, each optionally substituted with (C₁₋₃)alkyl or one or more halogens;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₉)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy; R₁₁ is independently selected from the group consisting of (C₁₋₆)alkyl, (C₂₋₆)alkenyl and (C₂₋₆)alkynyl, where each alkyl, alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or R₁₁ is (C₁₋₃)alkyl-C(O)—S—(C₁₋₃)alkyl; or
R₁₁ is (C₁₋₅)heteroaryl optionally substituted with one or more substituents selected from the group consisting of halogen or cyano;
R₁₂ and R₁₃ are independently selected from the group consisting of (C₂₋₆)alkenyl or (C₂₋₆)alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; or a (C₁₋₅)heteroaryl optionally substituted with one or more substituents selected from the group consisting of halogen and cyano; and
R₁₄ is independently selected from the group consisting of halogen, cyano, (C₂₋₆)alkenyl and (C₂₋₆)alkynyl, both optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, (C₁₋₅)heteroaryl and (C₃₋₇)heterocycloalkyl; with the proviso that:
0 to 2 atoms of X, Y, Z can simultaneously be a heteroatom;
when one atom selected from X, Y is O or S, then Z is a bond and the other atom selected from X, Y cannot be O or S;
when Z is C or N then Y is C(R₆) or N and X is C or N;
0 to 2 atoms of B₁, B₂, B₃ and B₄ are N;
with the terms used having the following meanings:
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined. (C₁₋₂)alkoxy groups are preferred;
(C₂₋₆)alkenyl means a branched or unbranched alkenyl group having 2-6 carbon atoms, such as ethenyl, 2-butenyl, and n-pentenyl, (C₂₋₄)alkenyl groups being most preferred;
(C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl. (C₂₋₄)alkynyl groups are preferred; (C₃₋₆)cycloalkyl means a cycloalkyl group having 3-6 carbon atoms, being cyclopropyl, cyclobutyl, cyclopentyl or cyclohexyl;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; also preferred are piperidine, morpholine, pyrrolidine and piperazine; with the most preferred (C₂₋₆)heterocycloalkyl being pyrrolidine; the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S. Preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S; the (C₁₋₅)heteroaryl may optionally be substituted; preferred (C₁₋₅)heteroaryl groups are tetrazolyl, imidazolyl, thiadiazolyl, pyridyl, pyrimidyl, triazinyl, thienyl or furyl, a more preferred (C₁₋₅)heteroaryl is pyrimidyl;
[(C₁₋₄)alkyl]amino means an amino group, monosubstituted with an alkyl group containing 1-4 carbon atoms having the same meaning as previously defined; preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; preferred di[(C₁₋₄)alkyl]amino group is dimethylamino;
   halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; preferred (C₂₋₆)heterocycloalkenyl groups are oxycyclohexenyl and azacyclohexenyl group.
In the above definitions with multifunctional groups, the attachment point is at the last group.
When, in the definition of a substituent, it is indicated that “all of the alkyl groups” of said substituent are optionally substituted, this also includes the alkyl moiety of an alkoxy group.
A circle in a ring of Formula (1) indicates that the ring is aromatic.
Depending on the ring formed, the nitrogen, if present in X or Y, may carry a hydrogen.

In a preferred embodiment, the BTK inhibitor is a compound of Formula (1) or a pharmaceutically acceptable salt thereof, wherein:
X is CH or S;
Y is C(R₆);
Z is CH or bond;
A is CH;
B₁ is N or C(R₇);
B₂ is N or C(R);
B₃ is N or CH;
B₄ is N or CH;
R₁ is R₁₁C(═O),
R₂ is (C₁₋₃)alkyl;
R₃ is (C₁₋₃)alkyl; or
R₂ and R₃ form, together with the N and C atom they are attached to, a (C₃₋₇)heterocycloalkyl ring selected from the group consisting of azetidinyl, pyrrolidinyl, piperidinyl, and morpholinyl, optionally substituted with one or more fluorine, hydroxyl, (C₁₋₃)alkyl, or (C₁₋₃)alkoxy;
R₄ is H;
R₅ is H, halogen, cyano, (C₁₋₄)alkyl, (C₁₋₃)alkoxy, (C₃₋₆)cycloalkyl, or an alkyl group which is optionally substituted with one or more halogen;
R₆ is H or (C₁₋₃)alkyl;
R₇ is H, halogen or (C₁₋₃)alkoxy;
R₈ is H or (C₁₋₃)alkyl; or
R₇ and R₈ form, together with the carbon atom they are attached to a (C₆₋₁₀)aryl or (C₁₋₉)heteroaryl;
R₅ and R₆ together may form a (C₃₋₇)cycloalkenyl or (C₂₋₆)heterocycloalkenyl, each optionally substituted with (C₁₋₃)alkyl or one or more halogen;
R₁₁ is independently selected from the group consisting of (C₂₋₆)alkenyl and (C₂₋₆)alkynyl, where each alkenyl or alkynyl is optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl and (C₃₋₇)heterocycloalkyl; with the proviso that 0 to 2 atoms of B₁, B₂, B₃ and B₄ are N.

In an embodiment of Formula (1), B₁ is C(R₇); B₂ is C(R₈); B₃ is C(R₉); B₄ is C(R₁₀); R₇, R₉, and R₁₀ are each H; and R₈ is hydrogen or methyl.

In an embodiment of Formula (1), the ring containing X, Y and Z is selected from the group consisting of pyridyl, pyrimidyl, pyridazyl, triazinyl, thiazolyl, oxazolyl and isoxazolyl.

In an embodiment of Formula (1), the ring containing X, Y and Z is selected from the group consisting of pyridyl, pyrimidyl and pyridazyl.

In an embodiment of Formula (1), the ring containing X, Y and Z is selected from the group consisting of pyridyl and pyrimidyl.

In an embodiment of Formula (1), the ring containing X, Y and Z is pyridyl.

In an embodiment of Formula (1), R₅ is selected from the group consisting of hydrogen, fluorine, methyl, methoxy and trifluoromethyl.

In an embodiment of Formula (1), R₅ is hydrogen.

In an embodiment of Formula (1), R₂ and R₃ together form a heterocycloalkyl ring selected from the group consisting of azetidinyl, pyrrolidinyl, piperidinyl, homopiperidinyl and morpholinyl, optionally substituted with one or more of fluoro, hydroxyl, (C₁₋₃)alkyl and (C₁₋₃)alkoxy.

In an embodiment of Formula (1), R₂ and R₃ together form a heterocycloalkyl ring selected from the group consisting of azetidinyl, pyrrolidinyl and piperidinyl.

In an embodiment of Formula (1), R₂ and R₃ together form a pyrrolidinyl ring.

In an embodiment of Formula (1), R₁ is independently selected from the group consisting of (C₁₋₆)alkyl, (C₂₋₆)alkenyl or (C₂₋₆)alkynyl, each optionally substituted with one or more substituents selected from the group consisting of hydroxyl, (C₁₋₄)alkyl, (C₃₋₇)cycloalkyl, [(C₁₋₄)alkyl]amino, di[(C₁₋₄)alkyl] amino, (C₁₋₃)alkoxy, (C₃₋₇)cycloalkoxy, (C₆₋₁₀)aryl and (C₃₋₇)heterocycloalkyl.

In an embodiment of Formula (1), B₁, B₂, B₃ and B₄ are CH; X is N; Y and Z are CH; R₅ is CH₃; A is N; R₂, R₃ and R₄ are H; and R₁ is CO—CH₃.

In an embodiment of Formula (1), B₁, B₂, B₃ and B₄ are CH; X and Y are N; Z is CH; R₅ is CH₃; A is N; R₂, R₃ and R₄ are H; and R₁ is CO—CH₃.

In an embodiment of Formula (1), B₁, B₂, B₃ and B₄ are CH; X and Y are N; Z is CH; R₅ is CH₃; A is CH; R₂ and R₃ together form a piperidinyl ring; R₄ is H; and R₁ is CO-ethenyl.

In an embodiment of Formula (1), B₁, B₂, B₃ and B₄ are CH; X, Y and Z are CH; R₅ is H; A is CH; R₂ and R₃ together form a pyrrolidinyl ring; R₄ is H; and R₁ is CO-propynyl.

In an embodiment of Formula (1), B₁, B₂, B₃ and B₄ are CH; X, Y and Z are CH; R₅ is CH₃; A is CH; R₂ and R₃ together form a piperidinyl ring; R₄ is H; and R₁ is CO-propynyl.

In an embodiment of Formula (1), B₁, B₂, B₃ and B₄ are CH; X and Y are N; Z is CH; R₅ is H; A is CH; R₂ and R₃ together form a morpholinyl ring; R₄ is H; and R₁ is CO-ethenyl.

In an embodiment of Formula (1), B₁, B₂, B₃ and B₄ are CH; X and Y are N; Z is CH; R₅ is CH₃; A is CH; R₂ and R₃ together form a morpholinyl ring; R₄ is H; and R₁ is CO-propynyl.

In a preferred embodiment, the BTK inhibitor is a compound of Formula (2):

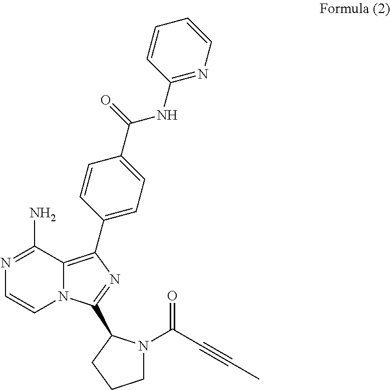 [SMILES: CC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is (S)-4-(8-amino-3-(1-(but-2-ynoyl)pyrrolidin-2-yl)imidazo[1,5-a]pyrazin-1-yl)-N-(pyridin-2-yl)benzamide or pharmaceutically acceptable salt, solvate, hydrate, cocrystal, or prodrug thereof.

In a preferred embodiment, the BTK inhibitor is a compound of Formula (3):

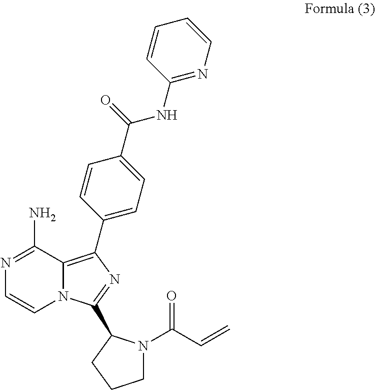 [SMILES: C=CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (4):

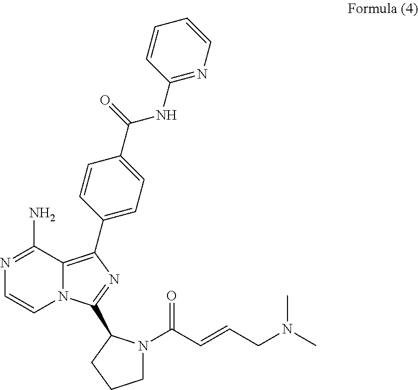 [SMILES: CN(C)C/C=C/C(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (5):

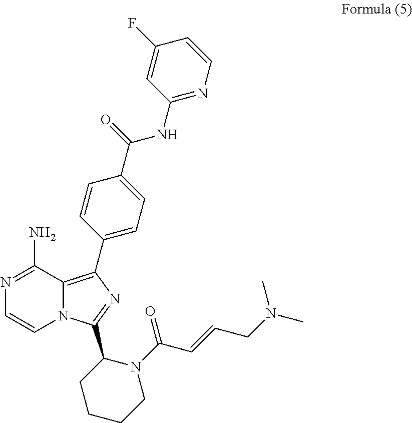 [SMILES: CN(C)C/C=C/C(=O)N1CCCC[C@H]1c1nc(-c2ccc(C(=O)Nc3cc(F)ccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (6):

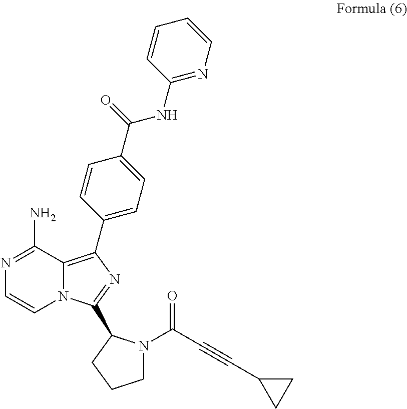 [SMILES: Nc1nccn2c([C@@H]3CCCN3C(=O)C#CC3CC3)nc(-c3ccc(C(=O)Nc4ccccn4)cc3)c12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In a preferred embodiment, the BTK inhibitor is a compound of Formula (7):

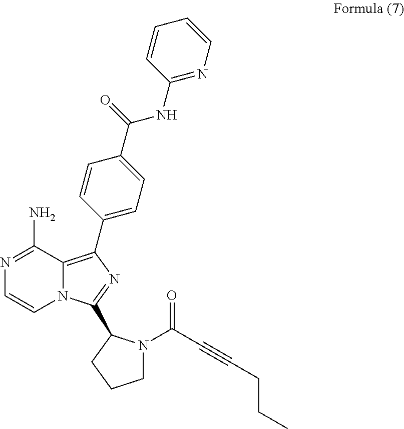 [SMILES: CCCC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. The preparation of this compound is described in International Patent Application Publication No. WO 2013/010868 and U.S. Patent Application Publication No. US 2014/0155385 A1, the disclosures of which are incorporated herein by reference.

In other embodiments, the BTK inhibitors include, but are not limited to, those compounds described in International Patent Application Publication No. WO 2013/010868 and U.S. Patent Application Publication No. US 2014/0155385 A1, the disclosures of each of which are specifically incorporated by reference herein.

In an embodiment, the BTK inhibitor is a compound of Formula (8):

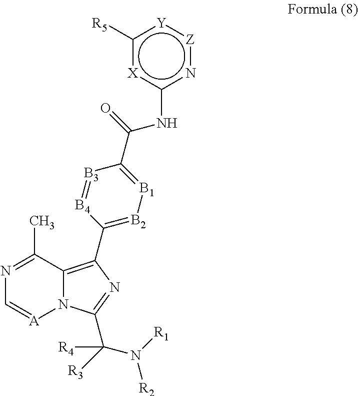

or a pharmaceutically acceptable salt, solvate, hydrate, cocrystal, or prodrug thereof, wherein:
X is CH, N, O or S;
Y is C(R₆), N, O or S;
Z is CH, N or bond;
A is CH or N;
B₁ is N or C(R₇);
B₂ is N or C(R₈);
B₃ is N or C(R₉);
B₄ is N or C(R₁₀);
R₁ is R₁₁C(O), R₁₂S(O), R₁₃SO₂ or (C₁₋₆)alkyl optionally substituted with R₁₄;
R₂ is H, (C₁₋₃)alkyl or (C₃₋₇)cycloalkyl;
R₃ is H, (C₁₋₆)alkyl or (C₃₋₇)cycloalkyl); or
R₂ and R₃ form, together with the N and C atom they are attached to, a (C₃₋₇)heterocycloalkyl optionally substituted with one or more fluorine, hydroxyl, (C₁₋₃)alkyl, (C₁₋₃)alkoxy or oxo;
R₄ is H or (C₁₋₃)alkyl;
R₅ is H, halogen, cyano, (C₁₋₄)alkyl, (C₁₋₃)alkoxy, (C₃₋₆)cycloalkyl; all alkyl groups of R5 are optionally substituted with one or more halogen; or R₅ is (C₆₋₁₀)aryl or (C₂₋₆)heterocycloalkyl;
R₆ is H or (C₁₋₃)alkyl; or R₅ and R6 together may form a (C₃₋₇)cycloalkenyl, or (C₂₋₆)heterocycloalkenyl; each optionally substituted with (C₁₋₃)alkyl, or one or more halogen;
R₇ is H, halogen, CF₃, (C₁₋₃)alkyl or (C₁₋₃)alkoxy;
R₈ is H, halogen, CF₃, (C₁₋₃)alkyl or (C₁₋₃)alkoxy; or
R₇ and R₈ together with the carbon atoms they are attached to, form (C₆₋₁₀)aryl or (C₁₋₅)heteroaryl;
R₉ is H, halogen, (C₁₋₃)alkyl or (C₁₋₃)alkoxy;
R₁₀ is H, halogen, (C₁₋₃)alkyl or (C₁₋₃)alkoxy;
R₁₁ is independently selected from a group consisting of (C₁₋₆)alkyl, (C₂₋₆)alkenyl and (C₂₋₆)alkynyl each alkyl, alkenyl or alkynyl optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl or (C₃₋₇)heterocycloalkyl, or
R₁₁ is (C₁₋₃)alkyl-C(O)—S—(C₁₋₃)alkyl; or
R₁₁ is (C₁₋₅)heteroaryl optionally substituted with one or more groups selected from halogen or cyano.
R₁₂ and R₁₃ are independently selected from a group consisting of (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, or (C₃₋₇)heterocycloalkyl; or
(C₁₋₅)heteroaryl optionally substituted with one or more groups selected from halogen or cyano;
R₁₄ is independently selected from a group consisting of halogen, cyano or (C₂₋₆)alkenyl or (C₂₋₆)alkynyl both optionally substituted with one or more groups selected from hydroxyl, (C₁₋₄)alkyl, (C₃₋₇)cycloalkyl, [(C₁₋₄)alkyl]amino, di[(C₁₋₄)alkyl]amino, (C₁₋₃)alkoxy, (C₃₋₇)cycloalkoxy, (C₆₋₁₀)aryl, (C₁₋₅)heteroaryl or (C₃₋₇)heterocycloalkyl;
   with the proviso that
   0 to 2 atoms of X, Y, Z can simultaneously be a heteroatom;
   when one atom selected from X, Y is O or S, then Z is a bond and the other atom selected from X, Y cannot be O or S;
   when Z is C or N then Y is C(R₆) or N and X is C or N;
   0 to 2 atoms of B₁, B₂, B₃ and B₄ are N;
      with the terms used having the following meanings:
(C₁₋₃)alkyl means a branched or unbranched alkyl group having 1-3 carbon atoms, being methyl, ethyl, propyl or isopropyl;
(C₁₋₄)alkyl means a branched or unbranched alkyl group having 1-4 carbon atoms, being methyl, ethyl, propyl, isopropyl, butyl, isobutyl, sec-butyl and tert-butyl, (C₁₋₃)alkyl groups being preferred;
(C₁₋₆)alkyl means a branched or unbranched alkyl group having 1-6 carbon atoms, for example methyl, ethyl, propyl, isopropyl, butyl, tert-butyl, n-pentyl and n-hexyl. (C₁₋₅)alkyl groups are preferred, (C₁₋₄)alkyl being most preferred;
(C₁₋₂)alkoxy means an alkoxy group having 1-2 carbon atoms, the alkyl moiety having the same meaning as previously defined;
(C₁₋₃)alkoxy means an alkoxy group having 1-3 carbon atoms, the alkyl moiety having the same meaning as previously defined, with (C₁₋₂)alkoxy groups preferred;
(C₂₋₄)alkenyl means a branched or unbranched alkenyl group having 2-4 carbon atoms, such as ethenyl, 2-propenyl, isobutenyl or 2-butenyl;
(C₂₋₆)alkenyl means a branched or unbranched alkenyl group having 2-6 carbon atoms, such as ethenyl, 2-butenyl, and n-pentenyl, with (C₂₋₄)alkenyl groups preferred, and (C₂₋₃)alkenyl groups even more preferred;
(C₂₋₄)alkynyl means a branched or unbranched alkynyl group having 2-4 carbon atoms, such as ethynyl, 2-propynyl or 2-butynyl;
(C₂₋₆)alkynyl means a branched or unbranched alkynyl group having 2-6 carbon atoms, such as ethynyl, propynyl, n-butynyl, n-pentynyl, isopentynyl, isohexynyl or n-hexynyl, with (C₂₋₄)alkynyl groups preferred, and (C₂₋₃)alkynyl groups more preferred;
(C₃₋₇)cycloalkyl means a cycloalkyl group having 3-7 carbon atoms, being cyclopropyl, cyclobutyl, cyclopentyl, cyclohexyl or cycloheptyl;
(C₂₋₆)heterocycloalkyl means a heterocycloalkyl group having 2-6 carbon atoms, preferably 3-5 carbon atoms, and one or two heteroatoms selected from N, O and/or S, which may be attached via a heteroatom if feasible, or a carbon atom; preferred heteroatoms are N or O; preferred groups are piperidine, morpholine, pyrrolidine and piperazine; a most preferred (C₂₋₆)heterocycloalkyl is pyrrolidine; and the heterocycloalkyl group may be attached via a heteroatom if feasible;
(C₃₋₇)heterocycloalkyl means a heterocycloalkyl group having 3-7 carbon atoms, preferably 3-5 carbon atoms, and one or two heteroatoms selected from N, O and/or S; preferred heteroatoms are N or O; preferred (C₃₋₇) heterocycloalkyl groups are azetidinyl, pyrrolidinyl, piperidinyl, homopiperidinyl or morpholinyl; more preferred (C₃₋₇)heterocycloalkyl groups are piperidine, morpholine and pyrrolidine; even more preferred are piperidine and pyrrolidine; and the heterocycloalkyl group may be attached via a heteroatom if feasible;
(C₃₋₇)cycloalkoxy means a cycloalkyl group having 3-7 carbon atoms, with the same meaning as previously defined, attached via a ring carbon atom to an exocyclic oxygen atom;
(C₆₋₁₀)aryl means an aromatic hydrocarbon group having 6-10 carbon atoms, such as phenyl, naphthyl, tetrahydronaphthyl or indenyl; the preferred (C₆₋₁₀)aryl group is phenyl;
(C₁₋₅)heteroaryl means a substituted or unsubstituted aromatic group having 1-5 carbon atoms and 1-4 heteroatoms selected from N, O and/or S, wherein the (C₁₋₅)heteroaryl may optionally be substituted.; preferred (C₁₋₅)heteroaryl groups are tetrazolyl, imidazolyl, thiadiazolyl, pyridyl, pyrimidyl, triazinyl, thienyl or furyl, and the more preferred (C₁₋₅)heteroaryl is pyrimidyl;
[(C₁₋₄)alkyl]amino means an amino group, monosubstituted with an alkyl group containing 1-4 carbon atoms having the same meaning as previously defined; the preferred [(C₁₋₄)alkyl]amino group is methylamino;
di[(C₁₋₄)alkyl]amino means an amino group, disubstituted with alkyl group(s), each containing 1-4 carbon atoms and having the same meaning as previously defined; the preferred di[(C₁₋₄)alkyl]amino group is dimethylamino;
halogen means fluorine, chlorine, bromine or iodine;
(C₁₋₃)alkyl-C(O)—S—(C₁₋₃)alkyl means an alkyl-carbonyl-thio-alkyl group, each of the alkyl groups having 1 to 3 carbon atoms with the same meaning as previously defined;
(C₃₋₇)cycloalkenyl means a cycloalkenyl group having 3-7 carbon atoms, preferably 5-7 carbon atoms; preferred (C₃₋₇)cycloalkenyl groups are cyclopentenyl or cyclohexenyl; and cyclohexenyl groups are most preferred;
(C₂₋₆)heterocycloalkenyl means a heterocycloalkenyl group having 2-6 carbon atoms, preferably 3-5 carbon atoms; and 1 heteroatom selected from N, O and/or S; the preferred (C₂₋₆)heterocycloalkenyl groups are oxycyclohexenyl and azacyclohexenyl groups.
In the above definitions with multifunctional groups, the attachment point is at the last group.
When, in the definition of a substituent, it is indicated that “all of the alkyl groups” of said substituent are optionally substituted, this also includes the alkyl moiety of an alkoxy group.
A circle in a ring of Formula (8) indicates that the ring is aromatic.
Depending on the ring formed, the nitrogen, if present in X or Y, may carry a hydrogen.

In a preferred embodiment, the invention relates to a compound according to Formula (8) wherein B₁ is C(R₇); B₂ is C(R₈); B₃ is C(R₉) and B₄ is C(R₁₀).

In other embodiments, the BTK inhibitors include, but are not limited to, those compounds described in International Patent Application Publication No. WO 2013/010869 and U.S. Patent Application Publication No. US 2014/0155406 A1, the disclosures of each of which are specifically incorporated by reference herein.

In an embodiment, the BTK inhibitor is a compound of Formula (9):

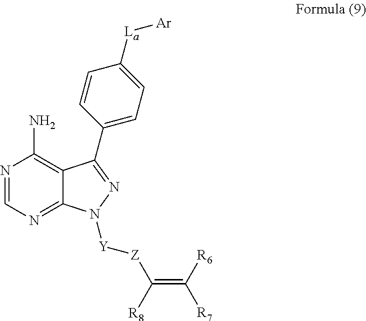

or a pharmaceutically acceptable salt, solvate, hydrate, cocrystal, or prodrug thereof, wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each independently H; or R⁷ and R⁸ taken together form a bond;
R⁶ is H; and
R is H or (C₁₋₆)alkyl.

In a preferred embodiment, the BTK inhibitor is ibrutinib or a pharmaceutically acceptable salt, solvate, hydrate, cocrystal, or prodrug thereof. In a preferred embodiment, the BTK inhibitor is (R)-1-(3-(4-amino-3-(4-phenoxyphenyl)-1H-pyrazolo[3,4-d]pyrimidin-1-yl)piperidin-1-yl)prop-2-en-1-one. In a preferred embodiment, the BTK inhibitor is 1-[(3R)-3-[4-amino-3-(4-phenoxyphenyl)-1H-pyrazolo[3,4-d]pyrimidin-1-yl]piperidin-1-yl]prop-2-en-1-one. In a preferred exemplary embodiment, the BTK inhibitor is (S)-1-(3-(4-amino-3-(4-phenoxyphenyl)-1H-pyrazolo[3,4-d]pyrimidin-1-yl)piperidin-1-yl)prop-2-en-1-one. In a preferred embodiment, the BTK inhibitor has the structure of Formula (10):

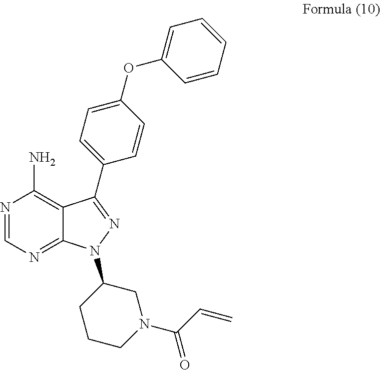 [SMILES: C=CC(=O)N1CCC[C@@H](n2nc(-c3ccc(Oc4ccccc4)cc3)c3c(N)ncnc32)C1]

or an enantiomer thereof, or a pharmaceutically acceptable salt, solvate, hydrate, cocrystal, or prodrug thereof.

In an exemplary embodiment, the BTK inhibitor is a compound of Formula (11):

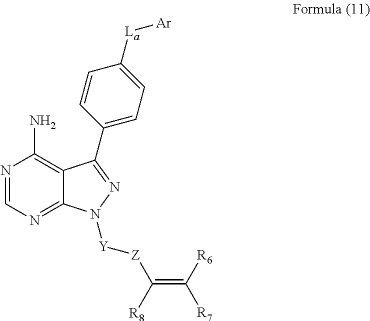

or a pharmaceutically acceptable salt, solvate, hydrate, cocrystal, or prodrug thereof,
wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (12):

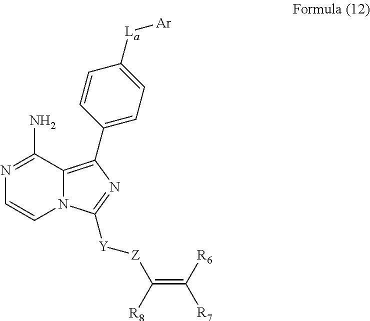

or a pharmaceutically acceptable salt, solvate, hydrate, cocrystal, or prodrug thereof,
wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound of Formula (13):

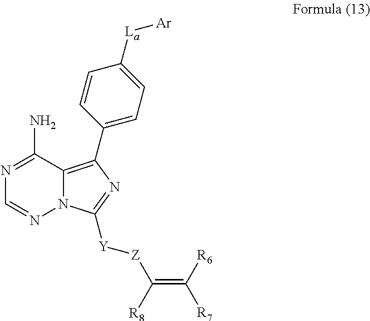

or a pharmaceutically acceptable salt, solvate, hydrate, cocrystal, or prodrug thereof,
wherein:
Lₐ is CH₂, O, NH or S;
Ar is a substituted or unsubstituted aryl, or a substituted or unsubstituted heteroaryl;
Y is an optionally substituted group selected from the group consisting of alkyl, heteroalkyl, cycloalkyl, heterocycloalkyl, aryl and heteroaryl;
Z is C(═O), OC(═O), NRC(═O), C(═S), S(═O)ₓ, OS(═O)ₓ or NRS(═O)ₓ, where x is 1 or 2;
R⁷ and R⁸ are each H; or R⁷ and R⁸ taken together form a bond;
R⁶ is H; and
R is H or (C₁₋₆)alkyl.

In an embodiment, the BTK inhibitor is a compound disclosed in U.S. Pat. No. 7,459,554, the disclosure of which is specifically incorporated herein by reference. In an embodiment, the BTK inhibitor is a compound of Formula (14):

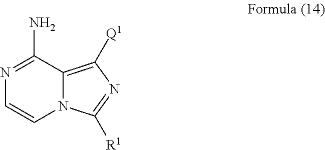

or a pharmaceutically acceptable salt, solvate, hydrate, cocrystal, or prodrug thereof, wherein:
Q¹ is aryl¹, heteroaryl¹, cycloalkyl, heterocyclyl, cycloalkenyl, or heterocycloalkenyl, any of which is optionally substituted by one to five independent G¹ substituents;
R¹ is alkyl, cycloalkyl, bicycloalkyl, aryl, heteroaryl, aralkyl, heteroaralkyl, heterocyclyl, or heterobicycloalkyl, any of which is optionally substituted by one or more independent G¹¹ substituents;
G¹ and G⁴¹ are each independently halo, oxo, —CF₃, —OCF₃, —OR², —NR²R³(R^{3a})ⱼ₁, —C(O)R², —CO₂R², —CONR²R³, —NO₂, —CN, —S(O)ⱼ₁R², —SO₂NR²R³, NR²(C═O)R³, NR²(C═O)OR³, NR²(C═O)NR²R³, NR²S(O)ⱼ₁R³, —(C═S)OR², —(C═O)SR², —NR²(C═NR³)NR^{2a}R^{3a}, —NR²(C═NR³)OR^{2a}, —NR²(C═NR³)SR^{3a}, —O(C═O)OR², —O(C═O)NR²R³, —O(C═O)SR², —S(C═O)OR², —S(C═O)NR²R³, (C₁₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₁₋₁₀)alkyl, heterocyclyl-(C₂₋₁₀)alkenyl, or heterocyclyl-(C₂₋₁₀)alkynyl, any of which is optionally substituted with one or more independent halo, oxo, —CF₃, —OCF₃, —OR²²², —NR²²²R³³³(R³³³a)ⱼ₁ₐ, —C(O)R²²², —CO₂R²²², —CONR²²²R³³³, —NO₂, —CN, —S(O)ⱼ₁ₐR²²², —SO₂NR²²²R³³³, NR²²²(C═O)R³³³, NR²²²(C═O)OR³³³, NR²²²(C═O)NR²²²R³³³, NR²²²S(O)ⱼ₁ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents; or —(X¹)ₙ—(Y¹)ₘ—R⁴; or aryl-(C₁₋₁₀)alkyl, aryl-(C₂₋₁₀)alkenyl, or aryl-(C₂₋₁₀) alkynyl, any of which is optionally substituted with one or more independent halo, —CF₃, —OCF₃, —OR²²², —NR²²²R³³³(R^{333a})ⱼ₂ₐ, —C(O)R²²², —CO₂R²²², —CONR²²²R³³³, —NO₂, —CN, —S(O)ⱼ₂ₐR²²², —SO₂NR²²²R³³³, NR²²²(C═O)R³³³, NR²²²(C═O)OR³³³, NR²²²(C═O)NR²²²R³³³, NR²²²S(O)ⱼ₂ₐR³³³, —(C═S)OR²²², —(C═O)SR²²², —NR²²²(C═NR³³³)NR^{222a}R^{333a}, —NR²²²(C═NR³³³)OR^{222a}, —NR²²²(C═NR³³³)SR^{333a}, —O(C═O)OR²²², —O(C═O)NR²²²R³³³, —O(C═O)SR²²², —S(C═O)OR²²², or —S(C═O)NR²²²R³³³ substituents; or hetaryl-(C₁₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR²²², —NR²²², R³³³(R^{333a})ⱼ₃ₐ, —C(O)R²²², —CO₂R²²², —CONR²²²R³³³, —NO₂, —CN, —S(O)ⱼ₃ₐR²²², —SO₂NR²²²R³³³, NR²²²(C═O)R³³³, NR²²²(C═O)OR³³³, NR²²²(C═O)NR²²²R³³³, NR²²²S(O)ⱼ₃ₐR³³³, —(C═S)OR²²², —(C═O)SR²²², —NR²²²(C═NR³³³)NR²²²aR³³³a, —NR²²²(C═NR³³³)OR^{222a}, —NR²²²(C═NR³³³)SR³³³a, —O(C═O)OR²²², —O(C═O)NR²²²R³³³, —O(C═O)SR²²², —S(C═O)OR²²², or —S(C═O)NR²²²R³³³ substituents;
G¹¹ is halo, oxo, —CF₃, —OCF₃, —OR²¹, —NR²¹R³¹(R^{3a1})ⱼ₄, —C(O)R²¹, —CO₂R²¹, —CONR²¹R³¹, —NO₂, —CN, —S(O)ⱼ₄R²¹, —SO₂NR²¹R³¹, NR²¹(C═O)R³¹, NR²¹(C═O)OR³¹, NR²¹(C═O)NR²¹R³¹, NR²¹S(O)₄R³¹, —(C═S)OR²¹, —(C═O)SR²¹, —²¹(C═NR³¹)NR^{2a1}R^{3a1}, —NR²¹(C═NR³¹)OR^{2a1}, —NR²¹(C═NR³¹)SR^{3a1}, —O(C═O)OR²¹, —O(C═O)NR²¹R³¹, —O(C═O)SR²¹, —S(C═O)OR²¹, —S(C═O)NR²¹R³¹, —P(O)OR²¹OR³¹, (C₁₋₁₀)alkyl, (C₂₋₁₀)alkenyl, (C₂₋₁₀)alkynyl, (C₁₋₁₀) alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀) alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀) alkyl, cyclo(C₃₋₈)alkyl(C₂₋₁₀)alkenyl, cyclo(C₃₋₈)alkenyl(C₂₋₁₀)alkenyl, cyclo(C₃₋₈) alkyl(C₂₋₁₀) alkynyl, cyclo(C₃₋₈)alkenyl(C₂₋₁₀)alkynyl, heterocyclyl-(C₁₋₁₀)alkyl, heterocyclyl-(C₂₋₁₀) alkenyl, or heterocyclyl-(C₂₋₁₀)alkynyl, any of which is optionally substituted with one or more independent halo, oxo, —CF₃, —OCF₃, —OR²²²¹, —NR²²²¹R³³³¹(R^{333a1})ⱼ₄ₐ, —C(O)R²²²¹, —CO₂R²²²¹, —CONR²²²¹R³³³¹, —NO₂, —CN, —S(O)ⱼ₄ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₄ₐR³³³¹, —(C═S)OR²²²¹, —(C═O)SR²²²¹, —NR²²²¹(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R³³³¹ substituents; or aryl-(C₀₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR²²²¹, —NR²²²¹R³³³¹(R^{333a1})ⱼ₅ₐ, —C(O)R²²²¹, —CO₂R²²²¹, —CONR²²²¹R³³³¹, —NO₂, —CN, —S(O)ⱼ₅ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₅ₐR³³³¹, —(C═S)OR²²²¹, —(C═O)SR²²²¹, —NR²²²¹(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═)OR²²²¹, —P(O)OR²²²¹R³³³¹, or —S(C═O)NR²²²¹R³³³¹ substituents; or hetaryl-(C₀₋₁₀) alkyl, hetaryl-(C₂₋₁₀)alkenyl, or hetaryl-(C₂₋₁₀)alkynyl, any of which is optionally substituted with one or more independent halo, —CF₃, —OCF₃, —OR²²²¹, —NR²²²¹R³³³¹(R^{333a1})ⱼ₆ₐ, —C(O)R²²²¹, —CO₂R²²²¹, —CONR²²²¹R³³³¹, —NO₂, —CN, —S(O)ⱼ₆ₐR²²²¹, —SO₂NR²²²¹R³³³¹, NR²²²¹(C═O)R³³³¹, NR²²²¹(C═O)OR³³³¹, NR²²²¹(C═O)NR²²²¹R³³³¹, NR²²²¹S(O)ⱼ₆ₐR³³³¹, —(C═S)OR²²²¹, —(C═O)SR²²²¹, —NR²²²¹(C═NR³³³¹)NR^{222a1}R^{333a1}, —NR²²²¹(C═NR³³³¹)OR^{222a1}, —NR²²²¹(C═NR³³³¹)SR^{333a1}, —O(C═O)OR²²²¹, —O(C═O)NR²²²¹R³³³¹, —O(C═O)SR²²²¹, —S(C═O)OR²²²¹, —P(O)OR²²²¹OR³³³¹, or —S(C═O)NR²²²¹R³³³¹ substituents; or G¹¹ is taken together with the carbon to which it is attached to form a double bond which is substituted with R⁵ and G¹¹¹;
R², R^{2a}, R³, R^{3a}, R²²², R²²²a, R³³³, R^{333a}, R²¹, R^{2a1}, R³¹, R^{3a1}, R²²²¹, R^{222a1}, R³³³¹, and R^{333a1} are each independently equal to (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted by one or more G¹¹¹ substituents; or aryl-(C₁₋₁₀)alkyl, aryl-(C₂₋₁₀)alkenyl, or aryl-(C₂₋₁₀)alkynyl, hetaryl-(C₀₋₁₀)alkyl, hetaryl-(C₂₋₁₀)alkenyl, or hetaryl-(C₂₋₁₀)alkynyl, any of which is optionally substituted by one or more G¹¹¹ substituents; or in the case of —NR²R³(R^{3a})ⱼ₁ or —NR²²²R³³³(R^{333a})ⱼ₁ₐ or —NR²²²R³³³(R^{333a})ⱼ₂ₐ or —NR²²²¹R³³³¹(R^{333a1})ⱼ₃ₐ or —NR²²²¹R³³³¹(R^{333a1})ⱼ₄ₐ or —NR²²²¹R³³³¹(R^{333a1})ⱼ₅ₐ or —NR²²²¹R³³³¹(R^{333a1})ⱼ₆ₐ, R² and R³ or R²²² and R³³³3 or R²²²¹ and R³³³¹ taken together with the nitrogen atom to which they are attached form a 3-10 membered saturated ring, unsaturated ring, heterocyclic saturated ring, or heterocyclic unsaturated ring, wherein said ring is optionally substituted by one or more G¹¹¹ substituents;
X¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C.ident.C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂— —OC(O)N(R⁷)—, —N(R⁷)C(O)N(R⁷)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁷)—, —N(R⁷)S(O)₂N(R⁷)—, —C(O)N(R⁷)C(O)—, —S(O)N(R⁷)C(O)—, —S(O)₂N(R)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁷)—, —N(R⁷)SO₂N(R⁷)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁷)—, —CH(R⁷)N(C(O)R⁷)—, —CH(R⁷)N(SO₂R⁷)—, —CH(R⁷)O—, —CH(R⁷)S—, —CH(R⁷)N(R⁷)—, —CH(R⁷)N(C(O)R⁷)—, —CH(R⁷)N(C(O)OR⁷)—, —CH(R⁷)N(SO₂R⁷)—, —CH(R⁷)C(═NOR⁷)—, —CH(R⁷)C(O)—, —CH(R)CH(OR⁷)—, —CH(R⁷)C(O)N(R⁷)—, —CH(R⁷)N(R⁷)C(O)—, —CH(R⁷)N(R⁷)S(O)—, —CH(R⁷)N(R⁷)S(O)₂—, —CH(R⁷)OC(O)N(R⁷)—, —CH(R⁷)N(R⁷)C(O)N(R⁷)—, —CH(R⁷)NR⁷C(O)O—, —CH(R⁷)S(O)N(R⁷)—, —CH(R⁷)S(O)₂N(R⁷)—, —CH(R⁷)N(C(O)R⁷) S(O)—, —CH(R⁷)N(C(O)R⁷)S(O)—, —CH(R⁷)N(R⁷)S(O)N(R⁷)—, —CH(R⁷)N(R⁷)S(O)₂N(R⁷)—, —CH(R⁷)C(O)N(R⁷)C(O)—, —CH(R⁷)S(O)N(R⁷)C(O)—, —CH(R⁷) S(O)₂N(R⁷)C(O)—, —CH(R⁷)O S(O)N(R⁷)—, —CH(R⁷)O S(O)₂N(R⁷)—, —CH(R⁷)N(R⁷)S(O)O—, —CH(R⁷)N(R⁷)S(O)₂O—, —CH(R⁷)N(R⁷)S(O)C(O)—, —CH(R⁷)N(R⁷)S(O)₂C(O)—, —CH(R⁷)SON(C(O)R⁷)—, —CH(R⁷)SO₂N(C(O)R⁷)—, —CH(R⁷)N(R⁷)SON(R⁷)—, —CH(R⁷)N(R⁷)SO₂N(R⁷)—, —CH(R⁷)C(O)O—, —CH(R⁷)N(R⁷)P(OR⁸)O—, —CH(R⁷)N(R⁷)P(OR⁸)—, —CH(R⁷)N(R⁷)P(O)(OR⁸)O—, —CH(R⁷)N(R⁷)P(O)(OR⁸)—, —CH(R⁷)N(C(O)R⁷)P(OR⁸)O—, —CH(R⁷)N(C(O)R⁷)P(OR⁸)—, —CH(R⁷)N(C(O)R⁷)P(O)(OR⁸)O—, or —CH(R⁷)N(C(O)R⁷)P(OR⁸)—;
or X¹ and Y¹ are each independently represented by one of the following structural formulas:

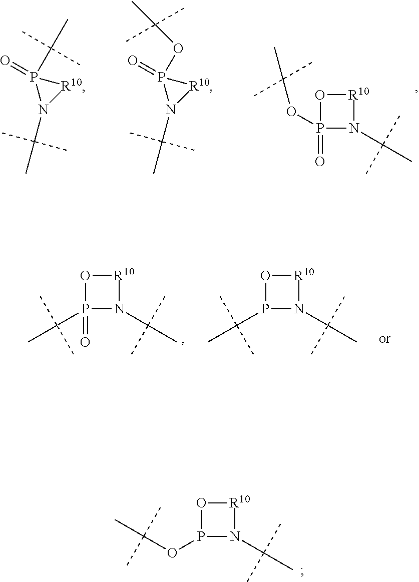

R¹⁰, taken together with the phosphinamide or phosphonamide, is a 5-, 6-, or 7-membered aryl, heteroaryl or heterocyclyl ring system;
R⁵, R⁶, and G¹¹¹ are each independently a (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or aryl-(C₀₋₁₀)alkyl, aryl-(C₂₋₁₀)alkenyl, or aryl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)₅ₐR⁸⁷, —(C═S)OR⁷⁷, —(C═O)SR⁷⁷, —NR⁷⁷(C═NR⁸⁷)NR⁷⁸R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷R⁸⁷, or —S(C═O)NR⁷⁷R⁸⁷ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, NR⁷⁷(C═O)R⁸⁷, NR⁷⁷(C═O)OR⁸⁷, NR⁷⁷(C═O)NR⁷⁸R⁸⁷, NR⁷⁷S(O)ⱼ₅ₐR⁸⁷, —(C═S)OR⁷⁷, —(C═O)SR⁷⁷, —NR⁷⁷(C═NR⁸⁷)N78R⁸⁸, —NR⁷⁷(C═NR⁸⁷)OR⁷⁸, —NR⁷⁷(C═NR⁸⁷)SR⁷⁸, —O(C═O)OR⁷⁷, —O(C═O)NR⁷⁷R⁸⁷, —O(C═O)SR⁷⁷, —S(C═O)OR⁷⁷, —P(O)OR⁷⁷OR⁸⁷, or —S(C═O)NR⁷⁷R⁸⁷ substituents; or R⁵ with R⁶ taken together with the respective carbon atom to which they are attached, form a 3-10 membered saturated or unsaturated ring, wherein said ring is optionally substituted with R⁶⁹; or R⁵ with R⁶ taken together with the respective carbon atom to which they are attached, form a 3-10 membered saturated or unsaturated heterocyclic ring, wherein said ring is optionally substituted with R⁶⁹;
R⁷ and R⁸ are each independently H, acyl, alkyl, alkenyl, aryl, heteroaryl, heterocyclyl or cycloalkyl, any of which is optionally substituted by one or more G¹¹¹ substituents;
R⁴ is H, alkyl, alkenyl, alkynyl, aryl, heteroaryl, cycloalkyl, heterocyclyl, cycloalkenyl, or heterocycloalkenyl, any of which is optionally substituted by one or more G⁴¹ substituents;
R⁶⁹ is equal to halo, —OR⁷⁸, —SH, —NR⁷⁸R⁸⁸, —CO₂R⁷⁸, —CONR⁷⁸R⁸⁸, —NO₂, —CN, —S(O)ⱼ₈R⁷⁸, —SO₂NR⁷⁸R⁸⁸, (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxy(C₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃-g)alkenyl(C₂₋₁₀)alkenyl, cyclo(C₃-g)alkyl(C₂₋₁₀)alkynyl, cyclo(C₃₋₈)alkenyl(C₂₋₁₀)alkynyl, heterocyclyl-(C₀₋₁₀)alkyl, heterocyclyl-(C₂₋₁₀)alkenyl, or heterocyclyl-(C₂₋₁₀)alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or hetaryl-(C₀₋₁₀)alkyl, hetaryl-(C₂₋₁₀)alkenyl, or hetaryl-(C₂₋₁₀)alkynyl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸, —SO₂NR⁷⁷⁸R⁸⁸⁸, or —NR⁷⁷⁸R⁸⁸⁸ substituents; or mono(C₁₋₆alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —OR⁷⁷⁸, (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CONR⁷⁷⁸R⁸⁸⁸ SO₂NR⁷⁷⁸R⁸⁸⁸, or —NR⁷⁷⁸R⁸⁸⁸ substituents; or in the case of —NR⁷⁸R⁸⁸, R⁷⁸ and R⁸⁸ taken together with the nitrogen atom to which they are attached form a 3-10 membered saturated ring, unsaturated ring, heterocyclic saturated ring, or heterocyclic unsaturated ring, wherein said ring is optionally substituted with one or more independent halo, cyano, hydroxy, nitro, (C₁₋₁₀)alkoxy, —SO₂NR⁷⁷⁸R⁸⁸⁸, or —NR⁷⁷⁸R⁸⁸⁸ substituents;
R⁷⁷, R⁷⁸, R⁸⁷, R⁸⁸, R⁷⁷⁸, and R⁸⁸⁸ are each independently (C₀₋₁₀)alkyl, (C₂₋₁₀)alkenyl, (C₂₋₁₀)alkynyl, (C₁₋₁₀)alkoxy(C₁₋₁₀)alkyl, (C₁₋₁₀)alkoxyC₂₋₁₀)alkenyl, (C₁₋₁₀)alkoxy(C₂₋₁₀)alkynyl, (C₁₋₁₀)alkylthio(C₁₋₁₀)alkyl, (C₁₋₁₀)alkylthio(C₂₋₁₀)alkenyl, (C₁₋₁₀)alkylthio(C₂₋₁₀)alkynyl, cyclo(C₃₋₈)alkyl, cyclo(C₃₋₈)alkenyl, cyclo(C₃₋₈)alkyl(C₁₋₁₀)alkyl, cyclo(C₃₋₈)alkenyl(C₁₋₁₀)alkyl, cyclo(C₃₋₈)alkyl(C₂₋₁₀)alkenyl, cyclo(C₃₋₈)alkenyl(C₂₋₁₀)alkenyl, cyclo(C₃₋₈)alkyl(C₂₋₁₀)alkynyl, cyclo(C₃₋₈)alkenyl(C₂₋₁₀)alkynyl, heterocyclyl-(C₁₋₁₀)alkyl, heterocyclyl-(C₂₋₁₀)alkenyl, heterocyclyl-(C₂₋₁₀)alkynyl, (C₁₋₁₀)alkylcarbonyl, (C₂₋₁₀)alkenylcarbonyl, (C₂₋₁₀)alkynylcarbonyl, (C₁₋₁₀)alkoxycarbonyl, (C₁₋₁₀)alkoxycarbonyl(C₁₋₁₀)alkyl, mono(C₁₋₆)alkylaminocarbonyl, di(C₁₋₆)alkylaminocarbonyl, mono(aryl)aminocarbonyl, di(aryl)aminocarbonyl, or (C₁₋₁₀)alkyl(aryl)aminocarbonyl, any of which is optionally substituted with one or more independent halo, cyano, hydroxy, nitro, (C₁₋₁₀)alkoxy, —SO₂N((C₀₋₄)alkyl)((C₀₋₄)alkyl), or —N((C₀₋₄)alkyl)((C₀₋₄)alkyl) substituents; or aryl-(C₀₋₁₀)alkyl, aryl-(C₂₋₁₀)alkenyl, or aryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₁₋₁₀)alkyl), —SO₂N((C₀₋₄)alkyl)((C₀₋₄)alkyl), or —N((C₀₋₄)alkyl)((C₀₋₄)alkyl) substituents; or hetaryl-(C₁₋₁₀)alkyl, hetaryl-(C₂₋₁₀)alkenyl, or hetaryl-(C₂₋₁₀)alkynyl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((C₀₋₄)alkyl)((C₀₋₄)alkyl), or —N((C₀₋₄)alkyl)((C₀₋₄)alkyl) substituents; or mono((C₁₋₆)alkyl)amino(C₁₋₆)alkyl, di((C₁₋₆)alkyl)amino(C₁₋₆)alkyl, mono(aryl)amino(C₁₋₆)alkyl, di(aryl)amino(C₁₋₆)alkyl, or —N((C₁₋₆)alkyl)-(C₁₋₆)alkyl-aryl, any of which is optionally substituted with one or more independent halo, cyano, nitro, —O((C₀₋₄)alkyl), (C₁₋₁₀)alkyl, (C₂₋₁₀)alkenyl, (C₂₋₁₀)alkynyl, halo(C₁₋₁₀)alkyl, halo(C₂₋₁₀)alkenyl, halo(C₂₋₁₀)alkynyl, —COOH, (C₁₋₄)alkoxycarbonyl, —CON((C₀₋₄)alkyl)((C₀₋₄)alkyl), —SO₂N((C₀₋₄)alkyl)((C₀₋₄)alkyl), or —N((C₀₋₄)alkyl)((C₀₋₄)alkyl) substituents; and
n, m, j, j11a, j2a, j3a, j4, j4a, j5a, j6a, j7, and j8 are each independently equal to 0, 1, or 2.

In an embodiment, the BTK inhibitor is a compound selected from the structures disclosed in U.S. Pat. Nos. 8,450,335 and 8,609,679, and U.S. Patent Application Publication Nos. 2010/0029610 A1, 2012/0077832 A1, 2013/0065879 A1, 2013/0072469 A1, and 2013/0165462 A1, the disclosures of which are incorporated by reference herein. In an embodiment, the BTK inhibitor is a compound of Formula (15) or Formula (16):

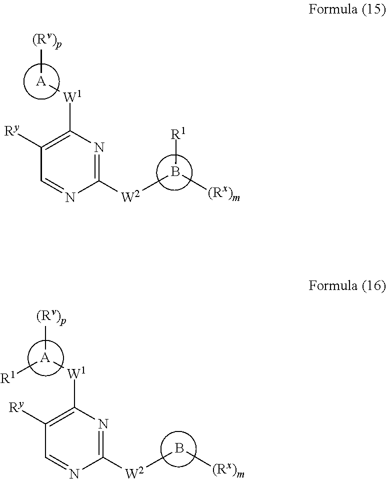

or a pharmaceutically acceptable salt, solvate, hydrate, cocrystal, or prodrug thereof, wherein:
Ring A is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is a warhead group;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²⁻, —N(R²)C(O)—, —C(O)N(R²)—, —N(R²)SO₂—, —SO₂N(R²)—, —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered saturated, partially unsaturated, or aromatic fused ring, or:
R² and R^{y} are taken together with their intervening atoms to form an optionally substituted 4-7 membered partially unsaturated or aromatic fused ring;
m and p are independently 0-4; and
R^{x} and R^{v} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein q is 1-4; or:
R^{x} and R¹ when concurrently present on Ring B are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form an optionally substituted 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic.

In an embodiment, the BTK inhibitor is a compound of Formula (15) or Formula (16), wherein:
Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is -L-Y, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—,
Y is hydrogen, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 groups independently selected from -Q-Z, oxo, NO₂, halogen, CN, or C₁₋₆ aliphatic, wherein:
Q is a covalent bond or a bivalent C₁₋₆ saturated or unsaturated, straight or branched, hydrocarbon chain, wherein one or two methylene units of Q are optionally and independently replaced by —NR, —S—, —O—, —C(O)—, —SO—, or —SO₂—; and
Z is hydrogen or C₁₋₆ aliphatic optionally substituted with oxo, halogen, or CN;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, an optionally substituted 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR²⁻, —N(R²)C(O)—, —C(O)N(R²)—, —N(R²)SO₂₋, —SO₂N(R²)—, —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered partially unsaturated or aromatic fused ring; or
R² and R^{y} are taken together with their intervening atoms to form a 4-6 membered saturated, partially unsaturated, or aromatic fused ring;
m and p are independently 0-4; and
R^{x} and R^{v} are independently selected from —R, halogen, —OR, —O(CH₂)_{q}OR, —CN, —NO₂, —SO₂R, —SO₂N(R)₂, —SOR, —C(O)R, —CO₂R, —C(O)N(R)₂, —NRC(O)R, —NRC(O)NR₂, —NRSO₂R, or —N(R)₂, wherein R is independently selected from the group consisting of hydrogen, cycloalkyl, alkenyl, cycloalkenyl, alkynyl, aryl, heteroaryl, and heterocycly; or:
R^{x} and R¹ when concurrently present on Ring B are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C₁₋₆ aliphatic; or
R^{v} and R¹ when concurrently present on Ring A are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, —CN, or C1-6 aliphatic.

As defined generally above, Ring A is selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, an optionally substituted 4-7 membered saturated or partially unsaturated heterocyclic ring having 1-3 heteroatoms independently selected from nitrogen, oxygen, or sulfur, an optionally substituted 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In preferred embodiments, Ring A is an optionally substituted phenyl group. In some embodiments, Ring A is an optionally substituted naphthyl ring or an optionally substituted bicyclic 8-10 membered heteroaryl ring having 1-4 heteroatoms independently selected from nitrogen, oxygen, or sulfur. In certain other embodiments, Ring A is an optionally substituted 3-7 membered carbocyclic ring. In yet other embodiments, Ring A is an optionally substituted 4-7 membered heterocyclic ring having 1-3 heteroatoms independently selected from nitrogen, oxygen, or sulfur. In preferred embodiments, Ring B is an optionally substituted phenyl group.

In certain embodiments, Ring A in Formula (15) or Formula (16) is substituted as defined herein. In some embodiments, Ring A is substituted with one, two, or three groups independently selected from halogen, R^{∘}, or —(CH₂)₀₋₄OR^{∘}, or —O(CH₂)O₄R^{∘}, wherein each R^{∘} is independently selected from the group consisting of cycloalkyl, alkenyl, cycloalkenyl, alkynyl, aryl, heteroaryl, and heterocyclyl. Exemplary substituents on Ring A include Br, I, Cl, methyl, —CF₃, —C≡CH, —OCH₂phenyl, —OCH₂(fluorophenyl), or —OCH₂pyridyl.

In a preferred embodiment, the BTK inhibitor is CC-292 (also known as AVL-292), or a pharmaceutically acceptable salt, solvate, hydrate, cocrystal, or prodrug thereof, preferably a hydrochloride salt or a besylate salt thereof. In a preferred embodiment, the BTK inhibitor is a compound of Formula (17):

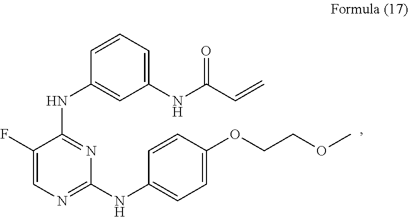 [SMILES: C=CC(=O)Nc1cccc(Nc2nc(Nc3ccc(OCCOC)cc3)ncc2F)c1]

which is N-(3-((5-fluoro-2-((4-(2-methoxyethoxy)phenyl)amino)pyrimidin-4-yl)amino)phenyl)acrylamide, or a pharmaceutically acceptable salt, solvate, hydrate, cocrystal, or prodrug thereof, or in an exemplary embodiment is a hydrochloride salt or a besylate salt thereof. The preparation of this compound is described in U.S. Patent Application Publication No. 2010/0029610 A1 at Example 20, the disclosure of which is incorporated by reference herein. The preparation of the besylate salt of this compound is described in U.S. Patent Application Publication No. 2012/0077832 A1, the disclosure of which is incorporated by reference herein. In an embodiment, the BTK inhibitor is a compound selected from the structures disclosed in U.S. Patent Application Publication No. 2010/0029610 A1 or No. 2012/0077832 A1, the disclosures of which are incorporated by reference herein.

In a preferred embodiment, the BTK inhibitor is N-(3-((5-fluoro-2-((4-(2-methoxyethoxy)phenyl)amino)pyrimidin-4-yl)amino)phenyl)acrylamide or a pharmaceutically acceptable salt, solvate, hydrate, cocrystal, or prodrug thereof, or a hydrochloride salt thereof. The preparation of this compound is described in U.S. Patent Application Publication Nos. 2010/0029610 A1 and 2012/0077832 A1, the disclosure of which is incorporated by reference herein.

In a preferred embodiment, the BTK inhibitor is (N-(3-(5-fluoro-2-(4-(2-methoxyethoxy)phenylamino)pyrimidin-4-ylamino)phenyl)acrylamide), or a pharmaceutically acceptable salt, solvate, hydrate, cocrystal, or prodrug thereof, or preferably a besylate salt thereof. The preparation of this compound is described in U.S. Patent Application Publication No. 2010/0029610 A1 at Example 20, the disclosure of which is incorporated by reference herein. The preparation of its besylate salt is described in U.S. Patent Application Publication No. 2012/0077832 A1, the disclosure of which is incorporated by reference herein.

In an embodiment, the BTK inhibitor is a compound of Formula (18):

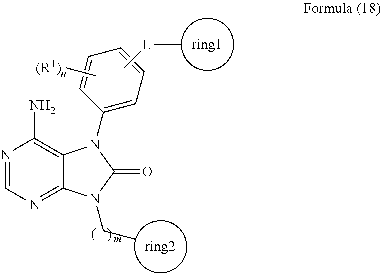

or a pharmaceutically acceptable salt, hydrate, solvate, cocrystal, or prodrug thereof, wherein
L represents (1) —O—, (2) —S—, (3) —SO—, (4) —SO₂— (5) —NH—, (6) —C(O)—, (7) —CH₂O—, (8) —O—CH₂—, (9) —CH₂—, or (10) —CH(OH)—;
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1 represents a 4- to 7-membered cyclic group, which may be substituted by from one to five substituents each independently selected from the group consisting of (1) halogen atoms, (2) C₁₋₄ alkyl groups, (3) C₁₋₄ alkoxy groups, (4) nitrile, (5) C₁₋₄ haloalkyl groups, and (6) C₁₋₄ haloalkoxy groups, wherein when two or more substituents are present on ring1, these substituents may form a 4- to 7-membered cyclic group together with the atoms in ring1 to which these substituents are bound;
ring2 represents a 4- to 7-membered saturated heterocycle, which may be substituted by from one to three —K—R²; K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR⁸;
R³ and R⁴ each independently represent (1) a hydrogen atom, or (2) a C₁₋₄ alkyl group which may be substituted by OR⁹ or CONR¹⁰R¹¹; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group; n represents an integer from 0 to 4;
m represents an integer from 0 to 2; and
when n is two or more, the R¹'s may be the same as each other or may differ from one another).

In an exemplary embodiment, the BTK inhibitor is a compound of Formula (19):

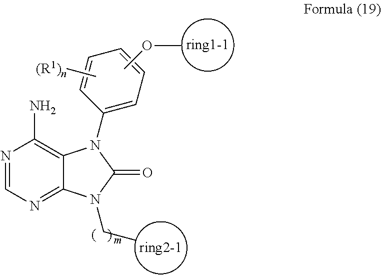

or a pharmaceutically acceptable salt, hydrate, solvate, cocrystal, or prodrug thereof, wherein
R¹ represents (1) a halogen atom, (2) a C₁₋₄ alkyl group, (3) a C₁₋₄ alkoxy group, (4) a C₁₋₄ haloalkyl group, or (5) a C₁₋₄ haloalkoxy group;
ring1 represents a benzene, cyclohexane, or pyridine ring, each of which may be substituted by from one to five substituents each independently selected from the group consisting of (1) halogen atoms, (2) C₁₋₄ alkyl groups, (3) C₁₋₄ alkoxy groups, (4) nitrile, (5) CF₃;
ring2 represents a 4- to 7-membered nitrogenous saturated heterocycle, which may be substituted by from one to three —K—R²; wherein K represents (1) a bond, (2) a C₁₋₄ alkylene, (3) —C(O)—, (4) —C(O)—CH₂—, (5) —CH₂—C(O)—, (6) —C(O)O—, or (7) —SO₂— (wherein the bond on the left is bound to the ring2);
R² represents (1) a C₁₋₄ alkyl, (2) a C₂₋₄ alkenyl, or (3) a C₂₋₄ alkynyl group, each of which may be substituted by from one to five substituents each independently selected from the group consisting of (1) NR³R⁴, (2) halogen atoms, (3) CONR⁵R⁶, (4) CO₂R⁷, and (5) OR⁸;
R³ and R⁴ each independently represent (1) a hydrogen atom, or (2) a C₁₋₄ alkyl group which may be substituted by OR⁹ or CONR¹⁰R¹¹; R³ and R⁴ may, together with the nitrogen atom to which they are bound, form a 4- to 7-membered nitrogenous saturated heterocycle, which may be substituted by an oxo group or a hydroxyl group;
R⁵ and R⁶ each independently represent (1) a hydrogen atom, (2) a C₁₋₄ alkyl group, or (3) a phenyl group;
R⁷ represents (1) a hydrogen atom or (2) a C₁₋₄ alkyl group;
R⁸ represents (1) a hydrogen atom, (2) a C₁₋₄ alkyl group, (3) a phenyl group, or (4) a benzotriazolyl group; R⁹ represents (1) a hydrogen atom or (2) a C₁₋₄ alkyl group;
R¹⁰ and R¹¹ each independently represent (1) a hydrogen atom or (2) a C₁₋₄ alkyl group;
n represents an integer from 0 to 4;
m represents an integer from 0 to 2; and
when n is two or more, the R¹'s may be the same as each other or may differ from one another).

In a preferred embodiment, the BTK inhibitor is a compound of Formula (20):

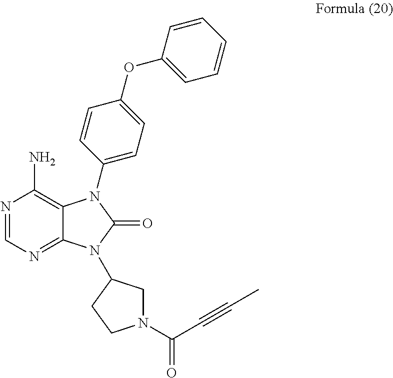 [SMILES: CC#CC(=O)N1CCC(n2c(=O)n(-c3ccc(Oc4ccccc4)cc3)c3c(N)ncnc32)C1]

or a pharmaceutically acceptable salt, solvate, hydrate, cocrystal, or prodrug thereof, preferably a hydrochloride salt thereof. The preparation of this compound is described in International Patent Application Publication No. WO 2013/081016 A1 and U.S. Patent Application Publication No. US 2014/0330015 A1, the disclosure of which is incorporated by reference herein. In an embodiment, the BTK inhibitor is 6-amino-9-(1-(but-2-ynoyl)pyrrolidin-3-yl)-7-(4-phenoxyphenyl)-7,9-dihydro-8H-purin-8-one or a pharmaceutically acceptable salt, solvate, hydrate, cocrystal, or prodrug thereof, or preferably a hydrochloride salt thereof. In an embodiment, the BTK inhibitor is 6-amino-9-[(3S)-1-(2-butynoyl)-3-pyrrolidinyl]-7-(4-phenoxyphenyl)-7,9-dihydro-8H-purin-8-one or a pharmaceutically acceptable salt, solvate, hydrate, cocrystal, or prodrug thereof, or a hydrochloride salt thereof.

The R-enantiomer of Formula (20) is also known as ONO-4059, and is given by Formula (21). In a preferred embodiment, the BTK inhibitor is a compound of Formula (21):

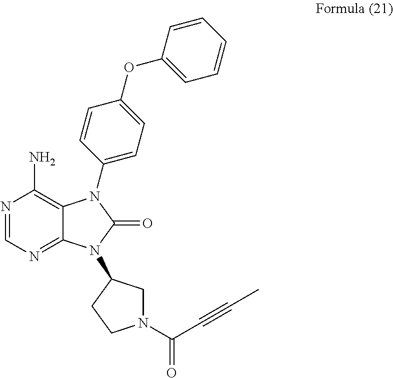 [SMILES: CC#CC(=O)N1CC[C@@H](n2c(=O)n(-c3ccc(Oc4ccccc4)cc3)c3c(N)ncnc32)C1]

or a pharmaceutically acceptable salt, solvate, hydrate, cocrystal, or prodrug thereof, preferably a hydrochloride salt thereof.

In an embodiment, the BTK inhibitor is 6-amino-9-[(3R)-1-(2-butynoyl)-3-pyrrolidinyl]-7-(4-phenoxyphenyl)-7,9-dihydro-8H-purin-8-one or a pharmaceutically acceptable salt, solvate, hydrate, cocrystal, or prodrug thereof, preferably a hydrochloride salt thereof.

The preparation of Formula (21) is described in International Patent Application Publication No. WO 2013/081016 A1, the disclosure of which is incorporated by reference herein. In brief, the BTK inhibitor of Formula (21) can be prepared by the following procedure.

Step 1: A solution of dibenzylamine (10.2 g) in dichloromethane (30 mL) is dripped into a solution of 4,6-dichloro-5-nitropyrimidine (10 g) in dichloromethane (70 mL) on an ice bath. Then triethylamine (14.4 mL) is added, and the mixture is stirred for 1 hour. Water is added to the reaction mixture, the organic layer is washed with a saturated aqueous sodium chloride solution and dried over anhydrous sodium sulfate, and the solvent is concentrated under reduced pressure to obtain N,N-dibenzyl-6-chloro-5-nitropyrimidine-4-amine (19.2 g).

Step 2: The compound prepared in Step 1 (19 g) and tert-butyl (3R)-3-aminopyrrolidine-1-carboxylate (10.5 g) are dissolved in dioxane (58 mL). Triethylamine (8.1 mL) is added, and the mixture is stirred for 5 hours at 50° C. The reaction mixture is returned to room temperature, the solvent is distilled off, water is added, and extraction is performed with ethyl acetate. The organic layer is washed with saturated aqueous sodium chloride solution, then dried over anhydrous sodium sulfate, and the solvent is distilled off. The residue is purified by silica gel column chromatography to obtain tert-butyl (3R)-3-{[6-(dibenzylamino)-5-nitropyrimidin-4-yl]amino}pyrrolidine-1-carboxylate (27.0 g).

Step 3: An ethyl acetate (360 mL) solution of the compound prepared in Step 2 (17.5 g) is dripped into a mixture of zinc (23.3 g) and a 3.0 M aqueous ammonium chloride solution (11.4 g) on an ice bath, and the temperature is immediately raised to room temperature. After stirring for 2 hours, the reaction mixture is filtered through CELITE and the solvent is distilled off. The residue is purified by silica gel column chromatography to obtain tert-butyl (3R)-3-{[5-amino-6-(dibenzylamino)pyrimidin-4-yl]amino}pyrrolidine-1-carboxylate (12.4 g).

Step 4: The compound prepared in Step 3 (8.4 g) and 1,1′-carbonyl diimidazole (5.9 g) are dissolved in tetrahydrofuran (120 mL) and the solution is stirred for 15 hours at 60° C. The solvent is distilled off from the reaction mixture, water is added, and extraction with ethyl acetate is performed. The organic layer is washed with saturated aqueous sodium chloride solution, dried over anhydrous sodium sulfate, and the solvent is distilled off. The residue is purified by silica gel column chromatography to obtain tert-butyl (3R)-3-[6-(dibenzylamino)-8-oxo-7,8-dihydro-9H-purin-9-yl]pyrrolidin-1-carboxylate (7.8 g).

Step 5: The compound prepared in Step 4 (7.8 g) is dissolved in methanol (240 mL) and ethyl acetate (50 mL), 20% Pearlman's catalyst (Pd(OH)₂/C) (8.0 g, 100 wt %) is added, hydrogen gas replacement is carried out, and stirring is performed for 7.5 hours at 60° C. The reaction mixture is filtered through CELITE and the solvent is distilled off to obtain tert-butyl (3R)-3-(6-amino-8-oxo-7,8-dihydro-9H-purin-9-yl)pyrrolidine-1-carboxylate (5.0 g).

Step 6: At room temperature p-phenoxy phenyl boronic acid (2.1 g), copper(II) acetate (1.48 g), molecular sieve 4A (2.5 g), and pyridine (0.82 mL) are added to a dichloromethane suspension (200 mL) of the compound prepared in Step 5 (2.5 g), followed by stirring for 21 hours. The reaction mixture is filtered through CELITE and the residue is purified by silica gel column chromatography to obtain tert-butyl (3R)-3-[6-amino-8-oxo-7-(4-phenoxyphenyl)-7,8-dihydro-9H-purin-9-yl]pyrrolidine-1-carboxylate (1.3 g).

Step 7: At room temperature 4 N HCl/dioxane (13 mL) is added to a methanol (13 mL) suspension of the compound prepared in Step 6 (1.3 g 2.76 mmol, 1.0 equivalent), and the mixture is stirred for 1 hour. The solvent is then distilled off to obtain (3R)-6-amino-9-pyrrolidin-3-yl-7-(4-phenoxyphenyl)-7,9-dihydro-8H-purin-8-one dihydrochloride (1.5 g).

Step 8: After 2-butylnoic acid (34 mg), 1-ethyl-3-(3-dimethylaminopropyl) carbodiimide hydrochloride (EDC) (78 mg), 1-hydroxybenzotriazole (HOBt) (62 mg), and triethylamine (114 mL) are added to a solution of the compound prepared in Step 7 (100 mg) in dimethyl formamide (3 mL), the mixture is stirred at room temperature for 3 hours. Water is added to the reaction mixture and extraction with ethyl acetate is performed. The organic layer is washed with saturated sodium carbonate solution and saturated aqueous sodium chloride solution, then dried over anhydrous sodium sulfate, and the solvent is distilled off. The residue is purified by thin layer chromatography (dichloromethane:methanol:28% ammonia water=90:10:1) to obtain 6-amino-9-[(3R)-1-(2-butynoyl)-3-pyrrolidinyl]-7-(4-phenoxyphenyl)-7,9-dihydro-8H-purin-8-one (Formula (21)) (75 mg).

The hydrochloride salt of the compound of Formula (21) can be prepared as follows: 6-amino-9-[(3R)-1-(2-butynoyl)-3-pyrrolidinyl]-7-(4-phenoxyphenyl)-7,9-dihydro-8H-purin-8-one (3.0 g) (which may be prepared as described above) is placed in a 300 mL 3-neck pear-shaped flask, ethyl acetate (30 mL) and 1-propanol (4.5 mL) are added, and the external temperature is set at 70° C. (internal temperature 61° C.). After it is confirmed that the compound prepared in Step 8 has dissolved completely, 10% HCl/methanol (3.5 mL) is added, and after precipitation of crystals is confirmed, the crystals are ripened by the following sequence: external temperature 70° C. for 30 min, external temperature 60° C. for 30 min, external temperature 50° C. for 60 min, external temperature 40° C. for 30 min, room temperature for 30 min, and an ice bath for 30 min. The resulting crystals are filtered, washed with ethyl acetate (6 mL), and dried under vacuum at 50° C. to obtain white crystals of 6-amino-9-[(3R)-1-(2-butynoyl)-3-pyrrolidinyl]-7-(4-phenoxyphenyl)-7,9-dihydro-8H-purin-8-one hydrochloride (2.76 g).

In an embodiment, the BTK inhibitor is a compound selected from the structures disclosed in U.S. Patent Application Publication No. US 2014/0330015 A1, the disclosure of which is incorporated by reference herein.

In an embodiment, the BTK inhibitor is a compound of Formula (22):

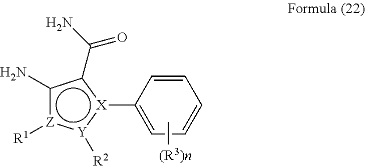

or a pharmaceutically acceptable salt, solvate, hydrate, cocrystal, or prodrug thereof, wherein:
X—Y—Z is N—C—C and R² is present, or C—N—N and R² is absent;
R¹ is a 3-8 membered, N-containing ring, wherein the N is unsubstituted or substituted with R⁴;
R² is H or lower alkyl, particularly methyl, ethyl, propyl or butyl; or
R¹ and R² together with the atoms to which they are attached, form a 4-8 membered ring, preferably a 5-6 membered ring, selected from cycloalkyl, saturated or unsaturated heterocycle, aryl, and heteroaryl rings unsubstituted or substituted with at least one substituent L-R⁴;
R³ is in each instance, independently halogen, alkyl, S-alkyl, CN, or OR⁵;
n is 1, 2, 3, or 4, preferably 1 or 2;
L is a bond, NH, heteroalkyl, or heterocyclyl;
R⁴ is COR′, CO₂R′, or SO₂R′, wherein R′ is substituted or unsubstituted alkyl, substituted or unsubstituted alkenyl, substituted or unsubstituted alkynyl;
R⁵ is H or unsubstituted or substituted heteroalkyl, alkyl, cycloalkyl, saturated or unsaturated heterocyclyl, aryl, or heteroaryl.

In some embodiments, the BTK inhibitor is one of the following particular embodiments of Formula (22):
X—Y—Z is C—N—N and R² is absent; and R¹ is 3-8 membered, N-containing ring, N-substituted with R⁴;
X—Y—Z is N—C—C and R² is present, R¹ is 3-8 membered, N-containing ring, N-substituted with R⁴; and R² is H or lower alkyl;
X—Y—Z is N—C—C and R² is present; and R¹ and R² together with the atoms to which they are attached, form a 4-8 membered ring selected from cycloalkyl, saturated or unsaturated heterocycle, aryl, and heteroaryl rings unsubstituted or substituted with at least one substituent L-R⁴, wherein preferred rings of R¹ and R² are 5-6-membered, particularly dihydropyrrole, tetrahydropyridine, tetrahydroazepine, phenyl, or pyridine;
X—Y—Z is N—C—C and R² is present; and R¹ and R² together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond;
R¹ is piperidine or azaspiro[3.3]heptane, preferably N-substituted with R⁴;
R⁴ is COR′ or SO₂R′, particularly wherein R′ is substituted or unsubstituted alkenyl, particularly substituted or unsubstituted ethenyl; or
R⁵ is unsubstituted or substituted alkyl or aryl, particularly substituted or unsubstituted phenyl or methyl, such as cyclopropyl-substituted methyl with or tetrabutyl-substituted phenyl.

In some embodiments, the BTK inhibitor is one of the following particular embodiments of Formula (22):
R¹ is piperidine or azaspiro[3.3]heptane, N-substituted with R⁴, wherein R⁴ is H, CO′ or SO₂R′, and R′ is substituted or unsubstituted alkenyl, particularly substituted or unsubstituted ethenyl;
R³ is —OR⁵, R⁵ is phenyl, and n is 1;
R¹ and R², together with the atoms to which they are attached, form a 5-6 membered ring, preferably (a) phenyl substituted with a single -L-R⁴, or (b) dihydropyrrole or tetrahydropyridine, N-substituted with a single -L-R⁴ wherein L is bond; R³ is —OR⁵; n is 1; R⁴ is COR′, and R′ is ethenyl; and R⁵ is phenyl; and
X—Y—Z is C—N—N and R² is absent; R¹ is piperidine, N-substituted with R⁴; R³ is —OR⁵; n is 1; R⁴ is COR′, and R′ is unsubstituted or substituted alkenyl, particularly ethenyl; and R⁵ is substituted or unsubstituted aryl, particularly phenyl.

In some embodiments, the BTK inhibitor is a compound selected from the group consisting of Formula (23), Formula (24), or Formula (25):

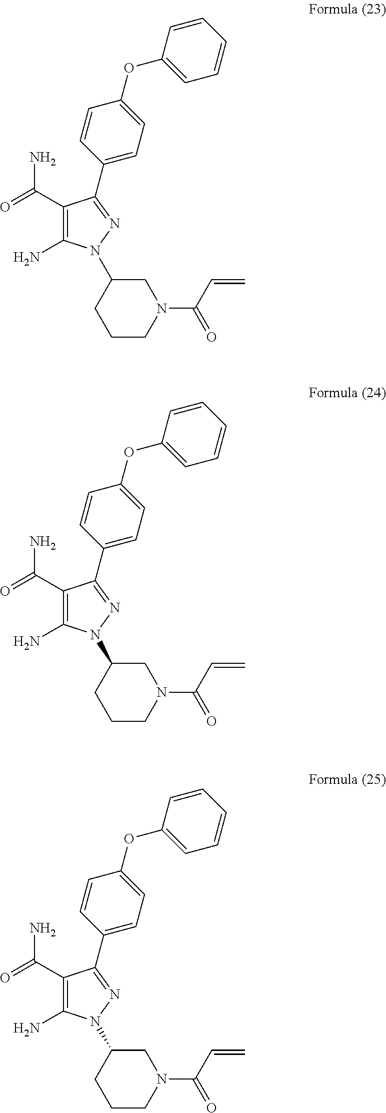 [SMILES: C=CC(=O)N1CCCC(n2nc(-c3ccc(Oc4ccccc4)cc3)c(C(N)=O)c2N)C1 ; C=CC(=O)N1CCC[C@@H](n2nc(-c3ccc(Oc4ccccc4)cc3)c(C(N)=O)c2N)C1 ; C=CC(=O)N1CCC[C@H](n2nc(-c3ccc(Oc4ccccc4)cc3)c(C(N)=O)c2N)C1]

or a pharmaceutically acceptable salt, solvate, hydrate, cocrystal, or prodrug thereof. Formula (24) is also known as BGB-3111. The preparation of these compounds is described in International Patent Application Publication No. WO 2014/173289 A1 and U.S. Patent Application Publication No. US 2015/0005277 A1, the disclosure of which is incorporated by reference herein.

In brief, the BTK inhibitor of Formula (23) can be prepared by the following procedure.

### Step 1. Preparation of 2-(hydroxy(4-phenoxyphenyl)methylene)malononitrile

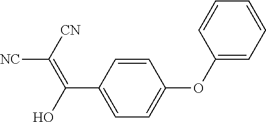 [SMILES: N#CC(C#N)=C(O)c1ccc(Oc2ccccc2)cc1]

A solution of 4-phenoxybenzoic acid (300 g, 1.4 mol) in SOCl₂ (1.2 L) is stirred at 80° C. under N₂ for 3 hours. The mixture is concentrated in vacuum to give the intermediate (315 g) which is used for next step without further purification.

To a solution of propanedinitrile (89.5 g, 1355 mmol) and N,N-diisopropylethylamine (DIEA) (350 g, 2710 mmol) in THF (800 mL) is added dropwise a solution of the intermediate (315 g) in toluene (800 mL) at 0-5° C. over 2 hours. The resultant mixture is allowed to warm to RT and stirred for 16 hours. The reaction is quenched with water (2.0 L) and extracted with of EA (2.0 L×3). The combined organic layers are washed with 1000 mL of 3 N HCl aqueous solution, brine (2.0 L×3), dried over Na₂SO₄ and concentrated to give the crude product (330 g, 93%).

### Step 2. Preparation of 2-(methoxy(4-phenoxyphenyl)methylene)malononitrile

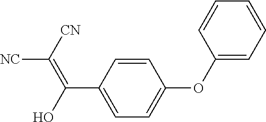 [SMILES: N#CC(C#N)=C(O)c1ccc(Oc2ccccc2)cc1]

A solution of 2-(hydroxy(4-phenoxyphenyl)methylene)malononitrile (50 g, 190.8 mmol) in CH(OMe₃) (500 mL) is heated to 75° C. for 16 hours. Then the mixture is concentrated to a residue and washed with MeOH (50 mL) to give 25 g (47.5%) of 2-(methoxy(4-phenoxyphenyl)methylene)malononitrile as a yellow solid.

### Step 3. Preparation of 5-amino-3-(4-phenoxyphenyl)-1H-pyrazole-4-carbonitrile

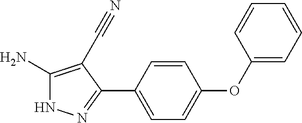 [SMILES: N#Cc1c(-c2ccc(Oc3ccccc3)cc2)n[nH]c1N]

To a solution of 2-(methoxy(4-phenoxyphenyl)methylene)malononitrile (80 g, 290 mmol) in ethanol (200 mL) is added hydrazine hydrate (20 mL). The mixture is stirred at RT for 16 hours then is concentrated to give the crude product and washed with MeOH (30 mL) to afford 55 g (68.8%) of 5-amino-3-(4-phenoxyphenyl)-1H-pyrazole-4-carbonitrile as an off-white solid.

### Step 4. Preparation of tert-butyl 3-(tosyloxy)piperidine-1-carboxylate

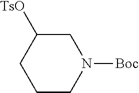 [SMILES: Cc1ccc(S(=O)(=O)OC2CCCN(C(=O)OC(C)(C)C)C2)cc1]

wherein “Boc” represents a tert-butyloxycarbonyl protecting group.

To a solution of tert-butyl 3-hydroxypiperidine-1-carboxylate (1.05 g, 5.0 mmol) in pyridine (8 mL) is added TsCl (1.425 g, 7.5 mmol). The mixture is stirred at RT under N₂ for two days. The mixture is concentrated and partitioned between 100 mL of EA and 100 mL of HCl (1 N) aqueous solution. The organic layer is separated from aqueous layer, washed with saturated NaHCO₃ aqueous solution (100 mL×2), brine (100 mL×3) and dried over Na₂SO₄. The organic layer is concentrated to afford 1.1 g (60%) of tert-butyl 3-(tosyloxy)piperidine-1-carboxylate as a colorless oil.

### Step 5. Preparation of tert-butyl 3-(5-amino-4-cyano-3-(4-phenoxyphenyl)-1H-pyrazol-1-yl)piperidine-1-carboxylate

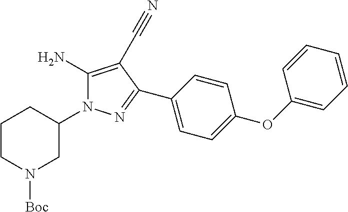 [SMILES: CC(C)(C)OC(=O)N1CCCC(n2nc(-c3ccc(Oc4ccccc4)cc3)c(C#N)c2N)C1]

To a solution of tert-butyl 3-(tosyloxy)piperidine-1-carboxylate (355 mg, 1.0 mmol) and 5-amino-3-(4-phenoxyphenyl)-1H-pyrazole-4-carbonitrile (276 mg, 1.0 mmol) in 5 mL of DMF is added Cs₂CO₃ (650 mg, 2.0 mmol). A tosyloxy leaving group is employed in this reaction. The mixture is stirred at RT for 16 hours, 75° C. for 3 hours and 60° C. for 16 hours. The mixture is concentrated washed with brine (100 mL×3) and dried over Na₂SO₄. The material is concentrated and purified by chromatography column on silica gel (eluted with petroleum ether/ethyl actate=3/1) to afford 60 mg (13%) of tert-butyl 3-(5-amino-4-cyano-3-(4-phenoxyphenyl)-1H-pyrazol-1-yl)piperidine-1-carboxylate as a yellow oil.

### Step 6. Preparation of tert-butyl 3-(5-amino-4-carbamoyl-3-(4-phenoxyphenyl)-1H-pyrazol-1-yl)piperidine-1-carboxylate

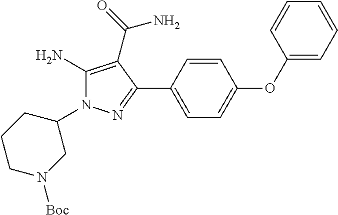 [SMILES: CC(C)(C)OC(=O)N1CCCC(n2nc(-c3ccc(Oc4ccccc4)cc3)c(C(N)=O)c2N)C1]

To a solution of tert-butyl 3-(5-amino-4-cyano-3-(4-phenoxyphenyl)-1H-pyrazol-1-yl)piperidine-1-carboxylate (100 mg, 0.22 mmol) in DMSO (2 mL) and ethanol (2 mL) was added the solution of NaOH (200 mg, 5 mmol) in water (1 mL) and H₂O₂ (1 mL). The mixture is stirred at 60° C. for 15 min and concentrated to remove EtOH, after which 10 mL of water and 50 mL of ethyl acetate are added. The organic layer is separated from aqueous layer, washed with brine (30 mL×3) and dried over Na₂SO₄. After concentration, 50 mg of residue is used directly in the next step, wherein 50 mg of residue is purified by pre-TLC (eluted with petroleum ether/ethyl actate=1/1) to afford 12 mg (30%) of tert-butyl 3-(5-amino-4-carbamoyl-3-(4-phenoxyphenyl)-1H-pyrazol-1-yl)piperidine-1-carboxylate as a white solid.

### Step 7. Preparation of 5-amino-3-(4-phenoxyphenyl)-1-(piperidin-3-yl)-1H-pyrazole-4-carboxamide

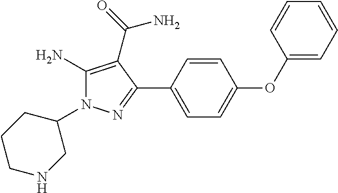 [SMILES: NC(=O)c1c(-c2ccc(Oc3ccccc3)cc2)nn(C2CCCNC2)c1N]

To a solution of tert-butyl 3-(5-amino-4-carbamoyl-3-(4-phenoxyphenyl)-1H-pyrazol-1-yl)piperidine-1-carboxylate (50 mg, 0.11 mmol) in ethyl acetate (1 mL) is added concentrated HCl (0.75 mL). The mixture is stirred at RT for 1 hour. Then saturated NaHCO₃ is added until pH>7, followed by ethyl acetate (50 mL). The organic layer is separated from aqueous layer, washed with brine (50 mL×3) and dried over Na₂SO₄. The resulting product is concentrated and purified by Pre-TLC (eluted with dichloromethane/MeOH/NH₃—H₂O=5/1/0.01) to afford 10 mg (25%) of 5-amino-3-(4-phenoxyphenyl)-1-(piperidin-3-yl)-1H-pyrazole-4-carboxamide as a white solid.

### Step 8. Preparation of 1-(1-acryloylpiperidin-3-yl)-5-amino-3-(4-phenoxyphenyl)-1H-pyrazole-4-carboxamide

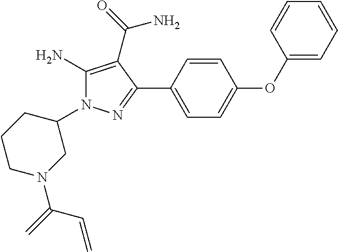 [SMILES: C=CC(=C)N1CCCC(n2nc(-c3ccc(Oc4ccccc4)cc3)c(C(N)=O)c2N)C1]

To a solution of 5-amino-3-(4-phenoxyphenyl)-1-(piperidin-3-yl)-1H-pyrazole-4-carboxamide (63 mg, 0.17 mmol) in dichloromethane (4 mL) is added pyridine (27 mg, 0.34 mmol). Then a solution of acryloyl chloride (12 mg, 0.17 mmol) in dichloromethane (1 mL) is added dropwise. After stirring at RT for 4 hours, the mixture is partitioned between 100 mL of dichloromethane and 100 mL of brine. The organic layer is separated from aqueous layer, washed with brine (100 mL×2) and dried over Na₂SO₄. The material is concentrated and purified by Pre-TLC (eluted with dichloromethane/MeOH=10/1) to afford 4 mg (5.5%) of 1-(1-acryloylpiperidin-3-yl)-5-amino-3-(4-phenoxyphenyl)-1H-pyrazole-4-carboxamide as a white solid.

The enantiomers of Formula (23) provided by the procedure above may be prepared from 5-amino-3-(phenoxyphenyl)-1H-pyrazole-4-carbonitrile and (S)-tert-butyl 3-hydroxypiperidine-1-carboxylate using a similar procedure (step 4 to 8) for Formula (24), or from (R)-tert-butyl 3-hydroxypiperidine-1-carboxylate using a similar procedure (step 4 to 8) for Formula (25). Under appropriate conditions recognized by one of ordinary skill in the art, a racemic mixture of Formula (23) may be separated by chiral HPLC, the crystallization of chiral salts, or other means described above to yield Formula (24) and Formula (25) of high enantiomeric purity.

In an embodiment, the BTK inhibitor is a compound selected from the structures disclosed in U.S. Patent Application Publication No. US 2015/0005277A1, the disclosure of which is incorporated by reference herein.

### IRAK4 Inhibitors

The interleukin-1 receptor-associated kinase 4 (IRAK4) inhibitor may be any IRAK4 inhibitor known in the art. In particular, it is one of the IRAK4 inhibitors described in more detail in the following paragraphs. In an embodiment, it is a selective IRAK4 inhibitor. In preferred embodiments, the compositions described herein provide a combination of an IRAK4 inhibitor with a BTK inhibitor, or methods of using a combination of an IRAK4 inhibitor with a BTK inhibitor. In an embodiment, the IRAK4 inhibitor is also a IRAK1 inhibitor, also called the dual IRAK1/4 inhibitor.

In an embodiment, the IRAK4 inhibitor is 1-(4-hydroxycyclohexyl)-N-(3-methylbutyl)-2-[[5-[2-(trifluoromethoxy)phenyl]-1H-indazol-3-yl]amino]benzimidazole-5-carboxamide (CAS No. 1012104-68-5) (Formula (44)):

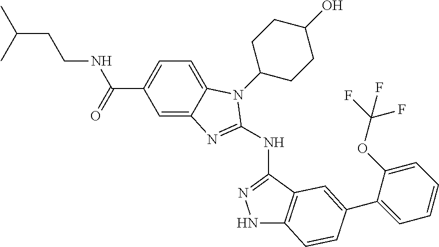 [SMILES: CC(C)CCNC(=O)c1ccc2c(c1)nc(Nc1n[nH]c3ccc(-c4ccccc4OC(F)(F)F)cc13)n2C1CCC(O)CC1]

or a pharmaceutically acceptable salt, solvate, hydrate, cocrystal, or prodrug thereof.

In an embodiment, the IRAK4 inhibitor is N-(1-(2-morpholinoethyl)-1H-benzo[d]imidazol-2-yl)-3-nitrobenzamide (CAS No. 509093-47-4) (Formula (45)):

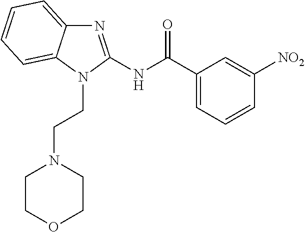 [SMILES: O=C(Nc1nc2ccccc2n1CCN1CCOCC1)c1cccc([N+](=O)[O-])c1]

or a pharmaceutically acceptable salt, solvate, hydrate, cocrystal, or prodrug thereof. The compound of Formula (45) is a dual IRAK1/4 inhibitor.

In an embodiment, the IRAK4 inhibitor is 1-(3-hydroxypropyl)-2-(3-nitrobenzamido)-1H-benzo[d]imidazol-5-yl pivalate (CAS No. 509093-60-1) (Formula (46)):

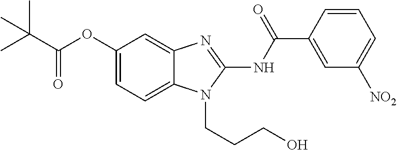 [SMILES: CC(C)(C)C(=O)Oc1ccc2c(c1)nc(NC(=O)c1cccc([N+](=O)[O-])c1)n2CCCO]

or a pharmaceutically acceptable salt, solvate, hydrate, cocrystal, or prodrug thereof.

In an embodiment, the IRAK4 inhibitor is N-[3-carbamoyl-1-(tetrahydro-2H-pyran-4-yl)-1H-pyrazol-4-yl]-2-(2-methylpyridin-4-yl)-1,3-oxazole-4-carboxamide (CAS No. 1287665-58-0) (Formula (46)):

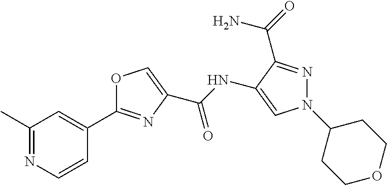 [SMILES: Cc1cc(-c2nc(C(=O)Nc3cn(C4CCOCC4)nc3C(N)=O)co2)ccn1]

or a pharmaceutically acceptable salt, solvate, hydrate, cocrystal, or prodrug thereof.

In an embodiment, the IRAK4 inhibitor is N-[3-carbamoyl-1-(tetrahydro-2H-pyran-4-yl)-1H-pyrazol-4-yl]-2-(2-methylpyridin-4-yl)-1,3-oxazole-4-carboxamide hydrochloride (AS2444697, CAS No. 1287665-60-4) (Formula (46), HCl salt):

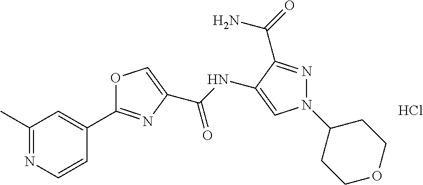 [SMILES: Cc1cc(-c2nc(C(=O)Nc3cn(C4CCOCC4)nc3C(N)=O)co2)ccn1 ; Cl]

or a solvate, hydrate, cocrystal, or prodrug thereof.

In an embodiment, the IRAK4 inhibitor is a compound selected from those disclosed in Chaudhary, et al., J. Med. Chem. 2015, 58, 96-110, which is incorporated herein by reference in its entirety; or a pharmaceutically acceptable salt, solvate, hydrate, cocrystal, or prodrug thereof. In an embodiment, the IRAK4 inhibitor is a compound selected from compounds 1-55 in Chaudhary, et al., J. Med. Chem. 2015, 58, 96-110; or a pharmaceutically acceptable salt, solvate, hydrate, cocrystal, or prodrug thereof.

In an embodiment, the IRAK4 inhibitor is a compound selected from those disclosed in Hynes and Nair, Annu. Rep. Med. Chem. 2014, 49, 117-133, which is incorporated herein by reference in its entirety; or a pharmaceutically acceptable salt, solvate, hydrate, cocrystal, or prodrug thereof. In an embodiment, the IRAK4 inhibitor is a compound selected from compounds 1-46 in Hynes and Nair, Annu. Rep. Med. Chem. 2014, 49, 117-133; or a pharmaceutically acceptable salt, solvate, hydrate, cocrystal, or prodrug thereof.

In an embodiment, the IRAK4 inhibitor is a compound selected from those listed in

TABLE 1
IRAK4 inhibitors.

| Structure | Name |
|---|---|
| 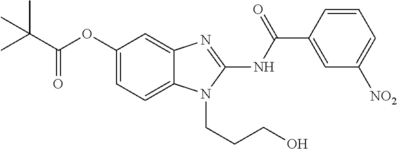 [SMILES: CC(C)(C)C(=O)Oc1ccc2c(c1)nc(NC(=O)c1cccc([N+](=O)[O-])c1)n2CCCO] | 1-(3-hydroxypropyl)-2-(3-nitrobenzamido)-1H- benzo[d]imidazol-5-yl pivalate |
| 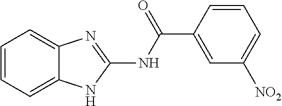 [SMILES: O=C(Nc1nc2ccccc2[nH]1)c1cccc([N+](=O)[O-])c1] | N-(1H-benzo[d]imidazol-2-yl)-3-nitrobenzamide |
| 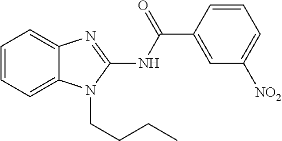 [SMILES: CCCCn1c(NC(=O)c2cccc([N+](=O)[O-])c2)nc2ccccc21] | N-(1-butyl-1H-benzo[d]imidazol-2-yl)-3-nitrobenzamide |
| 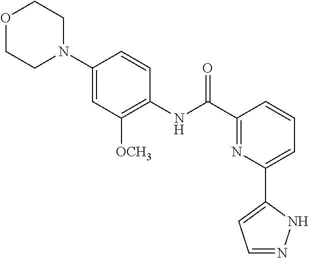 [SMILES: COc1cc(N2CCOCC2)ccc1NC(=O)c1cccc(-c2ccn[nH]2)n1] | N-(2-methoxy-4-morpholinophenyl)-6-(1H-pyrazol-5- yl)picolinamide |
| 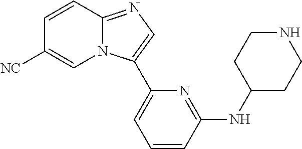 [SMILES: N#Cc1ccc2ncc(-c3cccc(NC4CCNCC4)n3)n2c1] | 3-(6-(piperidin-4-ylamino)pyridin-2-yl)imidazo[1,2- a]pyridine-6-carbonitrile |
| 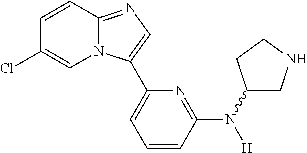 [SMILES: Clc1ccc2ncc(-c3cccc(NC4CCNC4)n3)n2c1] | 6-(6-chloroimidazo[1,2-a]pyridin-3-yl)-N-(pyrrolidin-3- yl)pyridin-2-amine |
| 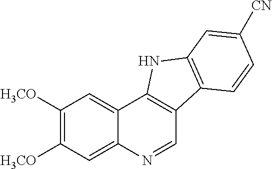 [SMILES: COc1cc2ncc3c4ccc(C#N)cc4[nH]c3c2cc1OC] | 2,3-dimethoxy-11H-indolo[3,2-c]quinoline-9-carbonitrile |
| 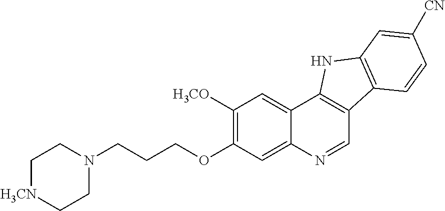 | 2-methoxy-3-(3-(4-methylpiperazin-1-yl)propoxy)-11H- indolo[3,2-c]quinoline-9-carbonitrile |
| 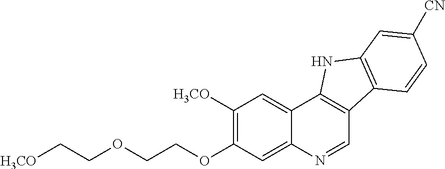 [SMILES: COCCOCCOc1cc2ncc3c4ccc(C#N)cc4[nH]c3c2cc1OC] | 2-methoxy-3-(2-(2-methoxyethoxy)ethoxy)-11H-indolo[3,2- c]quinoline-9-carbonitrile |
| 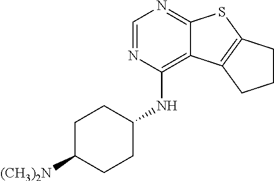 | (1r,4r)-N1-(6,7-dihydro-5H-cyclopenta[4,5]thieno[2,3- d]pyrimidin-4-yl)-N4,N4-dimethylcyclohexane-1,4-diamine |
| 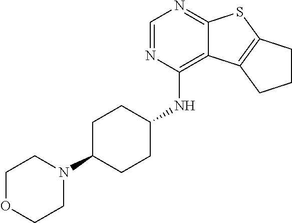 [SMILES: c1nc(N[C@H]2CC[C@H](N3CCOCC3)CC2)c2c3c(sc2n1)CCC3] | N-((1r,4r)-4-morpholinocyclohexyl)-6,7-dihydro-5H- cyclopenta[4,5]thieno[2,3-d]pyrimidin-4-amine |
| 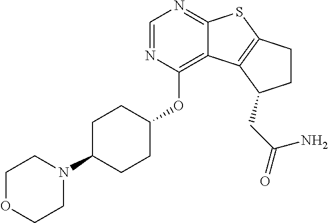 [SMILES: NC(=O)C[C@H]1CCc2sc3ncnc(O[C@H]4CC[C@H](N5CCOCC5)CC4)c3c21] | 2-((R)-4-(((1r,4R)-4-morpholinocyclohexyl)oxy)-6,7- dihydro-5H-cyclopenta[4,5]thieno[2,3-d]pyrimidin-5- yl)acetamide |
| 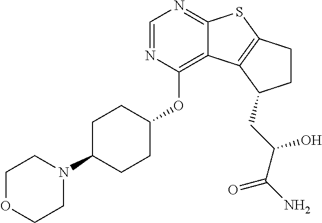 [SMILES: NC(=O)[C@@H](O)C[C@H]1CCc2sc3ncnc(O[C@H]4CC[C@H](N5CCOCC5)CC4)c3c21] | (S)-2-hydroxy-3-((R)-4-(((1r,4R)-4- morpholinocyclohexyl)oxy)-6,7-dihydro-5H- cyclopenta[4,5]thieno[2,3-d]pyrimidin-5-yl)propanamide |
| 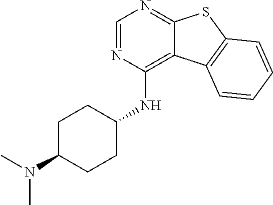 [SMILES: CN(C)[C@H]1CC[C@H](Nc2ncnc3sc4ccccc4c23)CC1] | (1r,4r)-N1-(benzo[4,5]thieno[2,3-d]pyrimidin-4-yl)-N4,N4- dimethylcyclohexane-1,4-diamine |
| 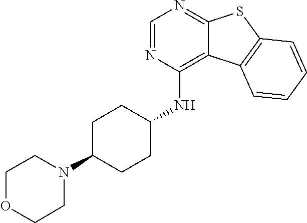 [SMILES: c1ccc2c(c1)sc1ncnc(N[C@H]3CC[C@H](N4CCOCC4)CC3)c12] | N-((1r,4r)-4-morpholinocyclohexyl)benzo[4,5]thieno[2,3- d]pyrimidin-4-amine |
| 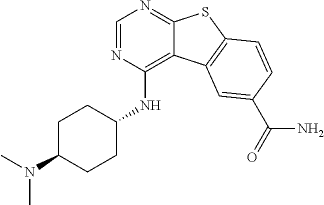 [SMILES: CN(C)[C@H]1CC[C@H](Nc2ncnc3sc4ccc(C(N)=O)cc4c23)CC1] | 4-(((1r,4r)-4- (dimethylamino)cyclohexyl)amino)benzo[4,5]thieno[2,3- d]pyrimidine-6-carboxamide |
| 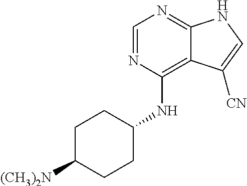 | 4-(((1r,4r)-4-(dimethylamino)cyclohexyl)amino)-7H- pyrrolo[2,3-d]pyrimidine-5-carbonitrile |
| 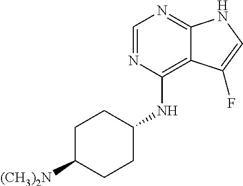 | (1r,4r)-N1-(5-fluoro-7H-pyrrolo[2,3-d]pyrimidin-4-yl)- N4,N4-dimethylcyclohexane-1,4-diamine |
| 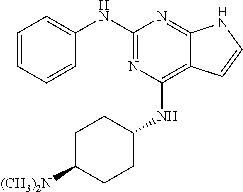 | N4-((1r,4r)-4-(dimethylamino)cyclohexyl)-N2-phenyl-7H- pyrrolo[2,3-d]pyrimidine-2,4-diamine |
| 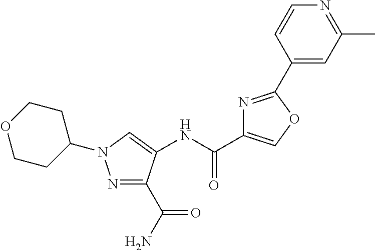 [SMILES: Cc1cc(-c2nc(C(=O)Nc3cn(C4CCOCC4)nc3C(N)=O)co2)ccn1] | N-(3-carbamoyl-1-(tetrahydro-2H-pyran-4-yl)-1H-pyrazol- 4-yl)-2-(2-methylpyridin-4-yl)oxazole-4-carboxamide |
| 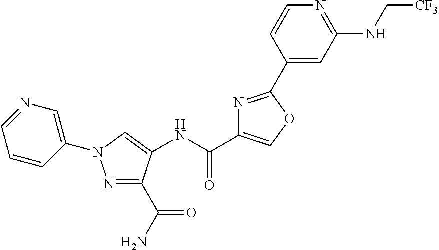 [SMILES: NC(=O)c1nn(-c2cccnc2)cc1NC(=O)c1coc(-c2ccnc(NCC(F)(F)F)c2)n1] | N-(3-carbamoyl-1-(pyridin-3-yl)-1H-pyrazol-4-yl)-2-(2- ((2,2,2-trifluoroethyl)amino)pyridin-4-yl)oxazole-4- carboxamide |
| 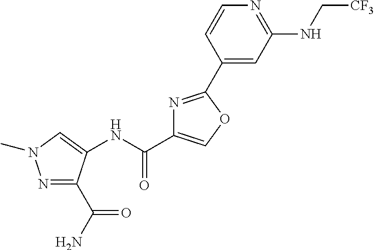 [SMILES: Cn1cc(NC(=O)c2coc(-c3ccnc(NCC(F)(F)F)c3)n2)c(C(N)=O)n1] | N-(3-carbamoyl-1-methyl-1H-pyrazol-4-yl)-2-(2-((2,2,2- trifluoroethyl)amino)pyridin-4-yl)oxazole-4-carboxamide |
| 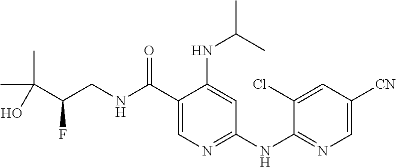 [SMILES: CC(C)Nc1cc(Nc2ncc(C#N)cc2Cl)ncc1C(=O)NC[C@@H](F)C(C)(C)O] | (R)-6-((3-chloro-5-cyanopyridin-2-yl)amino)-N-(2-fluoro-3- hydroxy-3-methylbutyl)-4-(isopropylamino)nicotinamide |
| 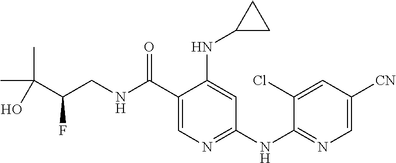 [SMILES: CC(C)(O)[C@H](F)CNC(=O)c1cnc(Nc2ncc(C#N)cc2Cl)cc1NC1CC1] | (R)-6-((3-chloro-5-cyanopyridin-2-yl)amino)-4- (cyclopropylamino)-N-(2-fluoro-3-hydroxy-3- methylbutyl)nicotinamide |
| 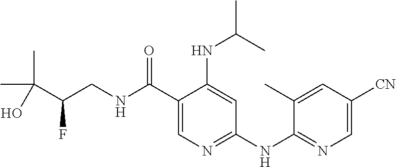 [SMILES: Cc1cc(C#N)cnc1Nc1cc(NC(C)C)c(C(=O)NC[C@@H](F)C(C)(C)O)cn1] | (R)-6-((5-cyano-3-methylpyridin-2-yl)amino)-N-(2-fluoro-3- hydroxy-3-methylbutyl)-4-(isopropylamino)nicotinamide |
| 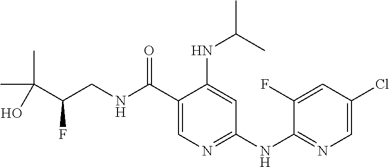 [SMILES: CC(C)Nc1cc(Nc2ncc(Cl)cc2F)ncc1C(=O)NC[C@@H](F)C(C)(C)O] | (R)-6-((5-chloro-3-fluoropyridin-2-yl)amino)-N-(2-fluoro-3- hydroxy-3-methylbutyl)-4-(isopropylamino)nicotinamide |
| 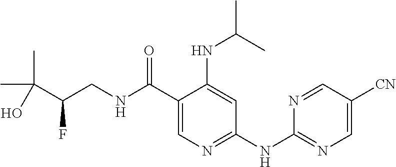 [SMILES: CC(C)Nc1cc(Nc2ncc(C#N)cn2)ncc1C(=O)NC[C@@H](F)C(C)(C)O] | (R)-6-((5-cyanopyrimidin-2-yl)amino)-N-(2-fluoro-3- hydroxy-3-methylbutyl)-4-(isopropylamino)nicotinamide |
| 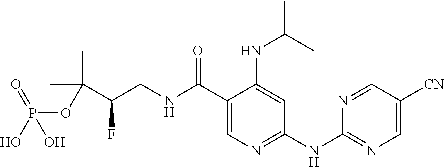 [SMILES: CC(C)Nc1cc(Nc2ncc(C#N)cn2)ncc1C(=O)NC[C@@H](F)C(C)(C)OP(=O)(O)O] | (R)-4-(6-((5-cyanopyrimidin-2-yl)amino)-4- (isopropylamino)nicotinamide)-3-fluoro-2-methylbutan-2-yl dihydrogen phosphate |
| 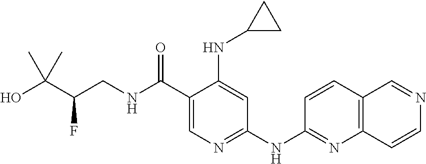 [SMILES: CC(C)(O)[C@H](F)CNC(=O)c1cnc(Nc2ccc3cnccc3n2)cc1NC1CC1] | (R)-6-((1,6-naphthyridin-2-yl)amino)-4-(cyclopropylamino)- N-(2-fluoro-3-hydroxy-3-methylbutyl)nicotinamide |
| 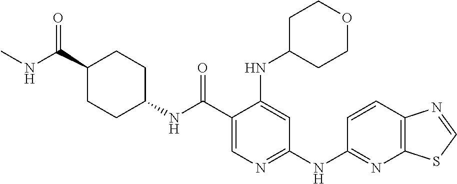 [SMILES: CNC(=O)[C@H]1CC[C@H](NC(=O)c2cnc(Nc3ccc4ncsc4n3)cc2NC2CCOCC2)CC1] | N-(1r,4r)-4-(methylcarbamoyl)cyclohexyl)-4-((tetrahydro- 2H-pyran-4-yl)amino)-6-(thiazolo[5,4-b]pyridin-5- ylamino)nicotinamide |
| 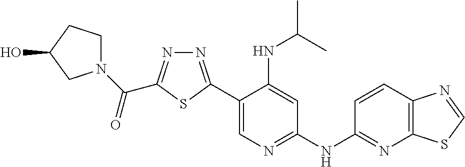 [SMILES: CC(C)Nc1cc(Nc2ccc3ncsc3n2)ncc1-c1nnc(C(=O)N2CC[C@H](O)C2)s1] | (S)-(5-(6-(benzo[d]thiazol-6-ylamino)-4- (isopropylamino)pyridin-3-yl)-1,3,4-thiazolo-2-yl)(3- hydroxypyrrolidin-1-yl)methanone |
| 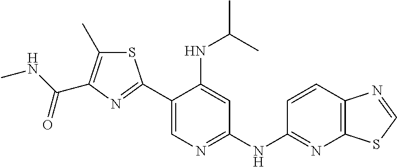 [SMILES: CNC(=O)c1nc(-c2cnc(Nc3ccc4ncsc4n3)cc2NC(C)C)sc1C] | 2-(6-(benzo[d]thiazol-6-ylamino)-4- (isopropylamino)pyridin-3-yl)-N,5-dimethylthiazole-4- carboxamide |
| 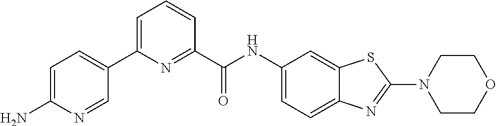 [SMILES: Nc1ccc(-c2cccc(C(=O)Nc3ccc4nc(N5CCOCC5)sc4c3)n2)cn1] | 6′-amino-N-(2-morpholinobenzo[d]thiazol-6-yl)-[2,3′- bipyridine]-6-carboxamide |
| 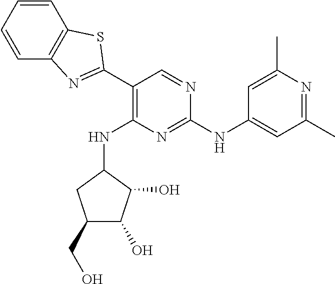 [SMILES: Cc1cc(Nc2ncc(-c3nc4ccccc4s3)c(NC3C[C@H](CO)[C@@H](O)[C@H]3O)n2)cc(C)n1] | (1R,2S,5R)-3-((5-(benzo[b]thiazol-2-yl)-2-((2,6- dimethylpyridin-4-yl)amino)pyrimidin-4-yl)amino)-5- (hydroxymethyl)cyclopentane-1,2-diol |
| 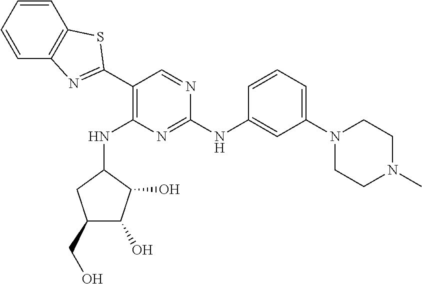 [SMILES: CN1CCN(c2cccc(Nc3ncc(-c4nc5ccccc5s4)c(NC4C[C@H](CO)[C@@H](O)[C@H]4O)n3)c2)CC1] | (1R,2S,5R)-3-((5-(benzo[b]thiazol-2-yl)-2-((3-(4- methylpiperazin-1-yl)phenyl)amino)pyrimidin-4-yl)amino)- 5-(hydroxymethyl)cyclopentane-1,2-diol |
| 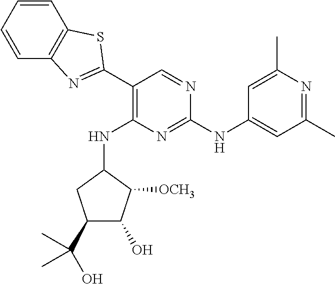 [SMILES: CO[C@H]1C(Nc2nc(Nc3cc(C)nc(C)c3)ncc2-c2nc3ccccc3s2)C[C@H](C(C)(C)O)[C@H]1O] | (1R,2S,5S)-3-((5-(benzo[b]thiazol-2-yl)-2-((2,6- dimethylpyridin-4-yl)amino)pyrimidin-4-yl)amino)-5-(2- hydroxypropan-2-yl)-2-methoxycyclopentan-1-ol |
| 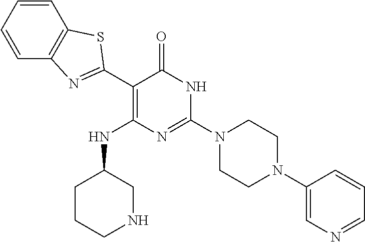 [SMILES: O=c1[nH]c(N2CCN(c3cccnc3)CC2)nc(N[C@@H]2CCCNC2)c1-c1nc2ccccc2s1] | (R)-5-(benzo[d]thiazol-2-yl)-6-(piperidin-3-ylamino)-2-(4- (pyridin-3-yl)piperazin-1-yl)pyrimidin-4(3H)-one |
| 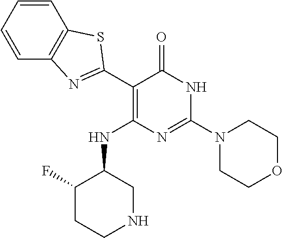 [SMILES: O=c1[nH]c(N2CCOCC2)nc(N[C@H]2CNCC[C@@H]2F)c1-c1nc2ccccc2s1] | 5-(benzo[d]thiazol-2-yl)-6-(((3S,4S)-4-fluoropiperidin-3- yl)amino)-2-morpholinopyrimidin-4(3H)-one |
| 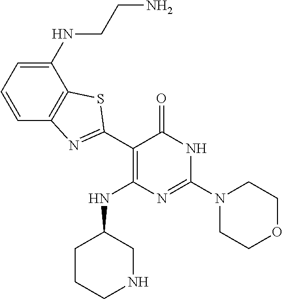 [SMILES: NCCNc1cccc2nc(-c3c(N[C@@H]4CCCNC4)nc(N4CCOCC4)[nH]c3=O)sc12] | (R)-5-(7-((2-aminoethyl)amino)benzo[d]thiazol-2-yl)-2- morpholino-6-(piperidin-3-ylamino)pyrimidin-4(3H)-one |
| 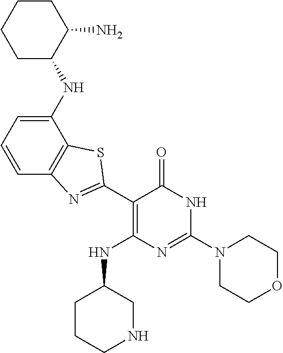 [SMILES: N[C@H]1CCCC[C@H]1Nc1cccc2nc(-c3c(N[C@@H]4CCCNC4)nc(N4CCOCC4)[nH]c3=O)sc12] | 5-(7-(((1R,2S)-2-aminocyclohexyl)amino)benzo[d]thiazol-2- yl)-2-morpholino-6-(((R)-piperidin-3-yl)amino)pyrimidin- 4(3H)-one |
| 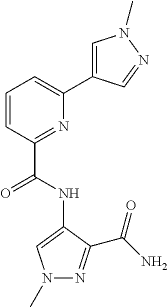 [SMILES: Cn1cc(-c2cccc(C(=O)Nc3cn(C)nc3C(N)=O)n2)cn1] | N-(3-carbamoyl-1-methyl-1H-pyrazol-4-yl)-6-(1-methyl- 1H-pyrazol-4-yl)picolinamide |
| 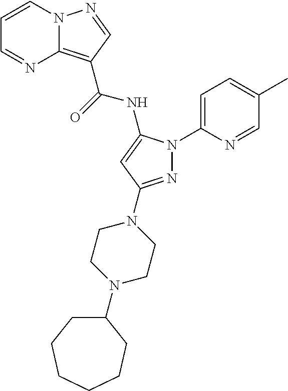 [SMILES: Cc1ccc(-n2nc(N3CCN(C4CCCCCC4)CC3)cc2NC(=O)c2cnn3cccnc23)nc1] | N-(3-(4-cycloheptylpiperazin-1-yl)-1-(5-methylpyridin-2- yl)-1H-pyrazol-5-yl)pyrazolo[1,5-a]pyrimidine-3- carboxamide |
| 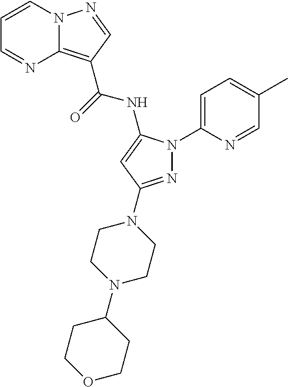 [SMILES: Cc1ccc(-n2nc(N3CCN(C4CCOCC4)CC3)cc2NC(=O)c2cnn3cccnc23)nc1] | N-(1-(5-methylpyridin-2-yl)-3-(4-(tetrahydro-2H-pyran-4- yl)piperazin-1-yl)-1H-pyrazol-5-yl)pyrazolo[1,5- a]pyrimidine-3-carboxamide |
| 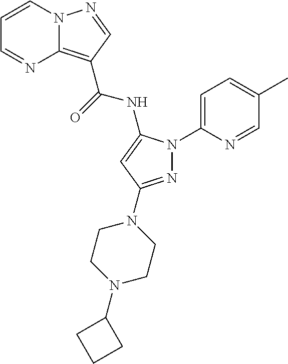 [SMILES: Cc1ccc(-n2nc(N3CCN(C4CCC4)CC3)cc2NC(=O)c2cnn3cccnc23)nc1] | N-(3-(4-cyclopiperazin-1-yl)-1-(5-methylpyridin-2-yl)- 1H-pyrazol-5-yl)pyrazolo[1,5-a]pyrimidine-3-carboxamide |
| 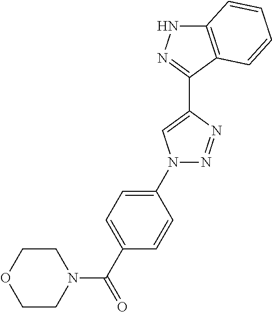 [SMILES: O=C(c1ccc(-n2cc(-c3n[nH]c4ccccc34)nn2)cc1)N1CCOCC1] | (4-(4-(1H-indazol-3-yl)-1H-1,2,3-triazol-1- yl)phenyl)(morpholino)methanone |
| 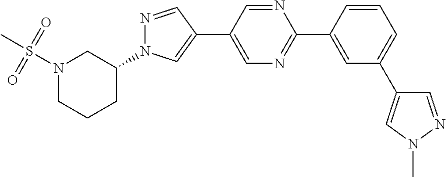 [SMILES: Cn1cc(-c2cccc(-c3ncc(-c4cnn([C@@H]5CCCN(S(C)(=O)=O)C5)c4)cn3)c2)cn1] | (R)-2-(3-(1-methyl-1H-pyrazol-4-yl)phenyl)-5-(1-(1- (methylsulfonyl)piperidin-3-yl)-1H-pyrazol-4-yl)pyrimidine |
| 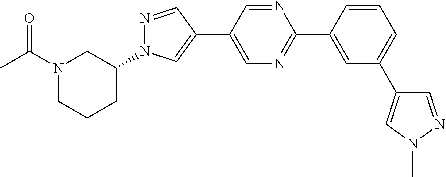 [SMILES: CC(=O)N1CCC[C@@H](n2cc(-c3cnc(-c4cccc(-c5cnn(C)c5)c4)nc3)cn2)C1] | (R)-1-(3-(4-(2-(3-(1-methyl-1H-pyrazol-4- yl)phenyl)pyrimidin-5-yl)-1H-pyrazol-1-yl)piperidin-1- yl)ethan-1-one |
| 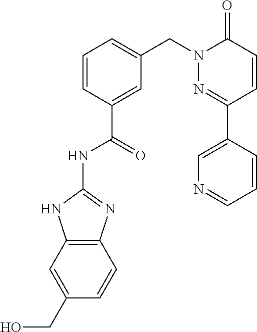 [SMILES: O=C(Nc1nc2ccc(CO)cc2[nH]1)c1cccc(Cn2nc(-c3cccnc3)ccc2=O)c1] | N-(6-(hydroxymethyl)-1H-benzo[d]imidazol-2-yl)-3-((6- oxo-3-(pyridin-3-yl)pyridazin-1(6H)-yl)methyl)benzamide |
| 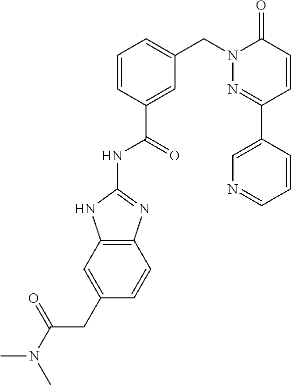 [SMILES: CN(C)C(=O)Cc1ccc2nc(NC(=O)c3cccc(Cn4nc(-c5cccnc5)ccc4=O)c3)[nH]c2c1] | N-(6-(2-(dimethylamino)-2-oxoethyl)-1H-benzo[d]imidazol- 2-yl)-3-((6-oxo-3-(pyridin-3-yl)pyridazin-1(6H)- yl)methyl)benzamide |
| 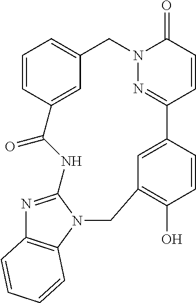 [SMILES: O=C1Nc2nc3ccccc3n2Cc2cc(ccc2O)-c2ccc(=O)n(n2)Cc2cccc1c2] | (E)-24-hydroxy-11,16-dihydro-41H-5-aza-4(1,2)- benzo[d]imidazola-1(3,1)-pyridazina-2,7(1,3)- dibenzenacyclooctaphane-16,6-dione |
| 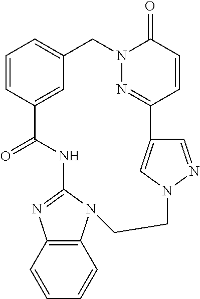 [SMILES: O=C1Nc2nc3ccccc3n2CCn2cc(cn2)-c2ccc(=O)n(n2)Cc2cccc1c2] | (14E,22E)-21,26-dihydro-11H,17H-6-aza-7(2,1)- benzo[d]imidazola-2(3,1)-pyridazina-1(4,1)-pyrazola-4(1,3)- benzenacyclononaphane-26,5-dione |
| 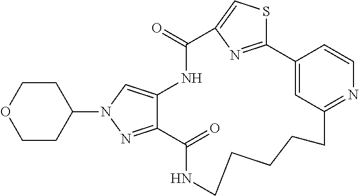 [SMILES: O=C1Nc2cn(C3CCOCC3)nc2C(=O)NCCCCCc2cc(ccn2)-c2nc1cs2] | (Z)-51-(tetrahydro-2H-pyran-4-yl)-51H-4,7-diaza-2(2,4)- thiazola-1(4,2)-pyridina-5(4,3)-pyrazolacyclododecaphane- 3,6-dione |
| 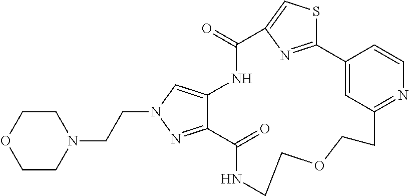 [SMILES: O=C1Nc2cn(CCN3CCOCC3)nc2C(=O)NCCOCCc2cc(ccn2)-c2nc1cs2] | (Z)-51-(2-morpholinoethyl)-51H-10-oxa-4,7-diaza-2(2,4)- thiazola-1(4,2)-pyridina-5(4,3)-pyrazolacyclododecaphane- 3,6-dione |
| 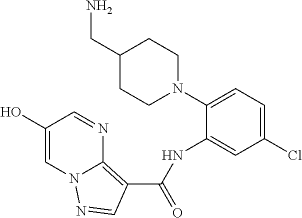 [SMILES: NCC1CCN(c2ccc(Cl)cc2NC(=O)c2cnn3cc(O)cnc23)CC1] | N-(2-(4-(aminomethyl)piperidin-1-yl)-5-chlorophenyl)-6- hydroxypyrazolo[1,5-a]pyrimidine-3-carboxamide |
| 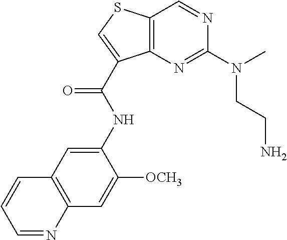 [SMILES: COc1cc2ncccc2cc1NC(=O)c1csc2cnc(N(C)CCN)nc12] | 2-((2-aminoethyl)(methyl)amino)-N-(7-methoxyquinolin-6- yl)thieno[3,2-d]pyrimidine-7-carboxamide |
| 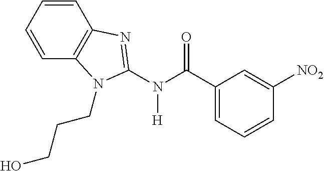 [SMILES: O=C(Nc1nc2ccccc2n1CCCO)c1cccc([N+](=O)[O-])c1] | N-(1-(3-hydroxypropyl)-1H-benzo[d]imidazol-2-yl)-3- nitrobenzamide |
| 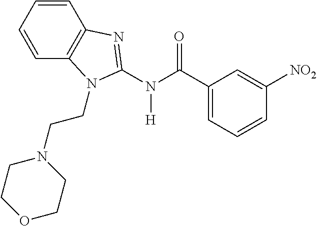 [SMILES: O=C(Nc1nc2ccccc2n1CCN1CCOCC1)c1cccc([N+](=O)[O-])c1] | N-(1-(2-morpholinoethyl)-1H-benzo[d]imidazol-2-yl)-3- nitrobenzamide |
| 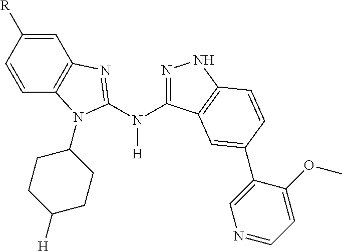 | See U.S. Pat. No. 8,293,923 for examples of R. |
| 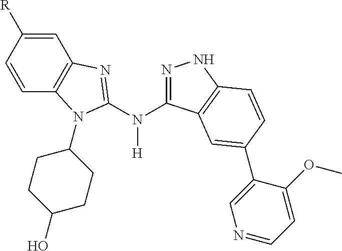 | See U.S. Pat. No. 8,293,923 for examples of R. |
| 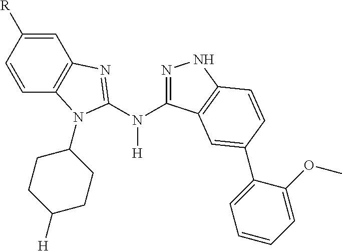 | See U.S. Pat. No. 8,293,923 for examples of R. |
| 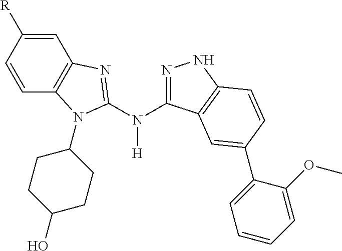 | See U.S. Pat. No. 8,293,923 for examples of R. |
| 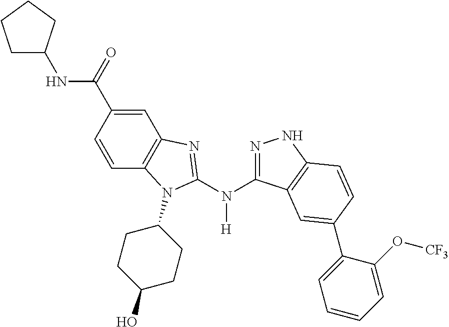 [SMILES: O=C(NC1CCCC1)c1ccc2c(c1)nc(Nc1n[nH]c3ccc(-c4ccccc4OC(F)(F)F)cc13)n2[C@H]1CC[C@H](O)CC1] | N-cyclopentyl-1-((1r,4r)-4-hydroxy-cyclohexyl)-2-((5-(2- (trifluoromethoxy)phenyl)-1H-indazol-3-yl)amino)-1H- benzo[d]imidazole-5-carboxamide |
| 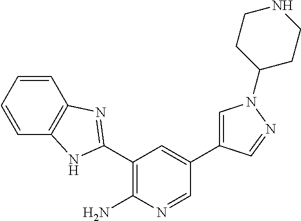 [SMILES: Nc1ncc(-c2cnn(C3CCNCC3)c2)cc1-c1nc2ccccc2[nH]1] | 3-(1H-benzo[d]imidazol-2-yl)-5-(1-(piperidin-4-yl)-1H- pyrazol-4-yl)pyridin-2-amine |
| 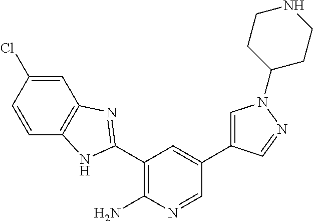 [SMILES: Nc1ncc(-c2cnn(C3CCNCC3)c2)cc1-c1nc2cc(Cl)ccc2[nH]1] | 3-(5-chloro-1H-benzo[d]imidazol-2-yl)-5-(1-(piperidin-4- yl)-1H-pyrazol-4-yl)pyridin-2-amine |
| 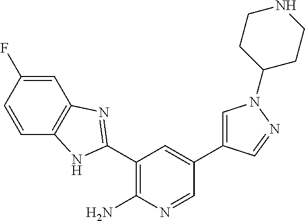 [SMILES: Nc1ncc(-c2cnn(C3CCNCC3)c2)cc1-c1nc2cc(F)ccc2[nH]1] | 3-(5-fluoro-1H-benzo[d]imidazol-2-yl)-5-(1-(piperidin-4- yl)-1H-pyrazol-4-yl)pyridin-2-amine |
| 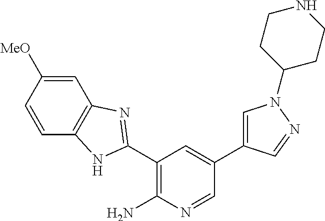 [SMILES: COc1ccc2[nH]c(-c3cc(-c4cnn(C5CCNCC5)c4)cnc3N)nc2c1] | 3-(5-methoxy-1H-benzo[d]imidazol-2-yl)-5-(1-(piperidin-4- yl)-1H-pyrazol-4-yl)pyridin-2-amine |
| 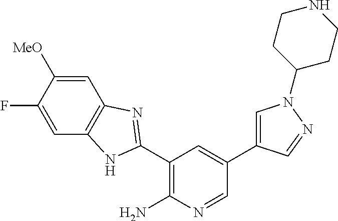 [SMILES: COc1cc2nc(-c3cc(-c4cnn(C5CCNCC5)c4)cnc3N)[nH]c2cc1F] | 3-(6-fluoro-5-methoxy-1H-benzo[d]imidazol-2-yl)-5-(1- (piperidin-4-yl)-1H-pyrazol-4-yl)pyridin-2-amine |
| 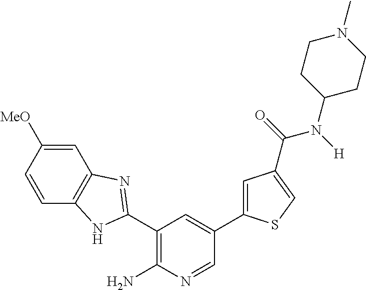 [SMILES: COc1ccc2[nH]c(-c3cc(-c4cc(C(=O)NC5CCN(C)CC5)cs4)cnc3N)nc2c1] | 5-(6-amino-5-(5-methoxy-1H-benzo[d]imidazol-2- yl)pyridin-3-yl)-N-(1-methylpiperidin-4-yl)thiophene-3- carboxamide |
| 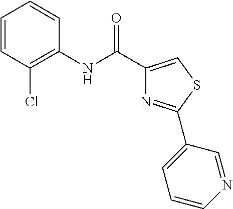 [SMILES: O=C(Nc1ccccc1Cl)c1csc(-c2cccnc2)n1] | N-(2-chlorophenyl)-2-(pyridin-3-yl)thiazole-4-carboxamide |
| 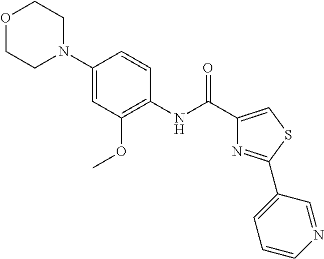 [SMILES: COc1cc(N2CCOCC2)ccc1NC(=O)c1csc(-c2cccnc2)n1] | N-(2-methoxy-4-morpholinophenyl)-2-(pyridin-3- yl)thiazole-4-carboxamide |
| 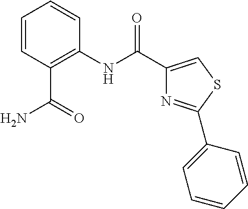 [SMILES: NC(=O)c1ccccc1NC(=O)c1csc(-c2ccccc2)n1] | N-(2-carbamoylphenyl)-2-phenylthiazole-4-carboxamide |
| 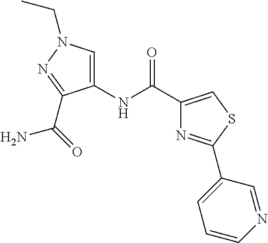 [SMILES: CCn1cc(NC(=O)c2csc(-c3cccnc3)n2)c(C(N)=O)n1] | N-(3-carbamoyl-1-ethyl-1H-pyrazol-4-yl)-2-(pyridin-4- yl)thiazole-4-carboxamide |
| 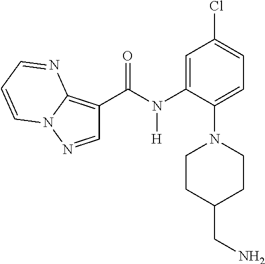 [SMILES: NCC1CCN(c2ccc(Cl)cc2NC(=O)c2cnn3cccnc23)CC1] | N-(2-(4-(aminomethyl)piperidin-1-yl)-5- chlorophenyl)pyrazolo[1,5-a]pyrimidine-3-carboxamide |
| 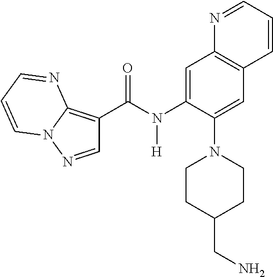 [SMILES: NCC1CCN(c2cc3cccnc3cc2NC(=O)c2cnn3cccnc23)CC1] | N-(6-(4-(aminomethyl)piperidin-1-yl)quinolin-7- yl)pyrazolo[1,5-a]pyrimidine-3-carboxamide |
| 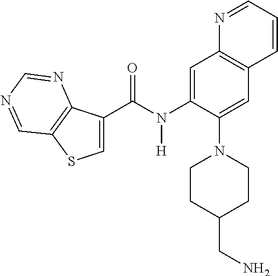 [SMILES: NCC1CCN(c2cc3cccnc3cc2NC(=O)c2csc3cncnc23)CC1] | N-(6-(4-(aminomethyl)piperidin-1-yl)quinolin-7- yl)thieno[3,2-d]pyrimidine-7-carboxamide |
| 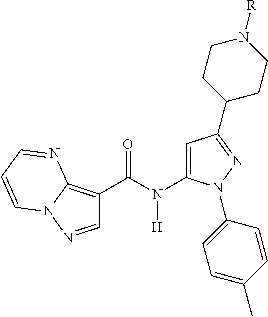 | R = H, alkyl cycloalkyl, CF₃CH₂, SO₂Me. See also WO 2012/129258. |
| 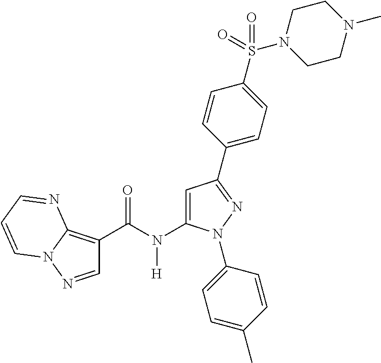 [SMILES: Cc1ccc(-n2nc(-c3ccc(S(=O)(=O)N4CCN(C)CC4)cc3)cc2NC(=O)c2cnn3cccnc23)cc1] | N-(3-(4-((4-methylpiperazin-1-yl)sulfonyl)phenyl)-1-(p- tolyl)-1H-pyrazol-5-yl)pyrazolo[1,5-a]pyrimidine-3- carboxamide |
| 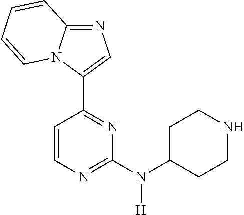 [SMILES: c1ccn2c(-c3ccnc(NC4CCNCC4)n3)cnc2c1] | 4-(imidazo[1,2-a]pyridin-3-yl)-N-(piperidin-4-yl)pyrimidin- 2-amine |
| 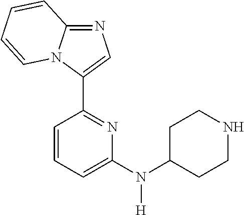 [SMILES: c1cc(NC2CCNCC2)nc(-c2cnc3ccccn23)c1] | 6-(imidazo[1,2-a]pyridin-3-yl)-N-(piperidin-4-yl)pyridin-2- amine |
| 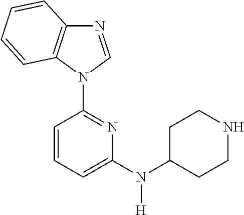 [SMILES: c1cc(NC2CCNCC2)nc(-n2cnc3ccccc32)c1] | 6-(1H-benzo[d]imidazol-1-yl)-N-(piperidin-4-yl)pyridin-2- amine |
| 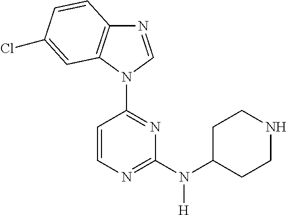 [SMILES: Clc1ccc2ncn(-c3ccnc(NC4CCNCC4)n3)c2c1] | 6-(6-chloro-1H-benzo[d]imidazol-1-yl)-N-(piperidin-4- yl)pyridin-2-amine |
| 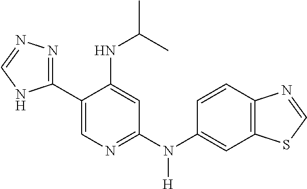 [SMILES: CC(C)Nc1cc(Nc2ccc3ncsc3c2)ncc1-c1nnc[nH]1] | N2-(benzo[d]thiazol-6-yl)-N4-isopropyl-5-(4H-1,2,4-triazol- 3-yl)pyridine-2,4-diamine |
| 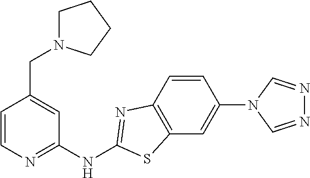 [SMILES: c1cc(CN2CCCC2)cc(Nc2nc3ccc(-n4cnnc4)cc3s2)n1] | N-(4-(pyrrolidin-1-ylmethyl)pyridin-2-yl)-6-(4H-1,2,4- triazol-4-yl)benzo[d]thiazol-2-amine |
| 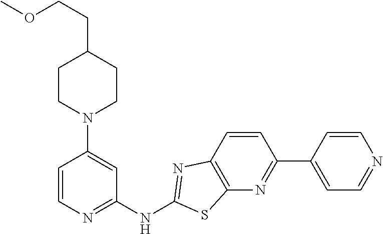 [SMILES: COCCC1CCN(c2ccnc(Nc3nc4ccc(-c5ccncc5)nc4s3)c2)CC1] | N-(4-(4-(2-methoxyethyl)piperidin-1-yl)pyridin-2-yl)-5- (pyridin-4-yl)thiazolo[5,4-b]pyridin-2-amine |
| 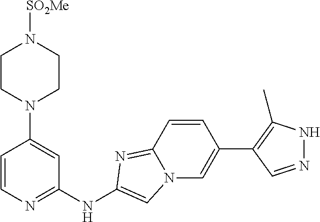 [SMILES: Cc1[nH]ncc1-c1ccc2nc(Nc3cc(N4CCN(S(C)(=O)=O)CC4)ccn3)cn2c1] | 6-(5-methyl-1H-pyrazol-4-yl)-N-(4-(4- (methylsulfonyl)piperazin-1-yl)pyridin-2-yl)imidazo[1,2- a]pyridin-2-amine |
| 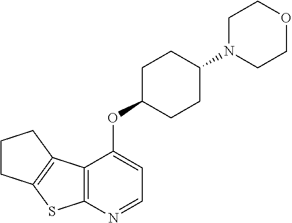 [SMILES: c1cc(O[C@H]2CC[C@H](N3CCOCC3)CC2)c2c3c(sc2n1)CCC3] | 4-((1r,4r)-4-((6,7-dihydro-5H-cyclopenta[4,5]thieno[2,3- b]pyridin-4-yl)oxy)cyclohexyl)morpholine |
| 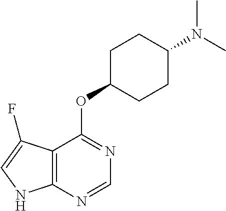 [SMILES: CN(C)[C@H]1CC[C@H](Oc2ncnc3[nH]cc(F)c23)CC1] | (1r,4r)-4-((5-fluoro-7H-pyrrolo[2,3-d]pyrimidin-4-yl)oxy)- N,N-dimethylcyclohexan-1-amine |
| 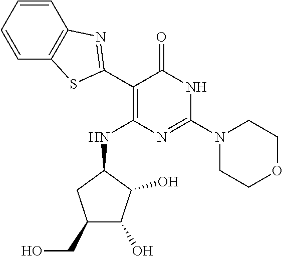 [SMILES: O=c1[nH]c(N2CCOCC2)nc(N[C@@H]2C[C@H](CO)[C@@H](O)[C@H]2O)c1-c1nc2ccccc2s1] | 5-(benzo[d]thiazol-2-yl)-6-(((1R,2S,3R,4R)-2,3-dihydroxy- 4-(hydroxymethyl)cyclopentyl)amino)-2- morpholinopyrimidin-4(3H)-one |
| 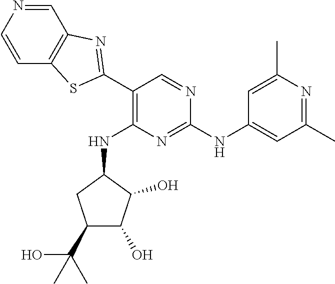 [SMILES: Cc1cc(Nc2ncc(-c3nc4cnccc4s3)c(N[C@@H]3C[C@H](C(C)(C)O)[C@@H](O)[C@H]3O)n2)cc(C)n1] | (1R,2S,3R,5S)-3-((2-((2,6-dimethylpyridin-4-yl)amino)-5- (thiazolo[4,5-c]pyridin-2-yl)pyrimidin-4-yl)amino)-5-(2- hydroxypropan-2-yl)cyclopentane-1,2-diol |
| 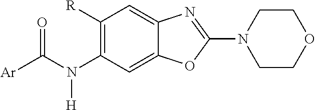 | Ar = 6-substituted pyridin-2-yl. R = small alkyl amines, hydrogen, pyrrolidines, piperidines, phenyl. See also WO 2013/042137. |
| 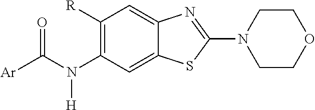 | Ar = 6-substituted pyridin-2-yl. R = small alkyl amines, hydrogen, pyrrolidines, piperidines, phenyl. See also WO 2013/042137. |
| 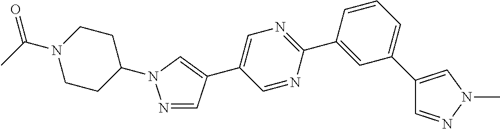 [SMILES: CC(=O)N1CCC(n2cc(-c3cnc(-c4cccc(-c5cnn(C)c5)c4)nc3)cn2)CC1] | 1-(4-(4-(2-(3-(1-methyl-1H-pyrazol-4-yl)phenyl)pyrimidin- 5-yl)-1H-pyrazol-1-yl)piperidin-1-yl)ethan-1-one |
| 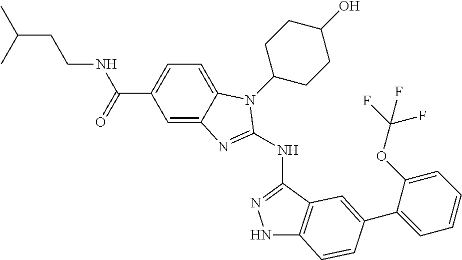 [SMILES: CC(C)CCNC(=O)c1ccc2c(c1)nc(Nc1n[nH]c3ccc(-c4ccccc4OC(F)(F)F)cc13)n2C1CCC(O)CC1] | 1-(4-hydroxycyclohexyl)-N-(3-methylbutyl)-2-[[5-[2- (trifluoromethoxy)phenyl]-1H-indazol-3- yl]amino]benzimidazole-5-carboxamide |

In an embodiment, the IRAK4 inhibitor is a compound selected from those disclosed in US 2015/0284405 A1, the disclosures of which are incorporated by reference herein, or a pharmaceutically acceptable salt, solvate, hydrate, cocrystal, or prodrug thereof. In an embodiment, the IRAK4 inhibitor is a compound selected from Examples 1-379 in Table 1 or Table 3 of US 2015/0284405 A1, the disclosures of which are incorporated by reference herein; or a pharmaceutically acceptable salt, solvate, hydrate, cocrystal, or prodrug thereof.

In an embodiment, the IRAK4 inhibitor is a compound of Formula (47):

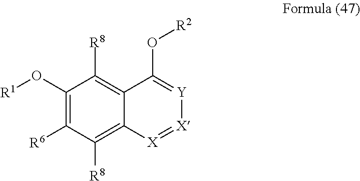

wherein X and X′ are each independently CR⁸, N or —N⁺—O⁻; Y is independently N, —N⁺—O⁻ or CR^{8′}; provided that at least one of X, X′ or Y is neither N nor —N⁺—O⁻ and that no more than one of X, X′ or Y is —N⁺—O⁻; R¹ is C₁-C₆alkyl; C₂-C₆alkenyl; C₂-C₆alkynyl; —(CR^{3a}R^{3b})ₘ-(3- to 7-membered cycloalkyl); —(CR^{3a}R^{3b})ₘ-(3- to 7-membered heterocycloalkyl) having one to three heteroatoms; —(CR^{3a}R^{3b})ₘ-(5- to 10-membered heteroaryl), having one to three heteroatoms; or —(CR^{3a}R^{3b})ₘ—C₆-C₁₂aryl; wherein said alkyl, alkenyl, alkynyl, cycloalkyl, heterocycloalkyl, heteroaryl or aryl is optionally substituted with one to five halogen, deuterium, —OR⁵, —SR⁵, —NR^{11a}R^{11b}, cyano, C₁-C₆alkyl, C₃-C₆cycloalkyl or —C₁-C₆alkoxy; R² is —(CR^{3a}R^{3b})ₘ-(3- to 10-membered cycloalkyl); —(CR^{3a}R^{3b})ₘ-(3- to 10-membered heterocycloalkyl) having one to three heteroatoms; —(CR^{3a}R^{3b})ₘ-(5- to 10 membered heteroaryl) having one to three heteroatoms; or —(CR^{3a}R^{3b})ₘ—C₆-C₁₂aryl; wherein said cycloalkyl, heterocycloalkyl, heteroaryl or aryl is optionally substituted with one to five R⁴; and wherein, if the heteroatom on said heterocycloalkyl and heteroaryl is N, said N is optionally substituted with R^{4′}; or R² is C₁-C₆alkyl, wherein said alkyl is optionally substituted with NH₂, OH or cyano; R^{3a} and R^{3b} for each occurrence are independently hydrogen or C₁-C₃alkyl; R⁴ for each occurrence is independently a bond, deuterium, halogen, cyano, C₁-C₆alkyl, C₂-C₆alkenyl, oxo, —OR⁵, —SR⁵, —S(O)R⁹, —S(O)₂R⁹, —NR^{a}R^{b}, —C(O)R¹⁰, —(CR^{3a}R^{3b})ₙ-(3- to 7-membered cycloalkyl), —(CR^{3a}R^{3b})ₙ-(4- to 10-membered heterocycloalkyl), having one to three heteroatoms, —(CR^{3a}R^{3b})ₙ-(5- to 10 membered heteroaryl), having one to three heteroatoms, or —(CR^{3a}R^{3b})ₙ—C₆-C₁₂aryl wherein said alkyl, cycloalkyl, heterocycloalkyl, heteroaryl or aryl is each optionally and independently substituted with one to five deuterium, halogen, OR⁵, —SR⁵, —NR^{11a}R^{11b}, cyano, C₁-C₆alkyl, C₃-C₆cycloalkyl or —C₁-C₆alkoxy; or two R⁴ taken together with the respective carbons to which each are bonded form a 3- to 6-membered cycloalkyl or 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano or C₁-C₆alkyl or C₁-C₆alkoxy, wherein the alkyl or alkoxy is optionally substituted with halogen, deuterium, —OR⁵, —SR⁵, —NR^{11a}R^{11b}, or cyano; and wherein, if a heteroatom on said heterocycloalkyl is N, said N is optionally substituted with R^{4′}; R^{4′} is independently C₁-C₆alkyl, C₂-C₆alkenyl, —C(O)R¹⁰, —S(O)₂R⁹, —(CR^{3a}R^{3b})ₙ-(3- to 7-membered cycloalkyl), —(CR^{3a}R^{3b})ₙ-(4- to 10-membered heterocycloalkyl) or C(O)(CH₂)ₜCN; wherein said alkyl, alkenyl, cycloalkyl, or heterocycloalkyl is each optionally and independently substituted with one to five deuterium, halogen, OH, cyano or C₁-C₆alkoxy; or R⁴ and R^{4′} taken together with the respective atoms to which each are bonded form a 3- to 6-membered cycloalkyl or 4- to 6-membered heterocycloalkyl, wherein said cycloalkyl or heterocycloalkyl is optionally substituted with one to three halogen, deuterium, —OR⁵, —SR⁵, —NR^{11a}R^{11b}, cyano, C₁-C₆alkyl or C₁-C₆alkoxy, wherein the alkyl or alkoxy is optionally substituted with halogen, deuterium, —OR⁵, —SR⁵, —NR^{11a}R^{11b}, or cyano; R⁵ is independently hydrogen or C₁-C₆alkyl, wherein said alkyl is optionally substituted with halogen, deuterium, C₁-C₆alkoxy, C₁-C₆alkylthiolyl, —NR^{11a}R^{11b}, cyano, C₁-C₆alkyl or C₃-C₆cycloalkyl; or two R⁵ taken together with the oxygen atoms to which they are bonded form a 5- or 6-membered heterocycloalkyl; R⁶ is —C(O)NHR⁷, CO₂R⁷ or cyano; R⁷ is hydrogen or C₁-C₆alkyl; each R⁸ is independently hydrogen, halogen, cyano, —OR⁵, —SR⁵, —NR^{11a}R^{11b}, C₁-C₆alkyl, C₃-C₆cycloalkyl, 3- to 10-membered heterocycloalkyl or 5- to 6-membered heteroaryl or aryl, wherein said alkyl, cycloalkyl, heterocycloalkyl, heteroaryl or aryl is optionally substituted with one to three halogen, —NR^{11a}R^{11b}, OR⁵, —SR⁵, cyano, C₁-C₃ alkyl, —C(O)R¹⁰ or oxo; R^{8′} is hydrogen, deuterium, halogen, cyano, —OR⁵, —SR⁵ or NR^{11a}NR^{11b}; R⁹ is —(CR^{3a}R^{3b})ₚ—(C₁-C₃alkyl), —(CR^{3a}R^{3b})ₚ-(4- to 6-membered cycloalkyl), —(CR^{3a}R^{3b})ₚ-(4- to 6-membered heterocycloalkyl) or —(CR^{3a}R^{3b})ₚ—(C5-C₉aryl), wherein said alkyl, cycloalkyl, heterocycloalkyl or aryl are each optionally substituted with fluoro or C₁-C₃alkyl; R¹⁰ is C₁-C₆alkyl, wherein said alkyl is optionally substituted with deuterium, halogen, OH, C₁-C₆alkoxy or cyano; R^{11a} and R^{11b} are each independently hydrogen or C₁-C₆alkyl, wherein said alkyl is optionally substituted with deuterium, C₁-C₆alkoxy or cyano; and if C₂-C₆alkyl, said alkyl is optionally substituted with deuterium, C₁-C₆alkoxy, cyano, halogen or OH; m is independently 0, 1, 2 or 3; n is independently 0, 1, 2 or 3; p is independently 0 or 1; and t is 1, 2 or 3;
or a pharmaceutically acceptable salt, solvate, hydrate, cocrystal, or prodrug thereof.

In an embodiment, the IRAK4 inhibitor is a compound selected from those listed in Table 2; or a pharmaceutically acceptable salt, solvate, hydrate, cocrystal, or prodrug thereof.

TABLE 2
IRAK4 inhibitors (IUPAC name).

4-(azetidin-3-ylmethoxy)-6-(propan-2-yloxy)quinoline-7-carboxamide
4-[(3S)-piperidin-3-ylmethoxy]-6-(propan-2-yloxy)quinoline-7-carboxamide
4-[(3R)-piperidin-3-ylmethoxy]-6-(propan-2-yloxy)quinoline-7-carboxamide
4-(piperidin-4-ylmethoxy)-6-(propan-2-yloxy)quinoline-7-carboxamide
4-[(1R,5S,6R)-3-azabicyclo[3.1.0]hex-6-ylmethoxy]-6-(propan-2-yloxy)quinoline-7-
carboxamide
4-(oxetan-3-ylmethoxy)-6-(propan-2-yloxy)quinoline-7-carboxamide
4-(cyclopentylmethoxy)-6-(propan-2-yloxy)quinoline-7-carboxamide
4-(1-cyclobutylethoxy)-6-(propan-2-yloxy)quinoline-7-carboxamide
4-(cyclobutylmethoxy)-6-(propan-2-yloxy)quinoline-7-carboxamide
6-(propan-2-yloxy)-4-(tetrahydrofuran-3-ylmethoxy)quinoline-7-carboxamide
6-(propan-2-yloxy)-4-(tetrahydrofuran-2-ylmethoxy)quinoline-7-carboxamide
4-[(3-methyloxetan-3-yl)methoxy]-6-(propan-2-yloxy)quinoline-7-carboxamide
4-[(1-methylcyclobutyl)methoxy]-6-(propan-2-yloxy)quinoline-7-carboxamide
4-[(2R)-bicyclo[2.2.1]hept-2-yloxy]-6-(propan-2-yloxy)quinoline-7-carboxamide
6-(propan-2-yloxy)-4-[(2R)-tetrahydrofuran-2-ylmethoxy]quinoline-7-carboxamide
4-(bicyclo[2.2.1]hept-2-yloxy)-6-(propan-2-yloxy)quinoline-7-carboxamide
6-(propan-2-yloxy)-4-(tricyclo[2.2.1.0~2,6~]hept-3-yloxy)quinoline-7-carboxamide
4-(1,3-dioxolan-4-ylmethoxy)-6-(propan-2-yloxy)quinoline-7-carboxamide
4-[(1S,2R)-bicyclo[2.2.1]hept-2-yloxy]-6-(propan-2-yloxy)quinoline-7-carboxamide
1-[(3aR,6aS)-octahydrocyclopenta[c]pyrrol-4-yloxy]-7-(propan-2-yloxy)isoquinoline-6-
carboxamide
4-[(3aR,6aS)-octahydrocyclopenta[c]pyrrol-4-yloxy]-6-(propan-2-yloxy)quinoline-7-
carboxamide
4-{[(3S)-1-(cyanoacetyl)pyrrolidin-3-yl]methoxy}-6-(propan-2-yloxy)quinoline-7-carboxamide
4-{[(3R)-1-(cyanoacetyl)pyrrolidin-3-yl]methoxy}-6-(propan-2-yloxy)quinoline-7-carboxamide
7-(propan-2-yloxy)-1-(tetrahydrofuran-3-ylmethoxy)isoquinoline-6-carboxamide
7-(propan-2-yloxy)-1-(tetrahydro-2H-pyran-2-ylmethoxy)isoquinoline-6-carboxamide
1-{[(2S)-5-oxopyrrolidin-2-yl]methoxy}-7-(propan-2-yloxy)isoquinoline-6-carboxamide
1-[(1,1-dioxido-1,2-thiazinan-3-yl)methoxy]-7-(propan-2-yloxy)isoquinoline-6-carboxamide
1-[(3S)-piperidin-3-ylmethoxy]-7-(propan-2-yloxy)isoquinoline-6-carboxamide
1-[(3-methyl-2-oxo-1,3-oxazolidin-4-yl)methoxy]-7-(propan-2-yloxy)isoquinoline-6-
carboxamide
7-(propan-2-yloxy)-1-[(2R)-tetrahydrofuran-2-ylmethoxy]isoquinoline-6-carboxamide
1-{[(2S)-1-methylpyrrolidin-2-yl]methoxy}-7-(propan-2-yloxy)isoquinoline-6-carboxamide
1-{[(2R)-5-oxopyrrolidin-2-yl]methoxy}-7-(propan-2-yloxy)isoquinoline-6-carboxamide
1-[(1-acetylpiperidin-4-yl)methoxy]-7-(propan-2-yloxy)isoquinoline-6-carboxamide
1-{[(3R,4R)-4-methoxypyrrolidin-3-yl]methoxy}-7-(propan-2-yloxy)isoquinoline-6-
carboxamide
1-[(2-oxo-1,3-oxazolidin-5-yl)methoxy]-7-(propan-2-yloxy)isoquinoline-6-carboxamide
7-(propan-2-yloxy)-1-(tetrahydro-2H-pyran-4-ylmethoxy)isoquinoline-6-carboxamide
1-[(2S)-morpholin-2-ylmethoxy]-7-(propan-2-yloxy)isoquinoline-6-carboxamide
1-[(4-fluoropiperidin-4-yl)methoxy]-7-(propan-2-yloxy)isoquinoline-6-carboxamide
1-(morpholin-2-ylmethoxy)-7-(propan-2-yloxy)isoquinoline-6-carboxamide
1-[(1S,5S)-3-azabicyclo[3.1.0]hex-1-ylmethoxy]-7-(propan-2-yloxy)isoquinoline-6-carboxamide
7-(propan-2-yloxy)-1-[(2R)-pyrrolidin-2-ylmethoxy]isoquinoline-6-carboxamide
1-[(1R,5S,6r)-3-azabicyclo[3.1.0]hex-6-ylmethoxy]-7-(propan-2-yloxy)isoquinoline-6-
carboxamide
1-(piperidin-2-ylmethoxy)-7-(propan-2-yloxy)isoquinoline-6-carboxamide
1-[(4-methylmorpholin-2-yl)methoxy]-7-(propan-2-yloxy)isoquinoline-6-carboxamide
1-[(1-methylpiperidin-3-yl)methoxy]-7-(propan-2-yloxy)isoquinoline-6-carboxamide
7-(propan-2-yloxy)-1-{[(3R,4R)-4-(trifluoromethyl)pyrrolidin-3-yl]methoxy}isoquinoline-6-
carboxamide
1-[(2R)-morpholin-2-ylmethoxy]-7-(propan-2-yloxy)isoquinoline-6-carboxamide
1-[(3R)-piperidin-3-ylmethoxy]-7-(propan-2-yloxy)isoquinoline-6-carboxamide
7-(propan-2-yloxy)-1-[(3S)-pyrrolidin-3-ylmethoxy]isoquinoline-6-carboxamide
6-(propan-2-yloxy)-4-[(3S)-pyrrolidin-3-ylmethoxy]quinoline-7-carboxamide
4-[(2S)-morpholin-2-ylmethoxy]-6-(propan-2-yloxy)quinoline-7-carboxamide
4-(7-azaspiro[3.5]non-1-yloxy)-6-(propan-2-yloxy)quinoline-7-carboxamide
4-[(2R)-morpholin-2-ylmethoxy]-6-(propan-2-yloxy)quinoline-7-carboxamide
4-[(4-fluoropiperidin-4-yl)methoxy]-6-(propan-2-yloxy)quinoline-7-carboxamide
4-{[(3R,4R)-3,4-dimethylpyrrolidin-3-yl]methoxy}-6-(propan-2-yloxy)quinoline-7-carboxamide
4-[(4-methylpiperidin-4-yl)methoxy]-6-(propan-2-yloxy)quinoline-7-carboxamide
4-{[(5R)-2-oxo-1,3-oxazolidin-5-yl]methoxy}-6-(propan-2-yloxy)quinoline-7-carboxamide
4-[(3-methylpiperidin-3-yl)methoxy]-6-(propan-2-yloxy)quinoline-7-carboxamide
4-(piperidin-3-ylmethoxy)-6-(propan-2-yloxy)quinoline-7-carboxamide
4-{[1-(cyanoacetyl)azetidin-3-yl]methoxy}-6-(propan-2-yloxy)quinoline-7-carboxamide
1-{[(2R)-1-(cyanoacetyl)pyrrolidin-2-yl]methoxy}-7-(propan-2-yloxy)isoquinoline-6-
carboxamide
1-{[1-(cyanoacetyl)piperidin-4-yl]methoxy}-7-(propan-2-yloxy)isoquinoline-6-carboxamide
1-{[(2S)-1-(cyanoacetyl)pyrrolidin-2-yl]methoxy}-7-(propan-2-yloxy)isoquinoline-6-
carboxamide
1-{[(3R)-4-(cyanoacetyl)morpholin-3-yl]methoxy}-7-(propan-2-yloxy)isoquinoline-6-
carboxamide
1-({1-[(cyanoacetyl)amino]cyclopentyl}methoxy)-7-(propan-2-yloxy)isoquinoline-6-
carboxamide
1-{[(3S)-1-(cyanoacetyl)pyrrolidin-3-yl]methoxy}-7-(propan-2-yloxy)isoquinoline-6-
carboxamide
1-{[(3R)-1-(cyanoacetyl)piperidin-3-yl]methoxy}-7-(propan-2-yloxy)isoquinoline-6-
carboxamide
1-{[(1R,5R,6R)-3-(cyanoacetyl)-3-azabicyclo[3.2.1]oct-6-yl]oxy}-7-(propan-2-
yloxy)isoquinoline-6-carboxamide
1-{[1-(cyanoacetyl)azetidin-3-yl]methoxy}-7-(propan-2-yloxy)isoquinoline-6-carboxamide
1-{[(3R)-1-(cyanoacetyl)pyrrolidin-3-yl]methoxy}-7-(propan-2-yloxy)isoquinoline-6-
carboxamide
4-{[(3aR,6aS)-2-(cyanoacetyl)octahydrocyclopenta[c]pyrrol-4-yl]oxy}-6-(propan-2-
yloxy)quinoline-7-carboxamide
1-[(1S,4R)-2-azabicyclo[2.2.1]hept-6-yloxy]-7-(propan-2-yloxy)isoquinoline-6-carboxamide
1-{[(2S)-1-(cyanoacetyl)azetidin-2-yl]methoxy}-7-(propan-2-yloxy)isoquinoline-6-carboxamide
1-{[(1S,4S,5S)-2-(cyanoacetyl)-2-azabicyclo[2.2.1]hept-5-yl]oxy}-7-(propan-2-
yloxy)isoquinoline-6-carboxamide
1-{[(2S)-4-(cyanoacetyl)morpholin-2-yl]methoxy}-7-(propan-2-yloxy)isoquinoline-6-
carboxamide
1-{[1-(cyanoacetyl)-4-fluoropiperidin-4-yl]methoxy}-7-(propan-2-yloxy)isoquinoline-6-
carboxamide
1-{[(1S,5S)-3-(cyanoacetyl)-3-azabicyclo[3.1.0]hex-1-yl]methoxy}-7-(propan-2-
yloxy)isoquinoline-6-carboxamide
1-{[(2R)-4-(cyanoacetyl)morpholin-2-yl]methoxy}-7-(propan-2-yloxy)isoquinoline-6-
carboxamide
1-{[(3aR,4S,6aS)-2-(cyanoacetyl)octahydrocyclopenta[c]pyrrol-4-yl]oxy}-7-(propan-2-
yloxy)isoquinoline-6-carboxamide
1-{[(3R,4R)-1-(cyanoacetyl)-4-ethylpyrrolidin-3-yl]methoxy}-7-(propan-2-yloxy)isoquinoline-
6-carboxamide
1-{[(1S,5S,6S)-3-(cyanoacetyl)-3-azabicyclo[3.2.1]oct-6-yl]oxy}-7-(propan-2-
yloxy)isoquinoline-6-carboxamide
1-{[1-(cyanoacetyl)-3-methylpyrrolidin-3-yl]methoxy}-7-(propan-2-yloxy)isoquinoline-6-
carboxamide
1-{[(3S)-4-(cyanoacetyl)morpholin-3-yl]methoxy}-7-(propan-2-yloxy)isoquinoline-6-
carboxamide
1-{[(3R,4R)-1-(cyanoacetyl)-4-methoxypyrrolidin-3-yl]methoxy}-7-(propan-2-
yloxy)isoquinoline-6-carboxamide
1-{[(3R,4R)-1-(cyanoacetyl)-4-methylpyrrolidin-3-yl]methoxy}-7-(propan-2-
yloxy)isoquinoline-6-carboxamide
1-{[1-(cyanoacetyl)-4-methylpiperidin-4-yl]methoxy}-7-(propan-2-yloxy)isoquinoline-6-
carboxamide
4-{[(1R,5S,6r)-3-(cyanoacetyl)-3-azabicyclo[3.1.0]hex-6-yl]methoxy}-6-(propan-2-
yloxy)quinoline-7-carboxamide
4-{[(3R,4R)-1-(cyanoacetyl)-4-methylpyrrolidin-3-yl]methoxy}-6-(propan-2-yloxy)quinoline-7-
carboxamide
4-{[(1R,5R,6R)-3-(cyanoacetyl)-3-azabicyclo[3.2.1]oct-6-yl]oxy}-6-(propan-2-yloxy)quinoline-
7-carboxamide
4-{[(1S,5S)-3-(cyanoacetyl)-3-azabicyclo[3.1.0]hex-1-yl]methoxy}-6-(propan-2-
yloxy)quinoline-7-carboxamide
4-{[1-(cyanoacetyl)-4-methylpiperidin-4-yl]methoxy}-6-(propan-2-yloxy)quinoline-7-
carboxamide
4-{[(1S,5S,6S)-3-(cyanoacetyl)-3-azabicyclo[3.2.1]oct-6-yl]oxy}-6-(propan-2-yloxy)quinoline-
7-carboxamide
4-{[(3S)-1-(cyanoacetyl)piperidin-3-yl]methoxy}-6-(propan-2-yloxy)quinoline-7-carboxamide
4-{[(1S,5S)-3-(cyanoacetyl)-3-azabicyclo[3.1.0]hex-1-yl]methoxy}-6-(propan-2-
yloxy)quinoline-7-carboxamide
4-{[1-(cyanoacetyl)-4-fluoropiperidin-4-yl]methoxy}-6-(propan-2-yloxy)quinoline-7-
carboxamide
4-{[(3R,4R)-1-(cyanoacetyl)-4-methoxypyrrolidin-3-yl]methoxy}-6-(propan-2-yloxy)quinoline-
7-carboxamide
4-{[(3R)-1-(cyanoacetyl)piperidin-3-yl]methoxy}-6-(propan-2-yloxy)quinoline-7-carboxamide
4-{[(2S)-4-(cyanoacetyl)morpholin-2-yl]methoxy}-6-(propan-2-yloxy)quinoline-7-carboxamide
4-{[1-(cyanoacetyl)piperidin-2-yl]methoxy}-6-(propan-2-yloxy)quinoline-7-carboxamide
4-{[1-(cyanoacetyl)piperidin-4-yl]methoxy}-6-(propan-2-yloxy)quinoline-7-carboxamide
1-[(1S,4S,5S)-2-azabicyclo[2.2.1]hept-5-yloxy]-7-(propan-2-yloxy)isoquinoline-6-carboxamide
1-[(1R,4R,5R)-2-azabicyclo[2.2.1]hept-5-yloxy]-7-(propan-2-yloxy)isoquinoline-6-carboxamide
1-{[(2S)-5-oxopyrrolidin-2-yl]methoxy}-7-(propan-2-yloxy)isoquinoline-6-carbonitrile
1-{[(4R)-2-oxo-1,3-oxazolidin-4-yl]methoxy}-7-(propan-2-yloxy)isoquinoline-6-carboxamide
4-methyl-1-{[(2S)-5-oxopyrrolidin-2-yl]methoxy}-7-(propan-2-yloxy)isoquinoline-6-
carboxamide
1-{[(2S)-6-oxopiperidin-2-yl]methoxy}-7-(propan-2-yloxy)isoquinoline-6-carboxamide
1-{[(2S)-4-methyl-5-oxopyrrolidin-2-yl]methoxy}-7-(propan-2-yloxy)isoquinoline-6-
carboxamide
1-[(1S,4R,6R)-2-azabicyclo[2.2.1]hept-6-yloxy]-7-(propan-2-yloxy)isoquinoline-6-carboxamide
1-[(1S,4R,6S)-2-azabicyclo[2.2.1]hept-6-yloxy]-7-(propan-2-yloxy)isoquinoline-6-carboxamide
1-{[(2S)-4,4-dimethyl-5-oxopyrrolidin-2-yl]methoxy}-7-(propan-2-yloxy)isoquinoline-6-
carboxamide
1-[(5-oxopyrrolidin-3-yl)methoxy]-7-(propan-2-yloxy)isoquinoline-6-carboxamide
1-{[(2S)-4-fluoro-5-oxopyrrolidin-2-yl]methoxy}-7-(propan-2-yloxy)isoquinoline-6-
carboxamide
1-{[(2S)-4-ethyl-5-oxopyrrolidin-2-yl]methoxy}-7-(propan-2-yloxy)isoquinoline-6-carboxamide
5-{[(2S)-5-oxopyrrolidin-2-yl]methoxy}-3-(propan-2-yloxy)naphthalene-2-carboxamide
3-(propan-2-yloxy)-5-[(3R)-pyrrolidin-3-ylmethoxy]naphthalene-2-carboxamide
1-[(5-oxomorpholin-3-yl)methoxy]-7-(propan-2-yloxy)isoquinoline-6-carboxamide
7-(propan-2-yloxy)-1-[(3R)-pyrrolidin-3-ylmethoxy]isoquinoline-6-carboxamide
1-[(3-oxooctahydro-1H-isoindol-1-yl)methoxy]-7-(propan-2-yloxy)isoquinoline-6-carboxamide
1-[(2S)-azetidin-2-ylmethoxy]-7-(propan-2-yloxy)isoquinoline-6-carboxamide
7-(cyclobutyloxy)-1-{[(2S)-5-oxopyrrolidin-2-yl]methoxy}isoquinoline-6-carboxamide
7-methoxy-1-{[(2S)-5-oxopyrrolidin-2-yl]methoxy}isoquinoline-6-carboxamide
7-ethoxy-1-{[(2S)-5-oxopyrrolidin-2-yl]methoxy}isoquinoline-6-carboxamide
1-[(3aR,6aR)-hexahydrocyclopenta[c]pyrrol-3a(1H)-ylmethoxy]-7-(propan-2-
yloxy)isoquinoline-6-carboxamide
1-{[(2S,4R)-4-methyl-5-oxopyrrolidin-2-yl]methoxy}-7-(propan-2-yloxy)isoquinoline-6-
carboxamide
1-{[(2S,4S)-4-methyl-5-oxopyrrolidin-2-yl]methoxy}-7-(propan-2-yloxy)isoquinoline-6-
carboxamide
1-{[(2S)-2-methyl-5-oxopyrrolidin-2-yl]methoxy}-7-(propan-2-yloxy)isoquinoline-6-
carboxamide
1-{[(2S)-4-(methoxymethyl)-5-oxopyrrolidin-2-yl]methoxy}-7-(propan-2-yloxy)isoquinoline-6-
carboxamide
1-{[(2S)-4,4-difluoro-5-oxopyrrolidin-2-yl]methoxy}-7-(propan-2-yloxy)isoquinoline-6-
carboxamide
7-(difluoromethoxy)-1-{[(2S)-5-oxopyrrolidin-2-yl]methoxy}isoquinoline-6-carboxamide
1-{[(3aS,6R,6aR)-2-oxooctahydrocyclopenta[b]pyrrol-6-yl]oxy}-7-(propan-2-
yloxy)isoquinoline-6-carboxamide
1-{[(2S,4S)-4-ethyl-4-fluoro-5-oxopyrrolidin-2-yl]methoxy}-7-(propan-2-yloxy)isoquinoline-6-
carboxamide
1-{[(2S,4R)-4-ethyl-4-fluoro-5-oxopyrrolidin-2-yl]methoxy}-7-(propan-2-yloxy)isoquinoline-6-
carboxamide
1-{[(2S,4R)-4-fluoro-4-methyl-5-oxopyrrolidin-2-yl]methoxy}-7-(propan-2-yloxy)isoquinoline-
6-carboxamide
1-{[(2S,4S)-4-fluoro-4-methyl-5-oxopyrrolidin-2-yl]methoxy}-7-(propan-2-yloxy)isoquinoline-
6-carboxamide
1-{[(2S,4S)-4-ethyl-5-oxopyrrolidin-2-yl]methoxy}-7-(propan-2-yloxy)isoquinoline-6-
carboxamide
1-{[(2S,4R)-4-ethyl-5-oxopyrrolidin-2-yl]methoxy}-7-(propan-2-yloxy)isoquinoline-6-
carboxamide
1-{[(2S,4R)-4-(2-hydroxypropan-2-yl)-5-oxopyrrolidin-2-yl]methoxy}-7-(propan-2-
yloxy)isoquinoline-6-carboxamide
1-[(2-oxopiperidin-4-yl)methoxy]-7-(propan-2-yloxy)isoquinoline-6-carboxamide
1-[(1S,5S)-3-azabicyclo[3.1.0]hex-1-ylmethoxy]-7-(propan-2-yloxy)isoquinoline-6-carboxamide
1-[(1R,5R)-3-azabicyclo[3.1.0]hex-1-ylmethoxy]-7-(propan-2-yloxy)isoquinoline-6-
carboxamide
1-{[(2S,3S)-3-methyl-5-oxopyrrolidin-2-yl]methoxy}-7-(propan-2-yloxy)isoquinoline-6-
carboxamide
1-[(6-oxopiperidin-3-yl)methoxy]-7-(propan-2-yloxy)isoquinoline-6-carboxamide
1-[(1,1-dioxido-1,2-thiazolidin-3-yl)methoxy]-7-(propan-2-yloxy)isoquinoline-6-carboxamide
1-{[(2S,4R)-4-fluoro-5-oxopyrrolidin-2-yl]methoxy}-7-(propan-2-yloxy)isoquinoline-6-
carboxamide
1-{[(2S,4S)-4-fluoro-5-oxopyrrolidin-2-yl]methoxy}-7-(propan-2-yloxy)isoquinoline-6-
carboxamide
1-{[(2S)-4,4-difluoro-5-oxopyrrolidin-2-yl]methoxy}-7-methoxyisoquinoline-6-carboxamide
1-{[2-(hydroxymethyl)-5-oxopyrrolidin-2-yl]methoxy}-7-(propan-2-yloxy)isoquinoline-6-
carboxamide
1-{[(2S)-4-(hydroxymethyl)-5-oxopyrrolidin-2-yl]methoxy}-7-(propan-2-yloxy)isoquinoline-6-
carboxamide
1-{[(2S,3S)-3-amino-5-oxopyrrolidin-2-yl]methoxy}-7-(propan-2-yloxy)isoquinoline-6-
carboxamide
1-{[(2S,4S)-4-hydroxy-5-oxopyrrolidin-2-yl]methoxy}-7-(propan-2-yloxy)isoquinoline-6-
carboxamide
1-{[(2S,4S)-5-oxo-4-(2,2,2-trifluoroethyl)pyrrolidin-2-yl]methoxy}-7-(propan-2-
yloxy)isoquinoline-6-carboxamide
1-{[(2S,4R)-5-oxo-4-(2,2,2-trifluoroethyl)pyrrolidin-2-yl]methoxy}-7-(propan-2-
yloxy)isoquinoline-6-carboxamide
1-{[(2S)-2-(hydroxymethyl)-5-oxopyrrolidin-2-yl]methoxy}-7-(propan-2-yloxy)isoquinoline-6-
carboxamide
1-{[(2R)-2-(hydroxymethyl)-5-oxopyrrolidin-2-yl]methoxy}-7-(propan-2-yloxy)isoquinoline-6-
carboxamide
1-{[(2S,3S)-5-oxo-3-(trifluoromethyl)pyrrolidin-2-yl]methoxy}-7-(propan-2-yloxy)isoquinoline-
6-carboxamide
1-{(1R)-1-[(2S)-5-oxopyrrolidin-2-yl]ethoxy}-7-(propan-2-yloxy)isoquinoline-6-carboxamide
1-{(1S)-1-[(2S)-5-oxopyrrolidin-2-yl]ethoxy}-7-(propan-2-yloxy)isoquinoline-6-carboxamide
1-[(3R)-morpholin-3-ylmethoxy]-7-(propan-2-yloxy)isoquinoline-6-carboxamide
7-(propan-2-yloxy)-1-(pyrrolidin-2-ylmethoxy)isoquinoline-6-carboxamide
1-[(3S)-morpholin-3-ylmethoxy]-7-(propan-2-yloxy)isoquinoline-6-carboxamide
1-[(1R,6S)-3-azabicyclo[4.1.0]hept-1-ylmethoxy]-7-(propan-2-yloxy)isoquinoline-6-
carboxamide
1-[(1S,6R)-3-azabicyclo[4.1.0]hept-1-ylmethoxy]-7-(propan-2-yloxy)isoquinoline-6-
carboxamide
1-{[(2S,4S)-4-fluoro-4-(hydroxymethyl)-5-oxopyrrolidin-2-yl]methoxy}-7-(propan-2-
yloxy)isoquinoline-6-carboxamide
1-{[(2S,4R)-4-fluoro-4-(hydroxymethyl)-5-oxopyrrolidin-2-yl]methoxy}-7-(propan-2-
yloxy)isoquinoline-6-carboxamide
1-{[(2S,4R)-4-(hydroxymethyl)-5-oxopyrrolidin-2-yl]methoxy}-7-(propan-2-yloxy)isoquinoline-
6-carboxamide
1-{[(2S,4S)-4-(hydroxymethyl)-5-oxopyrrolidin-2-yl]methoxy}-7-(propan-2-yloxy)isoquinoline-
6-carboxamide
1-{[(3aR,4R,6aR)-2,2-dimethyl-6-oxotetrahydro-3aH-[1,3]dioxolo[4,5-c]pyrrol-4-yl]methoxy}-
7-(propan-2-yloxy)isoquinoline-6-carboxamide
4-fluoro-1-{[(2S)-5-oxopyrrolidin-2-yl]methoxy}-7-(propan-2-yloxy)isoquinoline-6-
carboxamide
1-{[(2S)-5-oxopyrrolidin-2-yl]methoxy}-7-(propan-2-yloxy)isoquinoline-6-carboxylic acid
1-{[(2S,3S,4R)-4-fluoro-3-methyl-5-oxopyrrolidin-2-yl]methoxy}-7-(propan-2-
yloxy)isoquinoline-6-carboxamide
1-{[(2S,4R)-4-fluoro-4-(2-hydroxypropan-2-yl)-5-oxopyrrolidin-2-yl]methoxy}-7-(propan-2-
yloxy)isoquinoline-6-carboxamide
3-methoxy-5-{[(2S)-5-oxopyrrolidin-2-yl]methoxy}naphthalene-2-carboxamide
1-{[(1S,2S,5R)-4-oxo-3-azabicyclo[3.1.0]hex-2-yl]methoxy}-7-(propan-2-yloxy)isoquinoline-6-
carboxamide
1-{[(1S,5S)-4-oxo-3-azabicyclo[3.1.0]hex-1-yl]methoxy}-7-(propan-2-yloxy)isoquinoline-6-
carboxamide
8-fluoro-1-{[(2S)-5-oxopyrrolidin-2-yl]methoxy}-7-(propan-2-yloxy)isoquinoline-6-
carboxamide
1-{[(2S,4S)-4-fluoro-4-methyl-5-oxopyrrolidin-2-yl]methoxy}-7-methoxyisoquinoline-6-
carboxamide
1-{[(2S)-5-oxopyrrolidin-2-yl]methoxy}-7-(trifluoromethoxy)isoquinoline-6-carboxamide
1-{[(2S,4R)-4-hydroxy-5-oxopyrrolidin-2-yl]methoxy}-7-(propan-2-yloxy)isoquinoline-6-
carboxamide
1-{[(2S)-1-methyl-5-oxopyrrolidin-2-yl]methoxy}-7-(propan-2-yloxy)isoquinoline-6-
carboxamide
1-{[(2S)-4-(4-hydroxytetrahydro-2H-pyran-4-yl)-5-oxopyrrolidin-2-yl]methoxy}-7-(propan-2-
yloxy)isoquinoline-6-carboxamide
1-{[(2S,4R)-4-hydroxy-5-oxo-4-(2,2,2-trifluoroethyl)pyrrolidin-2-yl]methoxy}-7-(propan-2-
yloxy)isoquinoline-6-carboxamide
1-{[(2S,3S)-4,4-difluoro-3-methyl-5-oxopyrrolidin-2-yl]methoxy}-7-(propan-2-
yloxy)isoquinoline-6-carboxamide
1-{[(2S,4S)-4-hydroxy-5-oxo-4-(2,2,2-trifluoroethyl)pyrrolidin-2-yl]methoxy}-7-(propan-2-
yloxy)isoquinoline-6-carboxamide
1-{[(4S)-1-methyl-2-oxoimidazolidin-4-yl]methoxy}-7-(propan-2-yloxy)isoquinoline-6-
carboxamide
1-{[(5S,6R)-2-oxo-1-azaspiro[4.4]non-6-yl]oxy}-7-(propan-2-yloxy)isoquinoline-6-carboxamide
1-{[(2S,3S,4S)-4-fluoro-3-methyl-5-oxopyrrolidin-2-yl]methoxy}-7-(propan-2-
yloxy)isoquinoline-6-carboxamide
1-{[(2S,3S,4R)-4-fluoro-3-methyl-5-oxopyrrolidin-2-yl]methoxy}-7-methoxyisoquinoline-6-
carboxamide
1-{[(2S,3S,4S)-4-fluoro-3-methyl-5-oxopyrrolidin-2-yl]methoxy}-7-methoxyisoquinoline-6-
carboxamide
4-cyano-1-{[(2S)-5-oxopyrrolidin-2-yl]methoxy}-7-(propan-2-yloxy)isoquinoline-6-
carboxamide
7-(propan-2-yloxy)-1-(pyrrolidin-3-ylmethoxy)isoquinoline-6-carboxamide
1-{[(2S,4R)-4-(3-hydroxyoxetan-3-yl)-5-oxopyrrolidin-2-yl]methoxy}-7-(propan-2-
yloxy)isoquinoline-6-carboxamide
1-{[(2S,4S)-4-(3-hydroxyoxetan-3-yl)-5-oxopyrrolidin-2-yl]methoxy}-7-(propan-2-
yloxy)isoquinoline-6-carboxamide
5-{[(2S,4S)-4-fluoro-4-methyl-5-oxopyrrolidin-2-yl]methoxy}-3-methoxynaphthalene-2-
carboxamide
4-(aminomethyl)-1-{[(2S)-5-oxopyrrolidin-2-yl]methoxy}-7-(propan-2-yloxy)isoquinoline-6-
carboxamide
1-{[(2R,3R,4S)-3-ethyl-4-fluoro-3-hydroxy-5-oxopyrrolidin-2-yl]methoxy}-7-
methoxyisoquinoline-6-carboxamide
1-{[(3S,4S)-3-ethyl-4-fluoro-2-hydroxy-5-oxopyrrolidin-2-yl]methoxy}-7-methoxyisoquinoline-
6-carboxamide
1-{[(2S,3R,4S)-4-fluoro-3-(1-hydroxyethyl)-5-oxopyrrolidin-2-yl]methoxy}-7-
methoxyisoquinoline-6-carboxamide
7-(oxetan-3-yloxy)-1-{[(2S)-5-oxopyrrolidin-2-yl]methoxy}isoquinoline-6-carboxamide
7-tert-butoxy-1-{[(2S)-5-oxopyrrolidin-2-yl]methoxy}isoquinoline-6-carboxamide
1-{[(2S,3S)-3-ethyl-4,4-difluoro-5-oxopyrrolidin-2-yl]methoxy}-7-(propan-2-
yloxy)isoquinoline-6-carboxamide
1-{[(4S)-2-oxoimidazolidin-4-yl]methoxy}-7-(propan-2-yloxy)isoquinoline-6-carboxamide
1-{[(2S,3R,4S)-4-fluoro-3-methyl-5-oxopyrrolidin-2-yl]methoxy}-7-methoxyisoquinoline-6-
carboxamide
7-(cyclopropylmethoxy)-1-{[(2S)-5-oxopyrrolidin-2-yl]methoxy}isoquinoline-6-carboxamide
1-{[(2S,4R)-4-fluoro-4-(hydroxymethyl)-5-oxopyrrolidin-2-yl]methoxy}-7-
methoxyisoquinoline-6-carboxamide
6-methoxy-4-{[(2S)-5-oxopyrrolidin-2-yl]methoxy}isoquinoline-7-carboxamide
5-{[(2S,4R)-4-fluoro-4-(hydroxymethyl)-5-oxopyrrolidin-2-yl]methoxy}-3-
methoxynaphthalene-2-carboxamide
1-{[(2S,3S,4R)-4-fluoro-4-(hydroxymethyl)-3-methyl-5-oxopyrrolidin-2-yl]methoxy}-7-
methoxyisoquinoline-6-carboxamide
6-methoxy-4-{[(2S)-5-oxopyrrolidin-2-yl]methoxy}quinazoline-7-carboxamide
1-{[(2S,3S,4S)-4-fluoro-4-(hydroxymethyl)-3-methyl-5-oxopyrrolidin-2-yl]methoxy}-7-
methoxyisoquinoline-6-carboxamide
7-methoxy-1-{[(2S,3R)-3-methyl-5-oxopyrrolidin-2-yl]methoxy}isoquinoline-6-carboxamide
1-{[(2S,3S)-3-(hydroxymethyl)-5-oxopyrrolidin-2-yl]methoxy}-7-(propan-2-yloxy)isoquinoline-
6-carboxamide
1-{[(1S,3aS,6aR)-5-methyl-3-oxooctahydropyrrolo[3,4-c]pyrrol-1-yl]methoxy}-7-(propan-2-
yloxy)isoquinoline-6-carboxamide
1-{[(2S,3S)-3-(hydroxymethyl)-5-oxopyrrolidin-2-yl]methoxy}-7-methoxyisoquinoline-6-
carboxamide
1-{[(2S,4S)-4-fluoro-4-(fluoromethyl)-5-oxopyrrolidin-2-yl]methoxy}-7-methoxyisoquinoline-
6-carboxamide
3-methoxy-5-{[(2S,3R)-3-methyl-5-oxopyrrolidin-2-yl]methoxy}naphthalene-2-carboxamide
5-{[(2S,3S,4S)-4-fluoro-3-methyl-5-oxopyrrolidin-2-yl]methoxy}-3-methoxynaphthalene-2-
carboxamide
1-{[(2S,3R)-3-methyl-5-oxopyrrolidin-2-yl]methoxy}-7-(propan-2-yloxy)isoquinoline-6-
carboxamide
8-fluoro-5-{[(2S,3S,4S)-4-fluoro-3-methyl-5-oxopyrrolidin-2-yl]methoxy}-3-
methoxynaphthalene-2-carboxamide
1-{[(2S)-3,3-dimethyl-5-oxopyrrolidin-2-yl]methoxy}-7-methoxyisoquinoline-6-carboxamide
1-{[(2R)-3,3-dimethyl-5-oxopyrrolidin-2-yl]methoxy}-7-methoxyisoquinoline-6-carboxamide
1-{[(2R)-3,3-dimethyl-5-oxopyrrolidin-2-yl]methoxy}-7-(propan-2-yloxy)isoquinoline-6-
carboxamide
1-{[(2S,4R)-4-(cyanomethyl)-5-oxopyrrolidin-2-yl]methoxy}-7-methoxyisoquinoline-6-
carboxamide
1-{[(2S,4S)-4-(cyanomethyl)-5-oxopyrrolidin-2-yl]methoxy}-7-methoxyisoquinoline-6-
carboxamide
1-{[(1S,3aS,6aR)-3-oxooctahydropyrrolo[3,4-c]pyrrol-1-yl]methoxy}-7-(propan-2-
yloxy)isoquinoline-6-carboxamide
7-methoxy-1-{[(4R,5R)-5-methyl-2-oxo-1,3-oxazolidin-4-yl]methoxy}isoquinoline-6-
carboxamide
3-methoxy-5-{[(4R,5R)-5-methyl-2-oxo-1,3-oxazolidin-4-yl]methoxy}naphthalene-2-
carboxamide
7-methoxy-1-{[(4S)-2-oxo-1,3-oxazolidin-4-yl]methoxy}isoquinoline-6-carboxamide
7-methoxy-1-{[(4R)-2-oxo-1,3-oxazolidin-4-yl]methoxy}isoquinoline-6-carboxamide
3-methoxy-5-{[(2S,4S)-4-methoxy-5-oxopyrrolidin-2-yl]methoxy}naphthalene-2-carboxamide
3-methoxy-5-{[(2S,4R)-4-methoxy-5-oxopyrrolidin-2-yl]methoxy}naphthalene-2-carboxamide
3-methoxy-5-{[(4R)-2-oxo-1,3-oxazolidin-4-yl]methoxy}naphthalene-2-carboxamide
3-methoxy-5-{[(4R,5S)-5-methyl-2-oxo-1,3-oxazolidin-4-yl]methoxy}naphthalene-2-
carboxamide
7-methoxy-1-{[(5R)-2-oxo-1,3-oxazolidin-5-yl]methoxy}isoquinoline-6-carboxamide
7-methoxy-1-{[(4R,5S)-5-methyl-2-oxo-1,3-oxazolidin-4-yl]methoxy}isoquinoline-6-
carboxamide
1-{[(2S,3S,4R)-4-fluoro-3,4-dimethyl-5-oxopyrrolidin-2-yl]methoxy}-7-methoxyisoquinoline-6-
carboxamide
1-{[(2S,3S,4S)-4-fluoro-3,4-dimethyl-5-oxopyrrolidin-2-yl]methoxy}-7-methoxyisoquinoline-6-
carboxamide
7-methoxy-1-{[(5R)-3-methyl-2-oxo-1,3-oxazolidin-5-yl]methoxy}isoquinoline-6-carboxamide
5-{[(2S,4R)-4-fluoro-5-oxo-4-(2,2,2-trifluoroethyl)pyrrolidin-2-yl]methoxy}-3-
methoxynaphthalene-2-carboxamide
5-{[(2S,4S)-4-fluoro-5-oxo-4-(2,2,2-trifluoroethyl)pyrrolidin-2-yl]methoxy}-3-
methoxynaphthalene-2-carboxamide
7-methoxy-1-{[(2S,4S)-4-methyl-5-oxopyrrolidin-2-yl]methoxy}isoquinoline-6-carboxamide
7-methoxy-1-{[(2S,4R)-4-methyl-5-oxopyrrolidin-2-yl]methoxy}isoquinoline-6-carboxamide
1-{[(2S,3S)-3-ethyl-5-oxopyrrolidin-2-yl]methoxy}-7-methoxyisoquinoline-6-carboxamide
7-methoxy-1-{[(6S)-4-oxo-5-azaspiro[2.4]hept-6-yl]methoxy}isoquinoline-6-carboxamide
1-{[(2S,3R)-3-ethyl-5-oxopyrrolidin-2-yl]methoxy}-7-methoxyisoquinoline-6-carboxamide
1-{[(2S,3R,4S)-3,4-dimethyl-5-oxopyrrolidin-2-yl]methoxy}-7-methoxyisoquinoline-6-
carboxamide
1-{[(2S,3R,4R)-3,4-dimethyl-5-oxopyrrolidin-2-yl]methoxy}-7-methoxyisoquinoline-6-
carboxamide
1-{[(2S,3S)-3-(fluoromethyl)-5-oxopyrrolidin-2-yl]methoxy}-7-methoxyisoquinoline-6-
carboxamide
4-{[(2S,3S,4S)-4-fluoro-3-methyl-5-oxopyrrolidin-2-yl]methoxy}-6-methoxyquinoline-7-
carboxamide
1-{[(2S,3S)-3-ethenyl-5-oxopyrrolidin-2-yl]methoxy}-7-methoxyisoquinoline-6-carboxamide
1-{[(2S,4S)-4-fluoro-5-oxopyrrolidin-2-yl]methoxy}-7-methoxyisoquinoline-6-carboxamide
1-{[(2S,4R)-4-fluoro-5-oxopyrrolidin-2-yl]methoxy}-7-methoxyisoquinoline-6-carboxamide
1-{[(2S,3S,4S)-3-(fluoromethyl)-4-methyl-5-oxopyrrolidin-2-yl]methoxy}-7-
methoxyisoquinoline-6-carboxamide
7-methoxy-1-{[(2R)-5-oxopyrrolidin-2-yl]methoxy}isoquinoline-6-carboxamide
1-{[(2S,4R)-4-ethyl-4-fluoro-5-oxopyrrolidin-2-yl]methoxy}-7-methoxyisoquinoline-6-
carboxamide
1-{[(2S,4S)-4-ethyl-4-fluoro-5-oxopyrrolidin-2-yl]methoxy}-7-methoxyisoquinoline-6-
carboxamide
1-{[(2S)-4-benzyl-5-oxopyrrolidin-2-yl]methoxy}-7-methoxyisoquinoline-6-carboxamide
4-{[(2S,4S)-4-ethyl-4-fluoro-5-oxopyrrolidin-2-yl]methoxy}-6-methoxyquinoline-7-
carboxamide
4-{[(2S,4R)-4-ethyl-4-fluoro-5-oxopyrrolidin-2-yl]methoxy}-6-methoxyquinoline-7-
carboxamide
1-{[(2S,4R)-4-fluoro-4-(fluoromethyl)-5-oxopyrrolidin-2-yl]methoxy}-7-methoxyisoquinoline-
6-carboxamide
6-methoxy-4-{[(2S)-5-oxopyrrolidin-2-yl]methoxy}quinoline-7-carboxamide
1-{[(1R,2S,5S)-6,6-dimethyl-4-oxo-3-azabicyclo[3.1.0]hex-2-yl]methoxy}-7-
methoxyisoquinoline-6-carboxamide
7-methoxy-1-{[(1S,2S,5R)-4-oxo-3-azabicyclo[3.1.0]hex-2-yl]methoxy}isoquinoline-6-
carboxamide
7-methoxy-1-[(3-methyl-5-oxomorpholin-3-yl)methoxy]isoquinoline-6-carboxamide
7-methoxy-1-[(4-methyl-2-oxo-1,3-oxazolidin-4-yl)methoxy]isoquinoline-6-carboxamide
7-methoxy-1-{[(2S,4S)-5-oxo-4-(2,2,2-trifluoroethyl)pyrrolidin-2-yl]methoxy}isoquinoline-6-
carboxamide
4-{[(2S,4S)-4-fluoro-4-methyl-5-oxopyrrolidin-2-yl]methoxy}-6-methoxyquinoline-7-
carboxamide
4-{[(2S,4S)-4-fluoro-4-methyl-5-oxopyrrolidin-2-yl]methoxy}-6-(propan-2-yloxy)quinoline-7-
carboxamide
7-methoxy-1-{[(2S,4R)-5-oxo-4-(2,2,2-trifluoroethyl)pyrrolidin-2-yl]methoxy}isoquinoline-6-
carboxamide
7-methoxy-1-{[(1S,2S,5R)-6-methyl-4-oxo-3-azabicyclo[3.1.0]hex-2-yl]methoxy}isoquinoline-
6-carboxamide
1-{[(2S,3S,4S)-4-fluoro-3-methyl-5-oxopyrrolidin-2-yl]methoxy}-7-methoxyisoquinoline-6-
carbonitrile
1-(cyclopentylmethoxy)-7-methoxyisoquinoline-6-carboxamide
4-{[(2S,4R)-4-fluoro-4-(2-fluoroethyl)-5-oxopyrrolidin-2-yl]methoxy}-6-methoxyquinoline-7-
carboxamide
1-{[(2R,4R)-4-fluoro-5-oxopyrrolidin-2-yl]methoxy}-7-methoxyisoquinoline-6-carboxamide
1-{[(2S,4R)-4-fluoro-4-(2-fluoroethyl)-5-oxopyrrolidin-2-yl]methoxy}-7-methoxyisoquinoline-
6-carboxamide
7-methoxy-1-{[(2R,3S)-3-methyl-5-oxopyrrolidin-2-yl]methoxy}isoquinoline-6-carboxamide
7-ethoxy-1-{[(2S,4S)-4-fluoro-4-methyl-5-oxopyrrolidin-2-yl]methoxy}isoquinoline-6-
carboxamide
6-ethoxy-4-{[(2S,3S,4S)-4-fluoro-3-methyl-5-oxopyrrolidin-2-yl]methoxy}quinoline-7-
carboxamide
6-ethoxy-4-{[(1S,2S,5R)-4-oxo-3-azabicyclo[3.1.0]hex-2-yl]methoxy}quinoline-7-carboxamide
7-(cyclopropyloxy)-1-{[(2S,3S,4S)-4-fluoro-3-methyl-5-oxopyrrolidin-2-
yl]methoxy}isoquinoline-6-carboxamide
7-ethoxy-1-{[(1S,2S,5R)-4-oxo-3-azabicyclo[3.1.0]hex-2-yl]methoxy}isoquinoline-6-
carboxamide
7-ethoxy-1-{[(2S,3S,4S)-4-fluoro-3-methyl-5-oxopyrrolidin-2-yl]methoxy}isoquinoline-6-
carboxamide
1-{[(2S,4R)-4-fluoro-5-oxo-4-(tetrahydro-2H-pyran-4-yl)pyrrolidin-2-yl]methoxy}-7-
methoxyisoquinoline-6-carboxamide
7-methoxy-1-(((1R,2S,5R,6R)-6-methyl-4-oxo-3-azabicyclo[3.1.0]hexan-2-
yl)methoxy)isoquinoline-6-carboxamide
7-methoxy-1-{[(2S,4S)-5-oxo-4-(tetrahydro-2H-pyran-4-yl)pyrrolidin-2-
yl]methoxy}isoquinoline-6-carboxamide
6-ethoxy-4-{[(2S,4S)-4-fluoro-4-methyl-5-oxopyrrolidin-2-yl]methoxy}quinoline-7-
carboxamide
1-{[(2R,3R,4R)-4-fluoro-3-methyl-5-oxopyrrolidin-2-yl]methoxy}-7-methoxyisoquinoline-6-
carboxamide
1-{[(2S,4S)-4-(4-hydroxytetrahydro-2H-pyran-4-yl)-5-oxopyrrolidin-2-yl]methoxy}-7-
methoxyisoquinoline-6-carboxamide
7-methoxy-1-{[(2S)-4-(oxetan-3-ylidene)-5-oxopyrrolidin-2-yl]methoxy}isoquinoline-6-
carboxamide
6-ethoxy-4-{[(2S)-5-oxopyrrolidin-2-yl]methoxy}quinoline-7-carboxamide
1-{[(2S,4R)-4-fluoro-4-(methoxymethyl)-5-oxopyrrolidin-2-yl]methoxy}-7-
methoxyisoquinoline-6-carboxamide
6-ethoxy-4-{[(2S,4S)-4-fluoro-4-(fluoromethyl)-5-oxopyrrolidin-2-yl]methoxy}quinoline-7-
carboxamide
7-ethoxy-1-{[(2S,4S)-4-fluoro-4-(fluoromethyl)-5-oxopyrrolidin-2-yl]methoxy}isoquinoline-6-
carboxamide
7-methoxy-1-{[(1S,2S,5R)-1-methyl-4-oxo-3-azabicyclo[3.1.0]hex-2-yl]methoxy}isoquinoline-
6-carboxamide
1-{[(2S,3S,4S)-3-ethyl-4-fluoro-5-oxopyrrolidin-2-yl]methoxy}-7-methoxyisoquinoline-6-
carboxamide
7-methoxy-1-{[(1S,2S,5R)-5-methyl-4-oxo-3-azabicyclo[3.1.0]hex-2-yl]methoxy}isoquinoline-
6-carboxamide
7-methoxy-1-{[(2S,4S)-4-(oxetan-3-yl)-5-oxopyrrolidin-2-yl]methoxy}isoquinoline-6-
carboxamide
1-{[(1S,2S,5R)-1-ethyl-4-oxo-3-azabicyclo[3.1.0]hex-2-yl]methoxy}-7-methoxyisoquinoline-6-
carboxamide
1-{[(1S,2S,5R)-6-ethyl-4-oxo-3-azabicyclo[3.1.0]hex-2-yl]methoxy}-7-methoxyisoquinoline-6-
carboxamide
1-(((1R,2S,5R,6R)-6-ethyl-4-oxo-3-azabicyclo[3.1.0]hexan-2-yl)methoxy)-7-
methoxyisoquinoline-6-carboxamide
7-methoxy-1-{[(2S)-6-oxopiperidin-2-yl]methoxy}isoquinoline-6-carboxamide
1-(((1S,2S,5S,6R)-6-(fluoromethyl)-4-oxo-3-azabicyclo[3.1.0]hexan-2-yl)methoxy)-7-
methoxyisoquinoline-6-carboxamide
1-{[(1R,2S,5S)-6-(fluoromethyl)-4-oxo-3-azabicyclo[3.1.0]hex-2-yl]methoxy}-7-
methoxyisoquinoline-6-carboxamide
1-{[(2S,3S)-3-cyclopropyl-5-oxopyrrolidin-2-yl]methoxy}-7-methoxyisoquinoline-6-
carboxamide
1-(((1R,2S,5R,6R)-6-(2-fluoroethyl)-4-oxo-3-azabicyclo[3.1.0]hexan-2-yl)methoxy)-7-
methoxyisoquinoline-6-carboxamide
7-methoxy-1-{[(1R,2S,5S)-4-oxo-3-azabicyclo[3.1.0]hex-2-yl]methoxy}isoquinoline-6-
carboxamide
7-methoxy-1-{[(1S,2S,5R)-4-oxo-3-azabicyclo[3.2.0]hept-2-yl]methoxy}isoquinoline-6-
carboxamide
1-{[(1R,2S,5S)-5-fluoro-6-methyl-4-oxo-3-azabicyclo[3.1.0]hex-2-yl]methoxy}-7-
methoxyisoquinoline-6-carboxamide
1-(((1S,2S,5S,6R)-5-fluoro-6-methyl-4-oxo-3-azabicyclo[3.1.0]hexan-2-yl)methoxy)-7-
methoxyisoquinoline-6-carboxamide
1-{[(1S,2S,5R)-6,6-dichloro-4-oxo-3-azabicyclo[3.1.0]hex-2-yl]methoxy}-7-
methoxyisoquinoline-6-carboxamide
7-methoxy-1-{[(2S,3R)-5-oxo-3-propylpyrrolidin-2-yl]methoxy}isoquinoline-6-carboxamide
7-methoxy-1-{[(1S,2S,5S)-6-(methoxymethyl)-4-oxo-3-azabicyclo[3.1.0]hex-2-
yl]methoxy}isoquinoline-6-carboxamide
7-methoxy-1-{[(1S,2S,5S)-6-(methoxymethyl)-4-oxo-3-azabicyclo[3.1.0]hex-2-
yl]methoxy}isoquinoline-6-carboxamide
1-{[(1S,2S,5R)-6-fluoro-4-oxo-3-azabicyclo[3.1.0]hex-2-yl]methoxy}-7-methoxyisoquinoline-6-
carboxamide
4-{[(2S,3S,4S)-3-ethyl-4-fluoro-5-oxopyrrolidin-2-yl]methoxy}-6-methoxyquinoline-7-
carboxamide
1-{[(1R,2S,5S)-5-fluoro-4-oxo-3-azabicyclo[3.1.0]hex-2-yl]methoxy}-7-methoxyisoquinoline-6-
carboxamide
1-{[(2S)-5-oxopyrrolidin-2-yl]methoxy}-7-(prop-2-yn-1-yloxy)isoquinoline-6-carboxamide
1-{[(2S)-5-oxopyrrolidin-2-yl]methoxy}-7-(propadienyloxy)isoquinoline-6-carboxamide
1-{[(1R,2S,5S)-6-(difluoromethyl)-4-oxo-3-azabicyclo[3.1.0]hex-2-yl]methoxy}-7-
methoxyisoquinoline-6-carboxamide
3-chloro-6-methoxy-4-{[(1S,2S,5R)-6-methyl-4-oxo-3-azabicyclo[3.1.0]hex-2-
yl]methoxy}quinoline-7-carboxamide
1-{[(1R,2S,5S)-5-fluoro-4-oxo-3-azabicyclo[3.2.0]hept-2-yl]methoxy}-7-methoxyisoquinoline-
6-carboxamide
4-{[(1R,2S,5S)-5-fluoro-6-methyl-4-oxo-3-azabicyclo[3.1.0]hex-2-yl]methoxy}-6-
methoxyquinoline-7-carboxamide
4-{[(1S,2S,5R)-6-fluoro-4-oxo-3-azabicyclo[3.1.0]hex-2-yl]methoxy}-6-methoxyquinoline-7-
carboxamide
4-{[(1R,2S,5S)-5-fluoro-4-oxo-3-azabicyclo[3.1.0]hex-2-yl]methoxy}-6-methoxyquinoline-7-
carboxamide
6-methoxy-4-{[(1S,2S,5R)-4-oxo-3-azabicyclo[3.1.0]hex-2-yl]methoxy}quinoline-7-
carboxamide
1-{[(1S,2S,5S)-6-(hydroxymethyl)-4-oxo-3-azabicyclo[3.1.0]hex-2-yl]methoxy}-7-
methoxyisoquinoline-6-carboxamide
1-{[(1S,2S,5S)-6-(hydroxymethyl)-4-oxo-3-azabicyclo[3.1.0]hex-2-yl]methoxy}-7-
methoxyisoquinoline-6-carboxamide
1-{[(2S,3R)-3-ethenyl-5-oxopyrrolidin-2-yl]methoxy}-7-methoxyisoquinoline-6-carboxamide
7-methoxy-1-{[(4S)-6-oxo-5-azaspiro[2.4]hept-4-yl]methoxy}isoquinoline-6-carboxamide
4-{[(1R,2S,5S)-6-(fluoromethyl)-4-oxo-3-azabicyclo[3.1.0]hex-2-yl]methoxy}-6-
methoxyquinoline-7-carboxamide
1-(((1R,2S,5R,6R)-6-fluoro-4-oxo-3-azabicyclo[3.1.0]hexan-2-yl)methoxy)-7-
methoxyisoquinoline-6-carboxamide
1-{[(1S,2S,5R)-6-fluoro-6-methyl-4-oxo-3-azabicyclo[3.1.0]hex-2-yl]methoxy}-7-
methoxyisoquinoline-6-carboxamide
1-{[(1S,2S,5R)-6-fluoro-6-methyl-4-oxo-3-azabicyclo[3.1.0]hex-2-yl]methoxy}-7-
methoxyisoquinoline-6-carboxamide
1-{[(2S,3S,4R)-3-ethyl-4-fluoro-5-oxopyrrolidin-2-yl]methoxy}-7-methoxyisoquinoline-6-
carboxamide
1-{[(2S,3S,4S)-3-ethyl-4-fluoro-5-oxopyrrolidin-2-yl]methoxy}-7-
[(trideutero)methyloxy]isoquinoline-6-carboxamide
1-{[(2S,3S,4S)-3-ethyl-4-fluoro-5-oxopyrrolidin-2-yl]methoxy}-7-(2-
methoxyethoxy)isoquinoline-6-carboxamide
7-methoxy-1-{[(2S,3R)-3-(methoxymethyl)-5-oxopyrrolidin-2-yl]methoxy}isoquinoline-6-
carboxamide
4-{[(2S,3S,4S)-3-ethyl-4-fluoro-5-oxopyrrolidin-2-yl]methoxy}-6-methoxyquinazoline-7-
carboxamide
1-{[(2S,3S,4S)-3-ethyl-4-methoxy-5-oxopyrrolidin-2-yl]methoxy}-7-methoxyisoquinoline-6-
carboxamide
1-{[(2S,3S,4R)-3-ethyl-4-methoxy-5-oxopyrrolidin-2-yl]methoxy}-7-methoxyisoquinoline-6-
carboxamide
1-{[(2S,3S,4S)-3-(pentadeutero)ethyl-4-fluoro-5-oxopyrrolidin-2-yl]methoxy}-7-
methoxyisoquinoline-6-carboxamide
1-{[(2S,3S)-3-ethyl-4,4-difluoro-5-oxopyrrolidin-2-yl]methoxy}-7-methoxyisoquinoline-6-
carboxamide
1-{[(2S,3R,4R)-3-ethyl-4-fluoro-5-oxopyrrolidin-2-yl]methoxy}-7-methoxyisoquinoline-6-
carboxamide
1-{[(2S,3R)-4,4-difluoro-3-(methoxymethyl)-5-oxopyrrolidin-2-yl]methoxy}-7-
methoxyisoquinoline-6-carboxamide
1-{[(2S,3R,4S)-4-fluoro-3-(methoxymethyl)-5-oxopyrrolidin-2-yl]methoxy}-7-
methoxyisoquinoline-6-carboxamide
7-methoxy-1-{[(2S,3S,4S)-4-methoxy-3-methyl-5-oxopyrrolidin-2-yl]methoxy}isoquinoline-6-
carboxamide
7-methoxy-1-{[(2S,3S,4R)-4-methoxy-3-methyl-5-oxopyrrolidin-2-yl]methoxy}isoquinoline-6-
carboxamide
1-{[(2S,3R,4R)-4-fluoro-3-(methoxymethyl)-5-oxopyrrolidin-2-yl]methoxy}-7-
methoxyisoquinoline-6-carboxamide
1-{[(2S,3S,4S)-3-ethyl-4-hydroxy-5-oxopyrrolidin-2-yl]methoxy}-7-methoxyisoquinoline-6-
carboxamide
1-{[(2S,3S,4R)-3-ethyl-4-hydroxy-5-oxopyrrolidin-2-yl]methoxy}-7-methoxyisoquinoline-6-
carboxamide
7-methoxy-1-{(1S)-1-[(2S)-5-oxopyrrolidin-2-yl]ethoxy}isoquinoline-6-carboxamide
1-{[(2S,3S)-3-(2-fluoroethyl)-5-oxopyrrolidin-2-yl]methoxy}-7-methoxyisoquinoline-6-
carboxamide
1-{[(2S,3R,4S)-4-amino-3-ethyl-5-oxopyrrolidin-2-yl]methoxy}-7-methoxyisoquinoline-6-
carboxamide
1-{[(2S,3R,4R)-4-amino-3-ethyl-5-oxopyrrolidin-2-yl]methoxy}-7-methoxyisoquinoline-6-
carboxamide
1-{[(2S,3S,4S)-3-ethyl-4-fluoro-5-oxopyrrolidin-2-yl]methoxy}-7-(propan-2-
yloxy)isoquinoline-6-carboxamide
7-ethoxy-1-{[(2S,3S,4S)-3-ethyl-4-fluoro-5-oxopyrrolidin-2-yl]methoxy}isoquinoline-6-
carboxamide
1-{[(2S,3S,4S)-3-ethyl-4-fluoro-5-oxopyrrolidin-2-yl]methoxy}-4-fluoro-7-
methoxyisoquinoline-6-carboxamide
1-{[(2S,3S,4S)-3-ethyl-4-fluoro-5-oxopyrrolidin-2-yl]methoxy}-8-fluoro-7-
methoxyisoquinoline-6-carboxamide
1-{[(2S,3R)-3-ethyl-5-oxopyrrolidin-2-yl]methoxy}-4-fluoro-7-methoxyisoquinoline-6-
carboxamide
1-{[(2S,3R)-3-ethyl-5-oxopyrrolidin-2-yl]methoxy}-8-fluoro-7-methoxyisoquinoline-6-
carboxamide
4-fluoro-7-methoxy-1-{[(1S,2S,5R)-6-methyl-4-oxo-3-azabicyclo[3.1.0]hex-2-
yl]methoxy}isoquinoline-6-carboxamide
8-fluoro-7-methoxy-1-{[(1S,2S,5R)-6-methyl-4-oxo-3-azabicyclo[3.1.0]hex-2-
yl]methoxy}isoquinoline-6-carboxamide
1-{[(2S,3R)-3-(fluoromethyl)-5-oxopyrrolidin-2-yl]methoxy}-7-methoxyisoquinoline-6-
carboxamide
1-{[(2S,3R,4S)-4-fluoro-3-(fluoromethyl)-5-oxopyrrolidin-2-yl]methoxy}-7-
methoxyisoquinoline-6-carboxamide
1-{[(2S,3S,4S)-3-cyclopropyl-4-fluoro-5-oxopyrrolidin-2-yl]methoxy}-7-methoxyisoquinoline-
6-carboxamide
1-{[(2S,3S,4R)-3-cyclopropyl-4-fluoro-5-oxopyrrolidin-2-yl]methoxy}-7-methoxyisoquinoline-
6-carboxamide
1-{[(2S,3S,4S)-4-fluoro-3-(2-fluoroethyl)-5-oxopyrrolidin-2-yl]methoxy}-7-
methoxyisoquinoline-6-carboxamide
4-(1-methyl-1H-imidazol-4-yl)-1-{[(2S)-5-oxopyrrolidin-2-yl]methoxy}-7-(propan-2-
yloxy)isoquinoline-6-carboxamide
4-(1,2-dimethyl-1H-imidazol-4-yl)-1-{[(2S)-5-oxopyrrolidin-2-yl]methoxy}-7-(propan-2-
yloxy)isoquinoline-6-carboxamide
4-(2-methyl-1H-imidazol-4-yl)-1-{[(2S)-5-oxopyrrolidin-2-yl]methoxy}-7-(propan-2-
yloxy)isoquinoline-6-carboxamide
1-{[(2S,3S,4S)-3-ethyl-4-fluoro-5-oxo(3,4-bisdeutero)pyrrolidin-2-yl]methoxy}-7-
methoxyisoquinoline-6-carboxamide
1-{[(2S,3R,4R)-4-fluoro-3-(fluoromethyl)-5-oxopyrrolidin-2-yl]methoxy}-7-
methoxyisoquinoline-6-carboxamide
4-(4-methylpyrimidin-2-yl)-1-{[(2S)-5-oxopyrrolidin-2-yl]methoxy}-7-(propan-2-
yloxy)isoquinoline-6-carboxamide
4-(5-chloropyrimidin-2-yl)-1-{[(2S)-5-oxopyrrolidin-2-yl]methoxy}-7-(propan-2-
yloxy)isoquinoline-6-carboxamide
4-(6-oxo-1,6-dihydropyridin-2-yl)-1-{[(2S)-5-oxopyrrolidin-2-yl]methoxy}-7-(propan-2-
yloxy)isoquinoline-6-carboxamide
4-(2-methylpyrimidin-4-yl)-1-{[(2S)-5-oxopyrrolidin-2-yl]methoxy}-7-(propan-2-
yloxy)isoquinoline-6-carboxamide

In an embodiment, the IRAK4 inhibitor is 1-{[(2S)-5-oxopyrrolidin-2-yl]methoxy}-7-(propan-2-yloxy)isoquinoline-6-carboxamide:

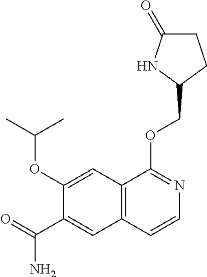

or a pharmaceutically acceptable salt, solvate, hydrate, cocrystal, or prodrug thereof.

In an embodiment, the IRAK4 inhibitor is a compound of Formula (48):

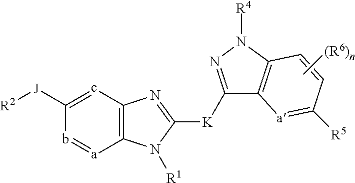

or a pharmaceutically acceptable salt, solvate, hydrate, cocrystal, or prodrug thereof.
R¹ is an optionally substituted aliphatic, an optionally substituted cycloaliphatic, an optionally substituted heterocycloaliphatic, an optionally substituted aryl, or an optionally substituted heteroaryl;
R² is H, an optionally substituted aliphatic, an optionally substituted cycloaliphatic, an optionally substituted heterocycloaliphatic, an optionally substituted aryl, or an optionally substituted heteroaryl;
Each of a, a′, b, and c is independently N or C(R³);
Each of R³, R⁵, and R⁶ is independently H, an optionally substituted aliphatic, an optionally substituted alkoxy, acyl, halo, hydroxy, amino, nitro, cyano, guanadino, amidino, carboxy, sulfo, mercapto, sulfanyl, sulfonyl, sulfonyl, sulfonamide, amido, sulfamide, urea, thiourea, carbamoyl, cycloaliphatic, cycloalkyloxy, heterocycloaliphatic, heterocycloalkyloxy, aryl, aralkyl, aryloxy, aroyl, heteroaryl, heteroaralkyl, heteroaryloxy, or heteroaroyl;

### R⁴ is H;

K is —O—, —S(O)ᵢ—, —N(R^{X′})—, —C(O)—, or C(R^{X′})(R^{Y′})—;
J is a bond, —O—, —S(O)ᵢ—, —N(R^{X′})—, alkylene, —C(O)—, —C(O)—O—, —CO—NR^{X′}, —(CH₂)ₚ—N(R^{X′})—, or —N(R^{X′})—C(O)—;
Each of R^{X′} and R^{Y′} is independently H or an optionally substituted aliphatic;
n is 0, 1, 2, or 3;
i is 0, 1, or 2; and
p is 1, 2, 3, or 4;
provided that when R¹ is an unsubstituted alkyl and J is —O—, then R² is H, an optionally substituted aliphatic, an optionally substituted aryl, or an unsubstituted heteroaryl.

In an embodiment, the IRAK4 inhibitor is a compound of Formula (48) selected from the group consisting of:
(2-(5-(3-Aminophenyl)-1H-indazol-3-ylamino)-1-cyclohexyl-1H-benzo[d]imidazol-6-yl)methanol;
(1-Cyclohexyl-2-(5-(3-(methoxymethyl)phenyl)-1H-indazol-3-ylamino)-1H-benzo[d]imidazol-5-yl)methanol;
4-(2-(5-(4-Methoxypyridin-3-yl)-1H-indazol-3-ylamino)-5-((4-(2-(pyridin-4-yl)ethyl)piperazin-1-yl)methyl)-1H-benzo[d]imidazol-1-yl)cyclohexanol;
Ethyl 1-((2-(5-(4-methoxypyridin-3-yl)-1H-indazol-3-ylamino)-1-(4-hydroxycyclohexyl)-1H-benzo[d]imidazol-5-yl)methyl)piperidine-4-carboxylate;
(2-(5-(4-Methoxypyridin-3-yl)-1H-indazol-3-ylamino)-1-phenyl-1H-benzo[d]imidazol-5-yl)methanol;
N-(1-cyclohexyl-5-(morpholinomethyl)-1H-benzo[d]imidazol-2-yl)-5-(4-methoxypyridin-3-yl)-1H-indazol-3-amine;
2-(5-(2-Methoxyphenyl)-1H-indazol-3-ylamino)-N-cyclopentyl-1-(4-hydroxycyclohexyl)-1H-benzo[d]imidazole-5-carboxamide;
1-(4-Hydroxycyclohexyl)-N-isopentyl-2-(5-(2-methoxyphenyl)-1H-indazol-3-ylamino)-1H-benzo[d]imidazole-5-carboxamide;
(1-(4-Hydroxycyclohexyl)-2-(5-(2-methoxyphenyl)-1H-indazol-3-ylamino)-1H-benzo[d]imidazol-5-yl)(pyrrolidin-1-yl)methanone;
N-Cyclopentyl-1-(4-hydroxycyclohexyl)-2-(5-(2-(trifluoromethoxy)phenyl)-1H-indazol-3-ylamino)-1H-benzo[d]imidazole-5-carboxamide;
2-(5-(2-(Trifluoromethoxy)phenyl)-1H-indazol-3-ylamino)-1-(4-hydroxycyclohexyl)-N-isopentyl-1H-benzo[d]imidazole-5-carboxamide;
Ethyl 1-(4-hydroxycyclohexyl)-2-(5-(4-methoxypyridin-3-yl)-1H-indazol-3-ylamino)-1H-benzo[d]imidazole-5-carboxylate;
4-(5-(Hydroxymethyl)-2-(5-(4-methoxypyridin-3-yl)-1H-indazol-3-ylamino)-1H-benzo[d]imidazol-1-yl)cyclohexanol;
(1-Cyclohexyl-2-(5-(4-methoxypyridin-3-yl)-1H-indazol-3-ylamino)-1H-benzo[d]imidazol-5-yl)methanol;
N-(1-Cyclohexyl-5-(piperidin-1-ylmethyl)-1H-benzo[d]imidazol-2-yl)-5-(4-methoxypyridin-3-yl)-1H-indazol-3-amine;
3-(2-(5-(4-Methoxypyridin-3-yl)-1H-indazol-3-ylamino)-1H-benzo[d]imidazol-1-yl)propan-1-1 ol;
3-(2-(5-Bromo-1H-indazol-3-ylamino)-1H-benzo[d]imidazol-1-yl)propan-1-ol;
   and pharmaceutically acceptable salts, solvates, hydrates, cocrystals, or prodrugs thereof.

In an embodiment, the IRAK4 inhibitor is PF-06650833 or a pharmaceutically acceptable salt, solvate, hydrate, cocrystal, or prodrug thereof.

In an embodiment, the IRAK4 inhibitor is selected from the compounds disclosed in U.S. Pat. Nos. 8,703,941; 9,085,586; 9,067,948; and 8,293,923; and U.S. Patent Application Publication Nos. US 2011/0152260; US 2011/0021513; US 2003/0059916; US 2005/0255549; US 2015/0094305; US 2014/0018357; US 2014/0018343; US 2014/0018361; US 2015/0025093; US 2012/0283238; US 2014/0194417; and US 2013/0231328; and International Patent Application Publication Nos. WO 2013/106535; WO 2013/106535; WO 2014/011902; WO 2014/011906; WO 2015/048281; WO 2014/011911; WO 2014/110114; WO 2012/097013; WO 2001/051641; WO 2010/014643; WO 2001/051641; WO 2008/030579; WO 2012/097013; WO 2013/106535; WO 2005/051314; WO 2013/106535; WO 2006/017621; WO 2012/007375; and WO 2008/030584, the disclosures of each of which are incorporated herein by reference.

### Pharmaceutical Compositions

In one embodiment, the invention provides a pharmaceutical composition for use in the treatment of the diseases and conditions described herein. In a preferred embodiment, the invention provides pharmaceutical compositions, including those described below, for use in the treatment of a hyperproliferative disease. In a preferred embodiment, the invention provides pharmaceutical compositions, including those described below, for use in the treatment of cancer.

In some embodiments, the invention provides pharmaceutical compositions for treating solid tumor cancers, lymphomas and leukemia.

In preferred embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor or a pharmaceutically acceptable salt, solvate, hydrate, cocrystal, or prodrug thereof, for use in the treatment of cancer. This composition is typically a pharmaceutical composition.

In preferred embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof; and (3) a compound selected from the group consisting of albumin-bound paclitaxel, bendamustine, fludarabine, cyclophosphamide, chlorambucil, an anticoagulant or antiplatelet active pharmaceutical ingredient, or combinations thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor or a pharmaceutically acceptable salt, solvate, hydrate, cocrystal, or prodrug thereof, for use in the treatment of cancer; (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof; and (4) a compound selected from the group consisting of albumin-bound paclitaxel, bendamustine, fludarabine, cyclophosphamide, chlorambucil, an anticoagulant or antiplatelet active pharmaceutical ingredient, and combinations thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor having the structure:

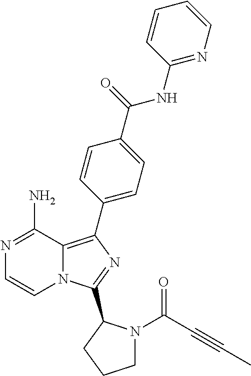 [SMILES: CC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor having the structure:

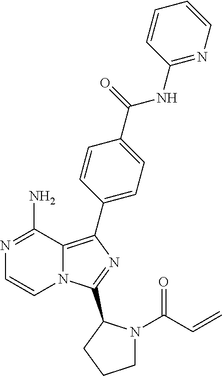 [SMILES: C=CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor having the structure:

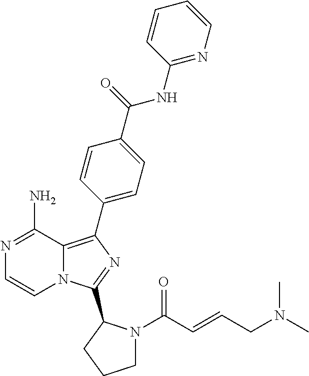 [SMILES: CN(C)C/C=C/C(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor having the structure:

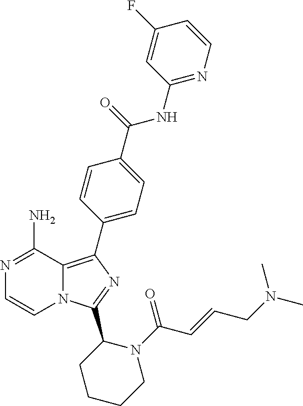 [SMILES: CN(C)C/C=C/C(=O)N1CCCC[C@H]1c1nc(-c2ccc(C(=O)Nc3cc(F)ccn3)cc2)c2c(N)nccn12]

or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor having the structure:

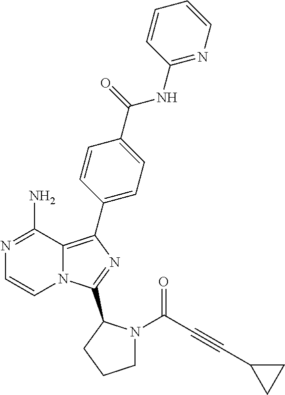 [SMILES: Nc1nccn2c([C@@H]3CCCN3C(=O)C#CC3CC3)nc(-c3ccc(C(=O)Nc4ccccn4)cc3)c12]

or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor having the structure:

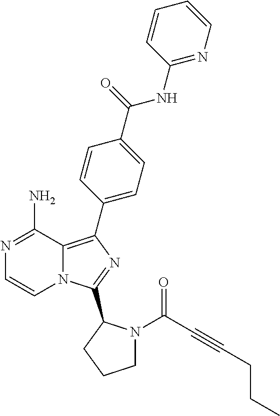 [SMILES: CCCC#CC(=O)N1CCC[C@H]1c1nc(-c2ccc(C(=O)Nc3ccccn3)cc2)c2c(N)nccn12]

or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor selected from the group consisting of:

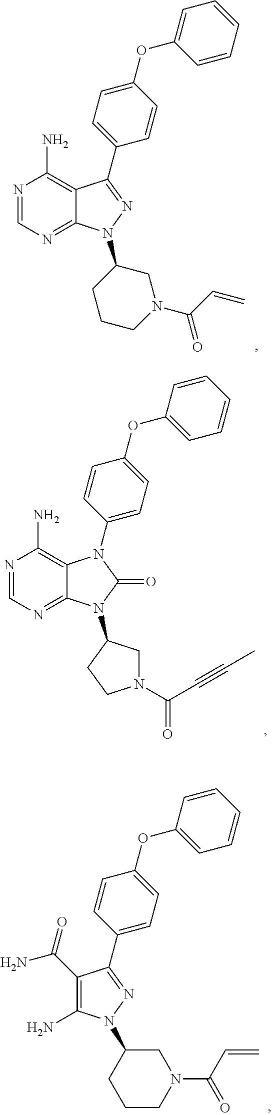

and pharmaceutically-acceptable salts, cocrystals, hydrates, solvates, and prodrugs thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor selected from the group consisting of:

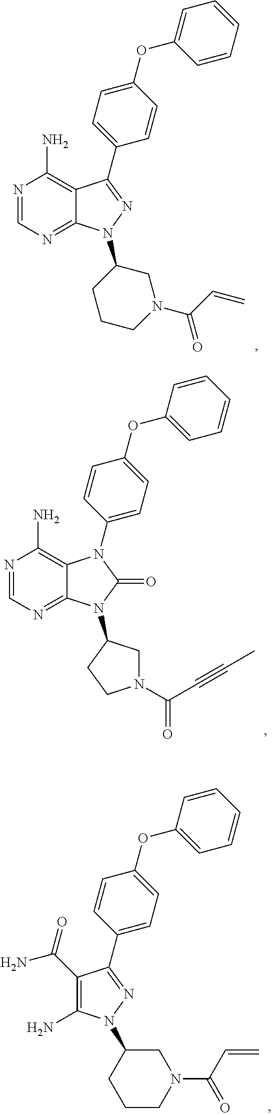

and pharmaceutically-acceptable salts, cocrystals, hydrates, solvates, and prodrugs thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; and (2) a BTK inhibitor selected from the group consisting of:

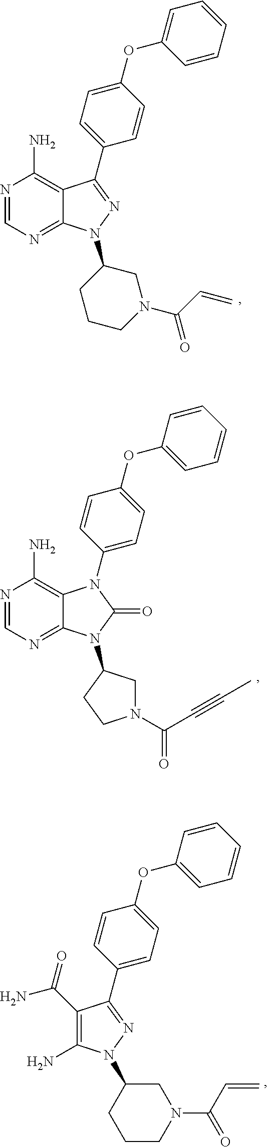 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor selected from the group consisting of:

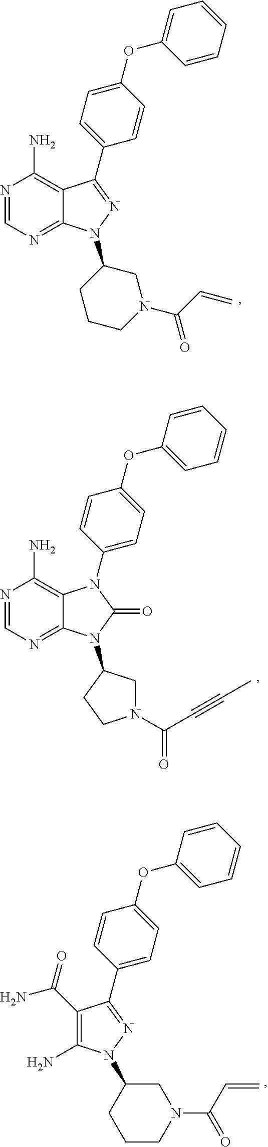 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor selected from the group consisting of:

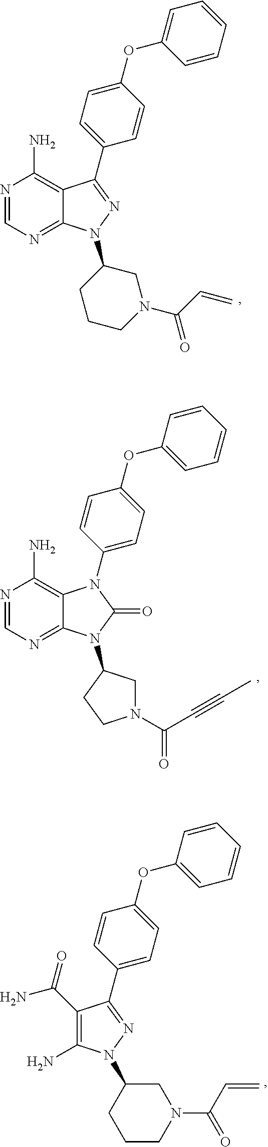 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof; and (4) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor or a pharmaceutically acceptable salt, solvate, hydrate, cocrystal, or prodrug thereof; (2) a BTK inhibitor selected from the group consisting of:

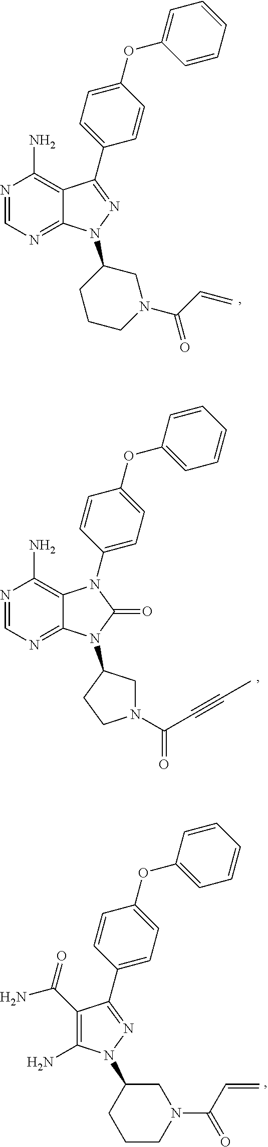 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]

and pharmaceutically-acceptable salts, cocrystals, hydrates, solvates, and prodrugs thereof; and (4) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of the compounds listed in Table 1 or Table 2, and pharmaceutically-acceptable salts, cocrystals, hydrates, solvates, and prodrugs thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor selected from the group consisting of:

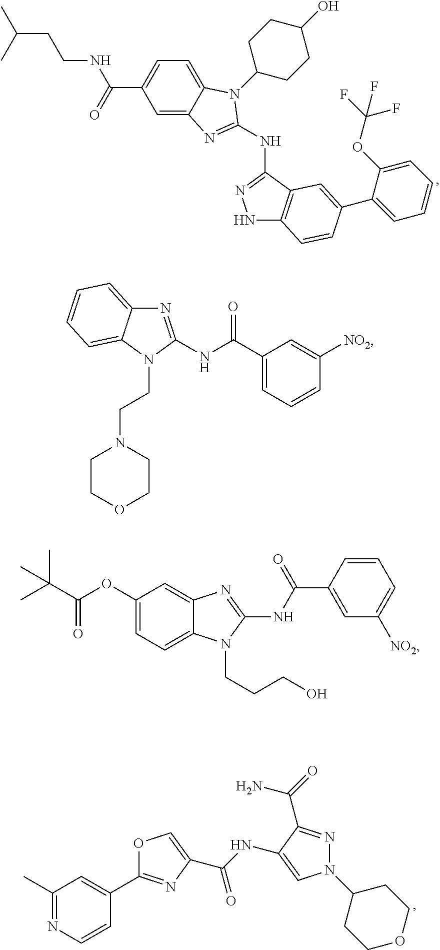 [SMILES: CC(C)CCNC(=O)c1ccc2c(c1)nc(Nc1n[nH]c3ccc(-c4ccccc4OC(F)(F)F)cc13)n2C1CCC(O)CC1 ; "," ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1 ; ","]

and pharmaceutically-acceptable salts, cocrystals, hydrates, solvates, and prodrugs thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor selected from the group consisting of the compounds listed in Table 1 or Table 2, and pharmaceutically-acceptable salts, cocrystals, hydrates, solvates, and prodrugs thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor selected from the group consisting of:

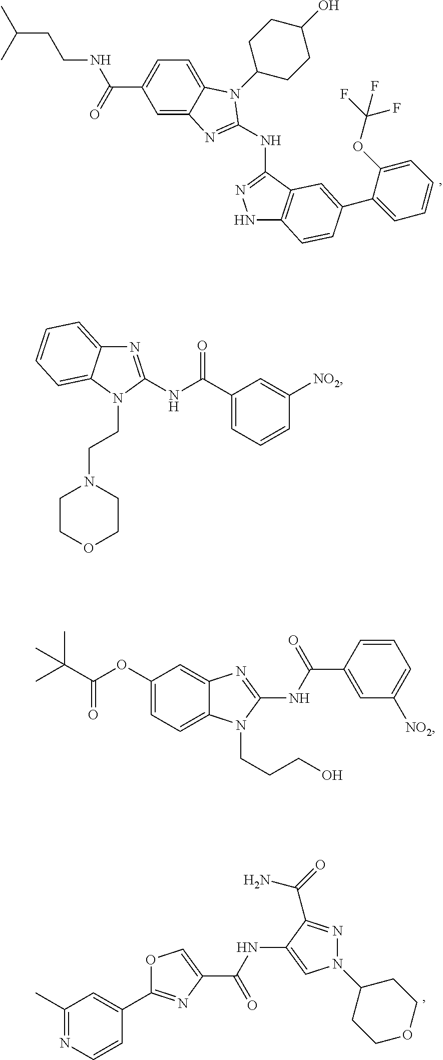 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]

and pharmaceutically-acceptable salts, cocrystals, hydrates, solvates, and prodrugs thereof; and (2) a BTK inhibitor or a pharmaceutically acceptable salt, solvate, hydrate, cocrystal, or prodrug thereof. This composition is typically a pharmaceutical composition.

In some embodiments, the invention provides a composition comprising therapeutically effective amounts of (1) an IRAK4 inhibitor selected from the group consisting of the compounds listed in Table 1 or Table 2, and pharmaceutically-acceptable salts, cocrystals, hydrates, solvates, and prodrugs thereof; (2) a BTK inhibitor or a pharmaceutically acceptable salt, solvate, hydrate, cocrystal, or prodrug thereof, for use in the treatment of cancer; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of

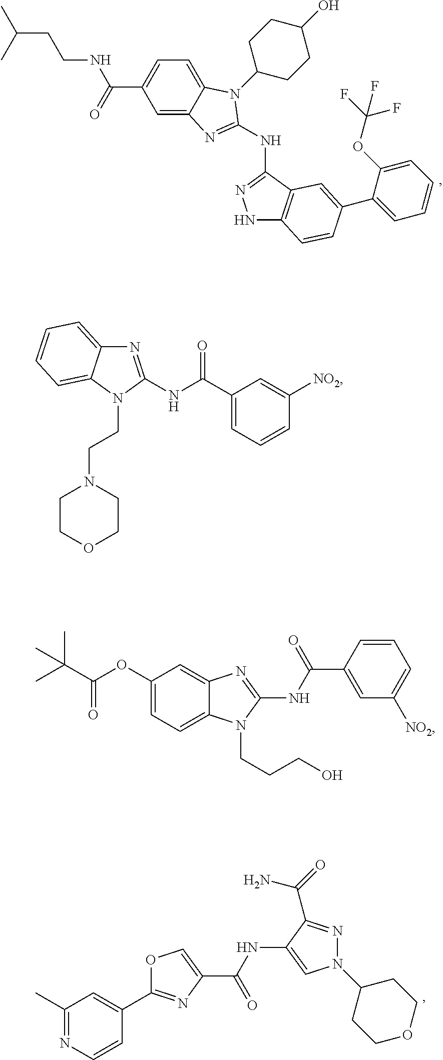 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]

and pharmaceutically-acceptable salts, cocrystals, hydrates, solvates, and prodrugs thereof; (2) a BTK inhibitor or a pharmaceutically acceptable salt, solvate, hydrate, cocrystal, or prodrug thereof, for use in the treatment of cancer; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of the compounds listed in Table 1 or Table 2, and pharmaceutically-acceptable salts, cocrystals, hydrates, solvates, and prodrugs thereof; (2) a BTK inhibitor or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of:

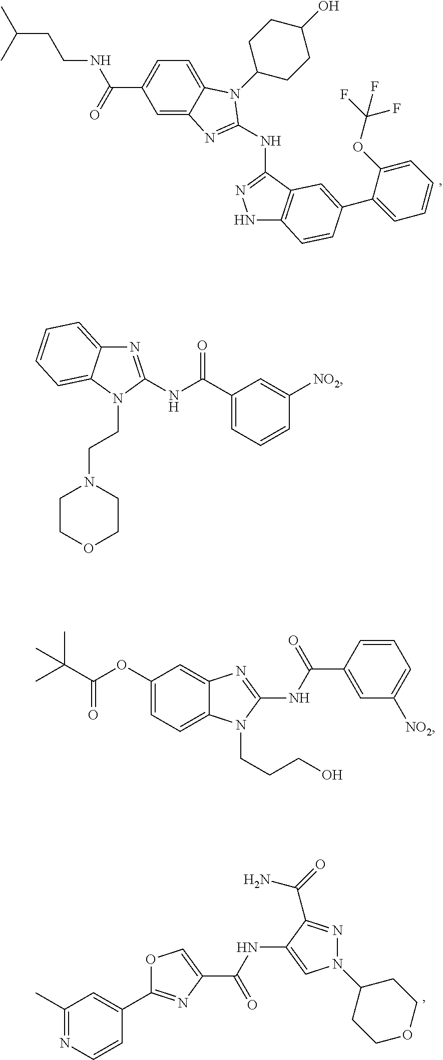 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]

the compounds listed in Table 1 or Table 2, and pharmaceutically-acceptable salts, cocrystals, hydrates, solvates, and prodrugs thereof; (2) a BTK inhibitor or a pharmaceutically acceptable salt, solvate, hydrate, cocrystal, or prodrug thereof; and (3) an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of the compounds listed in Table 1 or Table 2, and pharmaceutically-acceptable salts, cocrystals, hydrates, solvates, or prodrugs thereof; (2) a BTK inhibitor or a pharmaceutically acceptable salt, solvate, hydrate, cocrystal, or prodrug thereof; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

In some embodiments, the invention provides a composition comprising therapeutically effective amounts of (1) an IRAK4 inhibitor selected from the group consisting of:

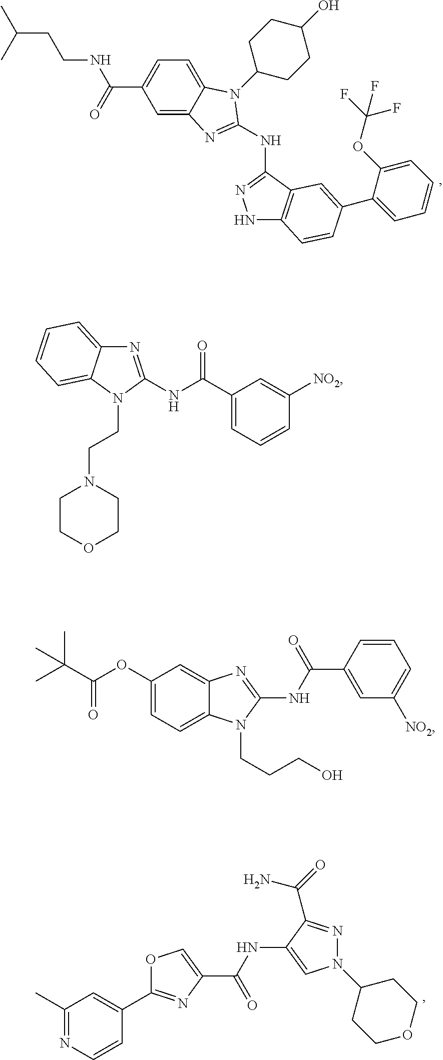 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]

and pharmaceutically-acceptable salts, cocrystals, hydrates, solvates, or prodrugs thereof; (2) a BTK inhibitor or a pharmaceutically acceptable salt, solvate, hydrate, cocrystal, or prodrug thereof; and (3) a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. This composition is typically a pharmaceutical composition.

The pharmaceutical compositions are typically formulated to provide a therapeutically effective amount of a combination as described herein, i.e., a combination of an IRAK4 inhibitor, and a BTK inhibitor as the active ingredients, or pharmaceutically acceptable salts, prodrugs, solvates, or hydrates thereof. Where desired, the pharmaceutical compositions contain a pharmaceutically acceptable salt and/or coordination complex of one or more of the active ingredients. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of the diseases and conditions described below. In a preferred embodiment, the pharmaceutical compositions are for use in the treatment of cancer. In preferred embodiments, the pharmaceutical compositions are for use in treating solid tumor cancers, lymphomas, and leukemias.

In a preferred embodiment, the pharmaceutical compositions of the present invention are for use in the treatment of cancer. In one embodiment, the pharmaceutical compositions of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

The pharmaceutical compositions may be administered as a combination of an IRAK4 inhibitor with a BTK inhibitor. Where desired, other active pharmaceutical ingredient(s) may be mixed into a preparation or two or more components of the combination may be formulated into separate preparations for use in combination separately or at the same time. A kit containing the components of the combination, formulated into separate preparations for said use, in also provided by the invention.

In an embodiment, the molar ratio of the IRAK4 inhibitor to the BTK inhibitor in the pharmaceutical compositions is in the range from about 10:1 to about 1:10, preferably from about 2.5:1 to about 1:2.5, and more preferably about 1:1. In an embodiment, the weight ratio of the IRAK4 inhibitor to the BTK inhibitor in the pharmaceutical compositions is selected from the group consisting of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, and about 1:20.

In some embodiments, the concentration of any one, two, or each of the IRAK4, and BTK inhibitors provided in the pharmaceutical compositions of the invention is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of any one, two, or each of the IRAK4, and BTK inhibitors provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of any one, two, or each of the IRAK4, and BTK inhibitors provided in the pharmaceutical compositions is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of any one, two or each of the IRAK4 and BTK inhibitors provided in the pharmaceutical compositions is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of any one, two, or each of the IRAK4 and BTK inhibitors provided in the pharmaceutical compositions is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of any one, two, or each of the IRAK4 and BTK inhibitors provided in the pharmaceutical compositions is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each IRAK4 and BTK inhibitor according to the invention is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

In a preferred embodiment, the pharmaceutical compositions of the present invention are for use in the treatment of cancer. In a preferred embodiment, the pharmaceutical compositions of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

Described below are non-limiting pharmaceutical compositions and methods for preparing the same.

### Pharmaceutical Compositions for Oral Administration

In preferred embodiments, the invention provides a pharmaceutical composition for oral administration containing the combination of an IRAK4, and a BTK, and a pharmaceutical excipient suitable for oral administration.

In preferred embodiments, the invention provides a solid pharmaceutical composition for oral administration containing: (i) an effective amount of each of an IRAK4, and a BTK inhibitor in combination and (ii) a pharmaceutical excipient suitable for oral administration. In some embodiments, the composition further contains (iii) an effective amount of a third active pharmaceutical ingredient.

In preferred embodiments, the invention provides a solid pharmaceutical composition for oral administration containing: (i) an effective amount of an IRAK4 inhibitor in combination with a BTK inhibitor and (ii) a pharmaceutical excipient suitable for oral administration. In selected embodiments, the composition further contains (iii) an effective amount of a third active pharmaceutical ingredient.

In some embodiments, the pharmaceutical composition may be a liquid pharmaceutical composition suitable for oral consumption.

Pharmaceutical compositions of the invention suitable for oral administration can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the IRAK4 and BTK inhibitors as active ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present invention and to minimize precipitation of the compound of the present invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, 6-valerolactone and isomers thereof, (3-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the invention provides a pharmaceutical composition for injection containing the combination of the IRAK4 and BTK inhibitors, most preferably a combination of the IRAK4 and BTK inhibitors, and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating the combination of the IRAK4 and BTK inhibitors in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the invention provides a pharmaceutical composition for transdermal delivery containing the combination of the IRAK4 and BTK inhibitors, and a pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of the combination of the IRAK4 and BTK inhibitors in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445; and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of the combination of the IRAK4 and BTK inhibitors or pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g. PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. The combination of the IRAK4, and BTK inhibitors may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. The combination of the IRAK4 and BTK inhibitors may additionally be administered intravascularly from a balloon used during angioplasty. Extravascular administration of the combination of the IRAK4 and BTK inhibitors via the pericard or via advential application of formulations of the invention may also be performed to decrease restenosis.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include each of the IRAK4 and BTK inhibitors, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, the IRAK4 and BTK inhibitors and another active pharmaceutical ingredient are provided as separate compositions in separate containers within the kit. In selected embodiments, the IRAK4 and BTK inhibitors and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; and (2) a composition comprising a therapeutically effective amount of a BTK inhibitor or a pharmaceutically acceptable salt, solvate, hydrate, cocrystal, or prodrug thereof. These compositions are typically pharmaceutical compositions. The kit is for co-administration of the IRAK4 and the BTK inhibitors, either simultaneously or separately.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; and (2) a composition comprising a therapeutically effective amount of a BTK inhibitor or a pharmaceutically acceptable salt, solvate, hydrate, cocrystal, or prodrug thereof. These compositions are typically pharmaceutical compositions. The kit is for co-administration of the IRAK4 and BTK inhibitors, either simultaneously or separately.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; and (2) a composition comprising a therapeutically effective amount of a BTK inhibitor or a pharmaceutically acceptable salt, solvate, hydrate, cocrystal, or prodrug thereof. These compositions are typically pharmaceutical compositions. The kit is for co-administration of the IRAK4 and BTK inhibitors, either simultaneously or separately.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. These compositions are typically pharmaceutical compositions. The kit is for co-administration of the IRAK4 inhibitor, the BTK inhibitor, and/or the anti-CD20 antibody, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. These compositions are typically pharmaceutical compositions. The kit is for co-administration of the IRAK4 inhibitor, the BTK inhibitor, and/or the anti-CD20 antibody, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, and biosimilars thereof. These compositions are typically pharmaceutical compositions. The kit is for co-administration of the IRAK4 inhibitor, the BTK inhibitor, and/or the anti-CD20 antibody, either simultaneously or separately.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of albumin-bound paclitaxel, bendamustine, fludarabine, cyclophosphamide, chlorambucil, an anticoagulant or antiplatelet active pharmaceutical ingredient, or combinations thereof. These compositions are typically pharmaceutical compositions. The kit is for co-administration of the IRAK4 inhibitor, BTK inhibitor, albumin-bound paclitaxel, bendamustine, fludarabine, cyclophosphamide, chlorambucil, and/or the anticoagulant or the antiplatelet active pharmaceutical ingredient, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of albumin-bound paclitaxel, bendamustine, an anticoagulant or antiplatelet active pharmaceutical ingredient, or combinations thereof. These compositions are typically pharmaceutical compositions. The kit is for co-administration of the IRAK4 inhibitor, BTK inhibitor, albumin-bound paclitaxel, bendamustine, fludarabine, cyclophosphamide, chlorambucil, and/or the anticoagulant or the antiplatelet active pharmaceutical ingredient, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; and/or (3) a composition comprising a therapeutically effective amount of albumin-bound paclitaxel, bendamustine, fludarabine, cyclophosphamide, chlorambucil, and/or an anticoagulant or antiplatelet active pharmaceutical ingredient. These compositions are typically pharmaceutical compositions. The kit is for co-administration of the IRAK4 inhibitor, BTK inhibitor, albumin-bound paclitaxel, bendamustine, fludarabine, cyclophosphamide, chlorambucil, and/or the anticoagulant or the antiplatelet active pharmaceutical ingredient, either simultaneously or separately.

In some embodiments, the invention provides a kit comprising (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof; and/or (4) a composition comprising a therapeutically effective amount of albumin-bound paclitaxel, bendamustine, fludarabine, cyclophosphamide, chlorambucil, an anticoagulant or antiplatelet active pharmaceutical ingredient, or combinations thereof. These compositions are typically pharmaceutical compositions. The kit is for co-administration of the IRAK4 inhibitor, BTK inhibitor, anti-CD20 antibody, albumin-bound paclitaxel, bendamustine, fludarabine, cyclophosphamide, chlorambucil, and/or the anticoagulant or the antiplatelet active pharmaceutical ingredient, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof; and/or (4) a composition comprising a therapeutically effective amount of albumin-bound paclitaxel, bendamustine, fludarabine, cyclophosphamide, chlorambucil, and/or an anticoagulant or antiplatelet active pharmaceutical ingredient. These compositions are typically pharmaceutical compositions. The kit is for co-administration of the IRAK4 inhibitor, BTK inhibitor, anti-CD20 antibody, albumin-bound paclitaxel, bendamustine, fludarabine, cyclophosphamide, chlorambucil, and/or the anticoagulant or the antiplatelet active pharmaceutical ingredient, either simultaneously or separately.

In some embodiments, the invention provides a kit comprising, (1) a composition comprising a therapeutically effective amount of an IRAK4 inhibitor or a pharmaceutically acceptable salt, solvate, hydrate, cocrystal, or prodrug thereof; (2) a composition comprising a therapeutically effective amount of a BTK inhibitor or a pharmaceutically acceptable salt, solvate, hydrate, cocrystal, or prodrug thereof; (3) a composition comprising a therapeutically effective amount of an anti-CD20 antibody selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof; and/or (4) a composition comprising a therapeutically effective amount of albumin-bound paclitaxel, bendamustine, fludarabine, cyclophosphamide, chlorambucil, and/or an anticoagulant or antiplatelet active pharmaceutical ingredient. These compositions are typically pharmaceutical compositions. The kit is for co-administration of the IRAK4 inhibitor, BTK inhibitor, anti-CD20 antibody, albumin-bound paclitaxel, bendamustine, fludarabine, cyclophosphamide, chlorambucil, and/or the anticoagulant or the antiplatelet active pharmaceutical ingredient, either simultaneously or separately.

The kits described above are preferably for use in the treatment of the diseases and conditions described herein. In a preferred embodiment, the kits are for use in the treatment of cancer. In preferred embodiments, the kits are for use in treating solid tumor cancers, lymphomas and leukemias.

In a preferred embodiment, the kits of the present invention are for use in the treatment of cancer. In a preferred embodiment, the kits of the present invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

### Dosages and Dosing Regimens

The amounts of BTK inhibitors and IRAK4 inhibitors administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of BTK inhibitors and IRAK4 inhibitors may be provided in units of mg/kg of body mass or in mg/m² of body surface area. In an embodiment, the ratio of the dose of the IRAK4 inhibitor to the dose of the BTK inhibitor in mg/kg or in mg/m² is in the range from 10:1 to 1:10, preferably from 2.5:1 to 1:2.5, and more preferably about 1:1. In an embodiment, the ratio of the IRAK4 inhibitor to the BTK inhibitor in mg/kg or in mg/m² is selected from the group consisting of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, and about 1:20.

In some embodiments, the combination of the IRAK4, and BTK inhibitors is administered in a single dose. Such administration may be by injection, e.g., intravenous injection, in order to introduce the IRAK4 and BTK inhibitors quickly. However, other routes, including the preferred oral route, may be used as appropriate. A single dose of the combination of the IRAK4 and BTK inhibitors may also be used for treatment of an acute condition.

In some embodiments, the combination of the IRAK4 and BTK inhibitors is administered in multiple doses. In a preferred embodiment, the combination of the IRAK4 and BTK inhibitors is administered in multiple doses. Dosing may be once, twice, three times, four times, five times, six times, or more than six times per day. Dosing may be once a month, once every two weeks, once a week, or once every other day. In other embodiments, the combination of the IRAK4 and BTK inhibitors is administered about once per day to about 6 times per day. In some embodiments, the combination of the IRAK4 and BTK inhibitors is administered once daily, while in other embodiments, the combination of the IRAK4 and BTK inhibitors is administered twice daily, and in other embodiments the combination of the IRAK4 and BTK inhibitors is administered three times daily. In some embodiments, a BTK inhibitor disclosed herein is administered in combination with an IRAK4 inhibitor once daily, while in other embodiments a BTK inhibitor disclosed herein is administered in combination with an IRAK4 inhibitor twice daily, and in other embodiments a BTK inhibitor disclosed herein is administered in combination with an IRAK4 inhibitor three times daily.

Administration of the active pharmaceutical ingredients of the invention may continue as long as necessary. In selected embodiments, the combination the IRAK4 and BTK inhibitors is administered for more than 1, 2, 3, 4, 5, 6, 7, 14, or 28 days. In some embodiments, the combination of the IRAK4 and BTK inhibitors is administered for less than 28, 14, 7, 6, 5, 4, 3, 2, or 1 day. In selected embodiments, the combination of the IRAK4 and BTK inhibitors is administered chronically on an ongoing basis—e.g., for the treatment of chronic effects. In another embodiment the administration of the combination the IRAK4 and BTK inhibitors continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of a BTK inhibitor disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a BTK inhibitor disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of a BTK inhibitor disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a BTK inhibitor disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an IRAK4 inhibitor disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an IRAK4 inhibitor disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an IRAK4 inhibitor disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an IRAK4 inhibitor disclosed herein is about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a combination of a BTK inhibitor and an IRAK4 inhibitor is administered at a dosage of 10 to 200 mg BID, including 10, 20, 30, 40, 50, 60, 70, 80, 90, 100, or 150 mg BID, for the BTK inhibitor, and 1 to 500 mg BID, including 1, 5, 10, 15, 25, 50, 75, 100, 150, 200, 300, 400, or 500 mg BID for the IRAK4 inhibitor.

In some embodiments, a combination of a BTK inhibitor and an IRAK4 inhibitor is administered at a dosage of 10 to 200 mg BID, including 10, 20, 30, 40, 50, 60, 70, 80, 90, 100, or 150 mg BID, for the BTK inhibitor, and 1 to 500 mg BID, including 1, 5, 10, 15, 25, 50, 75, 100, 150, 200, 300, 400, or 500 mg BID for the IRAK4 inhibitor.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the IRAK4 and BTK inhibitors may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Methods of Treating Solid Tumor Cancers, Hematological Malignancies, Inflammation, Immune and Autoimmune Disorders, and Other Diseases

The compositions and combinations of inhibitors described above can be used in a method for treating BTK-mediated disorders and diseases. In a preferred embodiment, they are for use in treating hyperproliferative disorders. They may also be used in treating other disorders as described herein and in the following paragraphs.

In some embodiments, the invention provides a method of treating a hyperproliferative disorder in a mammal that comprises administering to said mammal a therapeutically effective amount of an IRAK4 inhibitor and a BTK inhibitor, or a pharmaceutically acceptable salt, solvate, hydrate, cocrystal, or prodrug of either or both the IRAK4 inhibitor or the BTK inhibitor. In some embodiments, the invention provides a method of treating a hyperproliferative disorder in a mammal that comprises administering to said mammal a therapeutically effective amount of an IRAK4 inhibitor and a BTK inhibitor, or a pharmaceutically acceptable salt, solvate, hydrate, cocrystal, or prodrug of the IRAK4 inhibitor and the BTK inhibitor.

In some embodiments, the invention provides a method of treating a hyperproliferative disorder in a mammal that comprises administering to said mammal a therapeutically effective amount of an IRAK4 inhibitor and a BTK inhibitor, where the BTK inhibitor is selected from the group consisting of wherein the BTK inhibitor is selected from the group consisting of Formula (2), Formula (3), Formula (4), Formula (5), Formula (6), and Formula (7), or a pharmaceutically acceptable salt, solvate, hydrate, cocrystal, or prodrug of either or both the IRAK4 inhibitor or the BTK inhibitor.

In some embodiments, the hyperproliferative disorder is a solid tumor cancer selected from the group consisting of bladder cancer, squamous cell carcinoma, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity cancer, oropharyngeal cancer, gastric cancer, stomach cancer, cervical cancer, renal cancer, kidney cancer, liver cancer, ovarian cancer, prostate cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancers such as cervical carcinoma (human papillomavirus), B-cell lymphoproliferative disease, nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), glioblastoma, esophogeal tumors, head and neck tumor, metastatic colon cancer, head and neck squamous cell carcinoma, ovary tumor, pancreas tumor, renal cell carcinoma, hematological neoplasms, small-cell lung cancer, non-small-cell lung cancer, stage IV melanoma, and glioma.

In some embodiments, the hyperproliferative disorder is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, myelodysplatic syndromes, or myelofibrosis. In an embodiment, the invention relates to a method of treating a cancer in a mammal, wherein the cancer is chronic myelocytic leukemia, acute myeloid leukemia, DLBCL (including activated B-cell (ABC) and germinal center B-cell (GCB) subtypes), follicle center lymphoma, Hodgkin's disease, multiple myeloma, indolent non-Hodgkin's lymphoma, and mature B-cell ALL.

In some embodiments, the hyperproliferative disorder is a subtype of CLL. A number of subtypes of CLL have been characterized. CLL is often classified for immunoglobulin heavy-chain variable-region (IgV_{H}) mutational status in leukemic cells. R. N. Damle, et al., Blood 1999, 94, 1840-47; T. J. Hamblin, et al., Blood 1999, 94, 1848-54. Patients with IgV_{H} mutations generally survive longer than patients without IgV_{H} mutations. ZAP70 expression (positive or negative) is also used to characterize CLL. L. Z. Rassenti, et al., N. Engl. J. Med 2004, 351, 893-901. The methylation of ZAP-70 at CpG3 is also used to characterize CLL, for example by pyrosequencing. R. Claus, et al., J. Clin. Oncol. 2012, 30, 2483-91; J. A. Woyach, et al., Blood 2014, 123, 1810-17. CLL is also classfied by stage of disease under the Binet or Rai criteria. J. L. Binet, et al., Cancer 1977, 40, 855-64; K. R. Rai, T. Han, Hematol. Oncol. Clin. North Am. 1990, 4, 447-56. Other common mutations, such as 11q deletion, 13q deletion, and 17p deletion can be assessed using well-known techniques such as fluorescence in situ hybridization (FISH). In an embodiment, the invention relates to a method of treating a CLL in a human, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In some embodiments, the hyperproliferative disorder is a CLL wherein the CLL has undergone a Richter's transformation. Methods of assessing Richter's transformation, which is also known as Richter's syndrome, are described in P. Jain and S. O'Brien, Oncology, 2012, 26, 1146-52. Richter's transformation is a subtype of CLL that is observed in 5-10% of patients. It involves the development of aggressive lymphoma from CLL and has a generally poor prognosis.

In some embodiments, the hyperproliferative disorder is a CLL or SLL in a patient, wherein the patient is sensitive to lymphocytosis. In an embodiment, the invention relates to a method of treating CLL or SLL in a patient, wherein the patient exhibits lymphocytosis caused by a disorder selected from the group consisting of a viral infection, a bacterial infection, a protozoal infection, or a post-splenectomy state. In an embodiment, the viral infection in any of the foregoing embodiments is selected from the group consisting of infectious mononucleosis, hepatitis, and cytomegalovirus. In an embodiment, the bacterial infection in any of the foregoing embodiments is selected from the group consisting of pertussis, tuberculosis, and brucellosis.

In some embodiments, the hyperproliferative disorder is selected from the group consisting of myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis.

In some embodiments, the hyperproliferative disorder is an inflammatory, immune, or autoimmune disorder. In some embodiments, the hyperproliferative disorder is selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjogren's syndrome, psoriatic arthritis, juvenile rheumatoid arthritis, ankylosing spondylitis, Crohn's disease, lupus, and lupus nephritis.

In some embodiments, the hyperproliferative disorder is a disease related to vasculogenesis or angiogenesis in a mammal which can manifest as tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In some embodiments, provided herein is a method of treating, preventing and/or managing asthma. As used herein, “asthma” encompasses airway constriction regardless of the cause. Common triggers of asthma include, but are not limited to, exposure to an environmental stimulants (e.g., allergens), cold air, warm air, perfume, moist air, exercise or exertion, and emotional stress. Also provided herein is a method of treating, preventing and/or managing one or more symptoms associated with asthma. Examples of the symptoms include, but are not limited to, severe coughing, airway constriction and mucus production.

Efficacy of the methods, compounds, and combinations of compounds described herein in treating, preventing and/or managing the indicated diseases or disorders can be tested using various animal models known in the art. Efficacy in treating, preventing and/or managing asthma can be assessed using the ova induced asthma model described, for example, in Lee, et al., J. Allergy Clin. Immunol. 2006, 118, 403-9. Efficacy in treating, preventing and/or managing arthritis (e.g., rheumatoid or psoriatic arthritis) can be assessed using the autoimmune animal models described in, for example, Williams, et al., Chem. Biol. 2010, 17, 123-34, WO 2009/088986, WO 2009/088880, and WO 2011/008302. Efficacy in treating, preventing and/or managing psoriasis can be assessed using transgenic or knockout mouse model with targeted mutations in epidermis, vasculature or immune cells, mouse model resulting from spontaneous mutations, and immuno-deficient mouse model with xenotransplantation of human skin or immune cells, all of which are described, for example, in Boehncke, et al., Clinics in Dermatology, 2007, 25, 596-605. Efficacy in treating, preventing and/or managing fibrosis or fibrotic conditions can be assessed using the unilateral ureteral obstruction model of renal fibrosis, which is described, for example, in Chevalier, et al., Kidney International 2009, 75, 1145-1152; the bleomycin induced model of pulmonary fibrosis described in, for example, Moore, et al., Am. J. Physiol. Lung. Cell. Mol. Physiol. 2008, 294, L152-L160; a variety of liver/biliary fibrosis models described in, for example, Chuang, et al., Clin. Liver Dis. 2008, 12, 333-347 and Omenetti, et al., Laboratory Investigation, 2007, 87, 499-514 (biliary duct-ligated model); or any of a number of myelofibrosis mouse models such as described in Varicchio, et al., Expert Rev. Hematol. 2009, 2(3), 315-334. Efficacy in treating, preventing and/or managing scleroderma can be assessed using a mouse model induced by repeated local injections of bleomycin described, for example, in Yamamoto, et al., J. Invest. Dermatol. 1999, 112, 456-462. Efficacy in treating, preventing and/or managing dermatomyositis can be assessed using a myositis mouse model induced by immunization with rabbit myosin as described, for example, in Phyanagi, et al., Arthritis & Rheumatism, 2009, 60(10), 3118-3127. Efficacy in treating, preventing and/or managing lupus can be assessed using various animal models described, for example, in Ghoreishi, et al., Lupus, 2009, 19, 1029-1035; Ohl, et al., J. Biomed. Biotechnol., 2011, Article ID 432595; Xia, et al., Rheumatology, 2011, 50, 2187-2196; Pau, et al., PLoS ONE, 2012, 7(5), e36761; Mustafa, et al., Toxicology, 2011, 290, 156-168; Ichikawa, et al., Arthritis & Rheumatism, 2012, 62(2), 493-503; Rankin, et al., J. Immunology, 2012, 188, 1656-1667. Efficacy in treating, preventing and/or managing Sjogren's syndrome can be assessed using various mouse models described, for example, in Chiorini, et al., J. Autoimmunity, 2009, 33, 190-196. Models for determining efficacy of treatments for pancreatic cancer are described in Herreros-Villanueva, et al., WorldJ. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth et al., Adv. Immunol. 1994, 57, 281-351; Fearon, et al., Cancer Res. 1988, 48, 2975-2980.

In selected embodiments, the invention provides a method of treating a solid tumor cancer with a composition including a combination of an IRAK4 inhibitor, and a BTK inhibitor, wherein the dose is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In selected embodiments, the invention provides a method of treating pancreatic cancer, breast cancer, ovarian cancer, melanoma, lung cancer, squamous cell carcinoma including head and neck cancer, and colorectal cancer using a combination of a BTK inhibitor, and an IRAK4 inhibitor, wherein the dose is effective to inhibit signaling between the solid tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In an embodiment, the invention provides a method for treating pancreatic cancer, breast cancer, ovarian cancer, melanoma, lung cancer, head and neck cancer, and colorectal cancer using a combination of a BTK inhibitor and an IRAK4 inhibitor, or a pharmaceutically-acceptable salt, cocrystal, hydrate, solvate, or prodrug thereof. In an embodiment, the invention provides a method for treating pancreatic cancer, breast cancer, ovarian cancer, melanoma, lung cancer, head and neck cancer, and colorectal cancer using a combination of a BTK inhibitor and an IRAK4 inhibitor, or a pharmaceutically-acceptable salt, cocrystal, hydrate, solvate, or prodrug thereof, wherein the BTK inhibitor is a compound of Formula (1).

In some embodiments, the invention provides pharmaceutical compositions of a combination of an IRAK4 inhibitor and a BTK inhibitor for the treatment of hyperproliferative disorders as described herein. In some embodiments, the invention provides pharmaceutical compositions of a combination of an IRAK4 inhibitor and a BTK inhibitor for the treatment of disorders such as myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis, wherein the BTK inhibitor is selected from the group consisting of wherein the BTK inhibitor is selected from the group consisting of Formula (1), Formula (2), Formula (3), Formula (4), Formula (5), Formula (6), and Formula (7). The invention further provides a composition as described herein for the prevention of blastocyte implantation in a mammal.

### Methods of Treating Patients Intolerant to Bleeding Events

In selected embodiments, the invention provides a method of treating a disease in a human sensitive to or intolerant to bleeding events, comprising the step of administering a therapeutically effective amount of a BTK inhibitor, or a pharmaceutically-acceptable salt, cocrystal, hydrate, solvate, or prodrug thereof, and an IRAK4 inhibitor, or a pharmaceutically-acceptable salt, cocrystal, hydrate, solvate, or prodrug thereof. In a preferred embodiment, the invention provides a method of treating a cancer in a human sensitive to or intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof. In a preferred embodiment, the invention provides a method of treating a cancer in a human sensitive to or intolerant to bleeding events, comprising the step of administering a therapeutically effective amount of a BTK inhibitor and an IRAK4 inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof, and wherein the IRAK4 inhibitor is selected from the group consisting of the compounds listed in Table 1 or Table 2, and a pharmaceutically-acceptable salt, cocrystal, hydrate, solvate, and prodrug thereof. In some embodiments, the invention provides a method of treating a disease in a human sensitive to or intolerant to ibrutinib.

In selected embodiments, the invention provides a method of treating a disease in a human sensitive to or intolerant to bleeding events, comprising the step of administering a therapeutically effective amount of a BTK inhibitor, or a pharmaceutically-acceptable salt, cocrystal, hydrate, solvate, or prodrug thereof, and an IRAK4 inhibitor, or a pharmaceutically-acceptable salt, cocrystal, hydrate, solvate, or prodrug thereof. In a preferred embodiment, the invention provides a method of treating a cancer in a human sensitive to or intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof. In a preferred embodiment, the invention provides a method of treating a cancer in a human sensitive to or intolerant to bleeding events, comprising the step of administering a therapeutically effective amount of a BTK inhibitor and an IRAK4 inhibitor, wherein the BTK inhibitor is selected from the group consisting of Formula (1), Formula (2), Formula (3), Formula (4), Formula (5), Formula (6), and Formula (7), and a pharmaceutically-acceptable salt, cocrystal, hydrate, solvate, and prodrug thereof, and wherein the IRAK4 inhibitor is selected from the group consisting of:

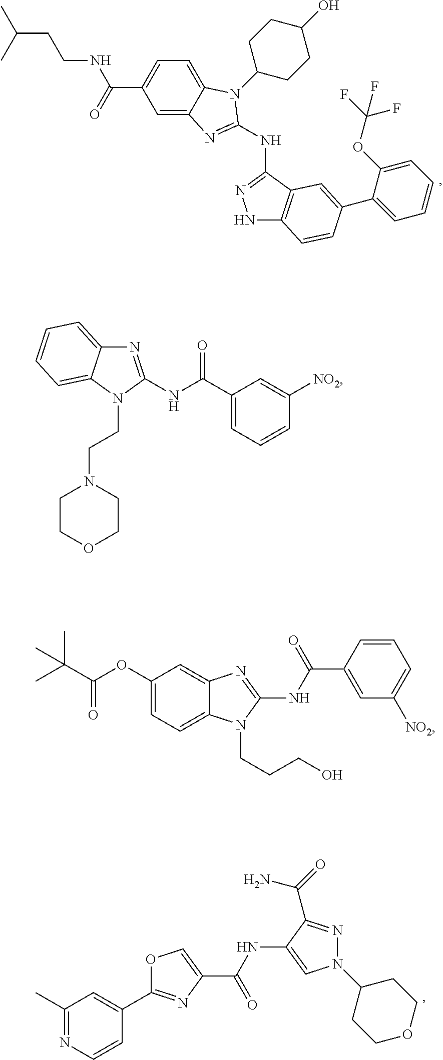 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]

and a pharmaceutically-acceptable salt, cocrystal, hydrate, solvate, and prodrug thereof.

In an embodiment, the invention provides a method of treating a cancer in a human intolerant to bleeding events, comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), or a pharmaceutically-acceptable salt, cocrystal, hydrate, solvate, or prodrug thereof, and an IRAK4 inhibitor, or a pharmaceutically-acceptable salt, cocrystal, hydrate, solvate, or prodrug thereof, further comprising the step of administering a therapeutically effective amount of an anticoagulant or antiplatelet active pharmaceutical ingredient.

In selected embodiments, the invention provides a method of treating a cancer in a human intolerant to bleeding events, comprising the step of administering a therapeutically effective amount of a BTK inhibitor, wherein the BTK inhibitor is preferably is selected from the group consisting of Formula (1), Formula (2), Formula (3), Formula (4), Formula (5), Formula (6), and Formula (7), and wherein the cancer is selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, head, neck, renal cancer, kidney cancer, liver cancer, colorectal cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophogeal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hogkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

In some embodiments, the invention provides a method of treating a cancer in a human intolerant to platelet-mediated thrombosis comprising the step of administering a therapeutically effective amount of a BTK inhibitor, wherein the BTK inhibitor is selected from the group consisting of Formula (1), Formula (2), Formula (3), Formula (4), Formula (5), Formula (6), and Formula (7), or a pharmaceutically-acceptable salt, cocrystal, hydrate, solvate, or prodrug thereof, and an IRAK4 inhibitor, or a pharmaceutically-acceptable salt, cocrystal, hydrate, solvate, or prodrug thereof.

In some embodiments, the BTK inhibitor and the anticoagulant or the antiplatelet active pharmaceutical ingredient are administered sequentially. In some embodiments, the BTK inhibitor and the anticoagulant or the antiplatelet active pharmaceutical ingredient are administered concomitantly. In selected embodiments, the BTK inhibitor is administered before the anticoagulant or the antiplatelet active pharmaceutical ingredient. In selected embodiments, the BTK inhibitor is administered after the anticoagulant or the antiplatelet active pharmaceutical ingredient. In selected embodiments, an IRAK4 inhibitor is co-administered with the BTK inhibitor and the anticoagulant or the antiplatelet active pharmaceutical ingredient at the same time or at different times.

Selected anti-platelet and anticoagulant active pharmaceutical ingredients for use in the methods of the present invention include, but are not limited to, cyclooxygenase inhibitors (e.g., aspirin), adenosine diphosphate (ADP) receptor inhibitors (e.g., clopidogrel and ticlopidine), phosphodiesterase inhibitors (e.g., cilostazol), glycoprotein IIb/IIIa inhibitors (e.g., abciximab, eptifibatide, and tirofiban), and adenosine reuptake inhibitors (e.g., dipyridamole). In other embodiments, examples of anti-platelet active pharmaceutical ingredients for use in the methods of the present invention include anagrelide, aspirin/extended-release dipyridamole, cilostazol, clopidogrel, dipyridamole, prasugrel, ticagrelor, ticlopidine, vorapaxar, tirofiban HCl, eptifibatide, abciximab, argatroban, bivalirudin, dalteparin, desirudin, enoxaparin, fondaparinux, heparin, lepirudin, apixaban, dabigatran etexilate mesylate, rivaroxaban, and warfarin.

In an embodiment, the invention provides a method of treating a cancer, comprising the step of orally administering, to a human in need thereof, a Bruton's tyrosine kinase (BTK) inhibitor, wherein the BTK inhibitor is (S)-4-(8-amino-3-(1-(but-2-ynoyl)pyrrolidin-2-yl)imidazo[1,5-a]pyrazin-1-yl)-N-(pyridin-2-yl)benzamide or a pharmaceutically acceptable salt, solvate, hydrate, cocrystal, or prodrug thereof, and an IRAK4 inhibitor, or pharmaceutically acceptable salt, solvate, hydrate, cocrystal, or prodrug thereof, further comprising the step of administering a therapeutically effective amount of an anticoagulant or antiplatelet active pharmaceutical ingredient, wherein the anticoagulant or antiplatelet active pharmaceutical ingredient is selected from the group consisting of acenocoumarol, anagrelide, anagrelide hydrochloride, abciximab, aloxiprin, antithrombin, apixaban, argatroban, aspirin, aspirin with extended-release dipyridamole, beraprost, betrixaban, bivalirudin, carbasalate calcium, cilostazol, clopidogrel, clopidogrel bisulfate, cloricromen, dabigatran etexilate, darexaban, dalteparin, dalteparin sodium, defibrotide, dicumarol, diphenadione, dipyridamole, ditazole, desirudin, edoxaban, enoxaparin, enoxaparin sodium, eptifibatide, fondaparinux, fondaparinux sodium, heparin, heparin sodium, heparin calcium, idraparinux, idraparinux sodium, iloprost, indobufen, lepirudin, low molecular weight heparin, melagatran, nadroparin, otamixaban, parnaparin, phenindione, phenprocoumon, prasugrel, picotamide, prostacyclin, ramatroban, reviparin, rivaroxaban, sulodexide, terutroban, terutroban sodium, ticagrelor, ticlopidine, ticlopidine hydrochloride, tinzaparin, tinzaparin sodium, tirofiban, tirofiban hydrochloride, treprostinil, treprostinil sodium, triflusal, vorapaxar, warfarin, warfarin sodium, ximelagatran, salts thereof, solvates thereof, hydrates thereof, prodrugs thereof, and combinations thereof.

Combinations of BTK Inhibitors and IRAK4 Inhibitors with Anti-CD20 Antibodies

The BTK inhibitors of the present invention and combinations of the BTK inhibitors with IRAK4 inhibitors may also be safely co-administered with immunotherapeutic antibodies such as the anti-CD20 antibodies rituximab, obinutuzumab, ofatumumab, veltuzumab, tositumomab, and ibritumomab, and or antigen-binding fragments, derivatives, conjugates, variants, and radioisotope-labeled complexes thereof, which may be given alone or with conventional chemotherapeutic active pharmaceutical ingredients such as those described herein. In an embodiment, the foregoing combinations exhibit synergistic effects that may result in greater efficacy, less side effects, the use of less active pharmaceutical ingredient to achieve a given clinical result, or other synergistic effects.

In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering an anti-CD20 antibody, wherein the anti-CD20 antibody is a monoclonal antibody or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering an anti-CD20 antibody, wherein the anti-CD20 antibody is an anti-CD20 monoclonal antibody or an antigen-binding fragment, derivative, conjugate, variant, or radioisotope-labeled complex thereof, and wherein the anti-CD20 antibody specifically binds to human CD20 with a K_{D} selected from the group consisting of 1×10⁻⁷ M or less, 5×10⁻⁸ M or less, 1×10⁻⁸ M or less, and 5×10⁻⁹ M or less. Anti-CD20 monoclonal antibodies are classified as Type I or Type II, as described in Klein, et al., mAbs 2013, 5, 22-33. Type I anti-CD20 monoclonal antibodies are characterized by binding to the Class I epitope, localization of CD20 to lipid rafts, high complement-dependent cytotoxicity, full binding capacity, weak homotypic aggregation, and moderate cell death induction. Type II anti-CD20 monoclonal antibodies are characterized by binding to the Class I epitope, a lack of localization of CD20 to lipid rafts, low complement-dependent cytotoxicity, half binding capacity, homotypic aggregation, and strong cell death induction. Both Type I and Type II anti-CD20 monoclonal antibodies exhibit antibody-dependent cytotoxiticy (ADCC) and are thus useful with BTK inhibitors described herein. Type I anti-CD20 monoclonal antibodies include but are not limited to rituximab, ocrelizumab, and ofatumumab. Type II anti-CD20 monoclonal antibodies include but are not limited to obinutuzumab and tositumomab. In an embodiment, the foregoing methods exhibit synergistic effects that may result in greater efficacy, less side effects, the use of less active pharmaceutical ingredient to achieve a given clinical result, or other synergistic effects.

In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and an IRAK4 inhibitor or a pharmaceutically acceptable salt, solvate, hydrate, cocrystal, or prodrug thereof, and further comprising the step of administering an anti-CD20 antibody, wherein the anti-CD20 antibody is a monoclonal antibody or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, an IRAK4 inhibitor or a pharmaceutically acceptable salt, solvate, hydrate, cocrystal, or prodrug thereof, and further comprising the step of administering an anti-CD20 antibody, wherein the anti-CD20 antibody is an anti-CD20 monoclonal antibody or an antigen-binding fragment, derivative, conjugate, variant, or radioisotope-labeled complex thereof, and wherein the anti-CD20 antibody specifically binds to human CD20 with a K_{D} selected from the group consisting of 1×10⁻⁷ M or less, 5×10⁻⁸ M or less, 1×10⁻⁸ M or less, and 5×10⁻⁹ M or less.

In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering an Type I anti-CD20 antibody, or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering an Type II anti-CD20 antibody, or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and an IRAK4 inhibitor or a pharmaceutically acceptable salt, solvate, hydrate, cocrystal, or prodrug thereof, and further comprising the step of administering an Type I anti-CD20 antibody, or an antigen-binding fragment, derivative, conjugate, variant, or radioisotope-labeled complex thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and an IRAK4 inhibitor or a pharmaceutically acceptable salt, solvate, hydrate, cocrystal, or prodrug thereof, and further comprising the step of administering an Type II anti-CD20 antibody, or an antigen-binding fragment, derivative, conjugate, variant, or radioisotope-labeled complex thereof.

In selected embodiments, the BTK inhibitors of the present invention and combinations of the BTK inhibitors with IRAK4 inhibitors, and the anti-CD20 monoclonal antibody are administered sequentially. In selected embodiments, the BTK inhibitors of the present invention and combinations of the BTK inhibitors with IRAK4 inhibitors, and the anti-CD20 monoclonal antibody are administered concomitantly. In selected embodiments, the BTK inhibitors of the present invention and combinations of the BTK inhibitors with IRAK4 inhibitors is administered before the anti-CD20 monoclonal antibody. In selected embodiments, the BTK inhibitors of the present invention and combinations of the BTK inhibitors with IRAK4 inhibitors is administered after the anti-CD20 monoclonal antibody. In selected embodiments, the BTK inhibitors of the present invention and combinations of the BTK inhibitors with IRAK4 inhibitors and the anti-CD20 monoclonal antibody are administered over the same time period, and the BTK inhibitor administration continues after the anti-CD20 monoclonal antibody administration is completed.

In an embodiment, the anti-CD20 monoclonal antibody is rituximab, or an antigen-binding fragment, derivative, conjugate, variant, or radioisotope-labeled complex thereof. Rituximab is a chimeric murine-human monoclonal antibody directed against CD20, and its structure comprises an IgG1 kappa immunoglobulin containing murine light- and heavy-chain variable region sequences and human constant region sequences. Rituximab is composed of two heavy chains of 451 amino acids and two light chains of 213 amino acids. The amino acid sequence for the heavy chains of rituximab is set forth in SEQ ID NO:1. The amino acid sequence for the light chains of rituximab is set forth in SEQ ID NO:2. Rituximab is commercially available, and its properties and use in cancer and other diseases is described in more detail in Rastetter, et al., Ann. Rev. Med. 2004, 55, 477-503, and in Plosker and Figgett, Drugs, 2003, 63, 803-43. In an embodiment, the anti-CD20 monoclonal antibody is an anti-CD20 biosimilar monoclonal antibody approved by drug regulatory authorities with reference to rituximab. In an embodiment, the anti-CD20 monoclonal antibody has a heavy chain sequence identity of greater than 90% to SEQ ID NO: 1. In an embodiment, the anti-CD20 monoclonal antibody has a light chain sequence identity of greater than 90% to SEQ ID NO:2. In an embodiment, the anti-CD20 monoclonal antibody has a heavy chain sequence identity of greater than 95% to SEQ ID NO: 1. In an embodiment, the anti-CD20 monoclonal antibody has a light chain sequence identity of greater than 95% to SEQ ID NO:2. In an embodiment, the anti-CD20 monoclonal antibody has a heavy chain sequence identity of greater than 98% to SEQ ID NO: 1. In an embodiment, the anti-CD20 monoclonal antibody has a light chain sequence identity of greater than 98% to SEQ ID NO:2. In an embodiment, the anti-CD20 monoclonal antibody has a heavy chain sequence identity of greater than 99% to SEQ ID NO: 1. In an embodiment, the anti-CD20 monoclonal antibody has a light chain sequence identity of greater than 99% to SEQ ID NO:2.

In an embodiment, the anti-CD20 monoclonal antibody is obinutuzumab, or an antigen-binding fragment, derivative, conjugate, variant, or radioisotope-labeled complex thereof. Obinutuzumab is also known as afutuzumab or GA-101. Obinutuzumab is a humanized monoclonal antibody directed against CD20. The amino acid sequence for the heavy chains of obinutuzumab is set forth in SEQ ID NO:3. The amino acid sequence for the light chains of obinutuzumab is set forth in SEQ ID NO:4. Obinutuzumab is commercially available, and its properties and use in cancer and other diseases is described in more detail in Robak, Curr. Opin. Investig. Drugs 2009, 10, 588-96. In an embodiment, the anti-CD20 monoclonal antibody is an anti-CD20 biosimilar monoclonal antibody approved by drug regulatory authorities with reference to obinutuzumab. In an embodiment, the anti-CD20 monoclonal antibody has a heavy chain sequence identity of greater than 90% to SEQ ID NO:3. In an embodiment, the anti-CD20 monoclonal antibody has a light chain sequence identity of greater than 90% to SEQ ID NO:4. In an embodiment, the anti-CD20 monoclonal antibody has a heavy chain sequence identity of greater than 95% to SEQ ID NO:3. In an embodiment, the anti-CD20 monoclonal antibody has a light chain sequence identity of greater than 95% to SEQ ID NO:4. In an embodiment, the anti-CD20 monoclonal antibody has a heavy chain sequence identity of greater than 98% to SEQ ID NO:3. In an embodiment, the anti-CD20 monoclonal antibody has a light chain sequence identity of greater than 98% to SEQ ID NO:4. In an embodiment, the anti-CD20 monoclonal antibody has a heavy chain sequence identity of greater than 99% to SEQ ID NO:3. In an embodiment, the anti-CD20 monoclonal antibody has a light chain sequence identity of greater than 99% to SEQ ID NO:4. In an embodiment, the anti-CD20 monoclonal antibody obinutuzumab is an immunoglobulin G1, anti-(human B-lymphocyte antigen CD20 (membrane-spanning 4-domains subfamily A member 1, B-lymphocyte surface antigen B1, Leu-16 or Bp35)), humanized mouse monoclonal obinutuzumab des-CH3107-K-γ1 heavy chain (222-219′)-disulfide with humanized mouse monoclonal obinutuzumab K light chain dimer (228-228″:231-231″)-bisdisulfide antibody.

In an embodiment, the anti-CD20 monoclonal antibody is ofatumumab, or an antigen-binding fragment, derivative, conjugate, variant, or radioisotope-labeled complex thereof. Ofatumumab is described in Cheson, J. Clin. Oncol. 2010, 28, 3525-30. The crystal structure of the Fab fragment of ofatumumab has been reported in Protein Data Bank reference 3GIZ and in Du, et al., Mol. Immunol. 2009, 46, 2419-2423. Ofatumumab is commercially available, and its preparation, properties, and use in cancer and other diseases are described in more detail in U.S. Pat. No. 8,529,202 B2, the disclosure of which is incorporated herein by reference. In an embodiment, the anti-CD20 monoclonal antibody is an anti-CD20 biosimilar monoclonal antibody approved by drug regulatory authorities with reference to ofatumumab. In an embodiment, the anti-CD20 monoclonal antibody has a variable heavy chain sequence identity of greater than 90% to SEQ ID NO:5. In an embodiment, the anti-CD20 monoclonal antibody has a variable light chain sequence identity of greater than 90% to SEQ ID NO:6. In an embodiment, the anti-CD20 monoclonal antibody has a variable heavy chain sequence identity of greater than 95% to SEQ ID NO:5. In an embodiment, the anti-CD20 monoclonal antibody has a variable light chain sequence identity of greater than 95% to SEQ ID NO:6. In an embodiment, the anti-CD20 monoclonal antibody has a variable heavy chain sequence identity of greater than 98% to SEQ ID NO:5. In an embodiment, the anti-CD20 monoclonal antibody has a variable light chain sequence identity of greater than 98% to SEQ ID NO:6. In an embodiment, the anti-CD20 monoclonal antibody has a variable heavy chain sequence identity of greater than 99% to SEQ ID NO:5. In an embodiment, the anti-CD20 monoclonal antibody has a variable light chain sequence identity of greater than 99% to SEQ ID NO:6. In an embodiment, the anti-CD20 monoclonal antibody has a Fab fragment heavy chain sequence identity of greater than 90% to SEQ ID NO:7. In an embodiment, the anti-CD20 monoclonal antibody has a Fab fragment light chain sequence identity of greater than 90% to SEQ ID NO:8. In an embodiment, the anti-CD20 monoclonal antibody has a Fab fragment heavy chain sequence identity of greater than 95% to SEQ ID NO:7. In an embodiment, the anti-CD20 monoclonal antibody has a Fab fragment light chain sequence identity of greater than 95% to SEQ ID NO:8. In an embodiment, the anti-CD20 monoclonal antibody has a Fab fragment heavy chain sequence identity of greater than 98% to SEQ ID NO:7. In an embodiment, the anti-CD20 monoclonal antibody has a Fab fragment light chain sequence identity of greater than 98% to SEQ ID NO:8. In an embodiment, the anti-CD20 monoclonal antibody has a Fab fragment heavy chain sequence identity of greater than 99% to SEQ ID NO:7. In an embodiment, the anti-CD20 monoclonal antibody has a Fab fragment light chain sequence identity of greater than 99% to SEQ ID NO:8. In an embodiment, the anti-CD20 monoclonal antibody ofatumumab is an immunoglobulin G1, anti-(human B-lymphocyte antigen CD20 (membrane-spanning 4-domains subfamily A member 1, B-lymphocyte surface antigen B1, Leu-16 or Bp35)); human monoclonal ofatumumab-CD20 yl heavy chain (225-214′)-disulfide with human monoclonal ofatumumab-CD20 K light chain, dimer (231-231″:234-234″)-bisdisulfide antibody.

In an embodiment, the anti-CD20 monoclonal antibody is veltuzumab, or an antigen-binding fragment, derivative, conjugate, variant, or radioisotope-labeled complex thereof. Veltuzumab is also known as hA20. Veltuzumab is described in Goldenberg, et al., Leuk. Lymphoma 2010, 51, 747-55. In an embodiment, the anti-CD20 monoclonal antibody is an anti-CD20 biosimilar monoclonal antibody approved by drug regulatory authorities with reference to veltuzumab. In an embodiment, the anti-CD20 monoclonal antibody has a heavy chain sequence identity of greater than 90% to SEQ ID NO:9. In an embodiment, the anti-CD20 monoclonal antibody has a light chain sequence identity of greater than 90% to SEQ ID NO: 10. In an embodiment, the anti-CD20 monoclonal antibody has a heavy chain sequence identity of greater than 95% to SEQ ID NO:9. In an embodiment, the anti-CD20 monoclonal antibody has a light chain sequence identity of greater than 95% to SEQ ID NO:10. In an embodiment, the anti-CD20 monoclonal antibody has a heavy chain sequence identity of greater than 98% to SEQ ID NO:9. In an embodiment, the anti-CD20 monoclonal antibody has a light chain sequence identity of greater than 98% to SEQ ID NO: 10. In an embodiment, the anti-CD20 monoclonal antibody has a heavy chain sequence identity of greater than 99% to SEQ ID NO:9. In an embodiment, the anti-CD20 monoclonal antibody has a light chain sequence identity of greater than 99% to SEQ ID NO: 10. In an embodiment, the anti-CD20 monoclonal antibody ofatumumab is an immunoglobulin G1, anti-(human B-lymphocyte antigen CD20 (membrane-spanning 4-domains subfamily A member 1, Leu-16, Bp35)); [218-arginine, 360-glutamic acid, 362-methionine]humanized mouse monoclonal hA20 γ1 heavy chain (224-213′)-disulfide with humanized mouse monoclonal hA20 K light chain (230-230″:233-233″)-bisdisulfide dimer

In an embodiment, the anti-CD20 monoclonal antibody is tositumomab, or an antigen-binding fragment, derivative, conjugate, variant, or radioisotope-labeled complex thereof. In an embodiment, the anti-CD20 monoclonal antibody is ¹³¹I-labeled tositumomab. In an embodiment, the anti-CD20 monoclonal antibody is an anti-CD20 biosimilar monoclonal antibody approved by drug regulatory authorities with reference to tositumomab. In an embodiment, the anti-CD20 monoclonal antibody has a heavy chain sequence identity of greater than 90% to SEQ ID NO: 11. In an embodiment, the anti-CD20 monoclonal antibody has a light chain sequence identity of greater than 90% to SEQ ID NO:12. In an embodiment, the anti-CD20 monoclonal antibody has a heavy chain sequence identity of greater than 95% to SEQ ID NO: 11. In an embodiment, the anti-CD20 monoclonal antibody has a light chain sequence identity of greater than 95% to SEQ ID NO: 12. In an embodiment, the anti-CD20 monoclonal antibody has a heavy chain sequence identity of greater than 98% to SEQ ID NO: 11. In an embodiment, the anti-CD20 monoclonal antibody has a light chain sequence identity of greater than 98% to SEQ ID NO: 12. In an embodiment, the anti-CD20 monoclonal antibody has a heavy chain sequence identity of greater than 99% to SEQ ID NO: 11. In an embodiment, the anti-CD20 monoclonal antibody has a light chain sequence identity of greater than 99% to SEQ ID NO: 12.

In an embodiment, the anti-CD20 monoclonal antibody is ibritumomab, or an antigen-binding fragment, derivative, conjugate, variant, or radioisotope-labeled complex thereof. The active form of ibritumomab used in therapy is ibritumomab tiuxetan. When used with ibritumomab, the chelator tiuxetan (diethylene triamine pentaacetic acid) is complexed with a radioactive isotope such as ⁹⁰Y or ¹¹¹In. In an embodiment, the anti-CD20 monoclonal antibody is ibritumomab tiuxetan, or radioisotope-labeled complex thereof. In an embodiment, the anti-CD20 monoclonal antibody is an anti-CD20 biosimilar monoclonal antibody approved by drug regulatory authorities with reference to tositumomab. In an embodiment, the anti-CD20 monoclonal antibody has a heavy chain sequence identity of greater than 90% to SEQ ID NO: 13. In an embodiment, the anti-CD20 monoclonal antibody has a light chain sequence identity of greater than 90% to SEQ ID NO:14. In an embodiment, the anti-CD20 monoclonal antibody has a heavy chain sequence identity of greater than 95% to SEQ ID NO: 13. In an embodiment, the anti-CD20 monoclonal antibody has a light chain sequence identity of greater than 95% to SEQ ID NO: 14. In an embodiment, the anti-CD20 monoclonal antibody has a heavy chain sequence identity of greater than 98% to SEQ ID NO: 13. In an embodiment, the anti-CD20 monoclonal antibody has a light chain sequence identity of greater than 98% to SEQ ID NO: 14. In an embodiment, the anti-CD20 monoclonal antibody has a heavy chain sequence identity of greater than 99% to SEQ ID NO:13. In an embodiment, the anti-CD20 monoclonal antibody has a light chain sequence identity of greater than 99% to SEQ ID NO: 14.

In an embodiment, an anti-CD20 antibody selected from the group consisting of obinutuzumab, ofatumumab, veltuzumab, tositumomab, and ibritumomab, and or antigen-binding fragments, derivatives, conjugates, variants, and radioisotope-labeled complexes thereof, is administered to a subject by infusing a dose selected from the group consisting of about 10 mg, about 20 mg, about 25 mg, about 50 mg, about 75 mg, 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In an embodiment, the anti-CD20 antibody is administered weekly. In an embodiment, the anti-CD20 antibody is administered every two weeks. In an embodiment, the anti-CD20 antibody is administered every three weeks. In an embodiment, the anti-CD20 antibody is administered monthly. In an embodiment, the anti-CD20 antibody is administered at a lower initial dose, which is escalated when administered at subsequent intervals administered monthly. For example, the first infusion can deliver 300 mg of anti-CD20 antibody, and subsequent weekly doses could deliver 2,000 mg of anti-CD20 antibody for eight weeks, followed by monthly doses of 2,000 mg of anti-CD20 antibody. During any of the foregoing embodiments, the BTK inhibitors of the present invention and combinations of the BTK inhibitors with IRAK4 inhibitors may be administered daily, twice daily, or at different intervals as described above, at the dosages described above.

In an embodiment, the invention provides a kit comprising a first composition comprising a BTK inhibitor and/or combinations of the BTK inhibitor with IRAK4 inhibitor and a second composition comprising an anti-CD20 antibody selected from the group consisting of rituximab, obinutuzumab, ofatumumab, veltuzumab, tositumomab, and ibritumomab, or an antigen-binding fragment, derivative, conjugate, variant, or radioisotope-labeled complex thereof, for use in the treatment of CLL or SLL, hematological malignancies, B cell malignancies or, or any of the other diseases described herein. The compositions are typically both pharmaceutical compositions. The kit is for use in co-administration of the anti-CD20 antibody and the BTK inhibitor, either simultaneously or separately, in the treatment of CLL or SLL, hematological malignancies, B cell malignancies, or any of the other diseases described herein.

The anti-CD20 antibody sequences referenced in the foregoing are summarized in Table 3.

TABLE 3
Anti-CD20 antibody sequences.

| Identifier | Sequence (One-Letter Amino Acid Symbols) | |
|---|---|---|
| SEQ ID NO: 1 | QVQLQQPGAE LVKPGASVKM SCKASGYTFT SYNMHWVKQT PGRGLEWIGA IYPGNGDTSY | 60 |
| rituximab heavy | NQKFKGKATL TADKSSSTAY MQLSSLTSED SAVYYCARST YYGGDWYFNV WGAGTTVTVS | 120 |
| chain | AASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKKVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRD | 360 |
| | ELTKNQVSLT CLVKGFYPSD IAVEWESNGQ PENNYETTPP VLDSDGSFFL YSKLTVDKSR | 420 |
| | WQQGNVFSCS VMHEALHNHY TQKSLSLSPG X | 451 |
| SEQ ID NO: 2 | QIVLSQSPAI LSASPGEKVT MTCRASSSVS YIHWFQQKPG SSPKPWIYAT SNLASGVPVR | 60 |
| rituximab light | FSGSGSGTSY SLTISRVEAE DAATYYCQQW TSNPPTFGGG TKLEIKRTVA APSVFIFPPS | 120 |
| chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTESFNR GEC | 213 |
| SEQ ID NO: 3 | QVQLVQSGAE VKKPGSSVKV SCKASGYAFS YSWINWVRQA PGQGLEWMGR IFPGDGDTDY | 60 |
| obinutuzumab | NGKFKGRVTI TADKSTSTAY MELSSLRSED TAVYYCARNV FDGYWLVYWG QGTLVTVSSA | 120 |
| heavy chain | STKGPSVFPL APSKESTSGG TAALGCLVKD YFPEPVTVSW NSGALTSGVH TFPAVLQSSG | 180 |
| | LYSLSSVVTV PSSSLGTQTY ICNVNHKPSN TKVDKKVEPK SCDKTHTCPP CPAPELLGGP | 240 |
| | SVFLFPPKPK DTLMISRTPE VTCVVVDVSH EDPEVKFNWY VDGVEVHNAK TKPREEQYNS | 300 |
| | TYRVVSVLTV LHQDWLNGKE YKCKVSNKAL PAPIEKTISK AKGQPREPQV YTLPPSRDEL | 360 |
| | TKNQVSLTCL VKGFYPSDIA VEWESNGQPE NNYKTTPPVL DSDGSFFLYS KLTVDKSRWQ | 420 |
| | QGNVFSCSVM HEALHNHYTQ KSLSLSPGK | 449 |
| SEQ ID NO: 4 | DIVMTQTPLS LPVTPGEPAS ISCRSSESLL HSNGITYLYW YLQKPGQSPQ LLIYQMSNLV | 60 |
| obinutuzumab | SGVPDRFSGS GSGTDFTLKI SRVEAEDVGV YYCAQNLELP YTFGGGTKVE IKRTVAAPSV | 120 |
| light chain | FIFPPSDEQL KSGTASVVCL LNNFYPREAK VQWKVDNALQ SGNSQESVTE QDSKDSTYSL | 180 |
| | SSTLTLSKAD YEKHKVYACE VTHQGLSSPV TKSFNRGEC | 219 |
| SEQ ID NO: 5 | EVQLVESGGG LVQPGRSLRL SCAASGFTFN DYAMHWVRQA PGKGLEWVST ISWNSGSIGY | 60 |
| ofatumumab | ADSVKGRFTI SRDNAKKSLY LQMNSLRAED TALYYCAKDI QYGNYYYGMD VWGQGTTVTV | 120 |
| variable heavy | SS | 122 |
| chain | | |
| SEQ ID NO: 6 | EIVLTQSPAT LSLSPGERAT LSCRASQSVS SYLAWYQQKP GQAPRLLIYD ASNRATGIPA | 60 |
| ofatumumab | RFSGSGSGTD FTLTISSLEP EDFAVYYCQQ RSNWPITFGQ GTRLEIK | 107 |
| variable light | | |
| chain | | |
| SEQ ID NO: 7 | EVQLVESGGG LVQPGRSLRL SCAASGFTFN DYAMHWVRQA PGKGLEWVST ISWNSGSIGY | 60 |
| ofatumumab Fab | ADSVEGRFTI SRDNAKKSLY LQMNSLRAED TALYYCAKDI QYGNYYYGMD VWGQGTTVTV | 120 |
| fragment heavy | SSASTKGPSV FPLAPGSSKS TSGTAALGCL VKDYFPEPVT VSWNSGALTS GVHTFPAVLQ | 180 |
| chain | SSGLYSLSSV VTVPSSSLGT QTYICNVNHK PSNTKVDKKV EP | 222 |
| SEQ ID NO: 8 | EIVLTQSPAT LSLSPGERAT LSCRASQSVS SYLAWYQQKP GQAPRLLIYD ASNRATGIPA | 60 |
| ofatumumab Fab | RFSGSGSGTD FTLTISSLEP EDFAVYYCQQ RSNWPITFGQ GTRLEIKRTV AAPSVFIFPP | 120 |
| fragment light | SDEQLKSGTA SVVCLLNNFY PREAKVQWKV DNALQSGNSQ ESVTEQDSKD STYSLSSTLT | 180 |
| chain | LSKADYEKHK VYACEVTHQG LSSPVTKSFN R | 211 |
| SEQ ID NO: 9 | QVQLQQSGAE VKKPGSSVKV SCKASGYTFT SYNMHWVKQA PGQGLEWIGA IYPGMGDTSY | 60 |
| veltuzumab heavy | NQKFKGKATL TADESTNTAY MELSSLRSED TAFYYCARST YYGGDWYFDV WGQGTTVTVS | 120 |
| chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDETHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYECKVSNK ALPAPIEKTI SKAKGQPREP QVYTLPPSRE | 360 |
| | EMTKNQVSLT CLVKGFYPSD IAVEWESNGQ PENNYETTPP VLDSDGSFFL YSKLTVDESR | 420 |
| | WQQGNVFSCS VMHEALHNHY TQKSLSLSPG K | 451 |
| SEQ ID NO: 10 | DIQLTQSPSS LSASVGDRVT MTCRASSSVS YIHWFQQFPG KAPKPWIYAT SNLASGVPVR | 60 |
| veltuzumab light | FSGSGSGTDY TFTISSLQPE DIATYYCQQW TSNPPTFGGG TKLEIKRTVA APSVFIFPPS | 120 |
| chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 11 | QAYLQQSGAE LVRPGASVKM SCKASGYTFT SYNMHWVKQT PRQGLEWIGA IYPGNGDTSY | 60 |
| tositumomab | NQKFKGKATL TVDKSSSTAY MQLSSLTSED SAVYFCARVV YYSNSYWYFD VWGTGTTVTV | 120 |
| heavy chain | SGPSVFPLAP SSKSTSGGTA ALGCLVKDYF PEPVTVSWNS GALTSGVHTF PAVLQSSGLY | 180 |
| | SLSSVVTVPS SSLGTQTYIC NVNHKPSNTK VDKKAEPESC DKTHTCPPCP APELLGGPSV | 240 |
| | FLFPPKPKDT LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSRDELTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID NO: 12 | QIVLSQSPAI LSASPGEKVT MTCRASSSVS YMHWYQQKPG SSPKPWIYAP SNLASGVPAR | 60 |
| tositumomab | FSGSGSGTSY SLTISRVEAE DAATYYCQQW SFNPPTFGAG TLLELKRTVA APSVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR | 210 |
| SEQ ID NO: 13 | QAYLQQSGAE LVRPGASVKM SCKASGYTFT SYNMHWVKQT PRQGLEWIGA IYPGNGDTSY | 60 |
| ibritumomab | NQKFKGKATL TVDKSSSTAY MQLSSLTSED SAVYFCARVV YYSNSYWYFD VWGTGTTVTV | 120 |
| heavy chain | SAPSVYPLAP VCGDTTGSSV TLGCLVKGYF PEPVTLTWNS GSLSSGVHTF PAVLQSDLYT | 180 |
| | LSSSVTVTSS TWPSQSITCN VAHPASSTKV DKKIEPRGPT IKPCPPCKCP APNLLGGPSV | 240 |
| | FIFPPKIKDV LMISLSPIVT CVVVDVSEDD PDVQISWFVN NVEVHTAQTQ THREDYNSTL | 300 |
| | RVVSALPIQH QDWMSGKEFK CKVNNKDLPA PIERTISKPK GSVRAPQVYV LPPPEEEMTK | 360 |
| | KQVTLTCMVT DFMPEDIYVE WTNNGKTELN YKNTEPVLDS DGSTFMYSKL RVEKKNWVER | 420 |
| | NSYSCSVVHE GLHNHHTTES FSR | 443 |
| SEQ ID NO: 14 | QIVLSQSPAI LSASPGEKVT MTCRASSSVS YMHWYQQKPG SSPKPWIYAP SNLASGVPAR | 60 |
| ibritumomab | FSGSGSGTSY SLTISRVEAE DAATYYCQQW SFNPPTFGAG TKLELKRADA APTVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFN | 209 |

Combinations of BTK Inhibitors with Chemotherapeutic Active Pharmaceutical Ingredients

The combinations of the BTK inhibitors with IRAK4 inhibitors may also be safely co-administered with chemotherapeutic active pharmaceutical ingredients such as albumin-bound paclitaxel (nab-paclitaxel), and bendamustine or bendamustine hydrochloride. In a preferred embodiment, the invention provides a method of treating a hematological malignancy or a solid tumor cancer in a human comprising the step of administering to said human a BTK inhibitor and an IRAK4 inhibitor, or a pharmaceutically acceptable salt, prodrug, cocrystal, solvate or hydrate thereof. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt, prodrug, cocrystal, solvate or hydrate thereof, and/or an IRAK4 inhibitor, or a pharmaceutically acceptable salt, prodrug, cocrystal, solvate or hydrate thereof. In an embodiment, the solid tumor cancer in any of the foregoing embodiments is pancreatic cancer.

In an embodiment, the invention provides a method of treating a hematological malignancy or a solid tumor cancer in a human comprising the step of administering to said human a BTK inhibitor and an IRAK4 inhibitor, and further comprising the step of administering a therapeutically-effective amount of albumin-bound paclitaxel. In an embodiment, the invention provides a method of treating a hematological malignancy or a solid tumor cancer in a human comprising the step of administering to said human a BTK inhibitor selected from the group consisting of Formula (1), Formula (2), Formula (3), Formula (4), Formula (5), Formula (6), and Formula (7), or a pharmaceutically acceptable salt or ester, prodrug, cocrystal, solvate or hydrate thereof, and/or an IRAK4 inhibitor, and further comprising the step of administering a therapeutically-effective amount of albumin-bound paclitaxel. In an embodiment, the solid tumor cancer in any of the foregoing embodiments is pancreatic cancer.

In an embodiment, the invention provides a method of treating a hematological malignancy or a solid tumor cancer in a human comprising the step of administering to said human a BTK inhibitor and an IRAK4 inhibitor, and further comprising the step of administering a therapeutically-effective amount of bendamustine hydrochloride. In an embodiment, the invention provides a method of treating a hematological malignancy or a solid tumor cancer in a human comprising the step of administering to said human a BTK inhibitor of Formula (1), Formula (2), Formula (3), Formula (4), Formula (5), Formula (6), and Formula (7), or a pharmaceutically acceptable salt or ester, prodrug, cocrystal, solvate or hydrate thereof, and/or an IRAK4 inhibitor, and further comprising the step of administering a therapeutically-effective amount of bendamustine hydrochloride.

In an embodiment, the invention provides a method of treating a hematological malignancy or a solid tumor cancer in a human comprising the step of administering to said human a BTK inhibitor and an IRAK4 inhibitor, and further comprising the step of administering a therapeutically-effective amount of vinblastine. Combination therapy with vinblastine and an IRAK4 inhibitor has been shown to improve survival in a mouse model. Srivastava, et al., Cancer Res. 2012, 72, 6209. Efficacy of the combinations of BTK inhibitors disclosed herein with the IRAK4 inhibitors disclosed herein and with vinblastine may be assess using similar mouse models. In an embodiment, the invention provides a method of treating a hematological malignancy or a solid tumor cancer in a human comprising the step of administering to said human a BTK inhibitor of Formula (1), Formula (2), Formula (3), Formula (4), Formula (5), Formula (6), and Formula (7), or a pharmaceutically acceptable salt or ester, prodrug, cocrystal, solvate or hydrate thereof, and/or an IRAK4 inhibitor, and further comprising the step of administering a therapeutically-effective amount of vinblastine.

In an embodiment, the invention provides a method of treating a hematological malignancy or a solid tumor cancer in a human comprising the step of administering to said human a BTK inhibitor and an IRAK4 inhibitor, and further comprising the step of administering a therapeutically-effective amount of a combination of fludarabine, cyclophosphamide, and rituximab (which collectively may be referred to as “FCR” or “FCR chemotherapy”). In an embodiment, the invention provides a method of treating a hematological malignancy or a solid tumor cancer in a human comprising the step of administering to said human a BTK inhibitor of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering a therapeutically-effective amount of FCR chemotherapy. In an embodiment, the invention provides a hematological malignancy or a solid tumor cancer comprising the step of administering to said human a BTK inhibitor and/or an IRAK4 inhibitor, and further comprising the step of administering a therapeutically-effective amount of FCR chemotherapy. FCR chemotherapy has been shown to improve survival in patients with cancer, as described in Hallek, et al., Lancet. 2010, 376, 1164-1174.

In an embodiment, the invention provides a method of treating a hematological malignancy or a solid tumor cancer in a human comprising the step of administering to said human a BTK inhibitor and an IRAK4 inhibitor, and further comprising the step of administering a therapeutically-effective amount of a combination of rituximab, cyclophosphamide, doxorubicin hydrochloride (also referred to as hydroxydaunomycin), vincristine sulfate (also referred to as oncovin), and prednisone (which collectively may be referred to as “R-CHOP” or “R-CHOP chemotherapy”). In an embodiment, the invention provides a method of treating a hematological malignancy or a solid tumor cancer in a human comprising the step of administering to said human a BTK inhibitor of Formula (1), Formula (2), Formula (3), Formula (4), Formula (5), Formula (6), and Formula (7), or a pharmaceutically acceptable salt or ester, prodrug, cocrystal, solvate or hydrate thereof, and further comprising the step of administering a therapeutically-effective amount of R-CHOP chemotherapy. In an embodiment, the invention provides a hematological malignancy or a solid tumor cancer comprising the step of administering to said human a BTK inhibitor and/or an IRAK4 inhibitor, and further comprising the step of administering a therapeutically-effective amount of R-CHOP chemotherapy. R-CHOP chemotherapy has been shown to improve the 10-year progression-free and overall survival rates for patients with cancer, as described in Sehn, Blood, 2010, 116, 2000-2001.

In any of the foregoing embodiments, the chemotherapeutic active pharmaceutical ingredient or combinations thereof may be administered before, concurrently, or after administration of the IRAK4 inhibitors and the BTK inhibitors.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

### Preclinical Characteristics of BTK Inhibitors

The BTK inhibitor ibrutinib (Formula (10), (1-[(3R)-3-[4-amino-3-(4-phenoxyphenyl)-1H-pyrazolo[3,4-d]pyrimidin-1-yl]piperidin-1-yl]prop-2-en-1-one) is a first-generation BTK inhibitor. In clinical testing as a monotherapy in subjects with hematologic malignancies, ibrutinib was generally well tolerated at dose levels through 840 mg (the highest dose tested). Advani, et al., J. Clin. Oncol. 2013, 31, 88-94; Byrd, et al., N. Engl. J. Med. 2013, 369, 32-42; Wang, et al., N. Engl. J. Med. 2013, 369, 507-16. No maximum tolerated dose (MTD) was apparent within the tested dose range. Furthermore, subjects typically found the drug tolerable over periods extending to >2 years. No subject had tumor lysis syndrome. No overt pattern of myelosuppression was associated with ibrutinib treatment. No drug-related reductions in circulating CD4⁺ T cells or serum immunoglobulins were noted. Adverse events with an apparent relationship to study drug included diarrhea and rash.

In subjects with heavily pretreated non-Hodgkin lymphoma (NHL), ibrutinib showed substantial antitumor activity, inducing durable regressions of lymphadenopathy and splenomegaly in most subjects. Improvements in disease-associated anemia and thrombocytopenia were observed. The pattern of changes in subjects with CLL was notable. Single-active pharmaceutical ingredient ibrutinib caused rapid and substantial reductions in lymph node size concomitant with a redistribution of malignant sites into the peripheral blood. An asymptomatic absolute lymphocyte count (ALC) increase was observed that was maximal during the first few months of treatment and generally decreased thereafter but could be persistent in some subjects or could be seen repeatedly in subjects who had interruption and resumption of drug therapy.

Collectively, these data with ibrutinib support the potential benefits of selective BTK inhibition in the treatment of subjects with relapsed lymphoid cancers. However, while highly potent in inhibiting BTK, ibrutinib has also shown in vitro activity against other kinases with a cysteine in the same position as Cys481 in BTK, to which the drug covalently binds. For example, ibrutinib inhibits epidermal growth factor receptor (EGFR), which may be the cause of ibrutinib-related diarrhea and rash. In addition, it is a substrate for both cytochrome P450 (CYP) enzymes 3A4/5 and 2D6, which increases the possibility of drug-drug interactions. These liabilities support the development of alternative BTK inhibitors for use in the therapy of lymphoid cancer.

The preclinical selectivity and potency characteristics of the second-generation BTK inhibitor of Formula (2) were compared to the first-generation BTK inhibitor of Formula (10) (ibrutinib). In Table 4, a kinome screen (performed by Life Technologies or based on literature data) is shown that compares these compounds.

TABLE 4
Kinome Screen for BTK Inhibitors (IC₅₀, nM)

| | | Ibrutinib (Formula |
|---|---|---|
| 3F-Cys Kinase | Formula (2) | (10)) |
| Btk | 3.1 | 0.5 |
| Tec | 29 | 78 |
| Bmx | 39 | 0.80 |
| ITK | >1000 | 10.7 |
| Txk | 291 | 2.0 |
| EGFR | >1000 | 5.6 |
| ErbB2 | 912 | 9.4 |
| ErbB4 | 13.2 | 2.7 |
| Blk | >1000 | 0.5 |
| JAK-3 | >1000 | 16.1 |

The results shown in Table 4 are obtained from a 10 point biochemical assay generated from 10 point concentration curves. The BTK inhibitor of Formula (2) shows much greater selectivity for BTK compared to other kinases than ibrutinib.

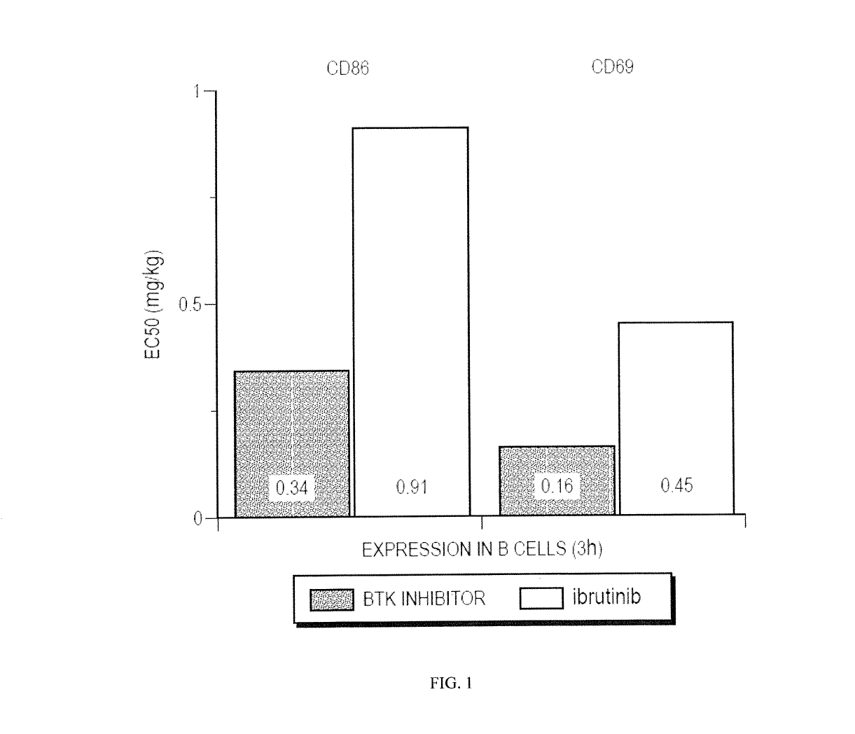

A comparison of the in vivo potency results for the BTK inhibitors of Formula (2) and ibrutinib is shown in FIG. 1. CD86 and CD69 are cell surface proteins that are BCR activation markers. To obtain the in vivo potency results, mice were gavaged at increasing drug concentration and sacrificed at one time point (3 hours post-dose). BCR was stimulated with IgM and the expression of activation marker CD69 and CD86 are monitored by flow cytometry to determine EC₅₀ values.

In vitro and in vivo safety pharmacology studies with Formula (2) have demonstrated a favorable nonclinical safety profile. When screened at 10 μM in binding assays evaluating interactions with 80 known pharmacologic targets such as G-protein-coupled receptors, nuclear receptors, proteases, and ion channels, Formula (2) shows significant activity only against the A3 adenosine receptor; follow-up dose-response experiments indicated an IC₅₀ of 2.7 μM, suggesting a low clinical risk of off-target effects. Formula (2) at 10 μM showed no inhibition of in vitro EGFR phosphorylation in an A431 human epidermoid cancer cell line whereas ibrutinib had an IC₅₀ of 66 nM. The in vitro effect of Formula (2) on human ether-a-go-go-related gene (hERG) channel activity was investigated in vitro in human embryonic kidney cells stably transfected with hERG. Formula (2) inhibited hERG channel activity by 25% at 10 μM, suggesting a low clinical risk that Formula (2) would induce clinical QT prolongation as predicted by this assay. Formula (2) was well tolerated in standard in vivo Good Laboratory Practices (GLP) studies of pharmacologic safety. A functional observation battery in rats at doses through 300 mg/kg (the highest dose level) revealed no adverse effects on neurobehavioral effects or body temperature at any dose level. A study of respiratory function in rats also indicated no treatment-related adverse effects at doses through 300 mg/kg (the highest dose level). In a cardiovascular function study in awake telemeterized male beagle dogs, single doses of Formula (2) at dose levels through 30 mg/kg (the highest dose level) induced no meaningful changes in body temperature, cardiovascular, or electrocardiographic (ECG) (including QT interval) parameters. The results suggest that Formula (2) is unlikely to cause serious off-target effects or adverse effects on critical organ systems.

The drug-drug interaction potential of Formula (2) was also evaluated. In vitro experiments evaluating loss of parent drug as catalyzed by CYPs indicated that Formula (2) is metabolized by CYP3A4. In vitro metabolism studies using mouse, rat, dog, rabbit, monkey, and human hepatocytes incubated with ¹⁴C-labeled Formula (2) indicated two mono-oxidized metabolites and a glutathione conjugate. No unique human metabolite was identified. Preliminary evaluations of metabolism in the plasma, bile, and urine of rats, dogs, and monkeys indicated metabolic processes of oxidation, glutathione binding, and hydrolysis. It was shown that Formula (2) binds to glutathione but does not deplete glutathione in vitro. Nonclinical CYP interaction studies data indicate that Formula (2) is very unlikely to cause clinical drug-drug interactions through alteration of the metabolism of drugs that are substrates for CYP enzymes.

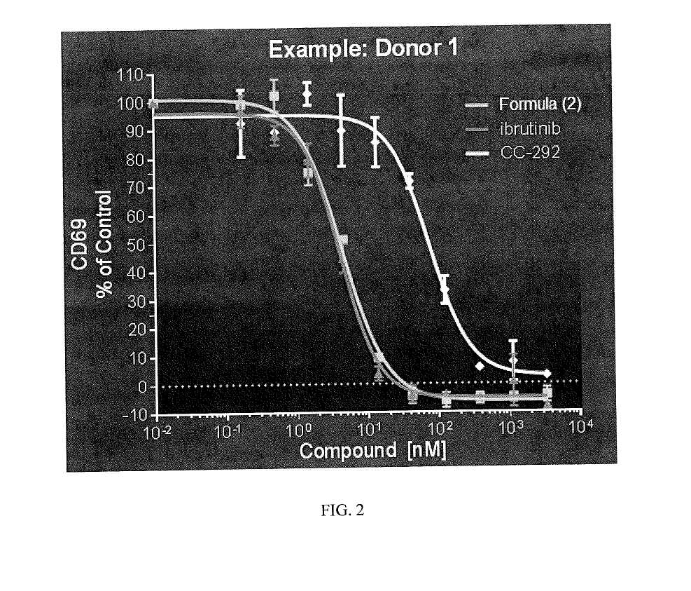

The in vitro potency in whole blood of Formula (2), Formula (10) (ibrutinib), and Formula (17) (CC-292) in inhibiting signals through the B cell receptor was also assessed. Blood from four healthy donors was incubated for 2 hours with the compounds shown over a concentration range, and then stimulated with anti-human IgD (10 μg/mL) for 18 hours. The mean fluorescent intensity (MFI) of CD69 (and CD86, data not shown) on gated CD19+ B cells was measured by flow cytometry. MFI values were normalized so that 100% represents CD69 level in stimulated cells without inhibitor, while 0% represents the unstimulated/no drug condition. The results are shown in FIG. 2. The EC₅₀ values obtained were 8.2 nM (95% confidence interval: 6.5-10.3), 6.1 nM (95% confidence interval: 5.2-7.2), and 121 nM (95% confidence interval: 94-155) for Formula (2), Formula (10) (ibrutinib), and Formula (17) (CC-292), respectively.

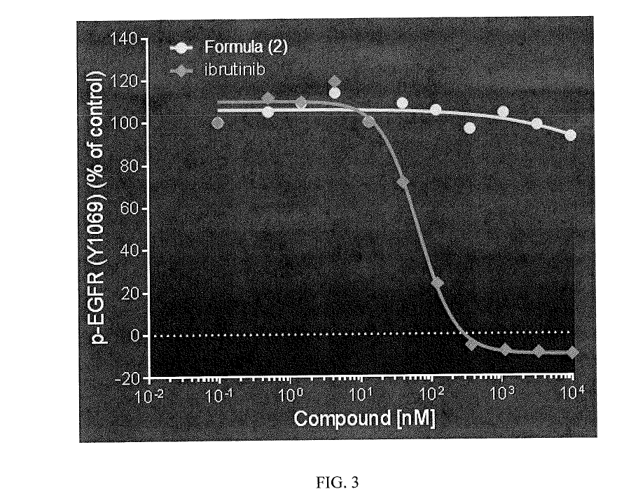

The EGF receptor phosphorylation in vitro was also determined for Formula (2) and Formula (10) (ibrutinib). Epidermoid carcinoma A431 cells were incubated for 2 h with a dose titration of Formula (2) or Formula (10) (ibrutinib), before stimulation with EGF (100 ng/mL) for 5 min to induce EGFR phosphorylation (p-EGFR). Cells were fixed with 1.6% paraformaldehyde and permeabilized with 90% MeOH. Phosphoflow cytometry was performed with p-EGFR (Y1069). MFI values were normalized so that 100% represents the p-EGFR level in stimulated cells without inhibitor, while 0% represents the unstimulated/no drug condition. The results are shown in FIG. 3. EGF-induced p-EGFR inhibition was determined to be 7% at 10 M for Formula (2), while ibrutinib has an EC₅₀ of 66 nM. The much more potent inhibition of EGF-induced p-EGFR by ibrutinib may be associated with increased side effects including diarrhea and rash.

### Example 2

### Synergistic Combinations of BTK Inhibitors and Selective Inhibitors

Ficoll purified mantle cell lymphoma (MCL) cells (2×10⁵) isolated from bone marrow or peripheral blood were treated with each drug alone and with six equimolar concentrations of a BTK inhibitor (Formula (2)) and a selective inhibitor ranging from 0.01 nM to 10 μM on 96-well plates in triplicate. Plated cells were then cultured in HS-5 conditioned media at 37° C. with 5% CO₂. After 72 hours of culture, cell viability was determined using an (3-(4,5-dimethylthiazol-2-yl)-5-(3-carboxymethoxyphenyl)-2-(4-sulfophenyl)-2H-tetrazolium) (MTS) assay (Cell Titer 96, Promega). Viability data were used to generate cell viability curves for each drug alone and in combination for each sample. The potential synergy of the combination of the BTK inhibitor of Formula (2) and the selective inhibitor at a given equimolar concentration was determined using the median effect model as described in Chou and Talalay, Adv Enzyme Regul. 1984, 22, 27-55. The statistical modeling was run in R using a script that utilizes the median effect model as described in Lee, et al., J. Biopharm. Stat. 2007, 17, 461-80. A value of 1, less than 1, and greater than 1 using R defines an additive interaction, a synergistic interaction, and an antagonistic interaction, respectively. The method of Lee, et al., calculates a 95% confidence interval for each data point. For each viability curve, to be considered synergistic, a data point must have an interaction index below 1 and the upper confidence interval must also be below 1. In order to summarize and demonstrate collective synergy results, an interaction dot blot was generated for the primary patient samples.

A similar approach was utilized to study diffuse large B cell lymphoma (DLBCL) (TMD8) and MCL (MINO) cell lines. Cells were treated with each drug alone and with six equimolar concentrations of the BTK inhibitor of Formula (2) and the selective inhibitor ranging from 0.003 nM to 1.0 μM (for TMD8) or 0.03 nM to 10 μM (for MINO) on 96-well plates in triplicate. Plated cells were then cultured in standard conditioned media plus FBS at 37° C. with 5% CO₂. After 72 hours of culture, viability was determined using an MTS assay (Cell Titer 96, Promega). Viability data were used to generate cell viability curves for each drug alone and in combination for each sample.

Additional combination experiments were performed to determine the synergistic, additive, or antagonistic behavior of drug combinations using the Chou/Talalay. Information about experimental design for evaluation of synergy is described in, e.g., Chou and Talalay, Adv. Enzyme Regul. 1984, 22, 27-55 and more generally in Greco, et al., Pharmacol. Rev. 1995, 47, 331-385. The synergy of the combinations was calculated using CalcuSyn software (Biosoft), which is based on the Median Effect methods described by Chou and Talalay, Trends Pharmacol. Sci. 1983, 4, 450-454. The study was performed using the BTK inhibitor of Formula (2) and the selective inhibitor. Single drug activities were first determined in the various cell lines and subsequently, the combination indexes were established using equimolar ratios taking the single agent drug EC50s into consideration. For individual drugs that displayed no single drug activity, equimolar ratios were used at fixed concentrations to establish combination indexes. The readout from 72 hour proliferation assays using Cell TiterGlo (ATP content of remaining cells) determined the fraction of cells that were effected as compared to untreated cells (Fa=fraction affected=(1−((cells+inhibitor)−background signal)/((cells+DMSO)−background signal)).

The combination index obtained may be ranked according to Table 5.

TABLE 5
Combination Index (CI) Ranking Scheme

| Range of CI | Description |
|---|---|
| <0.1 | Very strong synergism |
| 0.1-0.3 | Strong synergism |
| 0.3-0.7 | Synergism |
| 0.7-0.85 | Moderate synergism |
| 0.85-0.9 | Slight synergism |
| 0.9-1.1 | Nearly additive |
| 1.1-1.2 | Slight antagonism |
| 1.2-1.45 | Moderate antagonism |
| 1.45-3.3 | Antagonism |
| 3.3-10 | Strong antagonism |
| >10 | Very strong antagonism |

These data suggest that in companion dogs with naturally occurring B cell lymphomas, treatment with the combination of the BTK inhibitor of Formula (2) and the selective inhibitor may provide increased biological activity (tumor shrinkage and stable disease) and may possibly lead to deeper responses than treatment with the BTK inhibitor of Formula (2) alone.

### Example 3

### BTK Inhibitory Effects on Solid Tumor Microenvironment in an Ovarian Cancer Model

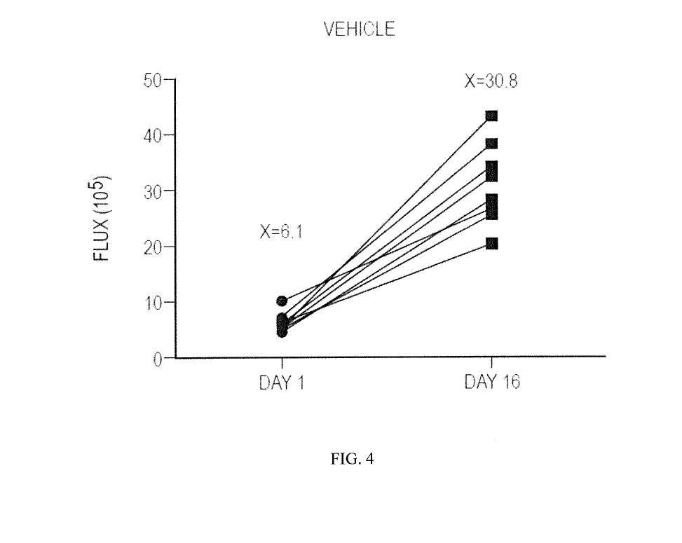

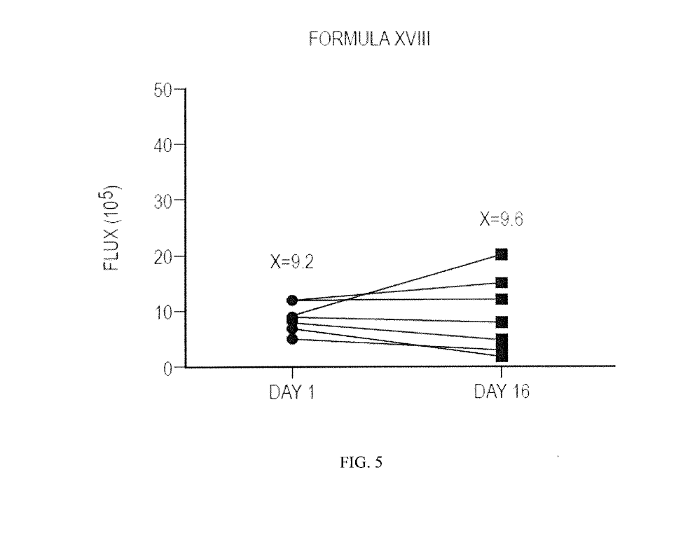

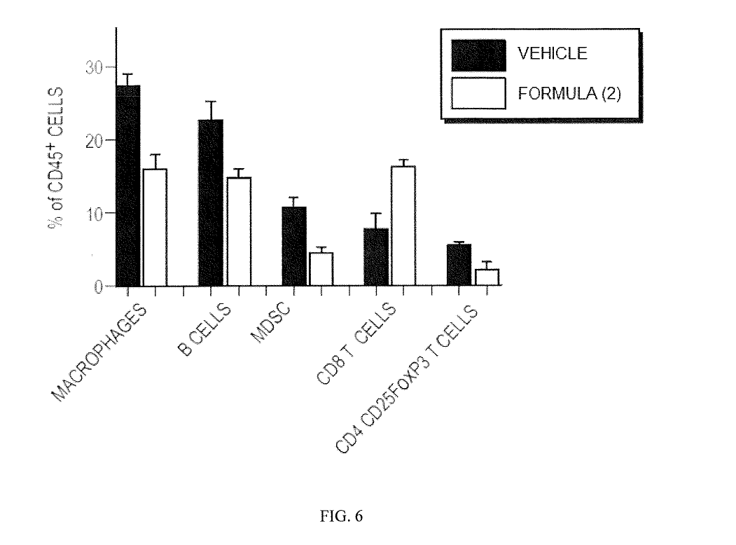

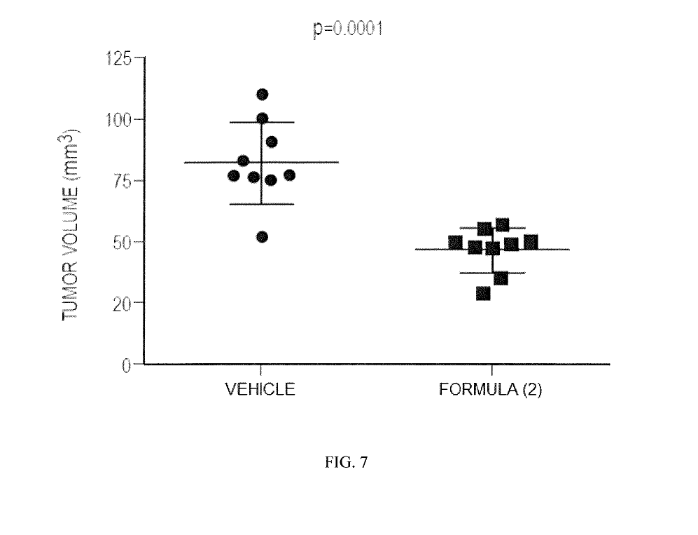

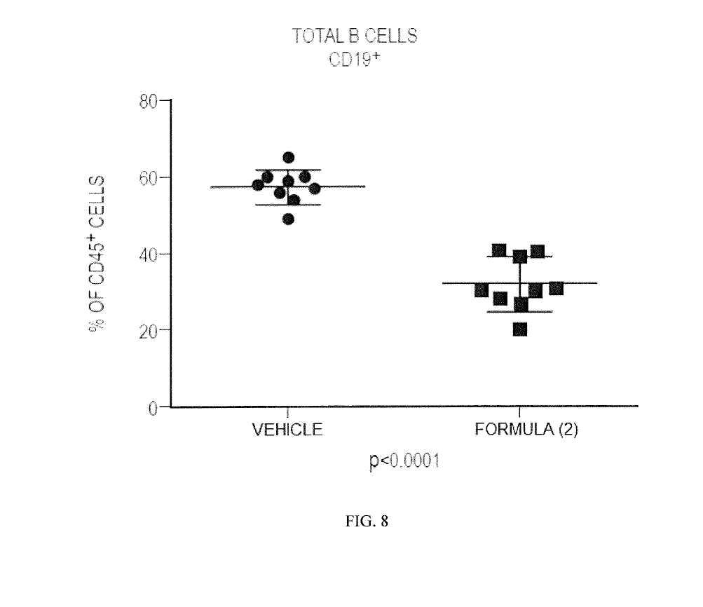

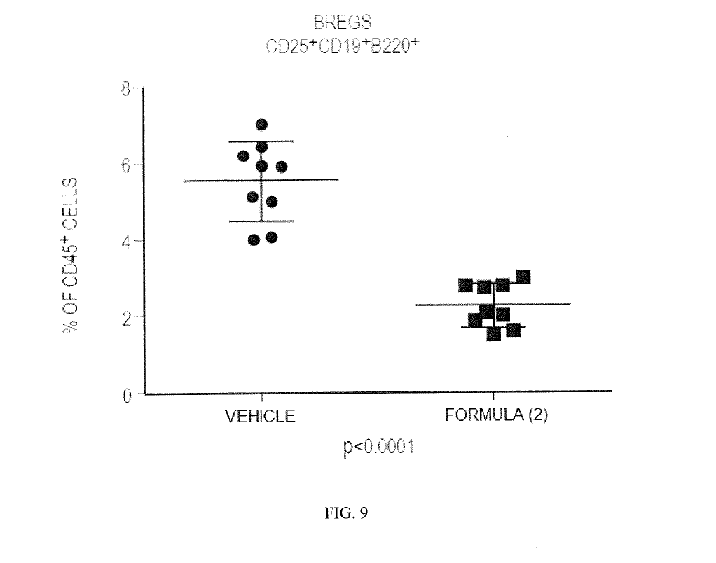

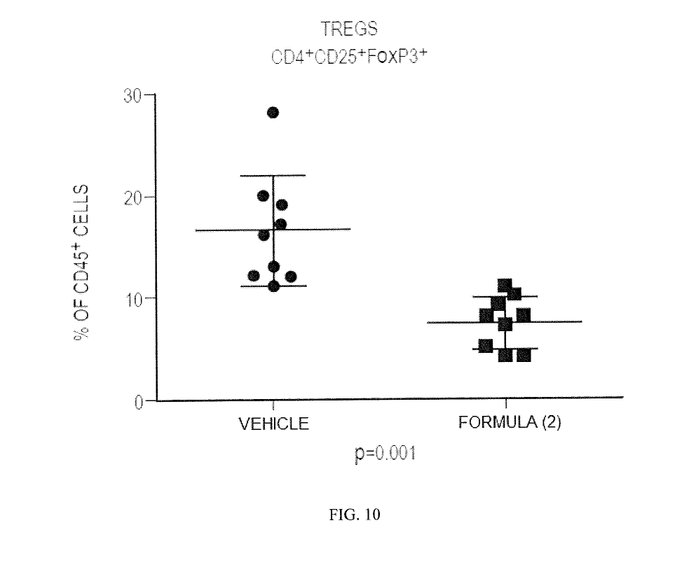

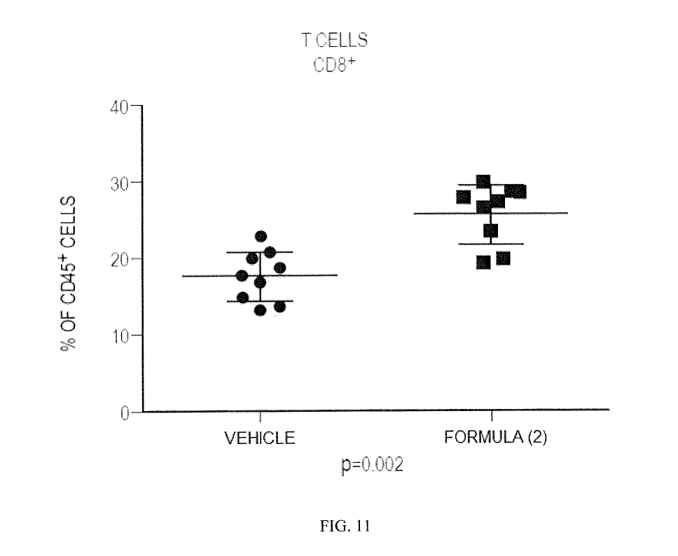

The ID8 syngeneic orthotropic ovarian cancer murine model was used to investigate the therapeutic efficacy of the BTK inhibitor of Formula (2) through treatment of the solid tumor microenvironment. Human ovarian cancer models, including the ID8 syngeneic orthotropic ovarian cancer model and other animal models, are described in Fong and Kakar, J. Ovarian Res. 2009, 2, 12; Greenaway, et al., Gynecol. Oncol. 2008, 108, 385-94; Urzua, et al., Tumour Biol. 2005, 26, 236-44; Janat-Amsbury, et al., Anticancer Res. 2006, 26, 3223-28; Janat-Amsbury, et al., Anticancer Res. 2006, 26, 2785-89. Animals were treated with vehicle or Formula (2), 15 mg/kg/BID given orally. The results of the study are shown in FIG. 4, FIG. 5, FIG. 6, FIG. 7, FIG. 8, FIG. 9, FIG. 10, and FIG. 11.

FIG. 4 and FIG. 5 demonstrate that the BTK inhibitor of Formula (2) impairs ID8 ovarian cancer growth in the ID8 syngeneic murine model. FIG. 6 shows that tumor response to treatment with the BTK inhibitor of Formula (2) correlates with a significant reduction in immunosuppressive tumor-associated lymphocytes in tumor-bearing mice. FIG. 7 shows treatment with the BTK inhibitor of Formula (2) impairs ID8 ovarian cancer growth (through reduction in tumor volume) in the syngeneic murine model. FIG. 8 and FIG. 9 show that the tumor response induced by treatment with the BTK inhibitor of Formula (2) correlates with a significant reduction in immunosuppressive B cells, including Bregs, in tumor-bearing mice. FIG. 10 and FIG. 11 show that the tumor response induced by treatment with the BTK inhibitor of Formula (2) correlates with a significant reduction in immunosuppressive tumor associated Tregs and an increase in CD8⁺ T cells.

The results shown in FIG. 4 to FIG. 11 illustrate the surprising performance of the BTK inhibitor of Formula (2) in modulating tumor microenvironment in a model predictive of efficacy of a treatment for ovarian cancer in humans.

### Example 4

### BTK Inhibitory Effects on Solid Tumor Microenvironment in a KPC Pancreatic Cancer Model

Given the potential for BTK inhibition to affect TAMs and MDSCs, single-active pharmaceutical ingredient Formula (2) was evaluated in mice with advanced pancreatic cancer arising as the result of genetic modifications of oncogenes KRAS and p53, and the pancreatic differentiation promoter PDX-1 (KPC mice). The KPC mouse model recapitulates many of the molecular, histopathologic, and clinical features of human disease (Westphalen and Olive, Cancer J. 2012, 18, 502-510). Mice were enrolled after identification of spontaneously appearing tumors in the pancreas that were ≧100 mm³ (as assessed by high-resolution ultrasonography). Mice were treated with (1) vehicle (N=6); or (2) Formula (2), 15 mg/kg BID given orally (N=6).

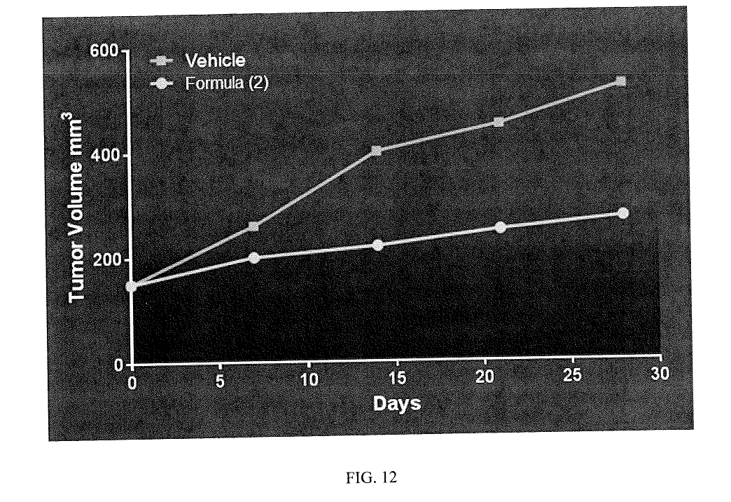

As shown in FIG. 12, treatment with single-active pharmaceutical ingredient Formula (2) substantially slowed pancreatic cancer growth and increased animal survival. With vehicle, tumor volumes predose averaged 152 mm³, and at day 28 averaged 525 mm³. In the cohort treated with Formula (2), tumor volumes predose averaged 165 mm³, and at day 28 averaged 272 mm³, indicating significant improvement. With vehicle, survival at day 14 was 5/6 animals, and at day 28 was 0/6 animals. With Formula (2), survival at day 14 was 6/6 animals, and at day 28 was 5/6 animals.

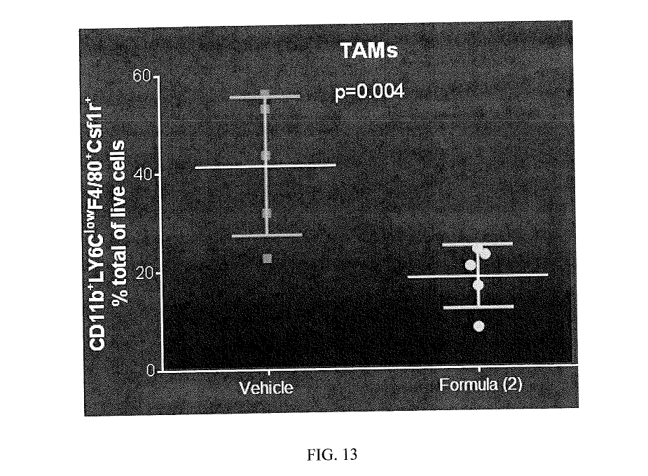

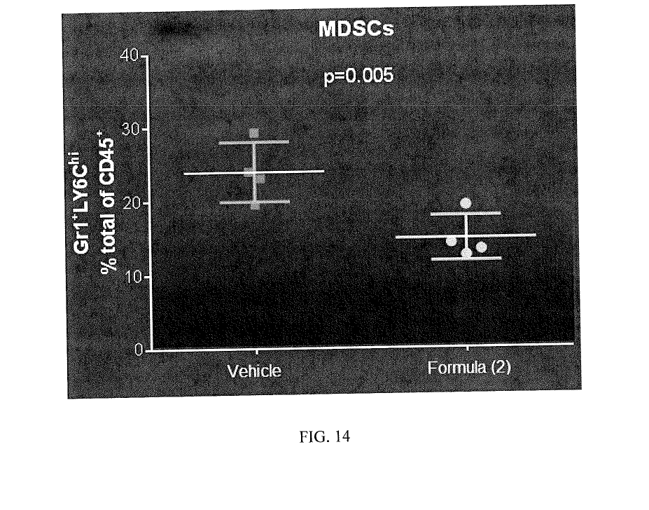

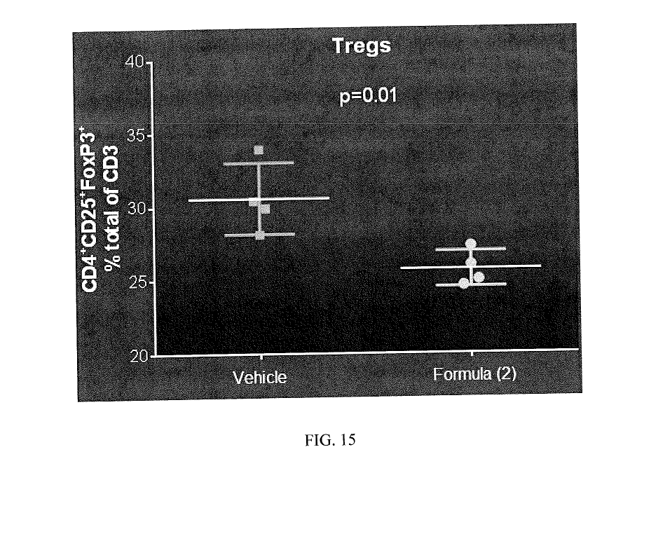

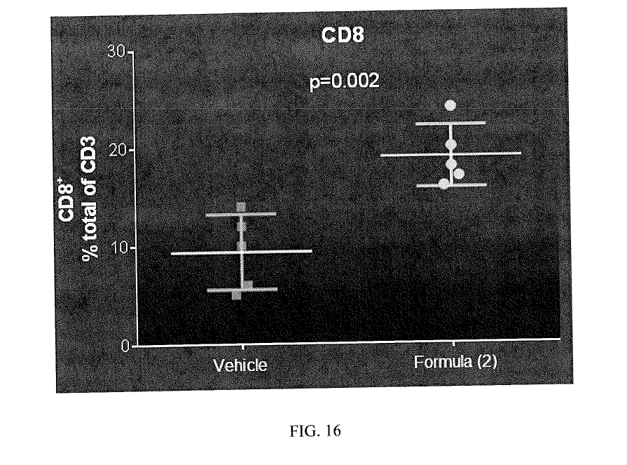

Analysis of tumor tissues showed that immunosuppressive TAMs (CD11b⁺Ly6ClowF4/80⁺Csf1r⁺), MDSCs (Gr1⁺Ly6CHi), and Tregs (CD4⁺CD25⁺FoxP3⁺) were significantly reduced with Formula (2) treatment (FIG. 13, FIG. 14, and FIG. 15). As expected, the decrease in these immunosuppressive cell subsets correlated with a significant increase in CD8⁺ cells (FIG. 16).

### Example 5

### BTK Inhibitory Effects on Solid Tumor Microenvironment in a Non-Small Cell Lung Cancer (NSCLC) Model

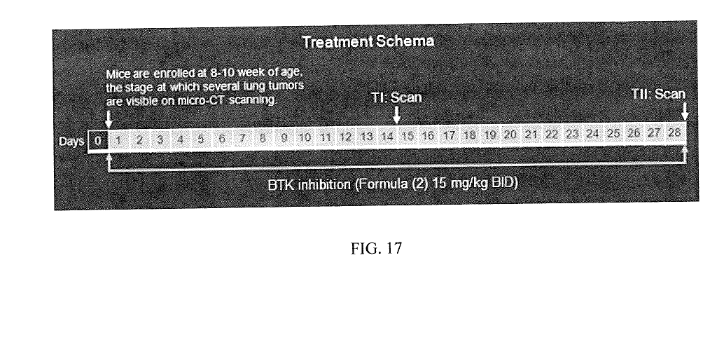

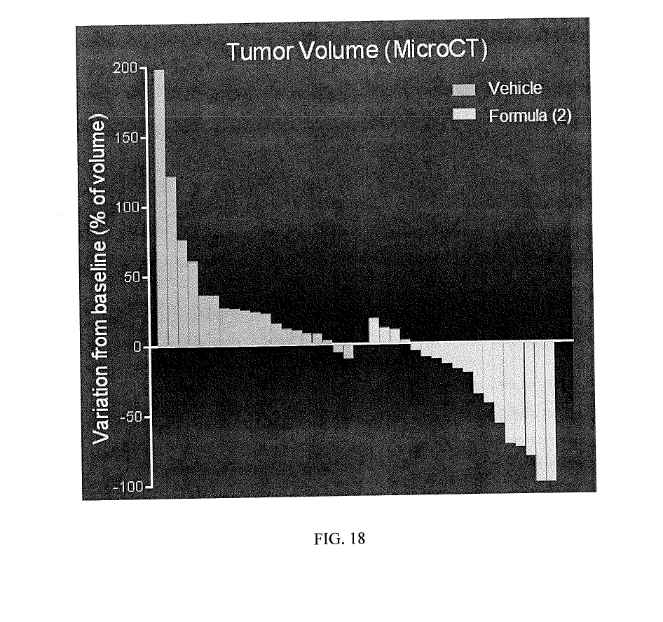

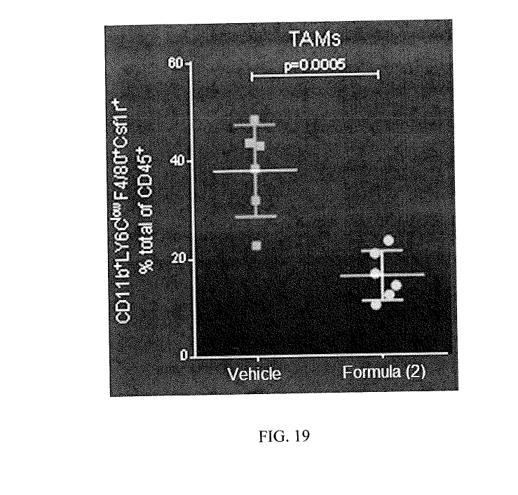

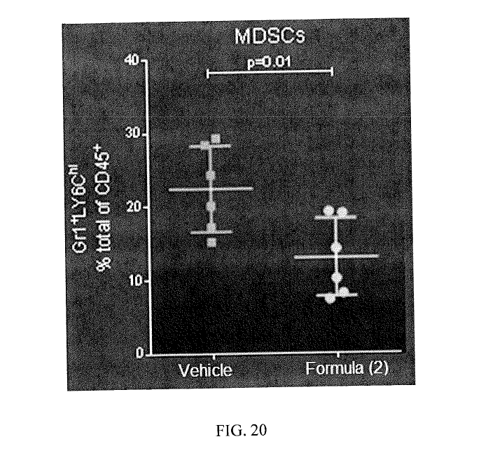

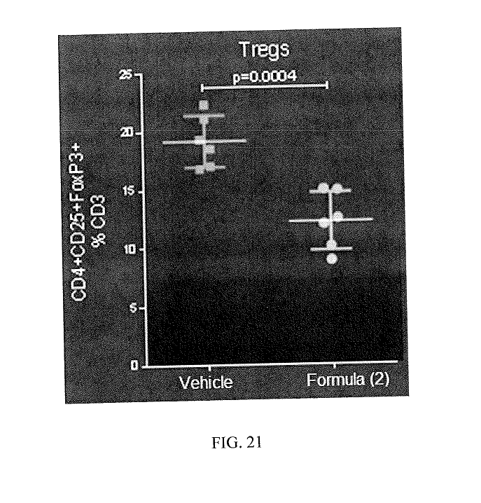

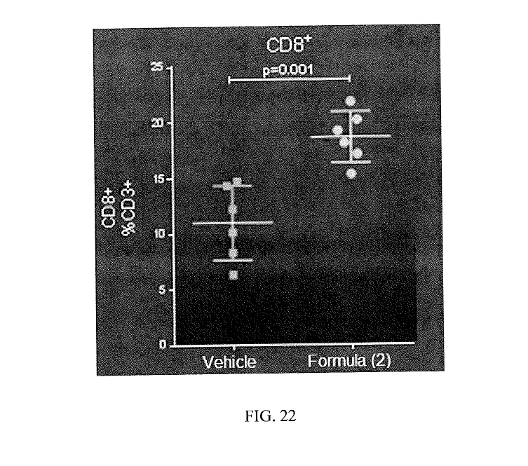

A genetic tumor model of NSCLC (KrasLA2) was studied as a model for lung cancer using the treatment schema shown in FIG. 17. The model is designed to have sporadic expression in single cells of G12D mutant Kras off its own promoter triggered by spontaneous intrachromosomal recombination. Johnson, et al. Nature 2001, 410, 1111-16. While the mutant Kras protein is expressed in a few cells in all tissues, tumor development is seen only in the lung at high penetrance. Mice treated with Formula (2) showed a significant decrease in tumor volumes versus vehicle (FIG. 18) and fewer overall tumors with dosing of 15 mg/kg. The effects on TAMs (FIG. 19), MDSCs (FIG. 20), Tregs (FIG. 21), and CD8+ cells (FIG. 22) were consistent with suppression of the solid tumor microenviroment as demonstrated previously.

### Example 6

### BTK Inhibitory Effects on MDSCs in the Solid Tumor Microenvironment

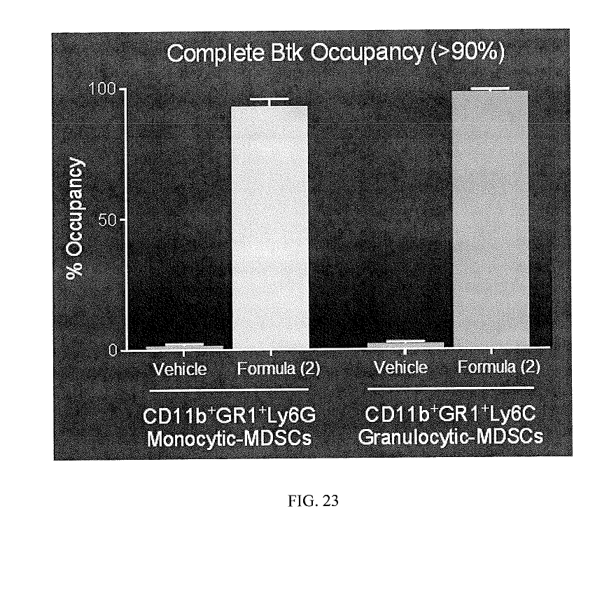

A molecular probe assay was used to calculate the percent irreversible occupancy of total BTK. MDSCs were purified from tumor bearing PDA mice (as described previously) dosed at 15 mg/kg BID of Formula (2). Complete BTK occupancy is observed for both the granulocytic and monocytic MDSC compartments on Day 8 at 4 hours post dose (N=5). The results are shown in FIG. 23.

### Example 7

### Effects of BTK Inhibitors on Antibody-Dependent NK Cell Mediated Cytotoxicity Using Rituximab

Rituximab-combination chemotherapy is today's standard of care in CD20⁺ B-cell malignancies. Previous studies investigated and determined that ibrutinib antagonizes rituximab antibody-dependent cell-mediated cytotoxicity (ADCC) mediated by NK cells. This may be due to ibrutinib's secondary irreversible binding to interleukin-2 inducible tyrosine kinase (ITK) which is required for FcR-stimulated NK cell function including calcium mobilization, granule release, and overall ADCC. Kohrt, et al., Blood 2014, 123, 1957-60.

In this example, the effects of Formula (2) and ibrutinib on NK cell function were evaluated in cell lines and primary NK cells from healthy volunteers and CLL patients. In summary, the results show that the activation of NK cells co-cultured with antibody-coated target cells was strongly inhibited by ibrutinib. The secretion of IFN-γ was reduced by 48% (p=0.018) and 72% (p=0.002) in cultures treated with ibrutinib at 0.1 and 1.0 μM respectively and NK cell degranulation was significantly (p=0.002) reduced, compared with control cultures. Formula (2) treatment at 1 μM, a clinically relevant concentration, did not inhibit IFN-γ or NK cell degranulation. Rituximab-mediated ADCC was evaluated in NK cells from healthy volunteers as well as assays of NK cells from CLL patients targeting autologous CLL cells. In both cases, ADCC was not inhibited by Formula (2) treatment at 1 μM. In contrast, addition of ibrutinib to the ADCC assays strongly inhibited the rituximab-mediated cytotoxicity of target cells, and no increase over natural cytotoxicity was observed at any rituximab concentration. This result indicates that the combination of rituximab and Formula (2) provides an unexpected benefit in the treatment of B cell malignancies and other diseases where NK cell inhibition is undesirable.

BTK is a non-receptor enzyme in the Tec kinase family that is expressed among cells of hematopoietic origin, including B cells, myeloid cells, mast cells and platelets, where it regulates multiple cellular processes including proliferation, differentiation, apoptosis, and cell migration. Khan, Immunol Res. 2001, 23, 147-56; Mohamed, et al., Immunol Rev. 2009, 228, 58-73; Bradshaw, Cell Signal. 2010, 22, 1175-84. Functional null mutations of BTK in humans cause the inherited disease, X linked agammaglobulinemia, which is characterized by a lack of mature peripheral B cells. Vihinen, et al., Front Biosci. 2000, 5, D917-28. Conversely, BTK activation is implicated in the pathogenesis of several B-cell malignancies. Herman, et al., Blood 2011, 117, 6287-96; Kil, et al., Am. J. Blood Res. 2013, 3, 71-83; Tai, et al., Blood 2012, 120, 1877-87; Buggy, and Elias, Int. Rev. Immunol. 2012, 31, 119-32 (Erratum in: Int. Rev. Immunol. 2012, 31, 428). In addition, BTK-dependent activation of mast cells and other immunocytes in peritumoral inflammatory stroma has been shown to sustain the complex microenvironment needed for lymphoid and solid tumor maintenance. Soucek, et al., Neoplasia 2011, 13, 1093-100; Ponader, et al., Blood 2012, 119, 1182-89; de Rooij, et al., Blood 2012, 119, 2590-94. Taken together, these findings have suggested that inhibition of BTK may offer an attractive strategy for treating B cell neoplasms, other hematologic malignancies, and solid tumors.

Ibrutinib (PCI-32765, IMBRUVICA), is a first-in-class therapeutic BTK inhibitor. This orally delivered, small-molecule drug is being developed by Pharmacyclics, Inc. for the therapy of B cell malignancies. As described above, in patients with heavily pretreated indolent non-Hodgkin lymphoma (iNHL), mantle cell lymphoma (MCL), and CLL, ibrutinib showed substantial antitumor activity, inducing durable regressions of lymphadenopathy and splenomegaly in the majority of patients. Advani, et al., J. Clin. Oncol. 2013, 31, 88-94; Byrd, et al., N. Engl. J. Med. 2013, 369, 32-42; Wang, et al., N. Engl. J. Med. 2013, 369, 507-16; O'Brien, et al., Blood 2012, 119, 1182-89. The pattern of changes in CLL was notable. Inhibition of BTK with ibrutinib caused rapid and substantial mobilization of malignant CLL cells from tissue sites into the peripheral blood, as described in Woyach, et al., Blood 2014, 123, 1810-17; this effect was consistent with decreased adherence of CLL to protective stromal cells. Ponader, et al., Blood 2012, 119, 1182-89; de Rooij, et al., Blood 2012, 119, 2590-94. Ibrutinib has been generally well tolerated. At dose levels associated with total BTK occupancy, no dose-limiting toxicities were identified and subjects found the drug tolerable over periods extending to >2.5 years.

Given the homology between BTK and ITK, it has been recently confirmed that ibrutinib irreversibly binds ITK. Dubovsky, et al., Blood 2013, 122, 2539-2549. ITK expression in Fc receptor (FcR)-stimulated NK cells leads to increased calcium mobilization, granule release, and cytotoxicity. Khurana, et al., J. Immunol. 2007, 178, 3575-3582. As rituximab is a backbone of lymphoma therapy, with mechanisms of action including ADCC, as well as direct induction of apoptosis and complement-dependent cytotoxicity, and FcR stimulation is requisite for ADCC, we investigated if ibrutinib or Formula (2) (the latter lacking ITK inhibition) influenced rituximab's anti-lymphoma activity in vitro by assessing NK cell IFN-γ secretion, degranulation by CD107a mobilization, and cytotoxicity by chromium release using CD20⁺ cell lines and autologous patient samples with chronic lymphocytic leukemia (CLL).

Formula (2) is a more selective inhibitor than ibrutinib, as shown previously. Formula (2) is not a potent inhibitor of ITK in contrast to ibrutinib, as shown herein (see, e.g., Table 4). ITK is required for FcR-stimulated NK cell function including calcium mobilization, granule release, and overall ADCC. Anti-CD20 antibodies like rituximab are currently the standard of care for the treatment of many CD20⁺ B cell malignancies, often as part of combination regimens with older chemotherapeutic agents. The potential of ibrutinib or Formula (2) to antagonize ADCC was evaluated in vitro. Previous studies have determined that ibrutinib undesirably antagonizes rituximab ADCC effects mediated by NK cells. Kohrt, et al., Blood 2014, 123, 1957-60. We hypothesized that the BTK inhibitor of Formula (2), which does not have activity against ITK, may preserve NK cell function and therefore synergize rather than antagonize rituximab-mediated ADCC. Rituximab-dependent NK-cell mediated cytotoxicity was assessed using lymphoma cell lines as well as autologous CLL tumor cells.

Cell culture conditions were as follows. Cell lines Raji and DHL-4 were maintained in RPMI 1630 supplemented with fetal bovine serum, L-glutamine, 2-mercaptoethanol and penicillin-streptomycin at 37° C. in a humidified incubator. The HER18 cells were maintained in DEM supplemented with fetal bovine serum, penicillin-streptomycin and. Prior to assay, HER18 cells were harvested using trypsin-EDTA, washed with phosphate-buffered saline (PBS) containing 5% serum and viable cells were counted. For culture of primary target cells, peripheral blood from CLL patients was subject to density centrifugation to obtain peripheral blood mononuclear cells (PBMC). Cell preparations were washed and then subject to positive selection of CD5⁺CD19⁺ CLL cells using magnetic beads (MACS, Miltenyi Biotech). Cell preparations were used fresh after selection. NK cells from CLL patients and healthy volunteers were enriched from peripheral blood collected in sodium citrate anti-coagulant tubes and then subject to density centrifugation. Removal of non NK cells was performed using negative selection by MACS separation. Freshly isolated NK cells were washed three times, enumerated, and then used immediately for ADCC assays.

Cytokine secretion was determined as follows. Rituximab and trastuzumab-dependent NK-cell mediated degranulation and cytokine release were assessed using lymphoma and HER2+ breast cancer cell lines (DHL-4 and HER18, respectively). Target cells were cultured in flat-bottom plates containing 10 μg/mL of rituximab (DHL-4) or trastuzumab (HER18) and test articles (0.1 or 1 μM ibrutinib, 1 μM Formula (2), or DMSO vehicle control). NK cells from healthy donors were enriched as described above and then added to the target cells and incubated for 4 hours at 37° C. Triplicate cultures were performed on NK cells from donors. After incubation, supernatants were harvested, centrifuged briefly, and then analyzed for interferon-γ using an enzyme-linked immunosorbent assay (ELISA) (R&D Systems, Minneapolis, Minn., USA).

Lytic granule release was determined as follows. NK cells from healthy donors were enriched and cultured in the presence of target cells, monoclonal antibodies and test articles as described above. After 4 hours, the cultures were harvested and cells were pelleted, washed, and then stained for flow cytometry evaluation. Degranulation was evaluated via by flow cytometery by externalization of CD107a, a protein normally present on the inner leaflet of lytic granules, and gating on NK cells (CD3-CD16⁺ lymphocytes). The percentage of CD107a positive NK cells was quantified by comparison with a negative control (isotype control, unstained cells/FMO). Control cultures (NK cells cultured without target cells, or NK, target cell co-cultures in the absence of appropriate monoclonal antibody) were also evaluated; all experiments were performed in triplicate.

ADCC assays were performed as follows. Briefly, target cells (Raji or primary CLL) were labeled by incubation at 37° C. with 100 μCi ⁵¹Cr for 4 hours prior to co-culture with NK cells. Cells were washed, enumerated, and then added in triplicate to prepared 96-well plates containing treated NK cells at an effector:target (E:T) ratio of 25:1. Rituximab (Genentech) was added to ADCC wells at concentrations of 0.1, 1.0 or 10 μg/mL and the assays were briefly mixed and then centrifuged to collect cells at the bottom of the wells. The effect of NK cell natural cytotoxicity was assessed in wells containing no rituximab. Cultures were incubated at 37° C. for 4 hours, and then centrifuged. Supernatants were harvested and ⁵¹Cr release was measured by liquid scintillation counting. All experiments were performed in triplicate.

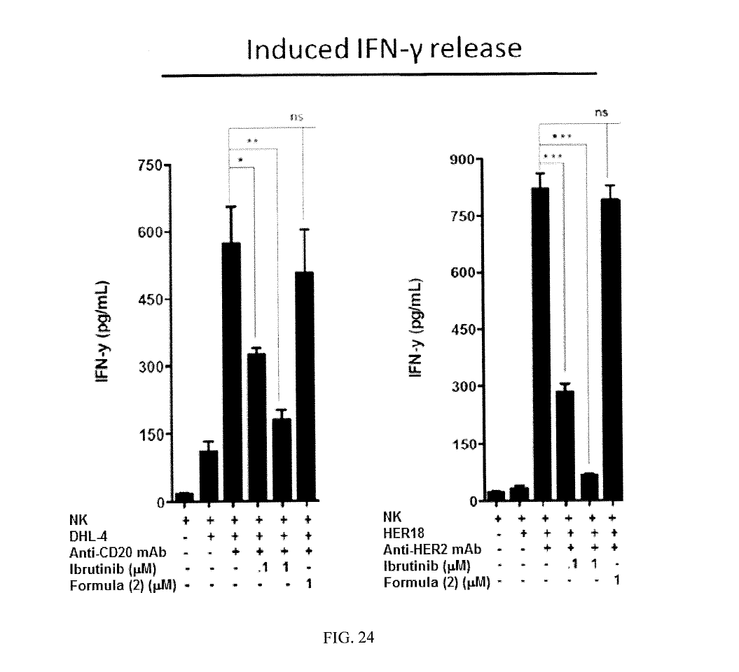

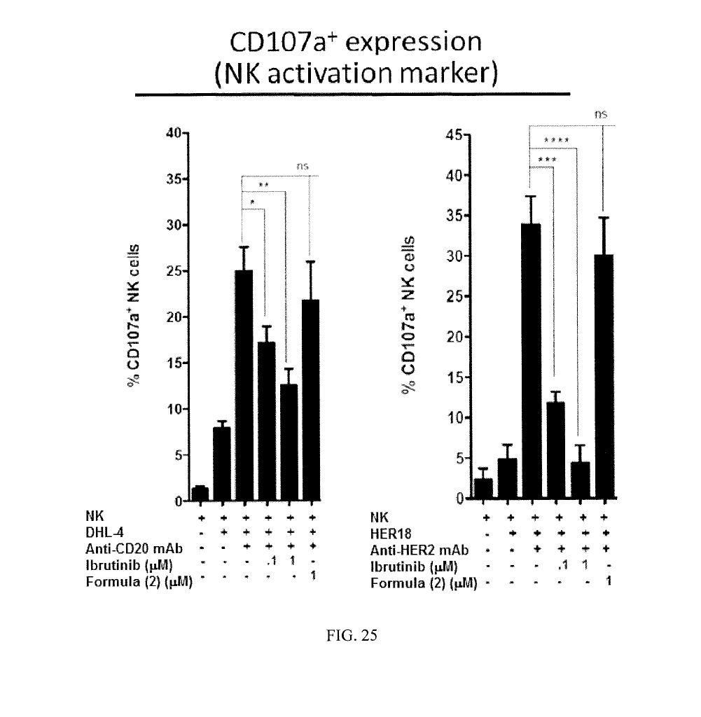

Ibrutinib inhibited rituximab-induced NK cell cytokine secretion in a dose-dependent manner (0.1 and 1 μM) (FIG. 24: 48% p=0.018; 72% p=0.002, respectively). At 1 μM, Formula (2) did not significantly inhibit cytokine secretion (FIG. 24: 3.5%). Similarly, Formula (2) had no inhibitory effect on rituximab-stimulated NK cell degranulation (<2%) while ibrutinib reduced degranulation by ˜50% (p=0.24, FIG. 25). Formula (2) had no inhibitory effect while ibrutinib prevented trastuzumab-stimulated NK cell cytokine release and degranulation by ˜92% and ˜84% at 1 μM, respectively (FIG. 24 and FIG. 25: ***p=0.004, **p=0.002).

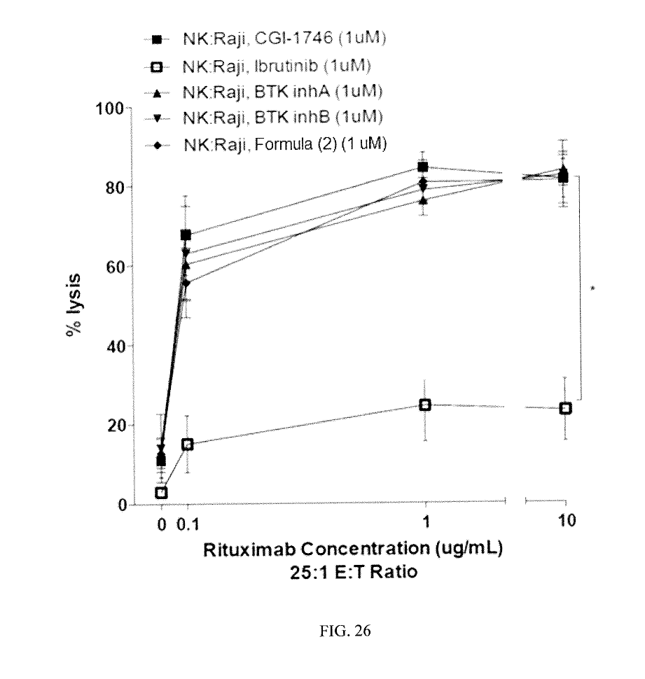

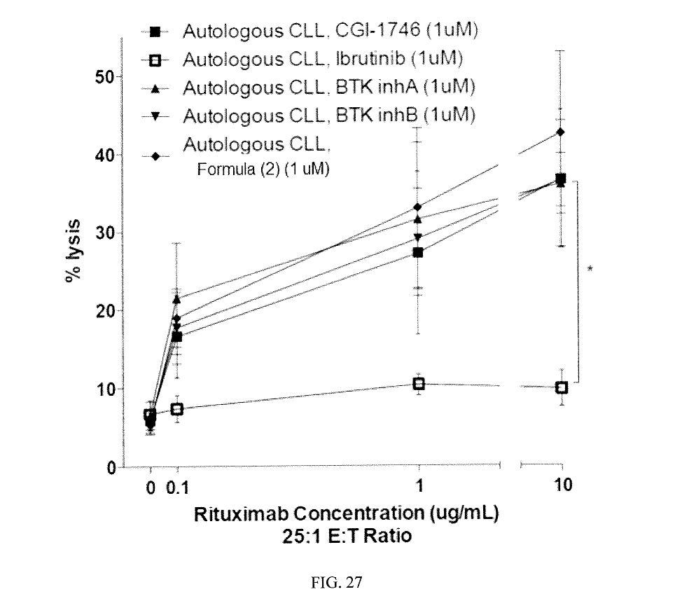

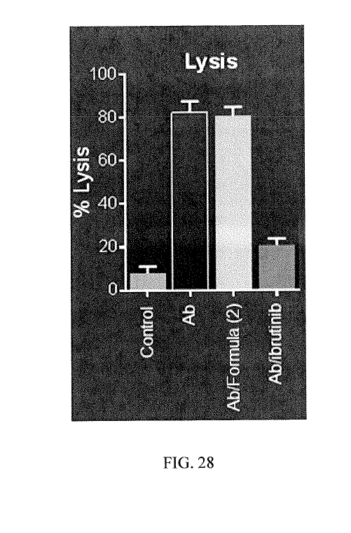

In Raji cell samples, ex vivo NK cell activity against autologous tumor cells was not inhibited by addition of Formula (2) at 1 μM, and increased cell lysis was observed with increasing concentrations of rituximab at a constant E:T ratio (FIG. 26). In primary CLL samples, ex vivo NK cell activity against autologous tumor cells was not inhibited by addition of Formula (2) at 1 μM, and increased cell lysis was observed with increasing concentrations of rituximab at a constant E:T ratio (FIG. 27). In contrast, addition of 1 μM ibrutinib completely inhibited ADCC, with less than 10% cell lysis at any rituximab concentration and no increase in cell lysis in the presence of rituximab, compared with cultures without rituximab. A graph highlighting the significant differences between Formula (2) and ibrutinib is shown in FIG. 28 (where “Ab” refers to rituximab). The difference between Formula (2) and ibrutinib was highly significant in this assay (p=0.001).

In ADCC assays using healthy donor NK cells, antibody-dependent lysis of rituximab-coated Raji cells was not inhibited by addition of 1 μM of Formula (2) (FIG. 28). In these experiments, addition of rituximab stimulated a 5- to 8-fold increase in cell lysis at 0.1 and 1 μg/mL, compared with low (<20%) natural cytotoxicity in the absence of rituximab. As previously reported, addition of 1 μM ibrutinib strongly inhibited the antibody-dependent lysis of target cells, with less than 20% cell lysis at all rituximab concentrations and no increase in ADCC with at higher rituximab concentrations.

Ibrutinib is clinically effective as monotherapy and in combination with rituximab, despite inhibition of ADCC in vitro and in vivo murine models due to ibrutinib's secondary irreversible binding to ITK. Preclinically, the efficacy of therapeutics which do not inhibit NK cell function, including Formula (2), is superior to ibrutinib. These results provide support for the unexpected property of Formula (2) as a synergistic and superior active pharmaceutical ingredient than ibrutinib to use in combinations with antibodies that have ADCC as a mechanism of action. The improved performance of Formula (2) in combination with anti-CD20 antibody therapies is expected to extend to its use in combination with PD-1/PD-L1 inhibitors in both hematological malignancies and solid tumors, as these combinations would also benefit from reduced inhibition of NK cell function.

### Example 8

### Effects of BTK Inhibition on Antibody-Dependent NK Cell Mediated Cytotoxicity Using Obinutuzumab

It has been shown above that ibrutinib undesirably antagonizes rituximab ADCC effects mediated by NK cells, and that Formula (2) does not antagonize rituximab ADCC effects and instead allows for a synergistic combination. As noted previously, this may be due to ibrutinib's secondary irreversible binding to ITK, which is required for FcR-stimulated NK cell function including calcium mobilization, granule release, and overall ADCC. H. E. Kohrt, et al., Blood 2014, 123, 1957-60. The potential for ibrutinib antagonization of obinutuzumab (GA-101) ADCC as mediated by NK cells was also explored and compared to the effects of Formula (2).

The NK cell degranulation/ADCC assay was performed using a whole blood assay with CLL targets added to normal donor whole blood, in the presence or absence of different doses of Formula (2) and ibrutinib, followed by opsonization with the anti-CD20 antibody obinutuzumab. Ibrutinib was used as a control, and two blinded samples of BTK inhibitors, Formula (2) and a second sample of ibrutinib, were provided to the investigators. Degranulation in whole blood was performed as follows. CLL targets (MEC-1 cells) were expanded in RPMI 1640 medium (Life Technologies, Inc.) with 10% fetal bovine serum (FBS). Exponentially growing cells were used. On the day of the experiment, 8 mL of blood was drawn from a normal volunteer into a test tube containing desirudin to obtain a final concentration of 50 μg/mL. A white blood cell (WBC) count of whole blood was performed. MEC-1 cells were re-suspended at the concentration of WBC in whole blood (e.g., if 6×10⁶ WBC/mL was measured, MEC-1 cells were re-suspended at 6×10⁶ cells/mL, to allow for a final WBC:MEC-1 cell ratio of 1:1). The ibrutinib control and two blinded BTK inhibitors were diluted in X-VIVO 15 serum-free hematopoietic cell medium (Lonza Group, Ltd.) to concentrations of 200 μM, 20 μM and 2 μM. 170 μL aliquots of unmanipulated whole blood were incubated with 10 μL BTK inhibitors or X-VIVO 15 medium for one hour into a plate. Cetuximab and obinutuzumab (GA-101) were diluted in X-VIVO 15 medium to a concentration of 20 μg/mL. Equal volumes of MEC-1 cells and antibodies were incubated for 5 minutes. After incubation, 20 μL of MEC-1 cells and antibodies was added to whole blood and the BTK inhibitors/X-VIVO 15 medium (for a final volume of 200 μL). The samples were placed in a 5% CO₂ incubator for 4 hours at 37° C. The experimental conditions thus achieved a WBC:MEC-1 cell ratio of 1:1, with final concentrations of the BTK inhibitors in the assay of 10 μM, 1 μM and 0.1 μM and final concentrations of the antibodies of 1 μg/mL.

After 4 hours, the samples were mixed gently and 50 μL aliquots were removed from each well and placed in fluorescence-activated cell sorting (FACS) test tubes. A 20 μL aliquot of anti-CD56-APC antibody and anti-CD107a-PE antibody was added. The samples were incubated for 20 minutes at room temperature in the dark. An aliquot of 2 mL of FACS lysing solution (BD Biosceinces) was added. The samples were again incubated for 5 minutes, and then centrifuged at 2000 rpm for 5 minutes. Supernatant was discarded and the cell pellet was resuspended in 500 μL of PBS. The samples were analyzed on the flow cytometer for CD107a⁺ NK cells (CD56⁺).

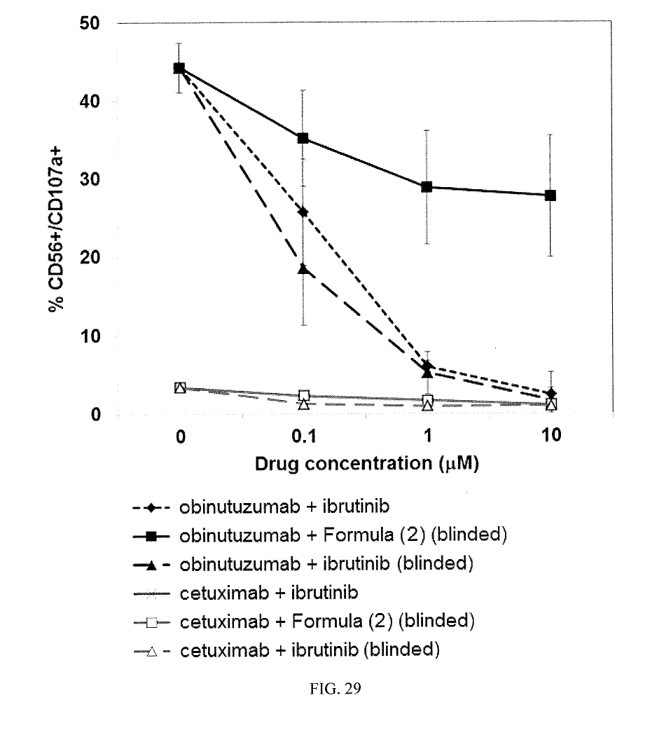

The NK cell degranulation results are summarized in FIG. 29 for n=3 experiments, which shows the effects on whole blood after pretreatment for 1 hour with the BTK inhibitors at the concentrations shown and subsequent stimulation with MEC-1 opsonised with obinutuzumab or cetuximab at 1 μg/mL for 4 hours. A strong reduction in the percentage of CD56⁺/CD107a⁺ NK cells is observed using ibrutinib (both as a control and blinded BTK inhibitor), which indicates that ibrutinib undesirably antagonizes NK cells. In contrast, Formula (2) shows little antagonism towards NK cells, and had a minimal effect on obinutuzumab-stimulated NK cell degranulation while ibrutinib reduced obinutuzumab-stimulated NK degranulation by greater than 40%. These results support the synergistic combination of obinutuzumab and Formula (2) in treatment of human B cell malignancies.

### Example 9

### Effects of BTK Inhibition on Generalized NK Cell Mediated Cytotoxicity

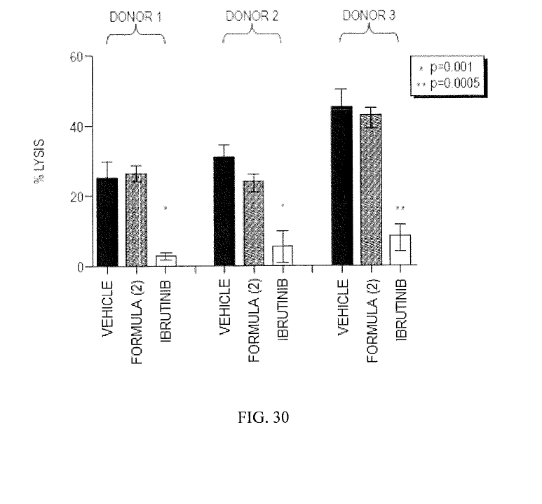

An assay was performed to assess the effects of BTK inhibition using Formula (2) on generalized NK killing (non-ADCC killing). The targets (K562 cells) do not express major histocompatibility complex (MHC) class I, so they do not inactivate NK cells. Target cells were grown to mid-log phase, and 5×10⁵ cells were labeled in 100 μL of assay medium (IMDM with 10% FCS and penicillin/streptomycin) with 100 μCi of ⁵¹Cr for 1 hour at 37° C. Cells were washed twice and resuspended in assay medium. A total of 5000 target cells/well was used in the assay. Effector cells were resuspended in assay medium, distributed on a V-bottom 96-well plate, and mixed with labeled target cells at 40:1 E:T ratios. Maximum release was determined by incubating target cells in 1% Triton X-100. For spontaneous release, targets were incubated without effectors in assay medium alone. After a 1 minute centrifugation at 1000 rpm, plates were incubated for 4 and 16 hours at 37° C. Supernatant was harvested and ⁵¹Cr release was measured in a gamma counter. Percentage of specific release was calculated as (experimental release-spontaneous release)/(maximum release-spontaneous release)×100. The results are shown in FIG. 30.

### Example 10

### Effects of BTK Inhibition on T Cells

An assay was performed to assess the effects of BTK inhibition using Formula (2) on T cells. Enriched CD4⁺ T cells are plated on 24-well culture dishes that have been precoated 2 hr with 250 μL anti-TCRβ (0.5 μg/mL) plus anti-CD28 (5 μg/mL) at 37° C. in PBS. The cells are then supplemented with media containing BTK inhibitors along with the skewing cytokines as indicated in the following. The Th17 and Treg cultures are grown for 4 days before analysis. The cells are maintained for an additional 3 days with skewing cytokines (Th17; 20 ng/mL IL-6, 0.5 ng/mL TGF-β, 5 μg/mL IL-4, 5 μg/mL IFN-γ and Treg; 0.5 ng/mL TGF-β, 5 μg/mL IL-4, 5 μg/mL IFN-γ) and are supplemented with IL2 as a growth factor.

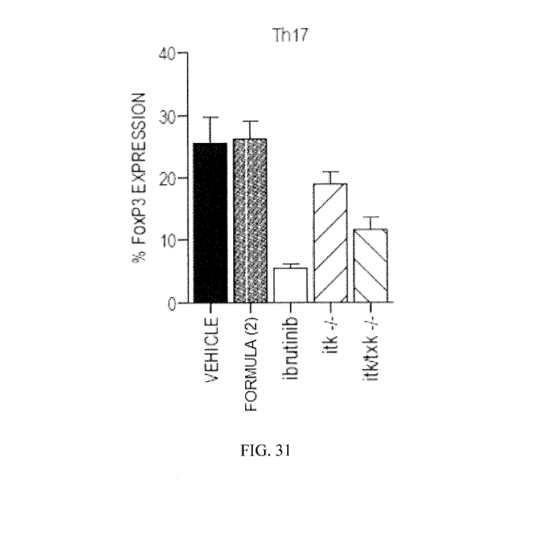

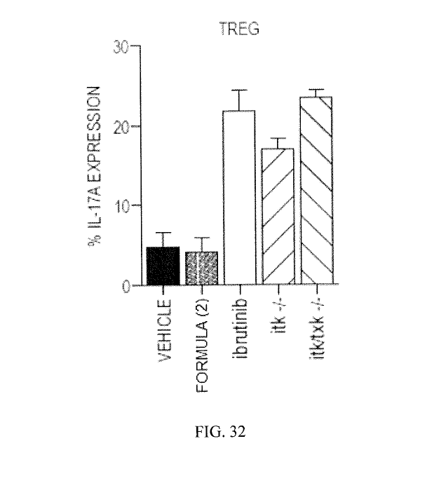

The results are shown in FIG. 31 and FIG. 32, and further illustrate the surprising properties of Formula (2) in comparison to Formula (10) (ibrutinib). Because of the lack of activity of Formula (2) on ITK and Txk, no adverse effect on Th17 and Treg development was observed. Since ibrutinib inhibits both ITK and Txk, a profound inhibition of Th17 cells and an increase in Treg development is observed, which is comparable to the murine ITK/Txk double knock-out cells which were used as a control.

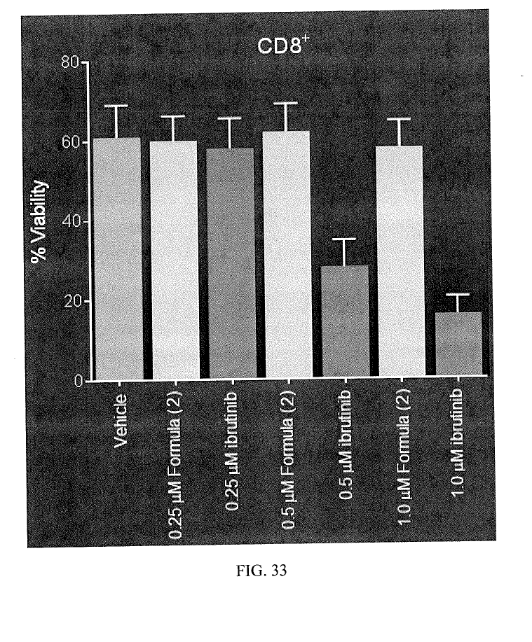

The effects of ibrutinib in comparison to Formula (2) on CD8⁺ T cell viability were also assessed. Total T cells were plated on anti-TCR and anti-CD28 coated wells in the presence of both BTK inhibitors. Neutral culture conditions were used that will not polarize T cells to a helper lineage. The cells are grown for 4 days and are then stained with anti-CD4, anti-CD8 and LIVE/DEAD reagent to determine if the drugs have selective effects on either the CD4⁺ or CD8⁺ cells. Statistical significance was calculated using the Mann Whitney T-test. The results, shown in FIG. 33, indicate that higher concentrations of Formula (10) (ibrutinib) have a strong, negative effect on CD8⁺ T cell viability that is not observed with Formula (2) at any concentration.

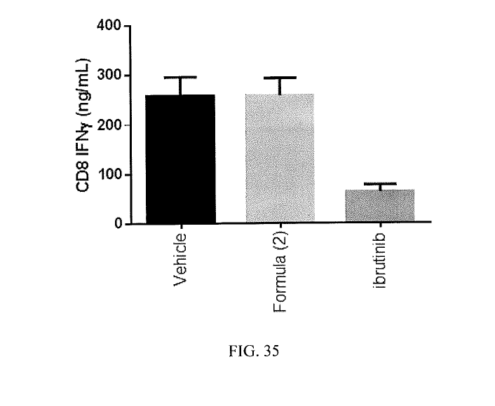

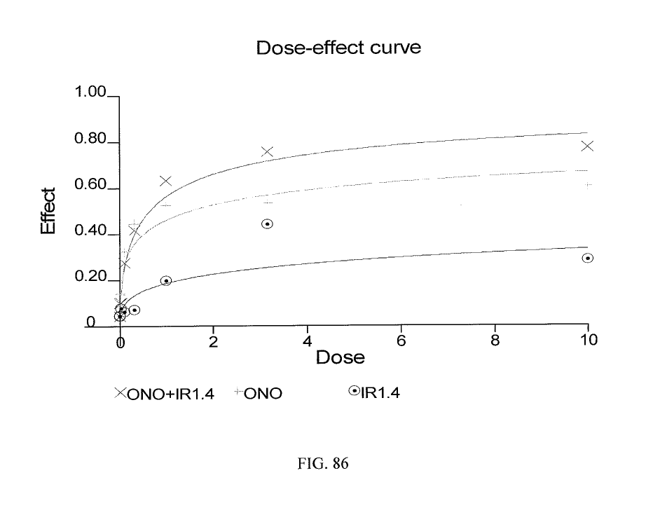

Without being bound by any theory, CD8⁺ T cells have two primary effector functions: (1) produce large amounts of IFN-γ (which activates macrophages), and (2) cytolytic activity. A cytotoxic T cell (CTL) assay was performed to compare the BTK inhibitors of Formula (2) and Formula (10) (ibrutinib). Effectors were prepared by generating CTL by culturing MHC mismatched splenocytes for 4 days with (500 nM) and without Formula (2) or Formula (10) (ibrutinib). The targets were B lymphoblasts from lipopolysaccharide (LPS) treated cultures. The assay was performed by incubating different ratios of effectors:targets for 4 hours. In FIG. 86, the results show that Formula (10) (ibrutinib) affects CD8⁺ T cell function as measured by % cytotoxicity. Formula (2), in contrast, has no effect on CD8⁺ T cell function as measured by % cytotoxicity relative to vehicle. The effect on CD8⁺ T cell function can also be observed by measurement of IFN-γ levels, as shown in FIG. 35, where Formula (10) (ibrutinib) again results in a significant loss of function relative to Formula (2) and vehicle.

### Example 11

### Clinical Study of a BTK Inhibitor in Leukemia/Lymphoma and Effects on Bone Marrow and Lymphoid Microenvironments

Clinical studies have shown that targeting the BCR signaling pathway by inhibiting BTK produces significant clinical benefit in patients with non-Hodgkin's lymphoma (NHL). The second generation BTK inhibitor, Formula (2), achieves significant oral bioavailability and potency, and has favorable preclinical characteristics, as described above. The purpose of this study is to evaluate the safety and efficacy of the second generation BTK inhibitor of Formula (2) in treating subjects with chronic lymphocytic leukemia (CLL) and small lymphocytic lymphoma (SLL).

The design and conduct of this study is supported by an understanding of the history and current therapies for subjects with lymphoid cancers; knowledge of the activity and safety of a first-generation BTK inhibitor, ibrutinib, in subjects with hematologic cancers; and the available nonclinical information regarding Formula (2). The collective data support the following conclusions. BTK expression plays an important role in the biology of lymphoid neoplasms, which represent serious and life-threatening disorders with continuing unmet medical need. Clinical evaluation of Formula (2) as a potential treatment for these disorders has sound scientific rationale based on observations that the compound selectively abrogates BTK activity and shows activity in nonclinical models of lymphoid cancers. These data are supported by clinical documentation that ibrutinib, a first-generation BTK inhibitor, is clinically active in these diseases. Ibrutinib clinical data and Formula (2) nonclinical safety pharmacology and toxicology studies support the safety of testing Formula (2) in subjects with B cell malignancies.

The primary objectives of the clinical study are as follows: (1) establish the safety and the MTD of orally administered Formula (2) in subjects with CLL/SLL; (2) determine pharmacokinetics (PK) of orally administered Formula (2) and identification of its major metabolite(s); and (3) measure pharmacodynamic (PD) parameters including drug occupancy of BTK, the target enzyme, and effect on biologic markers of B cell function.

The secondary objective of the clinical study is to evaluate tumor responses in patients treated with Formula (2).

This study is a multicenter, open-label, nonrandomized, sequential group, dose escalation study. The following dose cohorts will be evaluated:

Cohort 1: 100 mg/day for 28 days (=1 cycle)

Cohort 2: 175 mg/day for 28 days (=1 cycle)

Cohort 3: 250 mg/day for 28 days (=1 cycle)

Cohort 4: 350 mg/day for 28 days (=1 cycle)

Cohort 5: 450 mg/day for 28 days (=1 cycle)

Cohort 6: To be determined amount in mg/day for 28 days (=1 cycle)

Each cohort will be enrolled sequentially with 6 subjects per cohort. If ≦1 dose-limiting toxicity (DLT) is observed in the cohort during Cycle 1, escalation to the next cohort will proceed. Subjects may be enrolled in the next cohort if 4 of the 6 subjects enrolled in the cohort completed Cycle 1 without experiencing a DLT, while the remaining 2 subjects are completing evaluation. If ≧2 DLTs are observed during Cycle 1, dosing at that dose and higher will be suspended and the MTD will be established as the previous cohort. The MTD is defined as the largest daily dose for which fewer than 33% of the subjects experience a DLT during Cycle 1. Dose escalation will end when either the MTD is achieved or at 3 dose levels above full BTK occupancy, whichever occurs first. Full BTK occupancy is defined as Formula (2) active-site occupancy of >80% (average of all subjects in cohort) at 24 hours postdose. Should escalation to Cohort 6 be necessary, the dose will be determined based on the aggregate data from Cohorts 1 to 5, which includes safety, efficacy, and PK/PD results. The dose for Cohort 6 will not exceed 900 mg/day.

Treatment with Formula (2) may be continued for >28 days until disease progression or an unacceptable drug-related toxicity occurs. Subjects with disease progression will be removed from the study. All subjects who discontinue study drug will have a safety follow-up visit 30 (±7) days after the last dose of study drug unless they have started another cancer therapy within that timeframe. Radiologic tumor assessment will be done at screening and at the end of Cycle 2, Cycle 4, and Cycle 12 and at investigator discretion. Confirmation of complete response (CR) will require bone marrow analysis and radiologic tumor assessment. For subjects who remain on study for >11 months, a mandatory bone marrow aspirate and biopsy is required in Cycle 12 concurrent with the radiologic tumor assessment.

All subjects will have standard hematology, chemistry, and urinalysis safety panels done at screening. This study also includes pancreatic function assessment (serum amylase and serum lipase) due to the pancreatic findings in the 28-day GLP rat toxicity study. Once dosing commences, all subjects will be evaluated for safety once weekly for the first 4 weeks, every other week for Cycle 2, and monthly thereafter. Blood samples will be collected during the first week of treatment for PK/PD assessments. ECGs will be done at screening, and on Day 1-2, 8, 15, 22, 28 of Cycle 1, Day 15 and 28 of Cycle 2, and monthly thereafter through Cycle 6. ECGs are done in triplicate for screening only. Thereafter, single ECG tests are done unless a repeat ECG testing is required.

Dose-limiting toxicity is defined as any of the following events (if not related to disease progression): (1) any Grade ≧3 non-hematologic toxicity (except alopecia) persisting despite receipt of a single course of standard outpatient symptomatic therapy (e.g., Grade 3 diarrhea that responds to a single, therapeutic dose of Imodium® would not be considered a DLT); (2) grade ≧3 prolongation of the corrected QT interval (QTc), as determined by a central ECG laboratory overread; (3) grade 4 neutropenia (absolute neutrophil count [ANC]<500/μL) lasting >7 days after discontinuation of therapy without growth factors or lasting >5 days after discontinuation of therapy while on growth factors (i.e., Grade 4 neutropenia not lasting as long as specified will not be considered a DLT), (4) grade 4 thrombocytopenia (platelet count <20,000/μL) lasting >7 days after discontinuation of therapy or requiring transfusion (i.e., Grade 4 thrombocytopenia not lasting as long as specified will not be considered a DLT), and (5) dosing delay due to toxicity for >7 consecutive days.

The efficacy parameters for the study include overall response rate, duration of response, and progression-free survival (PFS). The safety parameters for the study include DLTs and MTD, frequency, severity, and attribution of adverse events (AEs) based on the Common Terminology Criteria for Adverse Events (CTCAE v4.03) for non-hematologic AEs. Hallek, et al., Blood 2008, 111, 5446-5456.

The schedule of assessments is as follows, with all days stated in the following meaning the given day or +/−2 days from the given day. A physical examination, including vital signs and weight, are performed at screening, during cycle 1 at 1, 8, 15, 22, and 28 days, during cycle 2 at 15 and 28 days, during cycles 3 to 24 at 28 days, and at follow up (after the last dose). The screening physical examination includes, at a minimum, the general appearance of the subject, height (screening only) and weight, and examination of the skin, eyes, ears, nose, throat, lungs, heart, abdomen, extremities, musculoskeletal system, lymphatic system, and nervous system. Symptom-directed physical exams are done thereafter. Vital signs (blood pressure, pulse, respiratory rate, and temperature) are assessed after the subject has rested in the sitting position. Eastern Cooperative Oncology Group (ECOG) status is assessed at screening, during cycle 1 at 1, 8, 15, 22, and 28 days, during cycle 2 at 15 and 28 days, during cycles 3 to 24 at 28 days, and at follow up, using the published ECOG performance status indications described in Oken, et al., Am. J. Clin. Oncol. 1982, 5, 649-655. ECG testing is performed at screening, during cycle 1 at 1, 2, 8, 15, 22, and 28 days, during cycle 2 at 15 and 28 days, during cycles 3 to 24 at 28 days, and at follow up. The 12-lead ECG test will be done in triplicate (≧1 minute apart) at screening. The calculated QTc average of the 3 ECGs must be <480 ms for eligibility. On cycle 1, day 1 and cycle 1, day 8, single ECGs are done predose and at 1, 2, 4, and 6 hours postdose. The single ECG on Cycle 1 Day 2 is done predose. On cycle 1, day 15, day 22, and day 28, a single ECG is done 2 hours post-dose. Starting with cycle 2, a single ECG is done per visit. Subjects should be in supine position and resting for at least 10 minutes before study-related ECGs. Two consecutive machine-read QTc>500 ms or >60 ms above baseline require central ECG review. Hematology, including complete blood count with differential and platelet and reticulocyte counts, is assesed at screening, during cycle 1 at 1, 8, 15, 22, and 28 days, during cycle 2 at 15 and 28 days, during cycles 3 to 24 at 28 days, and at follow up. Serum chemistry is assesed at screening, during cycle 1 at 1, 8, 15, 22, and 28 days, during cycle 2 at 15 and 28 days, during cycles 3 to 24 at 28 days, and at follow up. Serum chemistry includes albumin, alkaline phosphatase, ALT, AST, bicarbonate, blood urea nitrogen (BUN), calcium, chloride, creatinine, glucose, lactate dehydrogenase (LDH), magnesium, phosphate, potassium, sodium, total bilirubin, total protein, and uric acid. Cell counts and serum immunoglobulin are performed at screening, at cycle 2, day 28, and at every 6 months thereafter until last dose and include T/B/NK/monocyte cell counts (CD3, CD4, CD8, CD14, CD19, CD19, CD16/56, and others as needed) and serum immunoglobulin (IgG, IgM, IgA, and total immunoglobulin). Bone marrow aspirates are performed at cycle 12. Pharmacodynamics samples are drawn during cycle 1 at 1, 2, and 8 days, and at follow up. On days 1 and 8, pharmacodynamic samples are drawn pre-dose and 4 hours (+10 minutes) post-dose, and on day 2, pharmacodynamic samples are drawn pre-dose. Pharmacokinetics samples are drawn during cycle 1 at 1, 2, 8, 15, 22, and 28 days. Pharmacokinetic samples for Cycle 1 Day 1 are drawn pre-dose and at 0.5, 1, 2, 4, 6 and 24 hours (before dose on Day 2) post-dose. Samples for Cycle 1 Day 8 are drawn pre-dose and at 0.5, 1, 2, 4, and 6 hours post-dose. On Cycle 1 Day 15, 22, and 28, a PK sample is drawn pre-dose and the second PK sample must be drawn before (up to 10 minutes before) the ECG acquisition, which is 2 hours postdose. Pretreatment radiologic tumor assessments are performed within 30 days before the first dose. A computed tomography (CT) scan (with contrast unless contraindicated) is required of the chest, abdomen, and pelvis. In addition, a positron emission tomography (PET) or PET/CT must done for subjects with SLL. Radiologic tumor assessments are mandatory at the end of Cycle 2 (−7 days), Cycle 4 (−7 days), and Cycle 12 (−7 days). Otherwise, radiologic tumor assessments are done at investigator discretion. A CT (with contrast unless contraindicated) scan of the chest, abdomen, and pelvis is required for subjects with CLL. In addition, a PET/CT is required in subjects with SLL. Bone marrow and radiologic assessments are both required for confirmation of a complete response (CR). Clinical assessments of tumor response should be done at the end of Cycle 6 and every 3 months thereafter. Molecular markers are measured at screening, and include interphase cytogenetics, stimulated karyotype, IgHV mutational status, Zap-70 methylation, and beta-2 microglobulin levels. Urinalysis is performed at screening, and includes pH, ketones, specific gravity, bilirubin, protein, blood, and glucose. Other assessments, including informed consent, eligibility, medical history, and pregnancy test are done at the time of screening.

The investigator rates the subject's response to treatment based on recent guidelines for CLL, as given in Hallek, et al., Blood 2008, 111, 5446-56, and for SLL, as given in Cheson, et al., J. Clin. Oncol. 2007, 25, 579-586. The response assessment criteria for CLL are summarized in Table 6.

TABLE 6
Response Assessment Criteria for CLL. Abbreviations: ANC = absolute
neutrophil count; CR = complete remission; CRi = CR with
incomplete blood count recovery; PR = partial remission.

| | | Bone | |
|---|---|---|---|
| Re- | | Marrow (if | Nodes, Liver, and |
| sponse | Peripheral Blood | performed) | Spleen^{a} |
| CR | Lymphocytes <4 × 10⁹/L | Normocellular | Normal (e.g., no |
| | ANC >1.5 × 10⁹/L^{b} | <30% | lymph |
| | Platelets >100 × 10⁹/L^{b} | lymphocytes | nodes >1.5 cm) |
| | Hemoglobin >11.0 g/dL | No B-lymphoid | |
| | (untransfused)^{b} | nodules | |
| CRi | Lymphocytes <4 × 10⁹/L | Hypocellular | Normal (e.g., no |
| | Persistent anemia, | <30% | lymph |
| | thrombocytopenia, or | lymphocytes | nodes >1.5 cm) |
| | neutropenia related to | | |
| | drug toxicity | | |
| PR | Lymphocytes ≧50% | Not assessed | ≧50% reduction in |
| | decrease from baseline | | lymphadenopathy^{c} |
| | ANC >1.5 × 10⁹/L | | and/or in spleen or |
| | or | | liver enlargement |
| | Platelets >100 × 10⁹/L or | | |
| | 50% improvement over | | |
| | baseline^{b} | | |
| | or | | |
| | Hemoglobin >11.0 g/dL | | |
| | or | | |
| | 50% improvement over | | |
| | baseline (untransfused)^{b} | | |

^{a}Computed tomography (CT) scan of abdomen, pelvis, and chest is required for this evaluation
^{b}Without need for exogenous growth factors
^{c}In the sum products of ≦6 lymph nodes or in the largest diameter of the enlarged lymph node(s) detected before therapy and no increase in any lymph node or new enlarged lymph nodes

The response assessment criteria for SLL are summarized in Table 7.

TABLE 7
Response Assessment Criteria for SLL. Abbreviations: CR = complete remission,
CT = computed tomography, FDG = [¹⁸F]fluorodeoxyglucose, PET = positron-emission
tomography, PR = partial remission, SD = stable disease, SPD = sum of the product of the
diameters.

| Response | Definition | Nodal Masses | Spleen, Liver | Bone Marrow |
|---|---|---|---|---|
| CR | Disappearance | (a) FDG-avid or PET | Not palpable, | If infiltrate present |
| | of all evidence | positive prior to | nodules | at screening, |
| | of disease | therapy; mass of any | disappeared | infiltrate cleared on |
| | | size permitted if PET | | repeat biopsy; if |
| | | negative | | indeterminate by |
| | | (b) Variably FDG-avid | | morphology, |
| | | or PET negative; | | immunohisto- |
| | | regression to normal | | chemistry should be |
| | | size on CT | | negative |
| PR | Regression of | ≧50% decrease in SPD | ≧50% decrease | Irrelevant if |
| | measurable | of up to 6 largest | in SPD of | positive prior to |
| | disease and no | dominant masses; no | nodules (for | therapy; cell type |
| | new sites | increase in size of other | single nodule in | should be specified |
| | | nodes | greatest | |
| | | (a) FDG-avid or PET | transverse | |
| | | positive prior to | diameter); no | |
| | | therapy; ≧1 PET | increase in size | |
| | | positive at previously | of liver or | |
| | | involved site | spleen | |
| | | (b) Variably FDG-avid | | |
| | | or PET negative; | | |
| | | regression on CT | | |
| SD | Failure to | (a) FDG-avid or PET | | |
| | attain CR/PR | positive prior to | | |
| | or progressive | therapy; PET positive | | |
| | disease | at prior sites of disease, | | |
| | | and no new sites on CT | | |
| | | or PET | | |
| | | (b) Variably FDG avid | | |
| | | or PET negative; no | | |
| | | change in size of | | |
| | | previous lesions on CT | | |

The PK parameters of the study are as follows. The plasma PK of Formula (2) and a metabolite is characterized using noncompartmental analysis. The following PK parameters are calculated, whenever possible, from plasma concentrations of Formula (2):
AUC₍₀₋ₜ₎: Area under the plasma concentration-time curve calculated using linear trapezoidal summation from time 0 to time t, where t is the time of the last measurable concentration (Ct),
AUC₍₀₋₂₄₎: Area under the plasma concentration-time curve from 0 to 24 hours, calculated using linear trapezoidal summation,
AUC_{(0-∞)}: Area under the plasma concentration-time curve from 0 to infinity, calculated using the formula: AUC_{(0-∞)}=AUC₍₀₋ₜ₎+Ct/λz, where λz is the apparent terminal elimination rate constant,
Cₘₐₓ: Maximum observed plasma concentration,
Tₘₐₓ: Time of the maximum plasma concentration (obtained without interpolation),
t_{1/2}: Terminal elimination half-life (whenever possible),
λ_{z}: Terminal elimination rate constant (whenever possible),
Cl/F: Oral clearance.

The PD parameters of the study are as follows. The occupancy of BTK by Formula (2) are measured in peripheral blood mononuclear cells (PBMCs) with the aid of a biotin-tagged Formula (2) analogue probe. The effect of Formula (2) on biologic markers of B cell function will also be evaluated.

The statistical analysis used in the study is as follows. No formal statistical tests of hypotheses are performed. Descriptive statistics (including means, standard deviations, and medians for continuous variables and proportions for discrete variables) are used to summarize data as appropriate.

The following definitions are used for the safety and efficacy analysis sets: Safety analysis set: All enrolled subjects who receive ≧1 dose of study drug; Per-protocol (PP) analysis set: All enrolled subjects who receive ≧1 dose of study drug and with ≧1 tumor response assessment after treatment. The safety analysis set will be used for evaluating the safety parameters in this study. The PP analysis sets will be analyzed for efficacy parameters in this study.

No imputation of values for missing data is performed except for missing or partial start and end dates for adverse events and concomitant medication will be imputed according to prespecified, conservative imputation rules. Subjects lost to follow-up (or drop out) will be included in statistical analyses to the point of their last evaluation.

The safety endpoint analysis was performed as follows. Safety summaries will include summaries in the form of tables and listings. The frequency (number and percentage) of treatment emergent adverse events will be reported in each treatment group by Medical Dictionary for Regulatory Activities (MedDRA) System Organ Class and Preferred Term. Summaries will also be presented by the severity of the adverse event and by relationship to study drug. Laboratory shift tables containing counts and percentages will be prepared by treatment assignment, laboratory parameter, and time. Summary tables will be prepared for each laboratory parameter. Figures of changes in laboratory parameters over time will be generated. Vital signs, ECGs, and physical exams will be tabulated and summarized.

Additional analyses include summaries of subject demographics, baseline characteristics, compliance, and concurrent treatments. Concomitant medications will be coded according to the World Health Organization (WHO) Drug Dictionary and tabulated.

The analysis of efficacy parameters was performed as follows. The point estimate of the overall response rate will be calculated for the PP analysis set. The corresponding 95% confidence interval also will be derived. The duration of overall response is measured from the time measurement criteria are met for CR or PR (whichever is first recorded) until the first date that recurrent or progressive disease is objectively documented (taking as reference for progressive disease the smallest measurements recorded since the treatment started). Kaplan-Meier methodology will be used to estimate event-free curves and corresponding quantiles (including the median). Progression-free survival is measured from the time of first study drug administration until the first date that recurrent or progressive disease is objectively documented (taking as reference for progressive disease the smallest measurements recorded since the treatment started). Kaplan-Meier methodology will be used to estimate the event-free curves and corresponding quantiles (including the median).

The study scheme is a sequential cohort escalation. Each cohort consists of six subjects. The sample size of the study is 24 to 36 subjects, depending on dose escalation into subsequent cohorts. Cohort 1 (N=6) consists of Formula (2), 100 mg QD for 28 days. Cohort 2 (N=6) consists of Formula (2), 175 mg QD for 28 days. Cohort 3 (N=6) consists of Formula (2), 250 mg QD for 28 days. Cohort 4 (N=6) consists of Formula (2), 350 mg QD for 28 days. Cohort 5 (N=6) consists of Formula (2), 450 mg QD for 28 days. Cohort 6 (N=6) consists of Formula (2), at a dose to be determined QD for 28 days. The dose level for Cohort 6 will be determined based on the safety and efficacy of Cohorts 1 to 5, and will not exceed 900 mg/day. Escalation will end with either the MTD cohort or three levels above full BTK occupancy, whichever is observed first. An additional arm of the study will explore 100 mg BID dosing. Treatment with oral Formula (2) may be continued for greater than 28 days until disease progression or an unacceptable drug-related toxicity occurs.

The inclusion criteria for the study are as follows: (1) men and women ≧18 years of age with a confirmed diagnosis of CLL/SLL, which has relapsed after, or been refractory to, ≧2 previous treatments for CLL/SLL; however, subjects with 17p deletion are eligible if they have relapsed after, or been refractory to, 1 prior treatment for CLL/SLL; (2) body weight ≧60 kg, (3) ECOG performance status of ≦2; (4) agreement to use contraception during the study and for 30 days after the last dose of study drug if sexually active and able to bear children; (5) willing and able to participate in all required evaluations and procedures in this study protocol including swallowing capsules without difficulty; or (6) ability to understand the purpose and risks of the study and provide signed and dated informed consent and authorization to use protected health information (in accordance with national and local subject privacy regulations).

The dosage form and strength of Formula (2) used in the clinical study is a hard gelatin capsules prepared using standard pharmaceutical grade excipients (microcrystalline cellulose) and containing 25 mg of Formula (2) each. The color of the capsules is Swedish orange. The route of administration is oral (per os, or PO). The dose regimen is once daily or twice daily, as defined by the cohort, on an empty stomach (defined as no food 2 hours before and 30 minutes after dosing).

The baseline characteristics for the patients enrolled in the clinical study are given in Table 8.

TABLE 8
Relapsed/refractory CLL baseline characteristics.

| Characteristic | CLL (N = 44) |
|---|---|
| Patient Demographics | |
| Age (years), median (range) | 62 (45-84) |
| Sex, men (%) | 33 (75) |
| Prior therapies, median | 3 (1-10) |
| (range), n | |
| ≧3 prior therapies, n (%) | 26 (59) |
| Clinical Details | |
| ECOG performance status ≧1 | 28 (63) |
| (%) | |
| Rai stage III/IV | 16 (36) |
| Bulky disease ≧5 cm, n (%) | 15 (34) |
| Cytopenia at baseline | 33 (75) |
| Cytogenic Status | |
| Chromosome 11q22.3 deletion | 18 (41) |
| (Del 11q), n (%) | |
| Chromosome 17p13.1 (Del | 19 (34) |
| 17p), n (%) | |
| IgV_{H} status (unmutated), n (%) | 28 (64) |

The results of the clinical study in relapsed/refractory CLL patients are summarized in Table 9.

TABLE 9
Activity of Formula (2) in relapsed/refractory CLL.

| | n (%) | | | | | |
|---|---|---|---|---|---|---|
| | All Cohorts | 100 mg QD | 175 mg QD | 250 mg QD | 100 mg BID | 400 mg QD |
| | (N = 31) | (N = 8) | (N = 8) | (N = 7) | (N = 3) | (N = 5) |
| PR | 22 (71) | 7 (88) | 5 (63) | 5 (71) | 3 (100) | 2 (40) |
| PR + L | 7 (23) | 0 (0) | 3 (37) | 2 (29) | 0 (0) | 2 (40) |
| SD | 2 (6) | 1 (12) | 0 (0) | 0 (0) | 0 (0) | 1 (20) |
| PD | 0 (0) | 0 (0) | 0 (0) | 0 (0) | 0 (0) | 0 (0) |

Median (range) Cycles

| | | | | | |
|---|---|---|---|---|---|
| 7.3 | 10.0 | 8.6 | 7.0 | 5.2 | 5.0 |
| (3.0-10.8) | (9.0-10.8) | (3.0-8.8) | (7.0-7.3) | (4.7-5.5) | (4.8-5.5) |

(PR = partial response; PR + L = partial response with lymphocytosis; SD = stable disease; PD = progressive disease.)

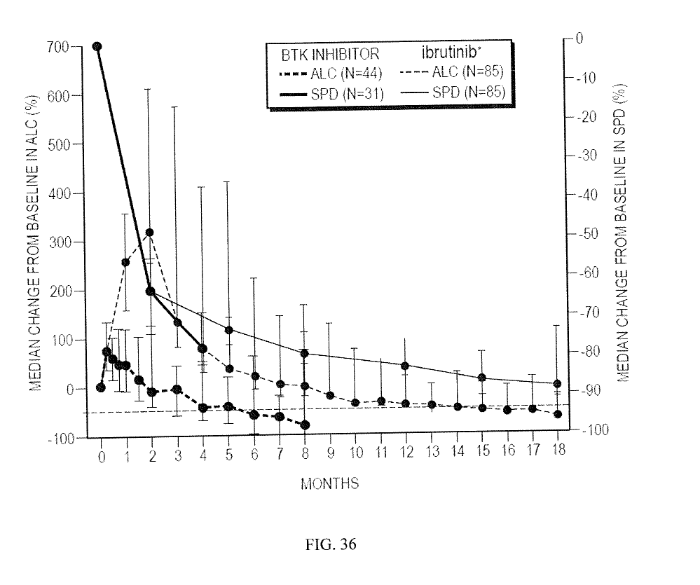

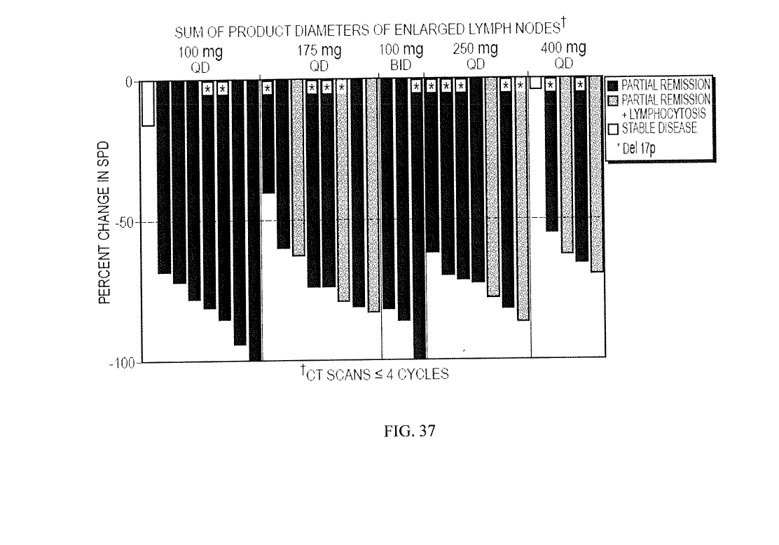

FIG. 36 shows the median % change in ALC and SPD from baseline in the clinical study of Formula (2), plotted in comparison to the results reported for ibrutinib in FIG. 1A of Byrd, et al., N. Engl. J. Med. 2013, 369, 32-42. The results show that Formula (2) leads to a more rapid patient response in CLL than corresponding treatment with ibrutinib. This effect is illustrated, for example, by the median % change in SPD, which achieved the same status in the present study at 7 months of treatment with Formula (2) as compared to 18 months for ibrutinib. The % change in SPD observed in the different cohorts (i.e. by dose and dosing regimen) is shown in FIG. 37, and in all cases shows significant responses.

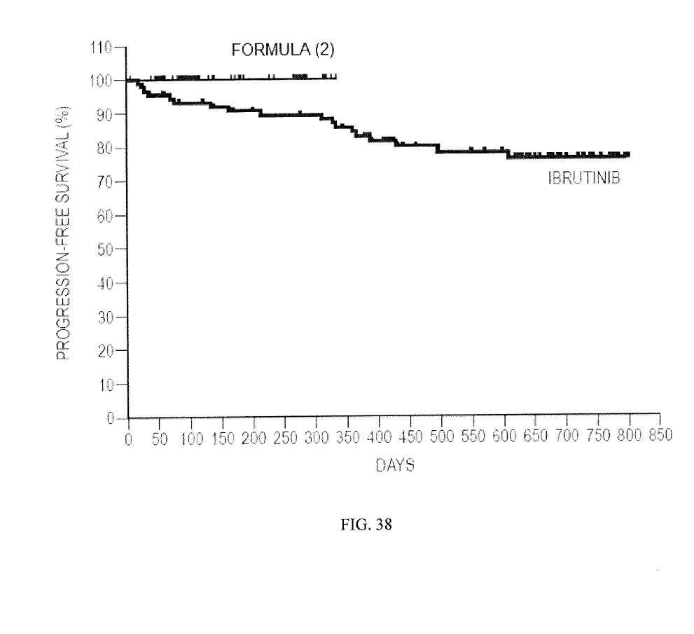

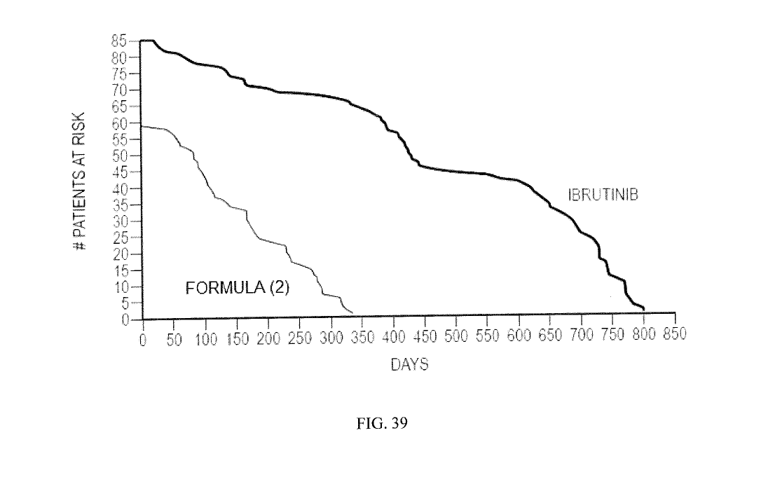

A Kaplan-Meier curve showing PFS from the clinical CLL study of Formula (2) is shown in FIG. 38. A comparison of survival curves was performed using the Log-Rank (Mantle-Cox) test, with a p-value of 0.0206 indicating that the survival curves are different. The number of patients at risk is shown in FIG. 39. Both FIG. 38 and FIG. 39 show the results for Formula (2) in comparison to the results reported for Formula (10) (ibrutinib) in Byrd, et al., N. Engl. J. Med. 2013, 369, 32-42. An improvement in survival and a reduction in risk are observed in CLL patients treated with Formula (2) in comparison to patients treated with ibrutinib.

Based on the data and comparisons shown above, the CLL study showed that the efficacy of Formula (2) was surprisingly superior to that of Formula (10) (ibrutinib).

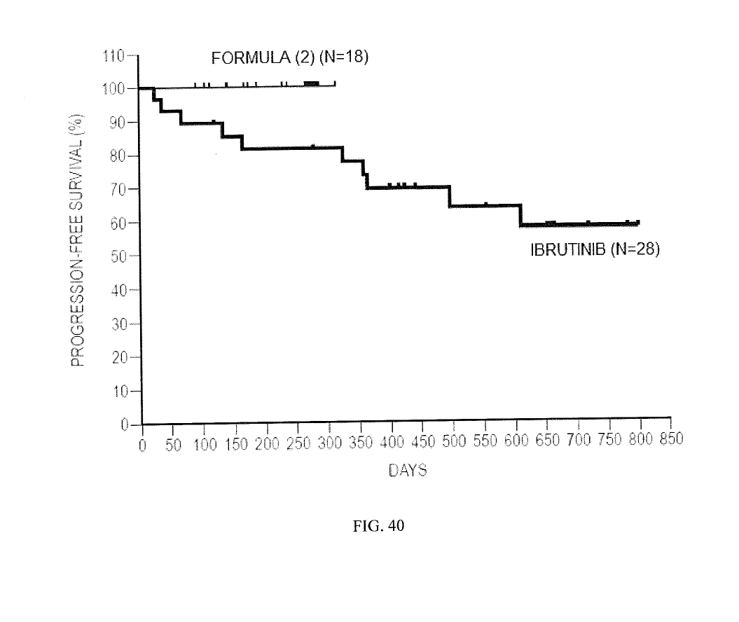

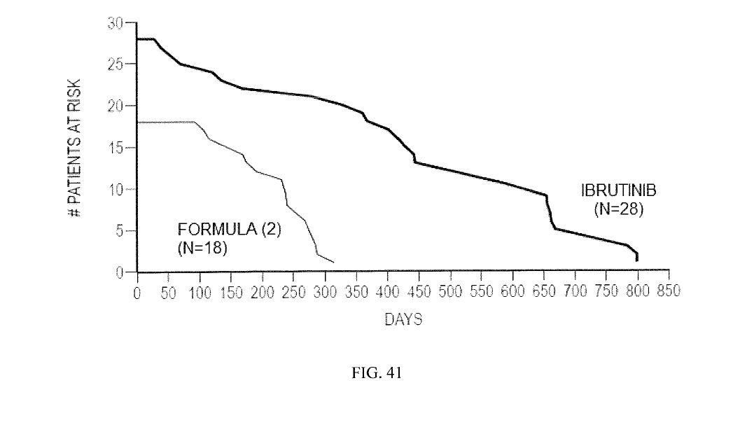

In the literature study of ibrutinib, increased disease progression was associated with patients with high-risk cytogenetic lesions (17p13.1 deletion or 11q22.3 deletion), as shown in FIG. 3A in Byrd, et al., N. Engl. J. Med. 2013, 369, 32-42, which shows ibrutinib PFS including PFS broken down by genetic abnormality. The 17p and 11q deletions are validated high-risk characteristics of CLL, and the 17p deletion is the highest risk. In FIG. 40, the PFS is shown for Formula (2) in patients with the 17p deletion in comparison to the results obtained for ibrutinib in Byrd, et al., N. Engl. J. Med. 2013, 369, 32-42. A p-value of 0.0696 was obtained. In FIG. 41, the number of patients at risk with the 17p deletion is compared. To date, no 17p patients have progressed on Formula (2).

The adverse events observed in the clinical study in relapsed/refractory CLL are given in Table 10. No DLTs were observed. The MTD was not reached. No treatment-related serious adverse events (SAEs) were observed. No prophylactic antivirals or antibiotics were needed.

TABLE 10
Treatment-related adverse events reported in the clinical study of
Formula (2) in relapsed/refractory CLL. (Reported in ≧5% of patients.)

| Adverse Events | | |
|---|---|---|
| (Treatment- | | |
| Related), n (%) | Grade | All (N = 44) |
| Headache | 1/2 | 7 (16) |
| Increased tendency | 1 | 6 (14) |
| to bruise | | |
| Diarrhea | 1 | 4 (9) |
| Petechiae | 1 | 3 (7) |

The clinical study of Formula (2) thus showed other unexpectedly superior results compared to ibrutinib therapy. A lack of lymphocytosis was observed in the study. Furthermore, only grade 1 AEs were observed, and these AEs were attributable to the high BTK selectivity of Formula (2).

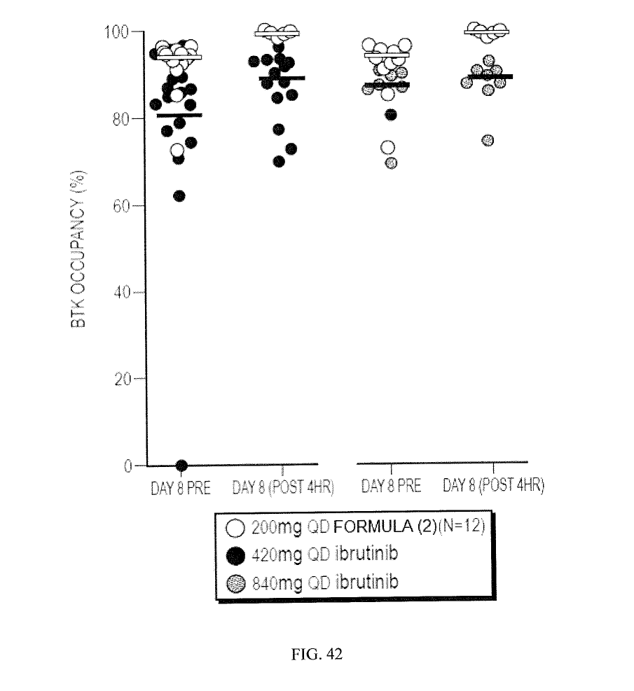

BTK target occupany was measured for relapsed/refractory CLL patients with the results shown in FIG. 42. For 200 mg QD dosing of the BTK inhibitor of Formula (2), about 94%-99% BTK occupancy was observed, with superior 24 hour coverage and less inter-patient variability also observed. For 420 mg and 840 mg QD of the BTK inhibitor ibrutinib, 80%-90% BTK occupancy was observed, with more inter-patient variability and capped occupancy. These results indicate that the BTK inhibitor of Formula (2) achieves superior BTK occupancy in CLL patients than ibrutinib.

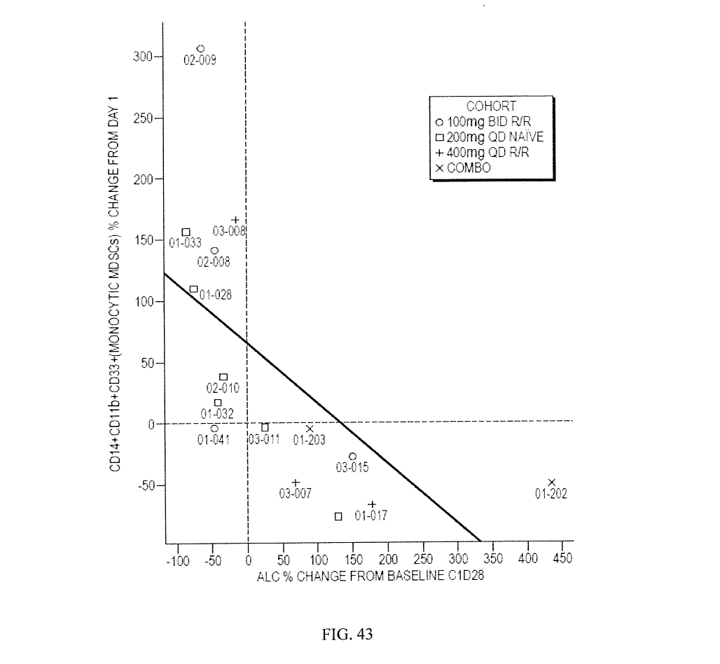

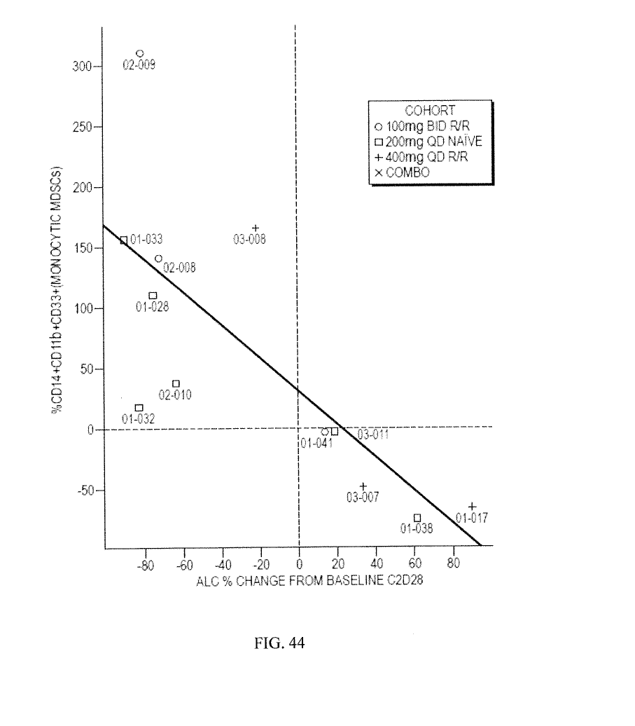

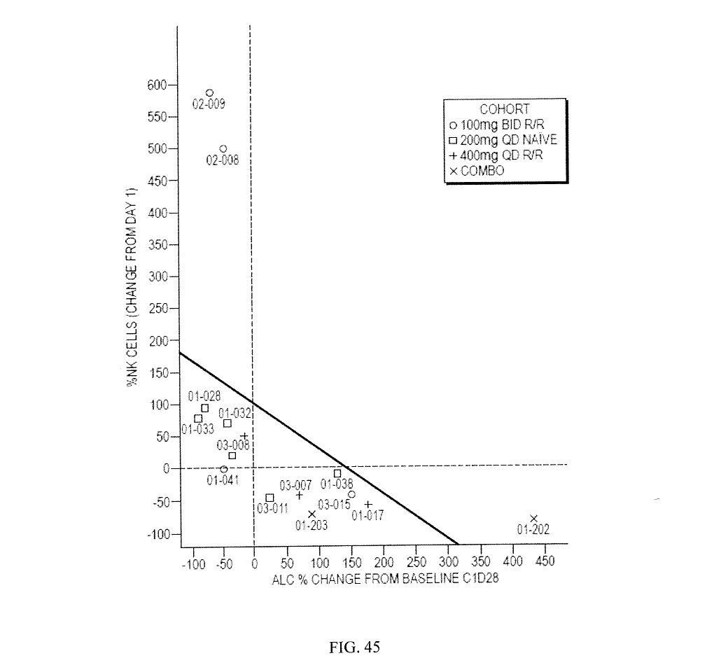

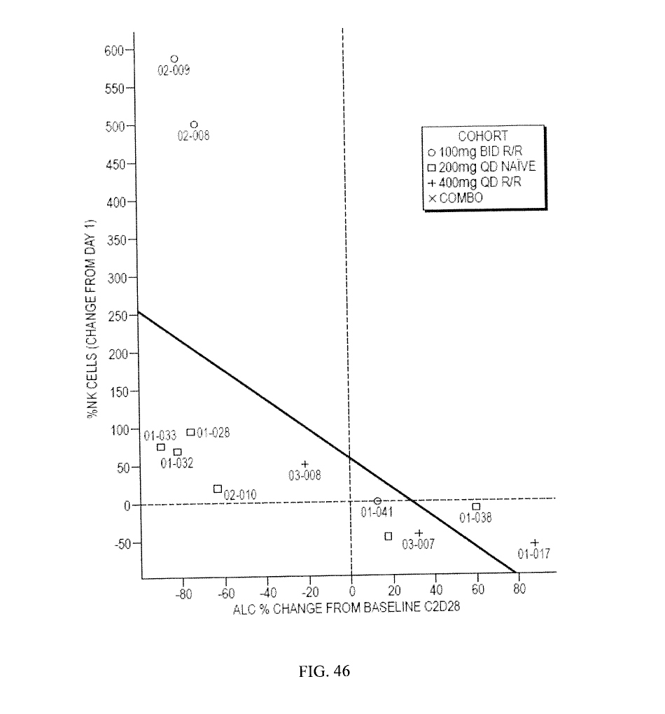

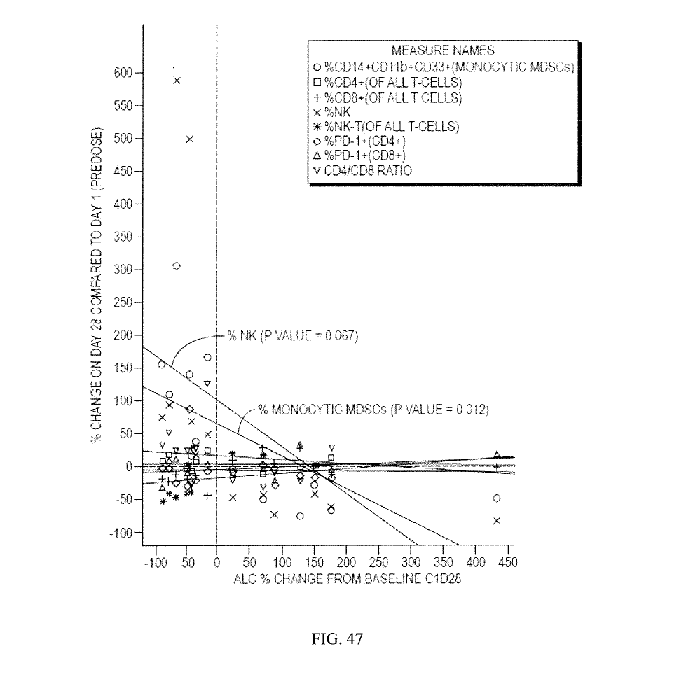

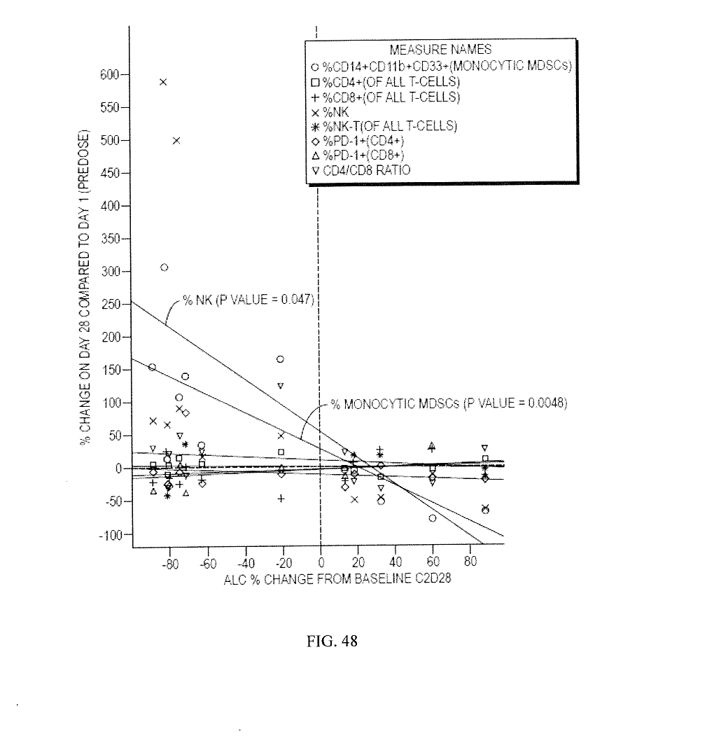

The effects of Formula (2) on cell subset percentages were also evaluated using flow cytometry analysis of peripheral blood, with the results shown in FIG. 43, FIG. 44, FIG. 45, FIG. 46, FIG. 47, and FIG. 48. PBMC samples from CLL patient samples drawn prior to (predose) and after 28 days of dosing with Formula (2) were compared for potential changes in cell subsets. PBMCs were stained with monoclonal antibodies conjugated to fluorescent tags (flourochromes) to identify cell subsets via flow cytometry. Non-viable cells were excluded from the analysis using the dye 7-aminoactinomycin D (7-AAD). To produce the metric of percent change, the following steps were taken. First, each cell subset was defined by hierarchical flow cytometry gating. Then, the change in frequency (between day 1 and day 28) was calculated for each cell subset. MDSC subsets were measured as a % of all myeloid cells. T cell subsets were measured as a % of all CD3⁺ cells, and NK cells were measured as a % of all live CD45⁺ cells. In FIG. 43 and FIG. 44, the results show the % change in MDSC (monocytic) level over 28 days versus % ALC change at cycle 1 day 28 (C1D28) and at cycle 2 day 28 (C2D28). A cycle is 28 days. A trend is observed wherein patients with decreasing ALC % had increasing MDSC (monocytic) %. This may include patients who had quickly resolving lymphocytosis and those with no initial lymphocytosis. This provides evidence that treatment with Formula (2) mobilizes MDSCs and thus affects the CLL tumor microenvironment in marrow and lymph nodes, which is an unexpected indication of superior efficacy. In FIG. 45 and FIG. 46, the results show the % change in NK cell level over 28 days versus % ALC change, measured at C1D28 or C2D28, and similar trends are observed wherein patients with decreasing ALC % had increasing NK cell %. This may include patients who had quickly resolving lymphocytosis and those having no initial lymphocytosis. The effects in FIG. 43 to FIG. 46 are observed in multiple cohorts, at doses including 100 mg BID, 200 mg QD, and 400 mg QD. In FIG. 47 and FIG. 48, the effects on NK cells and MDSC cells are compared to a number of other markers versus % change in ALC at C1D28 and C2D28. These other markers include CD4+ T cells, CD8+ T cells, CD4+/CD8+ T cell ratio, NK-T cells, PD-1+ CD4+ T cells, and PD-1+ CD8+ T cells. The effects on NK cells and MDSC cells are observed to be much more pronounced than on any of these other markers.

These results suggest that after Formula (2) administration, the CLL microenvironment undergoes a change wherein NK cells and monocytic MDSC subsets increase in frequency in the peripheral blood in patients with falling ALC counts, an important clinical parameter in CLL. The NK cell increase may reflect an overall increase in cytolytic activity against B-CLL resulting in the ALC % to drop. The increase in MDSC % in the blood may be due to a movement of these cells out of the lymph nodes, spleen, and bone marrow, which are all possible sites of CLL proliferation. Fewer MDSCs at the CLL proliferation centers would likely result in a reduced immunosuppressive microenvironment leading to an increase in cell-mediated immunity against the tumor, decreased tumor proliferation, and eventually lower ALC % in the circulation.

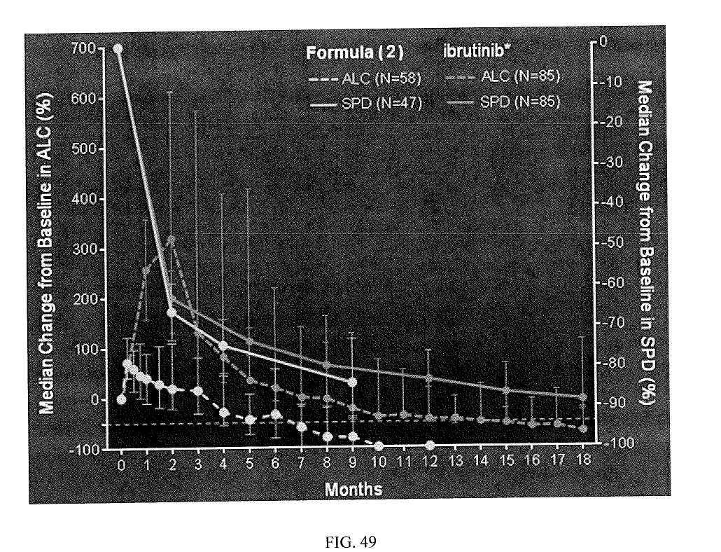

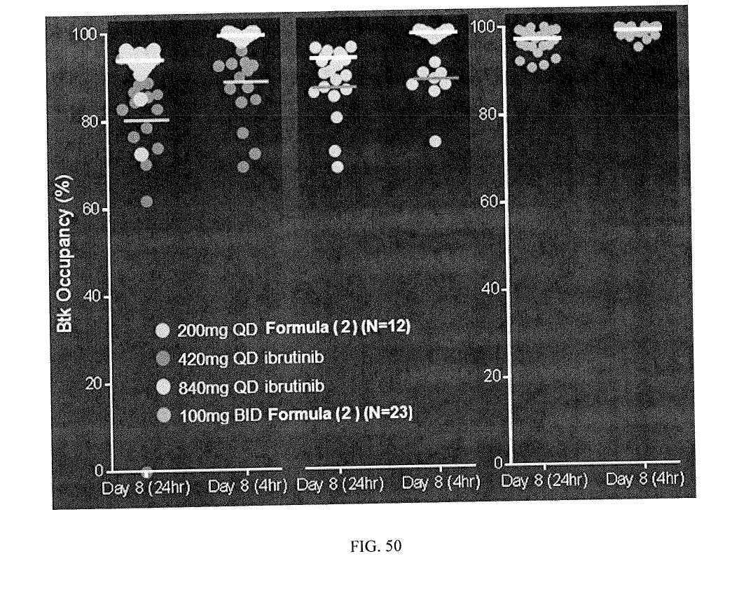

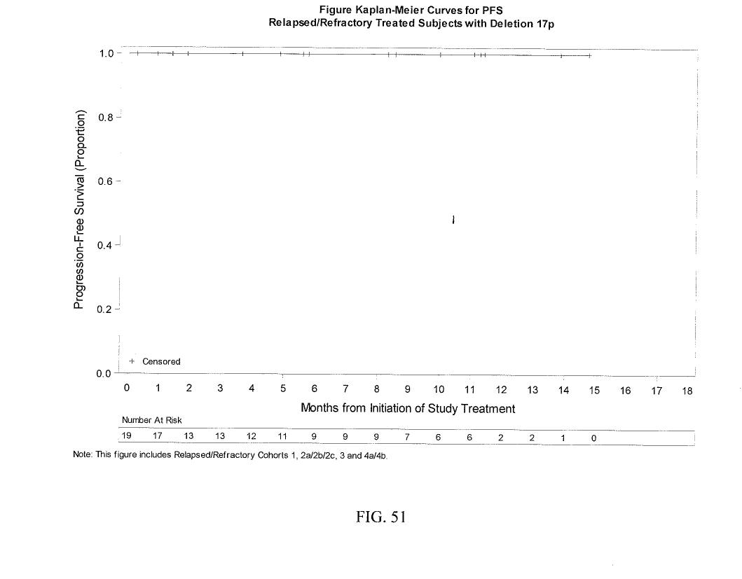

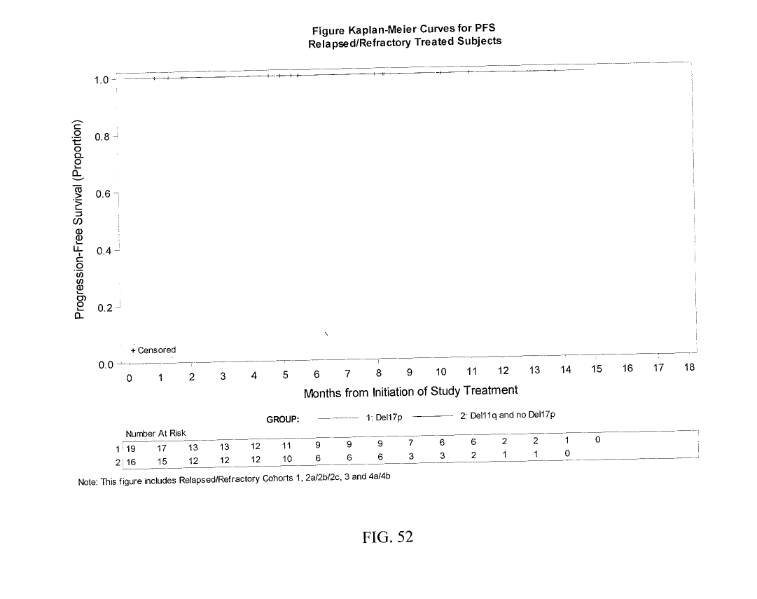

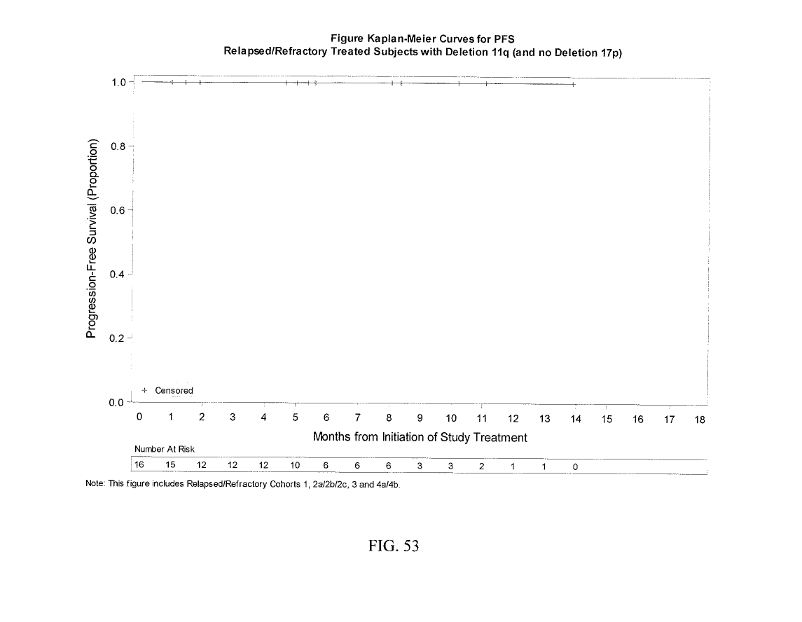

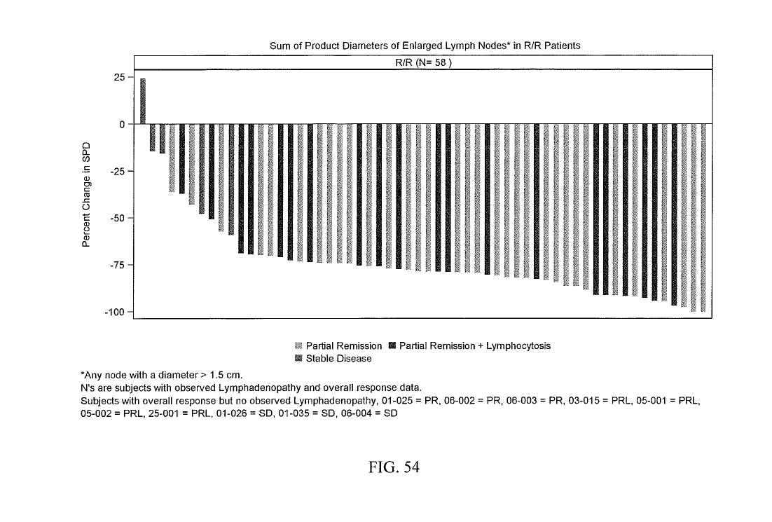

Updated clinical results from the CLL study are shown in FIG. 49 to FIG. 54. FIG. 49 shows an update of the data presented in FIG. 36. FIG. 50 shows an update of the data presented in FIG. 42, and includes BID dosing results. Formula (2) 200 mg QD dosing resulted in 94%-99% BTK occupancy, 24 hour coverage, and less inter-patient variability. Ibrutinib 420 mg and 840 mg QD dosing resulted in 80%-90% BTK occupancy, more inter-patient variability, and capped occupancy. Formula (2) 100 mg BID dosing resulted in 97%-99% BTK occupancy, complete BTK coverage, and less inter-patient variability. The PFS for patients with 17p deletions and 11q deletions are illustrated in FIG. 51, FIG. 52, and FIG. 53. Updated SPD results are illustrated in FIG. 54, and again show significant results across all cohorts and dosing regimens.

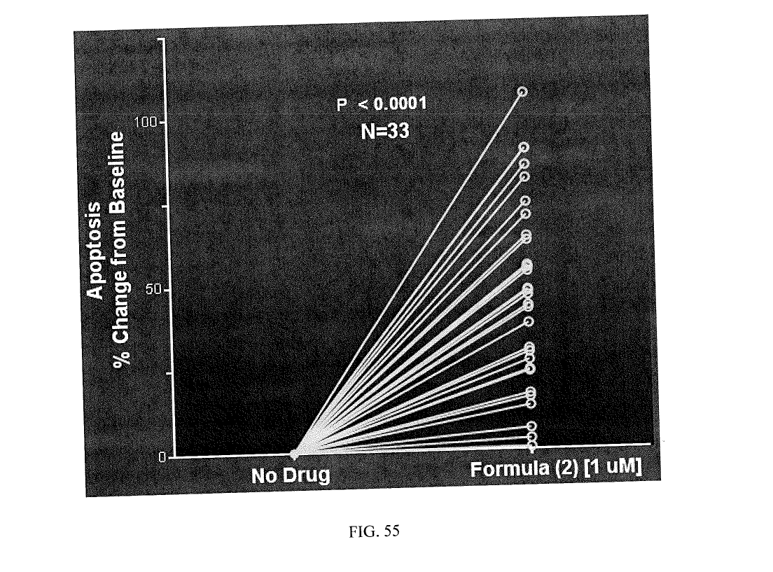

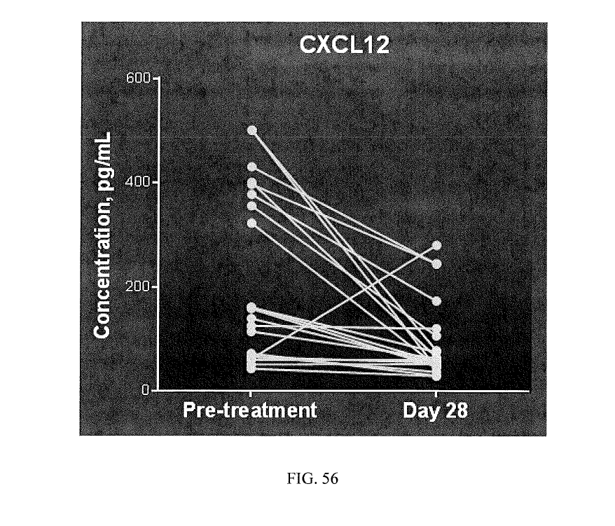

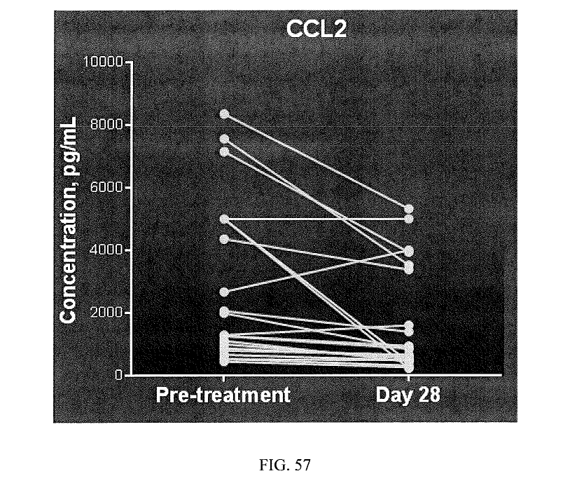

Treatment of CLL patients with Formula (2) also resulted in increased apoptotis, as illustrated in FIG. 55. Apoptotic B-CLL was defined by flow cytometry as having cleaved PARP⁺, Caspase 3⁺, CD19⁺, and CD5⁺phenotypes. 82% of samples tested had a baseline change greater than 25%. Treatment of CLL patients also showed that Formula (2) decreased plasma chemokines associated with MDSC homing and retention. A significant decrease in CXCL12 and CCL2 levels has been observed in patients treated with Formula (2), as shown in FIG. 56 and FIG. 57, respectively.

Overall, Formula (2) shows superior efficacy to first generation BTK inhibitors such as ibrutinib. Formula (2) has better target occupancy and better pharmacokinetic and metabolic parameters than ibrutinib, leading to improved B cell apoptosis. Furthermore, unlike treatment with ibrutinib, treatment with Formula (2) does not affect NK cell function. Finally, treatment with Formula (2) leads to a CLL tumor microenvironmental effect by excluding MDSC cells from the marrow and lymph nodes and reducing their number.

### Example 12

### Effects of BTK Inhibitors on Thrombosis

Clinical studies have shown that targeting the BCR signaling pathway by inhibiting BTK produces significant clinical benefit (Byrd, et al., N. Engl. J. Med. 2013, 369(1), 32-42, Wang, et al., N. Engl. J. Med. 2013, 369(6), 507-16). However, in these studies, bleeding has been reported in up to 50% of ibrutinib-treated patients. Most bleeding events were of grade 1-2 (spontaneous bruising or petechiae) but, in 5% of patients, they were of grade 3 or higher after trauma. These results are reflected in the prescribing information for ibrutinib, where bleeding events of any grade, including bruising and petechiae, were reported in about half of patients treated with ibrutinib (IMBRUVICA package insert and prescribing information, revised July 2014, U.S. Food and Drug Administration).

Constitutive or aberrant activation of the BCR signaling cascade has been implicated in the propagation and maintenance of a variety of B cell malignancies. Small molecule inhibitors of BTK, a protein early in this cascade and specifically expressed in B cells, have emerged as a new class of targeted agents. There are several BTK inhibitors, including Formula (17) (CC-292), and Formula (10) (ibrutinib), in clinical development. Importantly, early stage clinical trials have found ibrutinib to be particularly active in chronic lymphocytic leukemia (CLL) and mantle cell lymphoma (MCL), suggesting that this class of inhibitors may play a significant role in various types of cancers (Aalipour and Advani, Br. J. Haematol. 2013, 163, 436-43). However, their effects are not limited to leukemia or lymphomas as platelets also rely on the Tec kinases family members BTK and Tec for signal transduction in response to various thrombogenic stimuli (Oda, et al., Blood 2000, 95(5), 1663-70; Atkinson, et al., Blood 2003, 102(10), 3592-99). In fact, both Tec and BTK play an important role in the regulation of phospholipase Cγ2 (PLCγ2) downstream of the collagen receptor glycoprotein VI (GPVI) in human platelets. In addition, BTK is activated and undergoes tyrosine phosphorylation upon challenge of the platelet thrombin receptor, which requires the engagement of αIIbβ3 integrin (Laffargue, et al., FEBS Lett. 1999, 443(1), 66-70). It has also been implicated in GPIbα-dependent thrombus stability at sites of vascular injury (Liu, et al., Blood 2006, 108(8), 2596-603). Thus, BTK and Tec are involved in several processes important in supporting the formation of a stable hemostatic plug, which is critical for preventing significant blood loss in response to vascular injury. Hence, the effects of the BTK inhibitors of Formula (2) and Formula (10) (ibrutinib) were evaluated on human platelet-mediated thrombosis by utilizing the in vivo human thrombus formation in the VWF HA1 mice model described in Chen, et al., Nat. Biotechnol. 2008, 26(1), 114-19.

Administration of anesthesia, insertion of venous and arterial catheters, fluorescent labeling and administration of human platelets (5×10⁸/ml), and surgical preparation of the cremaster muscle in mice have been previously described (Chen et al., Nat Biotechnol. 2008, 26(1), 114-19). Injury to the vessel wall of arterioles (˜40-65 mm diameter) was performed using a pulsed nitrogen dye laser (440 nm, Photonic Instruments) applied through a 20× water-immersion Olympus objective (LUMPlanFl, 0.5 numerical aperature (NA)) of a Zeiss Axiotech vario microscope. Human platelet and wall interactions were visualized by fluorescence microscopy using a system equipped with a Yokogawa CSU-22 spinning disk confocal scanner, iXON EM camera, and 488 nm and 561 nm laser lines to detect BCECF-labeled and rhodamine-labeled platelets, respectively (Revolution XD, Andor Technology). The extent of thrombus formation was assessed for 2 minutes after injury and the area (μm²) of coverage determined (Image IQ, Andor Technology). For the Formula (2), Formula (17) (CC-292), and Formula (10) (ibrutinib) inhibition studies, the BTK inhibitors were added to purified human platelets for 30 minutes before administration.

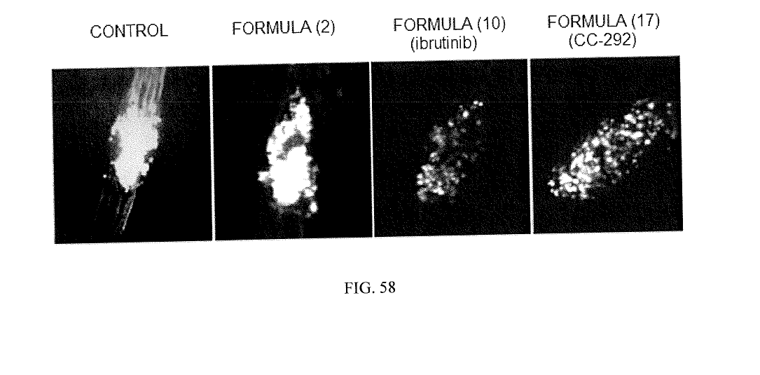

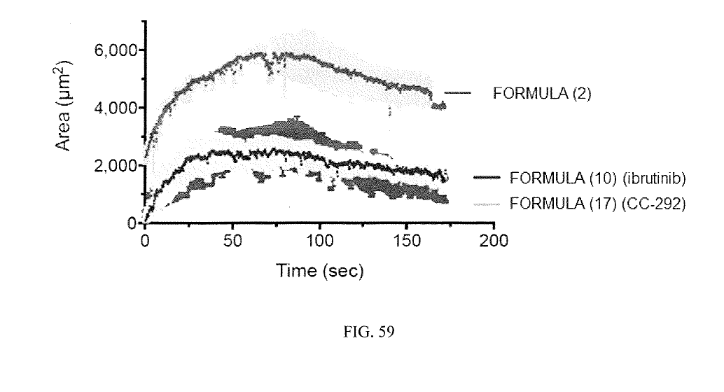

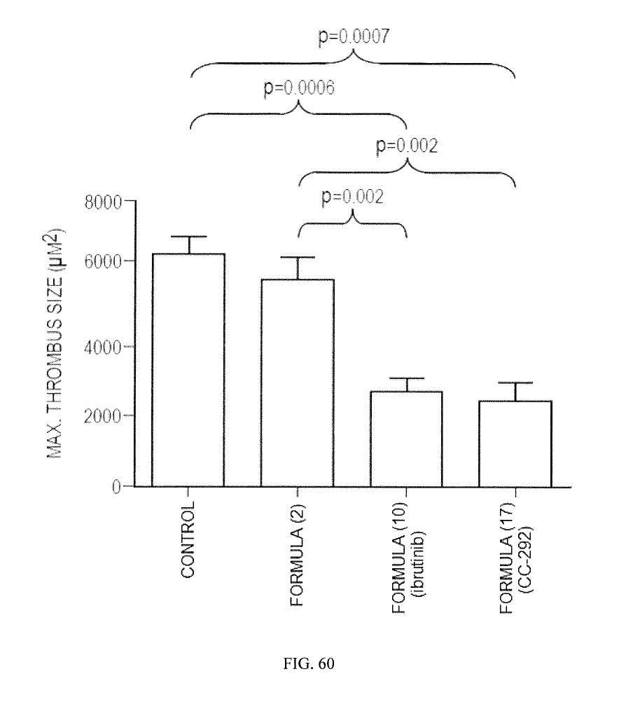

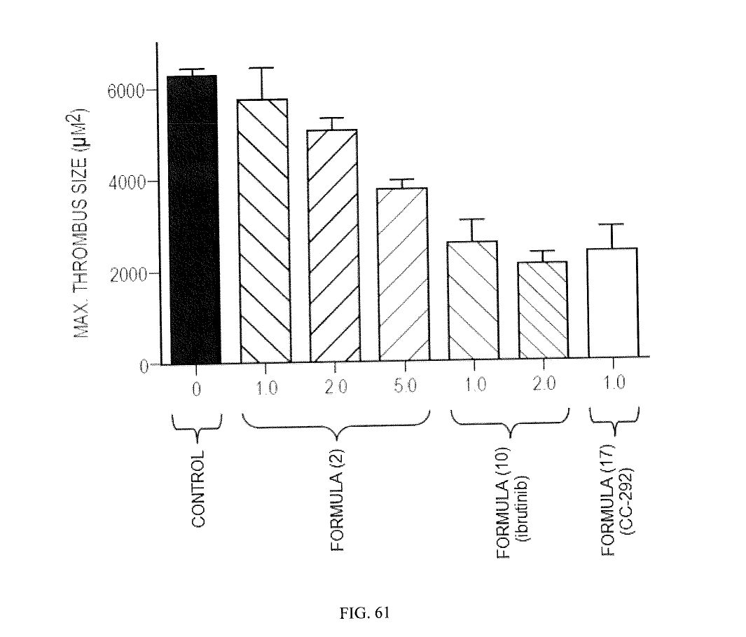

The in vivo throbus effects of the BTK inhibitors, Formula (2), Formula (17) (CC-292), and Formula (10) (ibrutinib), were evaluated on human platelet-mediated thrombosis by utilizing the in vivo human thrombus formation in the VWF HA1 mice model, which has been previously described (Chen, et al., Nat Biotechnol. 2008, 26(1), 114-19). Purified human platelets were preincubated with various concentrations of the BTK inhibitors (0.1 μM, 0.5 μM, or 1 μM) or DMSO and then administered to VWF HA1 mice, followed by laser-induced thrombus formation. The BTK inhibitor-treated human platelets were fluorescently labeled and infused continuously through a catheter inserted into the femoral artery. Their behavior in response to laser-induced vascular injury was monitored in real time using two-channel confocal intravital microscopy (Furie and Furie, J. Clin. Invest. 2005, 115(12), 2255-62). Upon induction of arteriole injury untreated platelets rapidly formed thrombi with an average thrombus size of 6,450±292 mm² (mean±s.e.m.), as shown in FIG. 58 and FIG. 59. Similarly, Formula (2) (1 μM) treated platelets formed a slightly smaller but not significantly different thrombi with an average thrombus size of 5733±393 mm² (mean±s.e.m.). In contrast, a dramatic reduction in thrombus size occurred in platelets pretreated with 1 μM of Formula (10) (ibrutinib), 2600±246 mm² (mean±s.e.m.), resulting in a reduction in maximal thrombus size by about 61% compared with control (P>0.001) (FIG. 58 and FIG. 60), Similar results were obtained with platelets pretreated with 500 nM of Formula (2) or ibrutinib: thrombus size of 5946±283 mm², and 2710±325 mm² respectively. These initial results may provide some mechanic background and explanation on the reported 44% bleeding related adverse event rates in the Phase III RESONATE study comparing ibrutinib with ofatumumab. The results obtained for Formula (17) (CC-292) were similar to that for Formula (10) (ibrutinib), as shown in FIGS. 58, 111, and 112. The effect of the BTK inhibitor concentration is shown in FIG. 61. These results demonstrate the surprising advantage of the BTK inhibitor of Formula (2), which does not interfere with thrombus formation, while the BTK inhibitors of Formula (17) (CC-292) and Formula (10) (ibrutinib) interfere with thrombus formation.

The objective of this study was to evaluate in vivo thrombus formation in the presence of BTK inhibitors. In vivo testing of novel antiplatelet agents requires informative biomarkers. By utilizing a genetic modified mouse von Willebrand factor (VWFR1326H) model that supports human but not mouse platelet-mediated thrombosis, we evaluated the effects of Formula (2), Formula (17) (CC-292), and Formula (10) (ibrutinib) on thrombus formation. These results show that Formula (2) had no significant effect on human platelet-mediated thrombus formation while Formula (10) (ibrutinib) was able to limit this process, resulting in a reduction in maximal thrombus size by 61% compared with control. Formula (17) (CC-292) showed an effect similar to Formula (10) (ibrutinib). These results, which show reduced thrombus formation for ibrutinib at physiologically relevant concentrations, may provide some mechanistic background for the Grade ≧3 bleeding events (e.g., subdural hematoma, gastrointestinal bleeding, hematuria and postprocedural hemorrhage) that have been reported in ≦6% of patients treated with Formula (10) (ibrutinib).

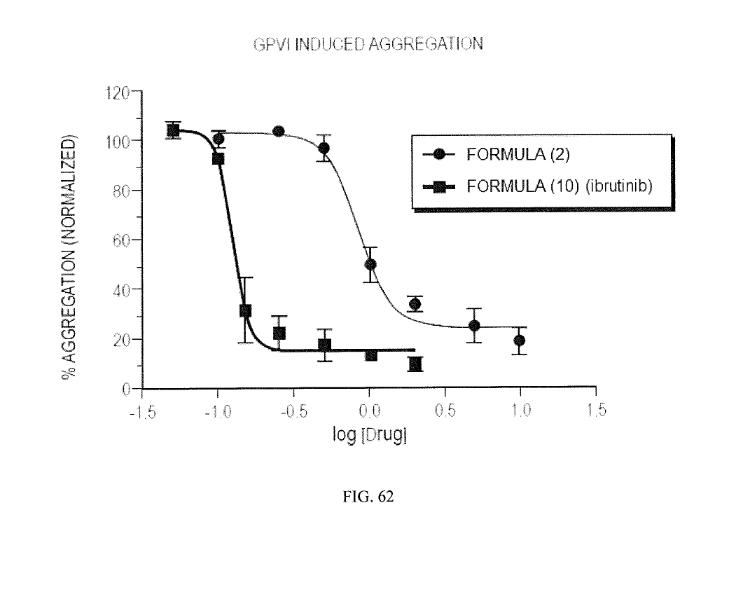

GPVI platelet aggregation was measured for Formula (2) and Formula (10) (ibrutinib). Blood was obtained from untreated humans, and platelets were purified from plasma-rich protein by centrifugation. Cells were resuspended to a final concentration of 350,000/μL in buffer containing 145 mmol/L NaCl, 10 mmol/L HEPES, 0.5 mmol/L Na₂HPO₄, 5 mmol/L KCl, 2 mmol/L MgCl₂, 1 mmol/L CaCl₂, and 0.1% glucose, at pH 7.4. Stock solutions of Convulxin (CVX) GPVI were prepared on the day of experimentation and added to platelet suspensions 5 minutes (37° C., 1200 rpm) before the induction of aggregation. Aggregation was assessed with a Chronolog Lumi-Aggregometer (model 540 VS; Chronolog, Havertown, Pa.) and permitted to proceed for 6 minutes after the addition of agonist. The results are reported as maximum percent change in light transmittance from baseline with platelet buffer used as a reference. The results are shown in FIG. 62.

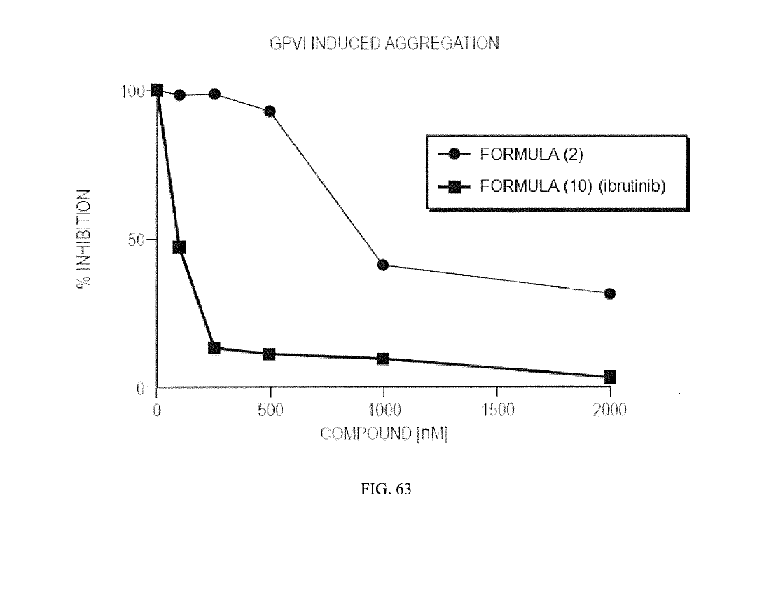

In FIG. 63, the results of CVX-induced (250 ng/mL) human platelet aggregation results before and 15 minutes after administration of the BTK inhibitors to 6 healthy individuals are shown.

The results depicted in FIG. 62 and FIG. 63 indicate that the BTK inhibitor of Formula (10) (ibrutinib) significantly inhibits GPVI platelet aggregation, while the BTK inhibitor of Formula (2) does not, further illustrating the surprising benefits of the latter compound.

### Example 13

### Synergistic Combinations of BTK Inhibitors and IRAK4 Inhibitors

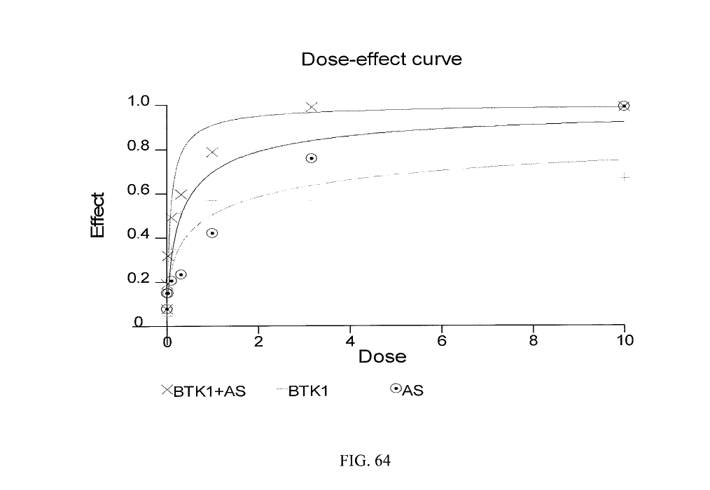

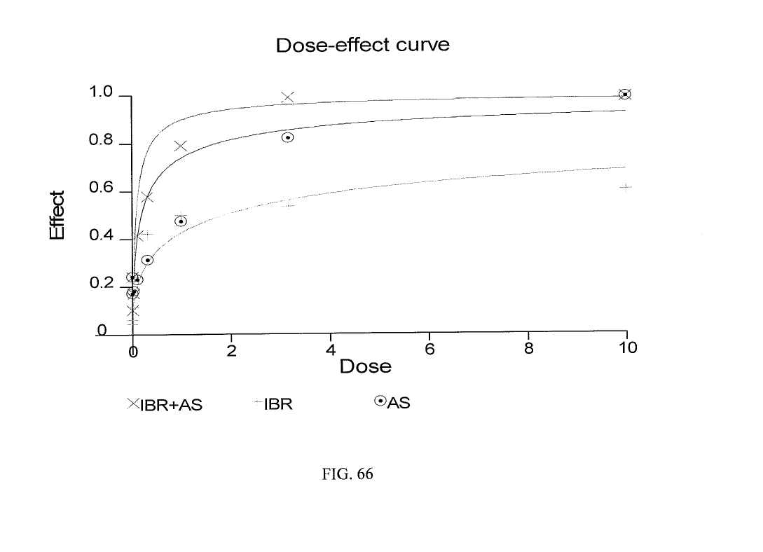

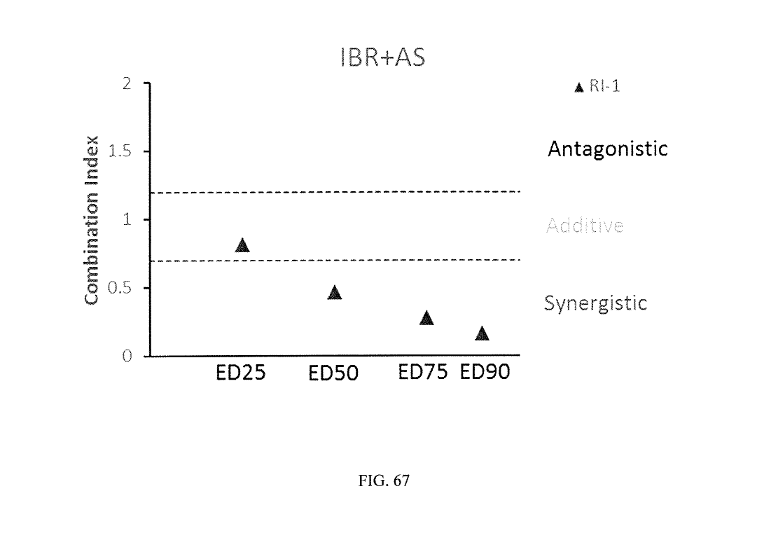

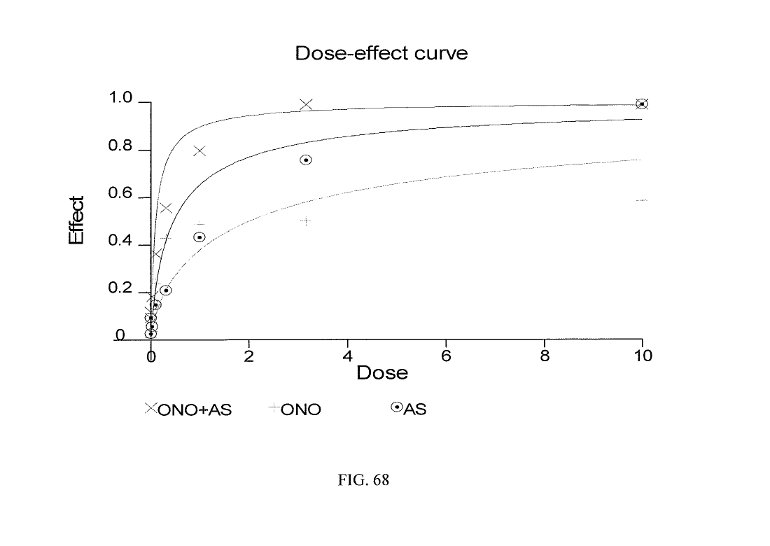

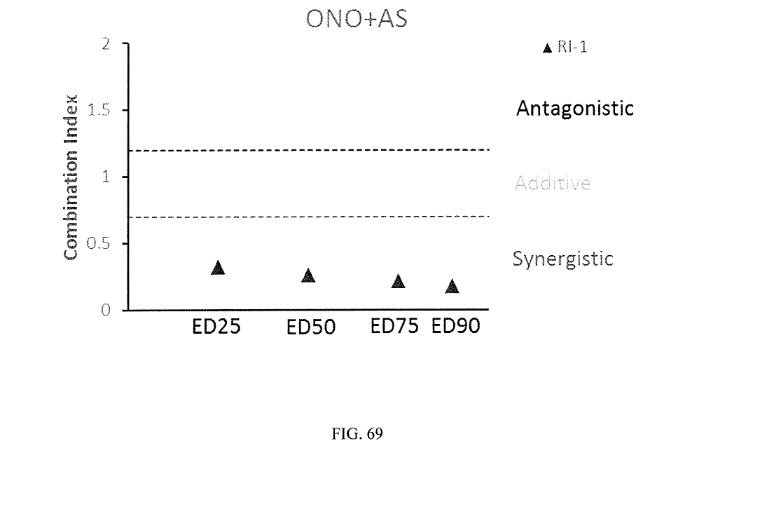

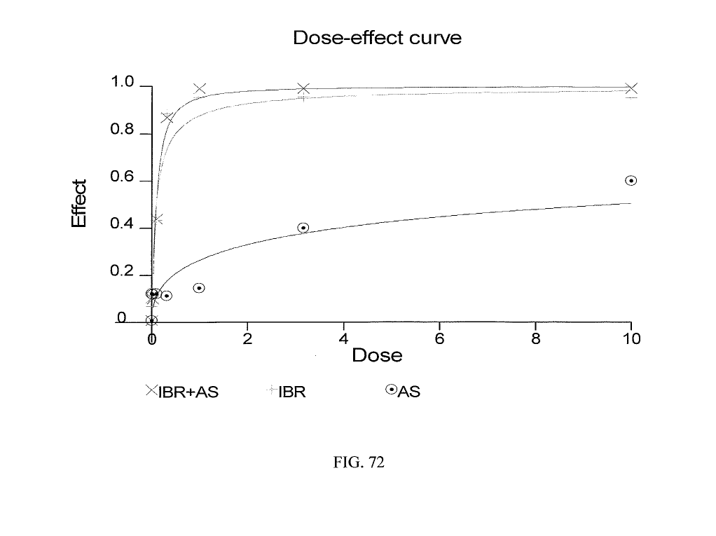

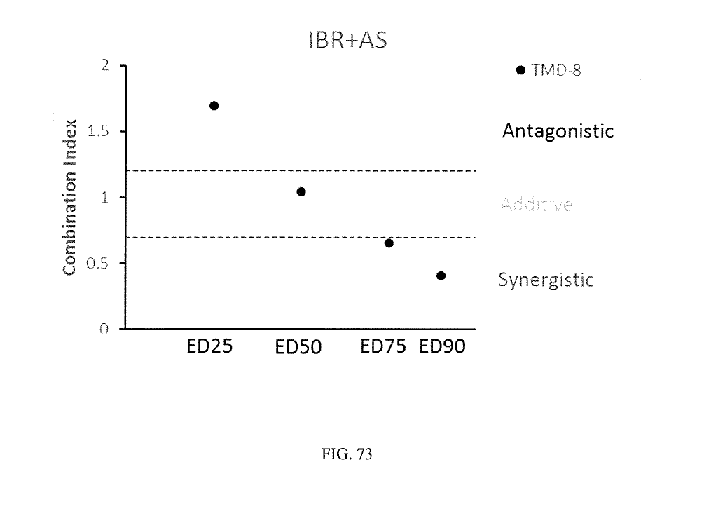

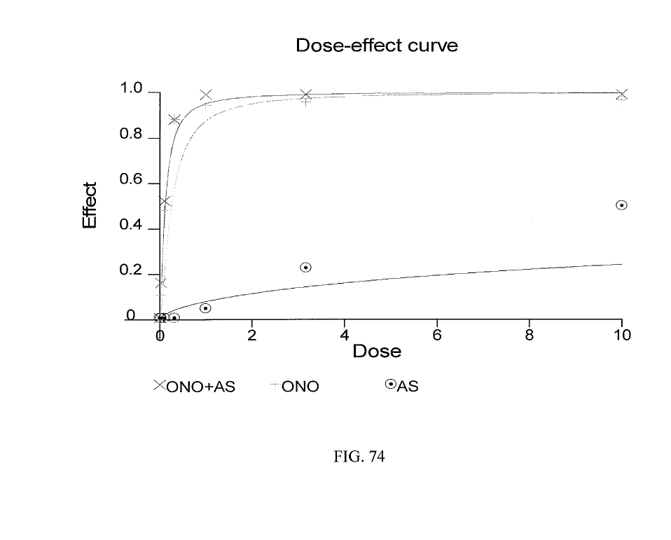

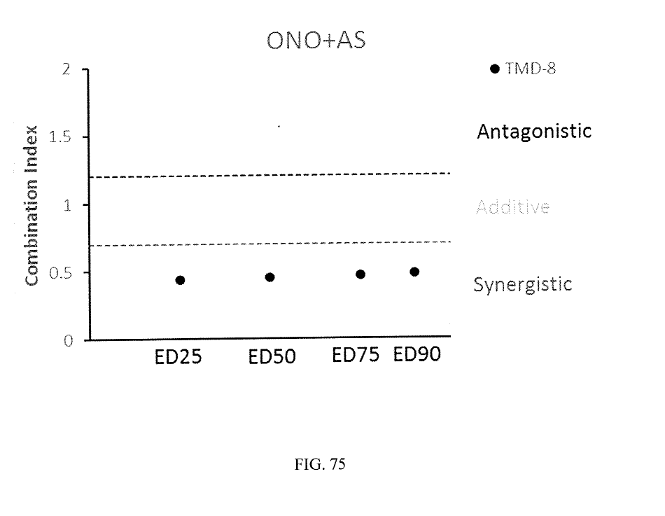

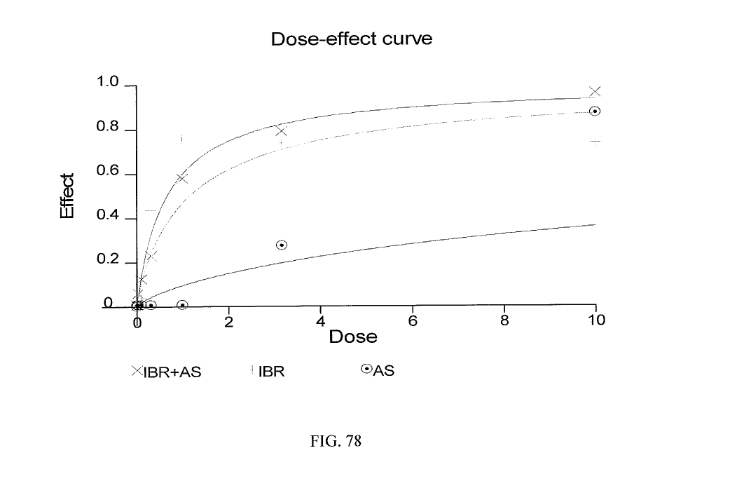

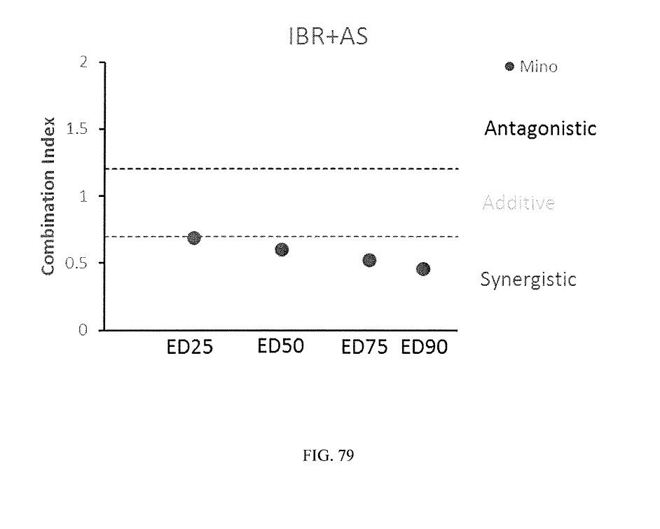

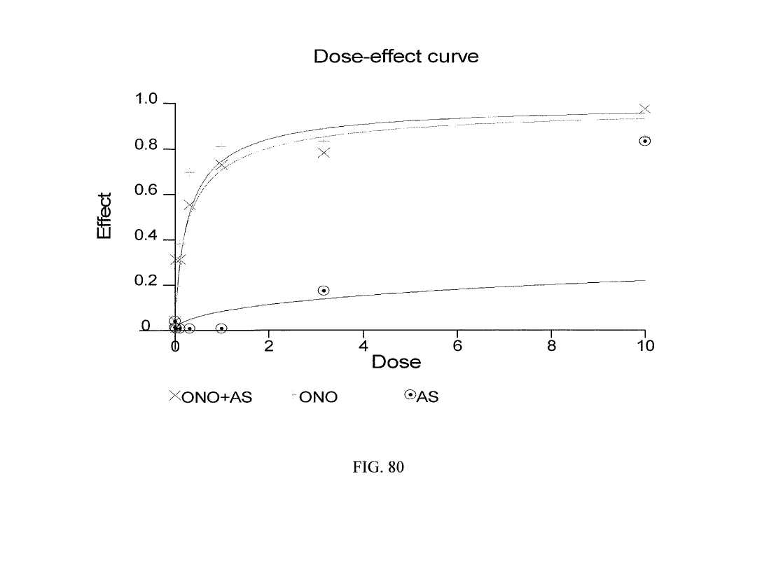

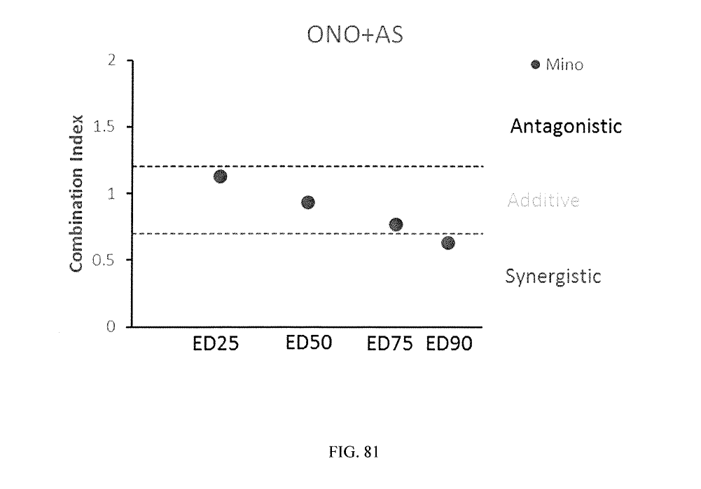

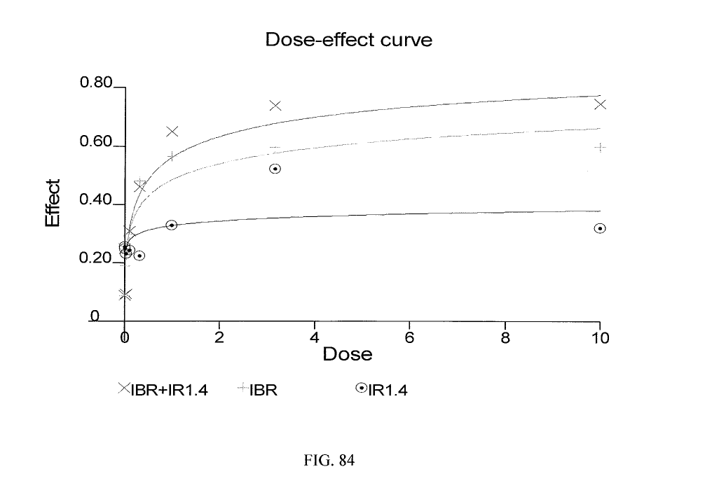

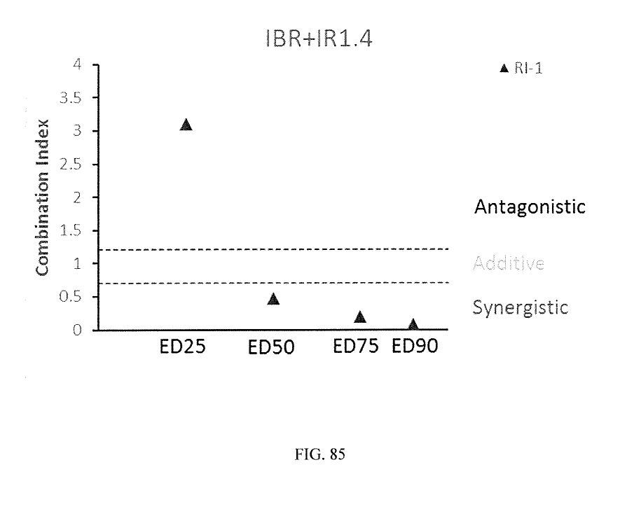

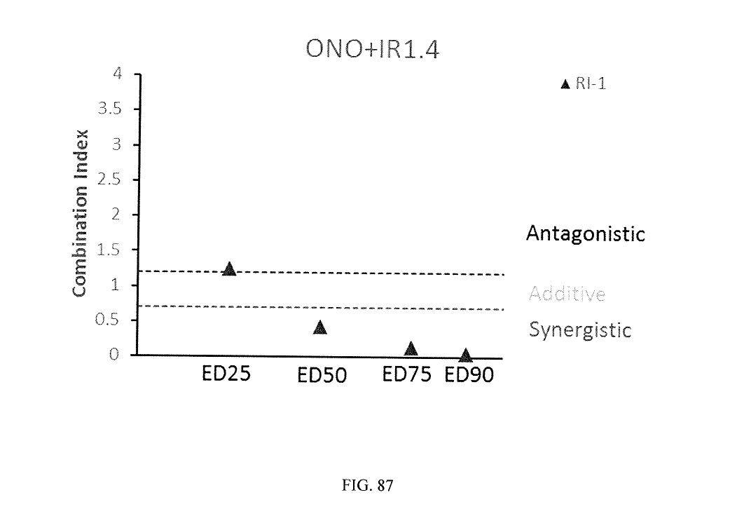

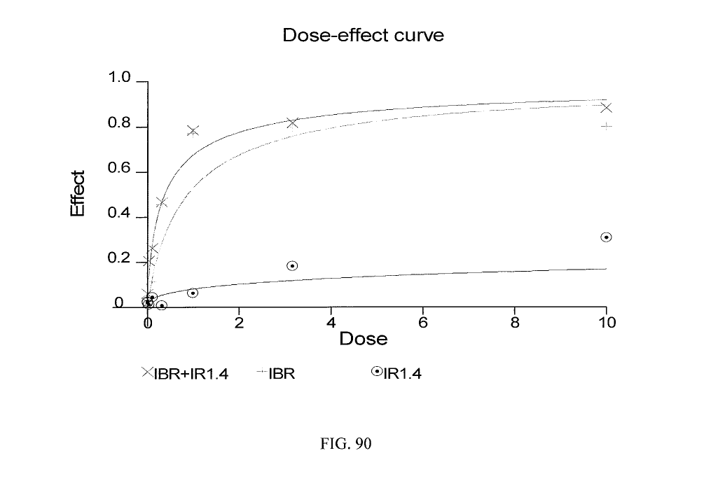

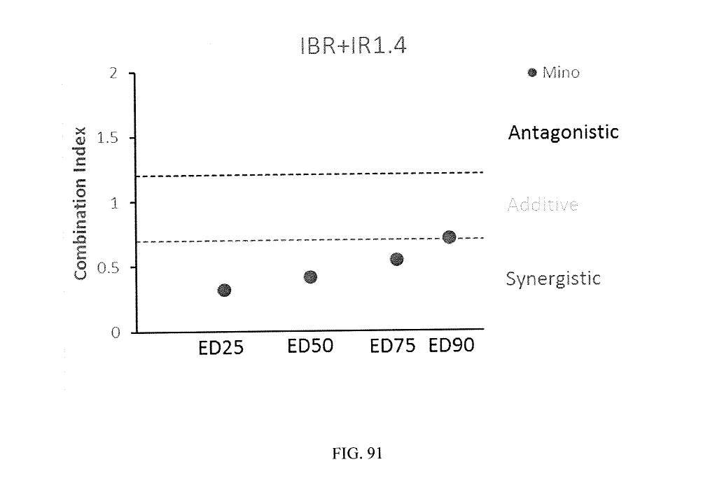

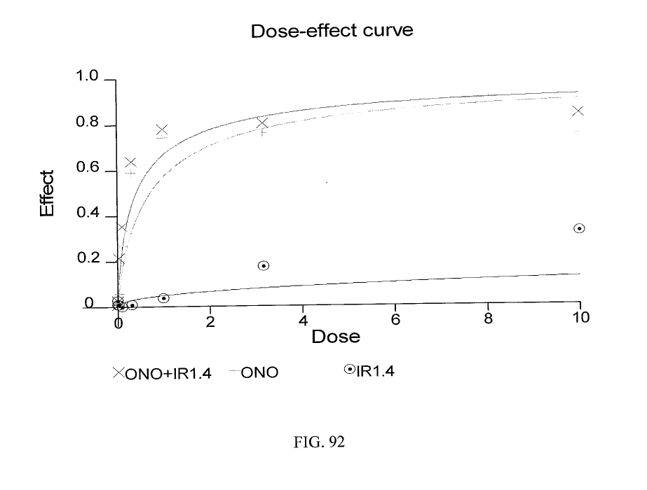

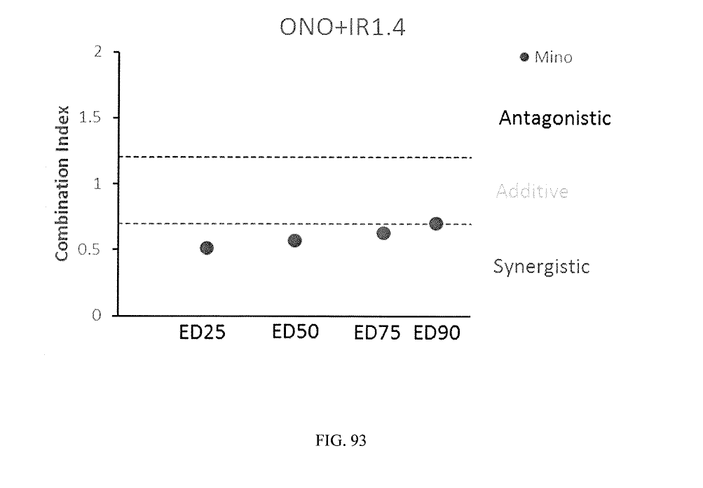

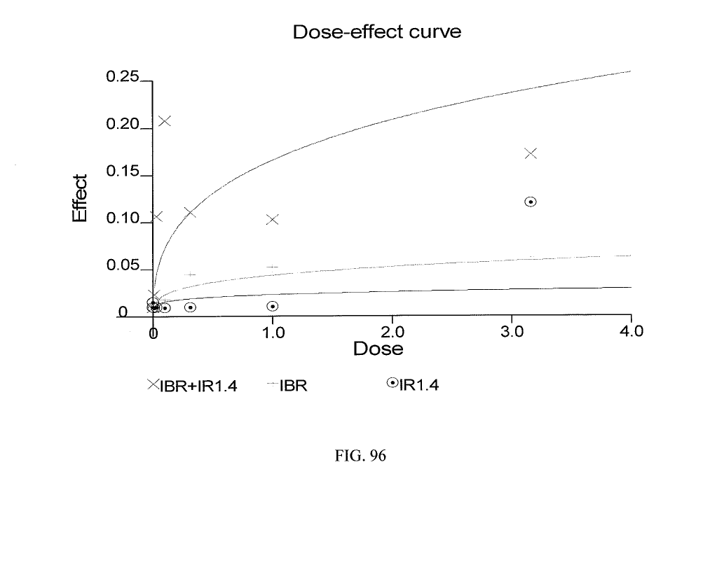

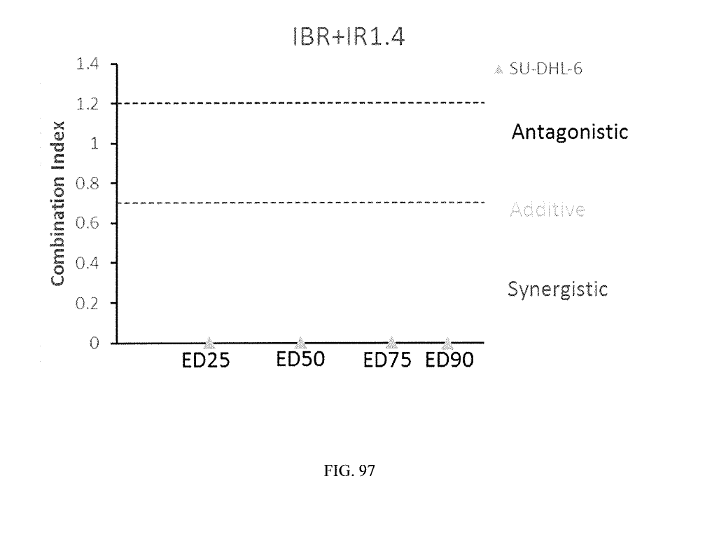

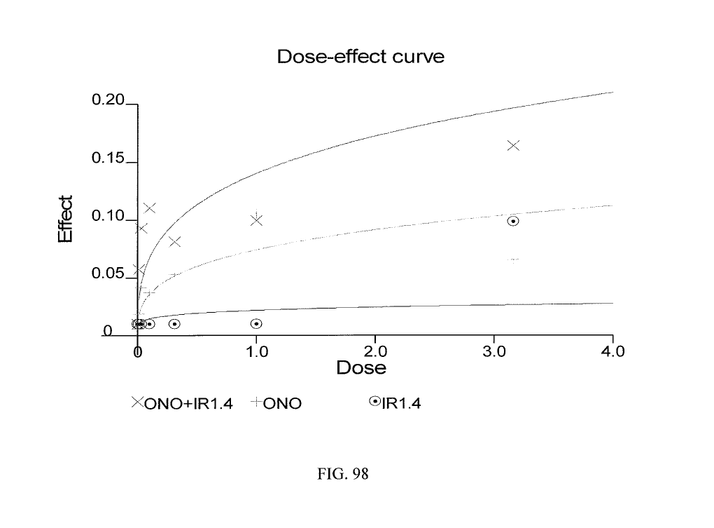

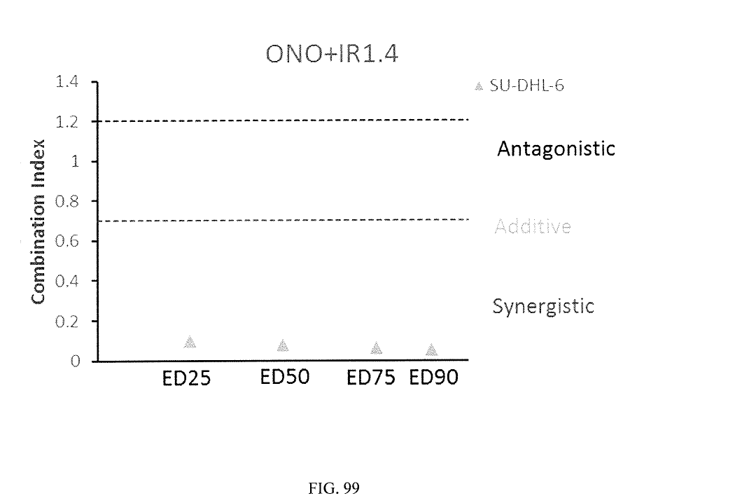

Combination experiments was performed to determine the synergistic, additive, or antagonistic behavior of drug combinations of BTK inhibitors and IRAK4 inhibitors. The IRAK4 inhibitors studied are N-(1-(2-morpholinoethyl)-1H-benzo[d]imidazol-2-yl)-3-nitrobenzamide (CAS No. 509093-47-4) (Formula (45); N-[3-carbamoyl-1-(tetrahydro-2H-pyran-4-yl)-1H-pyrazol-4-yl]-2-(2-methylpyridin-4-yl)-1,3-oxazole-4-carboxamide hydrochloride (AS2444697, CAS No. 1287665-60-4)(Formula (46)). The compound of Formula (45) is a dual IRAK1/4 inhibitor. The BTK inhibitors studies are (S)-4-(8-amino-3-(1-(but-2-ynoyl)pyrrolidin-2-yl)imidazo[1,5-a]pyrazin-1-yl)-N-(pyridin-2-yl)benzamide (Formula(2), CAS No. 1420477-60-6), 1-[(3R)-3-[4-Amino-3-(4-phenoxyphenyl)-1H-pyrazolo[3,4-d]pyrimidin-1-yl]piperidin-1-yl]prop-2-en-1-one (Formula (10), CAS No. 936563-96-1), and (R)-6-amino-9-(1-(but-2-ynoyl)pyrrolidin-3-yl)-7-(4-phenoxyphenyl)-7,9-dihydro-8H-purin-8-one (ONO-4059, CAS No. 1351636-18-4, Formula (21)). The combinations studies were assessed using the Chou-Talalay method of determining combination indexes for drug combinations, as described in Example 2. The MTS substrate Cell Titer 96 (Promega) was used. The synergy of the combinations were calculated using CalcuSyn software (Biosoft), which is based on the Median Effect methods described by Chou and Talalay, Trends Pharmacol. Sci. 1983, 4, 450-454. The combination index obtained was ranked according to Table 5, as discussed previously in Example 2. The test results are shown in FIGS. 64-99.

FIGS. 64, 66, and 68 illustrate the dose-effect curves obtained for the tested RI-1 cell line using combined dosing of the BTK inhibitor of Formula (2) (“BTK1”) and the IRAK4 inhibitor of Formula (46) (“AS”); BTK inhibitor of ibrutinib (Formula (10)) (“BR”) and the IRAK4 inhibitor of Formula (46) (“AS”); and the BTK inhibitor of Formula (21) (“ONO” or “ONO-4059”) and the IRAK4 inhibitor of Formula (46) (“AS”) respectively.

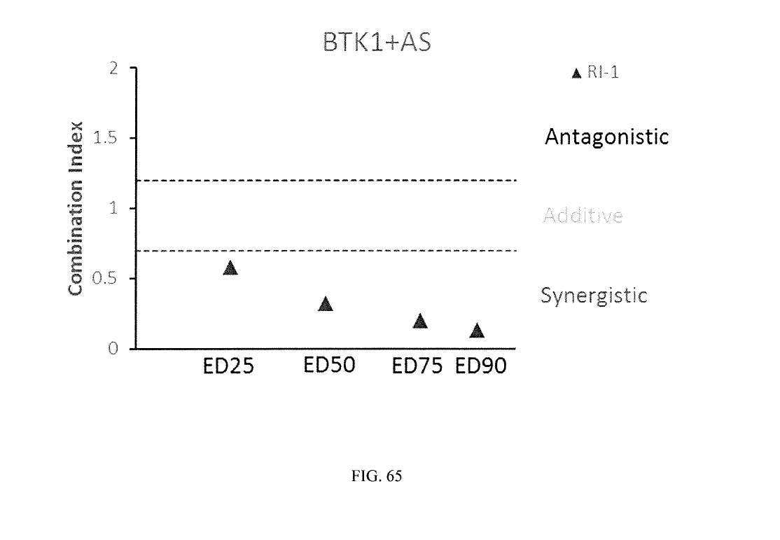

FIGS. 65, 67, and 69 illustrate the synergy observed in the RI-1 cell line when the BTK inhibitor of Formula (2) and the IRAK4 inhibitor of Formula (46) (“AS”); BTK inhibitor of ibrutinib (Formula (10)) (“IBR”) and the IRAK4 inhibitor of Formula (46) (“AS”); and the BTK inhibitor of Formula (21) (“ONO” or “ONO-4059”) and the IRAK4 inhibitor of Formula (46) (“AS”) are combined respectively.

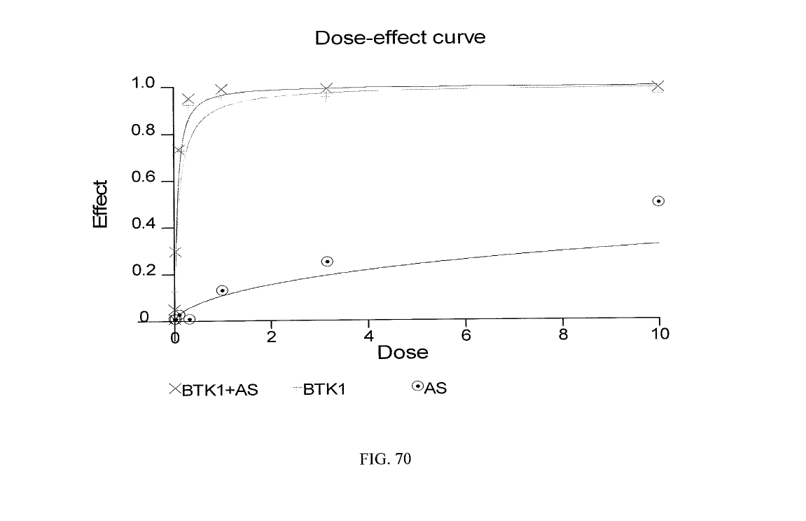

FIGS. 70, 72, and 74 illustrate the dose-effect curves obtained for the tested TMD-8 cell line using combined dosing of the BTK inhibitor of Formula (2) (“BTK1”) and the IRAK4 inhibitor of Formula (46) (“AS”); BTK inhibitor of ibrutinib (Formula (10)) (“IBR”) and the IRAK4 inhibitor of Formula (46) (“AS”); and the BTK inhibitor of Formula (21) (“ONO” or “ONO-4059”) and the IRAK4 inhibitor of Formula (46) (“AS”) respectively.

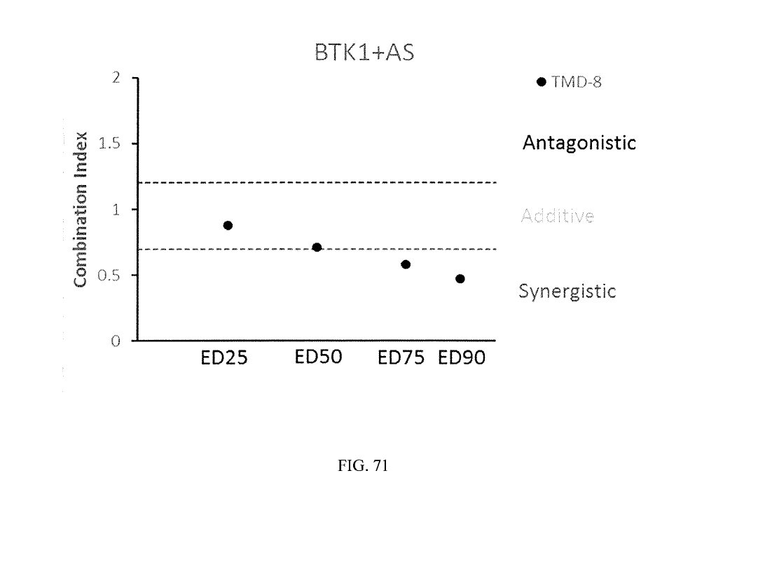

FIGS. 71, 73, and 75 illustrate the synergy observed in the TMD-8 cell line when the BTK inhibitor of Formula (2) and the IRAK4 inhibitor of Formula (46) (“AS”); BTK inhibitor of ibrutinib (Formula (10)) (“BR”) and the IRAK4 inhibitor of Formula (46) (“AS”); and the BTK inhibitor of Formula (21) (“ONO” or “ONO-4059”) and the IRAK4 inhibitor of Formula (46) (“AS”) are combined respectively.

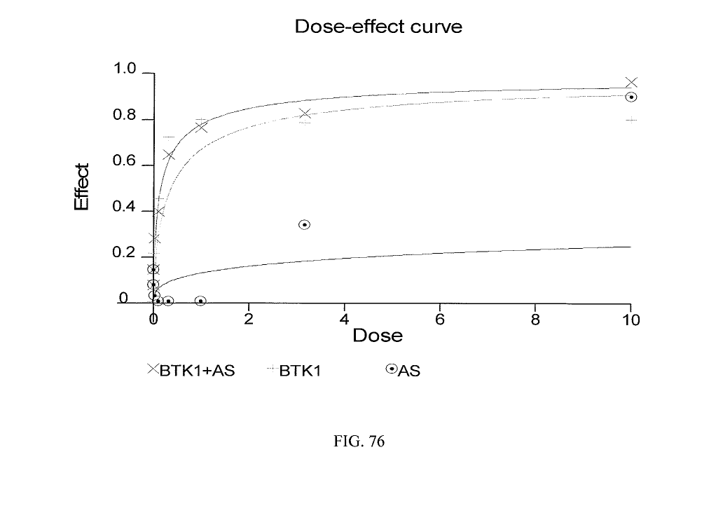

FIGS. 76, 78, and 80 illustrate the dose-effect curves obtained for the tested Mino cell line using combined dosing of the BTK inhibitor of Formula (2) (“BTK1”) and the IRAK4 inhibitor of Formula (46) (“AS”); BTK inhibitor of ibrutinib (Formula (10)) (“IBR”) and the IRAK4 inhibitor of Formula (46) (“AS”); and the BTK inhibitor of Formula (21) (“ONO” or “ONO-4059”) and the IRAK4 inhibitor of Formula (46) (“AS”) respectively.

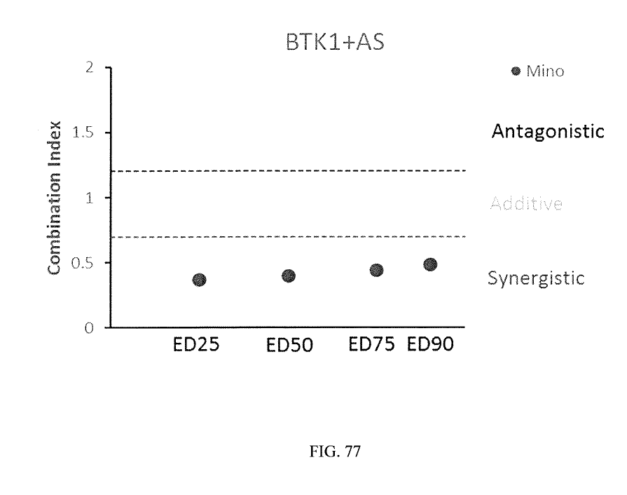

FIGS. 77, 79, and 81 illustrate the synergy observed in the Mino cell line when the BTK inhibitor of Formula (2) and the IRAK4 inhibitor of Formula (46) (“AS”); BTK inhibitor of ibrutinib (Formula (10)) (“IBR”) and the IRAK4 inhibitor of Formula (46) (“AS”); and the BTK inhibitor of Formula (21) (“ONO” or “ONO-4059”) and the IRAK4 inhibitor of Formula (46) (“AS”) are combined respectively.

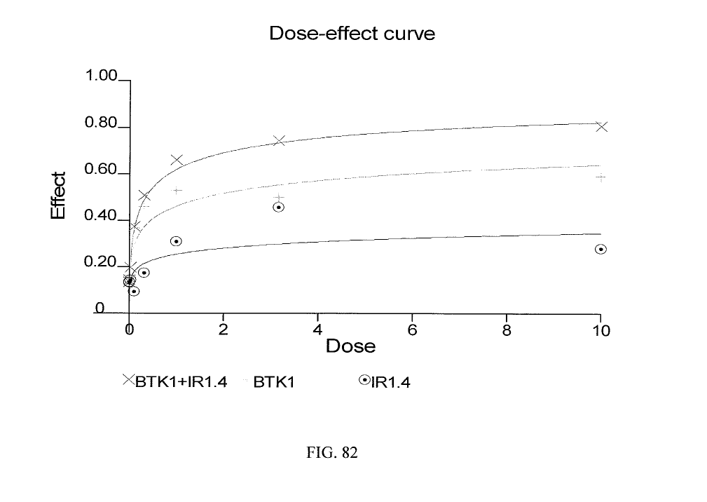

FIGS. 82, 84, and 86 illustrate the dose-effect curves obtained for the tested RI-1 cell line using combined dosing of the BTK inhibitor of Formula (2) (“BTK1”) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”)respectively.

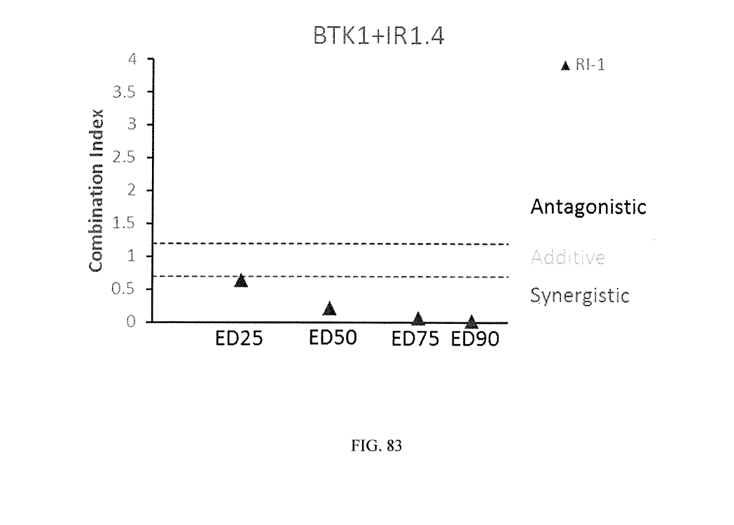

FIGS. 83, 85, and 87 illustrate the synergy observed in the RI-1 cell line when the BTK inhibitor of Formula (2) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”) are combined respectively.

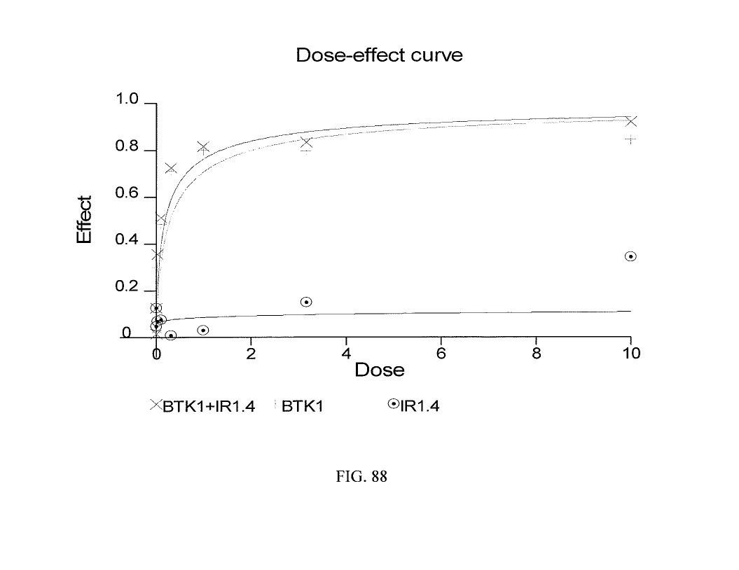

FIGS. 88, 90, and 92 illustrate the dose-effect curves obtained for the tested Mino cell line using combined dosing of the BTK inhibitor of Formula (2) (“BTK1”) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”)respectively.

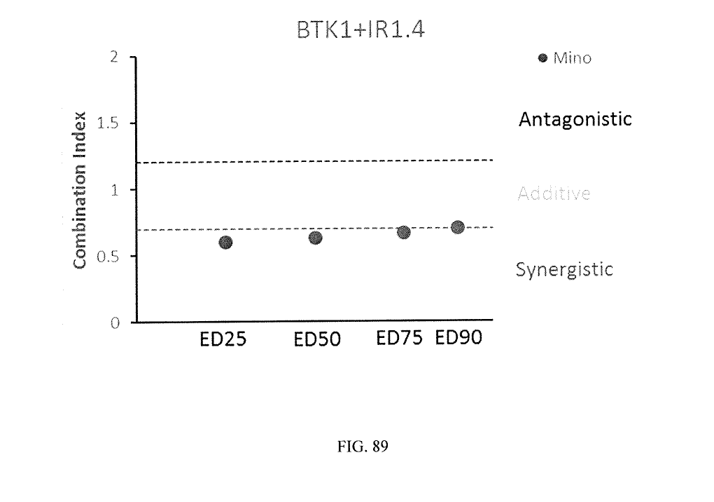

FIGS. 89, 91, and 93 illustrate the synergy observed in the Mino cell line when the BTK inhibitor of Formula (2) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”) are combined respectively.

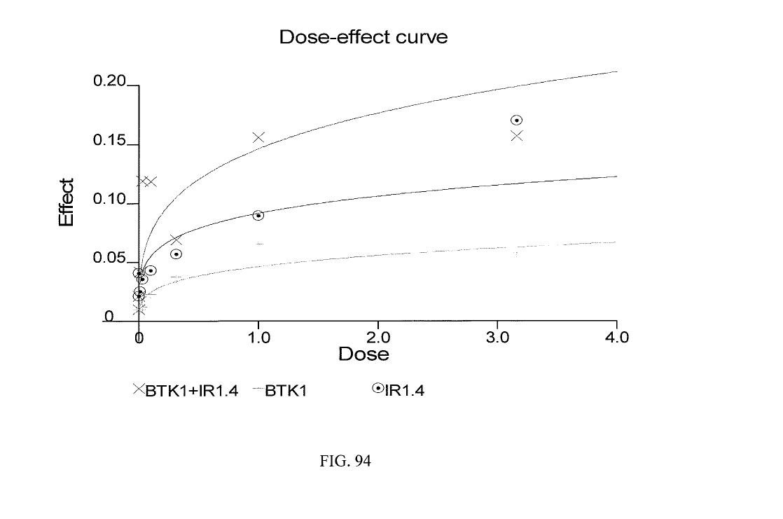

FIGS. 94, 96, and 98 illustrate the dose-effect curves obtained for the tested SU-DHL-6 cell line using combined dosing of the BTK inhibitor of Formula (2) (“BTK1”) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”)respectively.

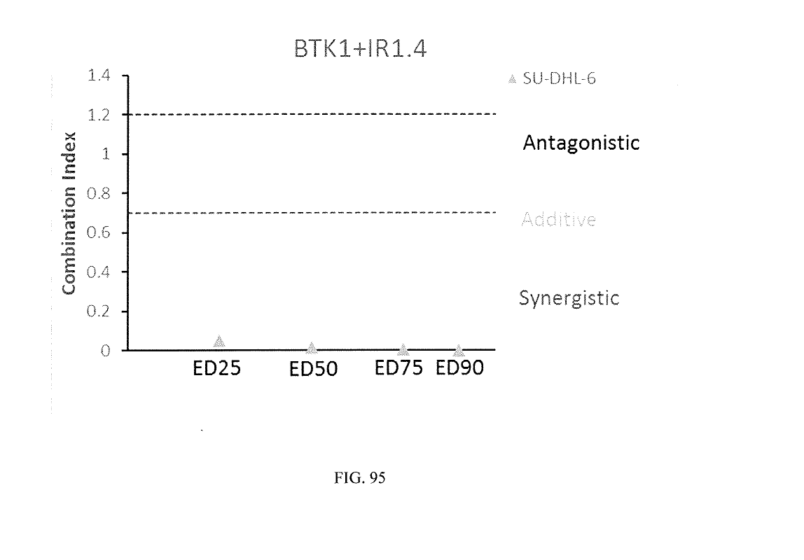

FIGS. 95, 97, and 99 illustrate the synergy observed in the SU-DHL-6 cell line when the BTK inhibitor of Formula (2) and the IRAK1/4 inhibitor of Formula (45) (“IR1.4”); BTK inhibitor of ibrutinib (Formula (10)) (“IBR”) and the IRAK1/4 inhibitor of Formula (45) (“IR1.4”); and the BTK inhibitor of Formula (21) (“ONO” or “ONO-4059”) and the IRAK1/4 inhibitor of Formula (45) (“IR1.4”) are combined respectively.

We claim:

1. A method of treating a hyperproliferative disease, comprising co-administering, to a mammal in need thereof, therapeutically effective amounts of (1) an IRAK4 inhibitor or a pharmaceutically acceptable salt, solvate, hydrate, cocrystal, or prodrug thereof, and (2) a Bruton's tyrosine kinase (BTK) inhibitor or a pharmaceutically acceptable salt, solvate, hydrate, cocrystal, or prodrug thereof.

2. The method of claim 1, wherein the IRAK4 inhibitor is administered to the mammal before administration of the BTK inhibitor.

3. The method of claim 1, wherein the IRAK4 inhibitor is administered to the mammal simultaneously with the administration of the BTK inhibitor.

4. The method of claim 1, wherein the IRAK4 inhibitor is administered to the mammal after administration of the BTK inhibitor.

5. The method of claim 1, wherein the BTK inhibitor is selected from the group consisting of: 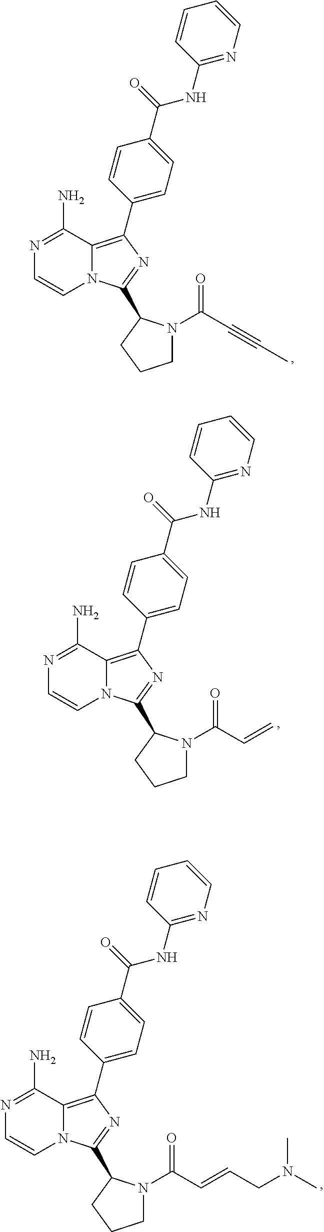 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","] 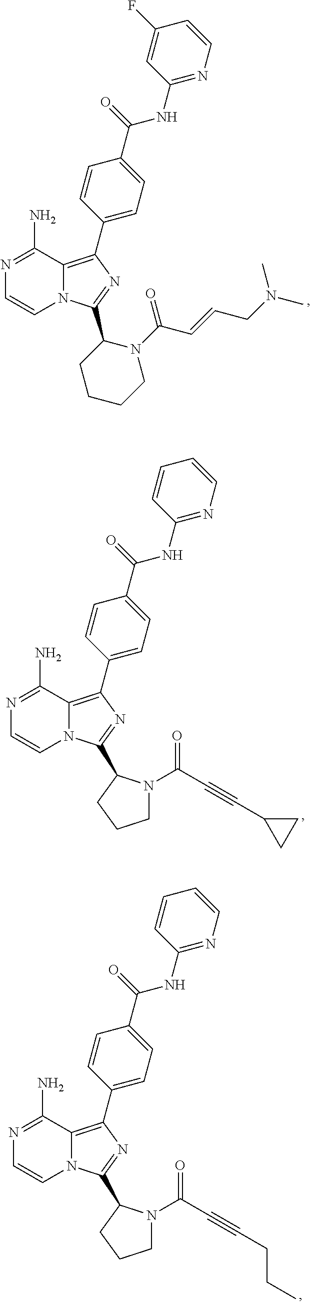 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]
and pharmaceutically-acceptable salts, cocrystals, hydrates, solvates, and prodrugs thereof.

6. The method of claim 1, wherein the BTK inhibitor is selected from the group consisting of: 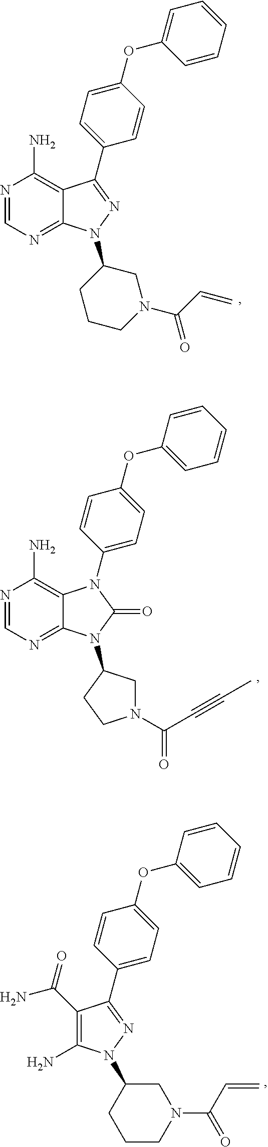 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]
and pharmaceutically-acceptable salts, cocrystals, hydrates, solvates, and prodrugs thereof.

7. The method of claim 1, wherein the IRAK4 inhibitor is selected from the group consisting of: 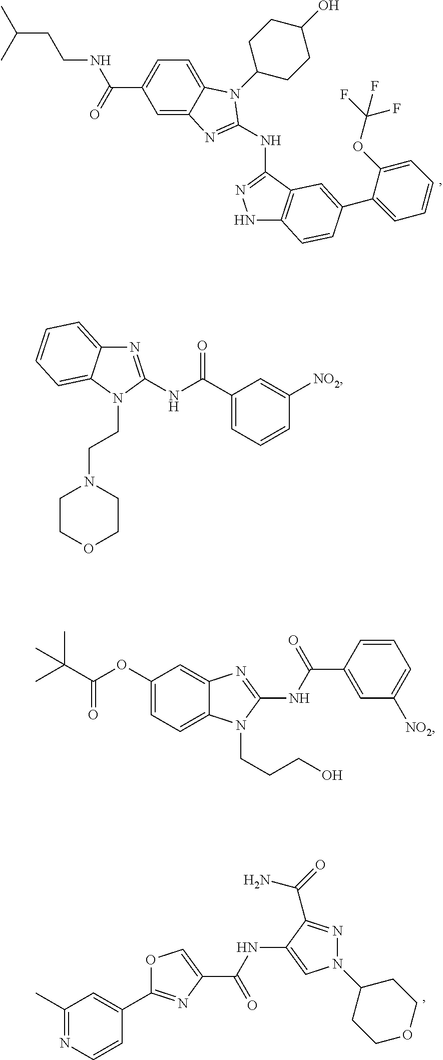 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]
and pharmaceutically-acceptable salts, cocrystals, hydrates, solvates, or prodrugs thereof.

8. The method of claim 1, further comprising the step of administering a therapeutically effective amount of an anti-CD20 antibody.

9. The method of claim 8, wherein the anti-CD20 antibody is selected from the group consisting of rituximab, obinutuzumab, ofatumumab, veltuzumab, tositumomab, ibritumomab, and fragments, derivatives, conjugates, variants, radioisotope-labeled complexes, biosimilars thereof, and combinations thereof.

10. The method of claim 1, wherein the hyperproliferative disease is a B cell hematological malignancy.

11. The method of claim 10, wherein the B cell hematological malignancy is selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, and myelofibrosis.

12. A method of treating a cancer in a human comprising the step of co-administering (1) a therapeutically effective amount of an IRAK4 inhibitor or a pharmaceutically acceptable salt, solvate, hydrate, cocrystal, or prodrug thereof, and (2) a therapeutically effective amount of a Bruton's tyrosine kinase (BTK) inhibitor or a pharmaceutically acceptable salt, solvate, hydrate, cocrystal, or prodrug thereof, wherein the therapeutically effective amount is effective to inhibit signaling between a tumor cell of the cancer and at least one tumor microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts.

13. The method of claim 12, wherein the cancer is a solid tumor cancer selected from the group consisting of bladder cancer, non-small cell lung cancer, cervical cancer, anal cancer, pancreatic cancer, squamous cell carcinoma including head and neck cancer, renal cell carcinoma, melanoma, ovarian cancer, small cell lung cancer, glioblastoma, gastrointestinal stromal tumor, breast cancer, lung cancer, colorectal cancer, thyroid cancer, bone sarcoma, stomach cancer, oral cavity cancer, oropharyngeal cancer, gastric cancer, kidney cancer, liver cancer, prostate cancer, esophageal cancer, testicular cancer, gynecological cancer, colon cancer, and brain cancer.

14. The method of claim 12, wherein the BTK inhibitor is selected from the group consisting of: 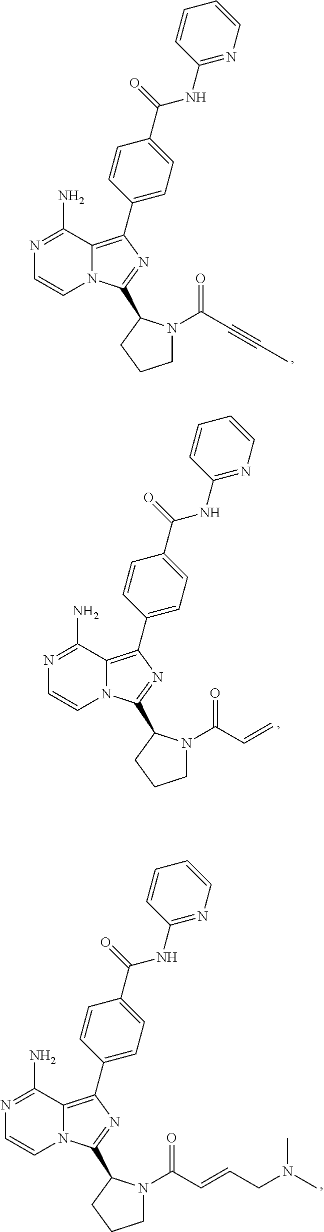 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","] 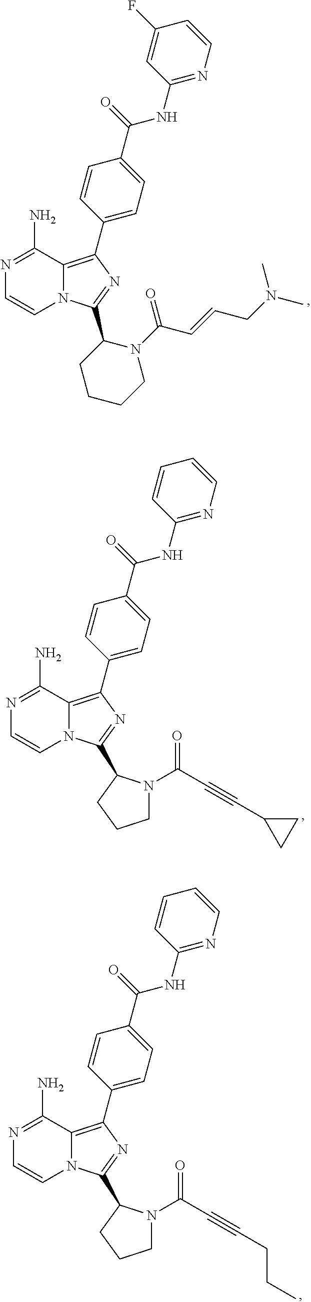 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]
and pharmaceutically-acceptable salts, cocrystals, hydrates, solvates, or prodrugs thereof.

15. The method of claim 12, wherein the BTK inhibitor is selected from the group consisting of: 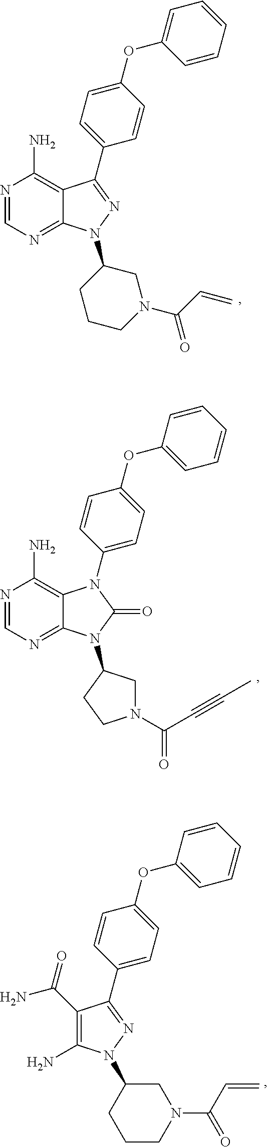 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]
and pharmaceutically-acceptable salts, cocrystals, hydrates, solvates, and prodrugs thereof.

16. The method of claim 12, wherein the IRAK4 inhibitor is selected from the group consisting of: 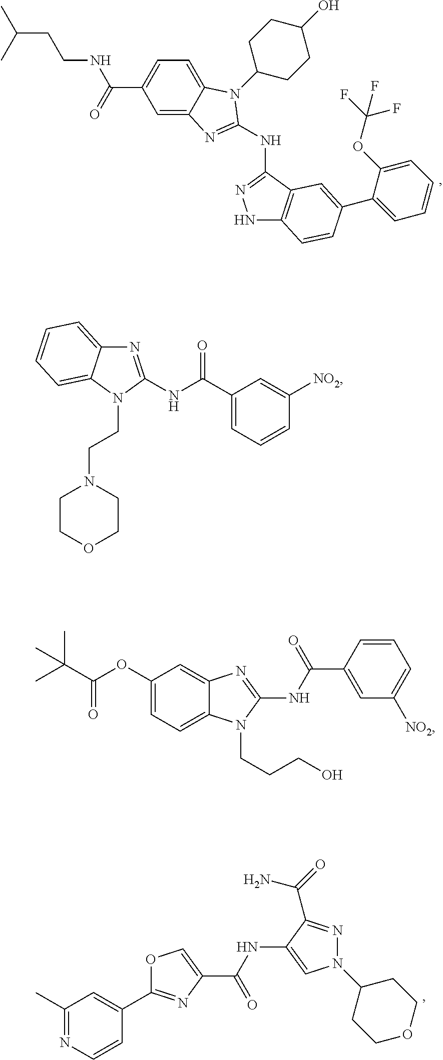 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]
and pharmaceutically-acceptable salts, cocrystals, hydrates, solvates, and prodrugs thereof.

17. A composition comprising therapeutically effective amounts of (1) an IRAK4 inhibitor or a pharmaceutically acceptable salt, solvate, hydrate, cocrystal, or prodrug thereof; and (2) a Bruton's tyrosine kinase (BTK) inhibitor or a pharmaceutically acceptable salt, solvate, hydrate, cocrystal, or prodrug thereof, for use in the treatment of cancer.

18. The composition of claim 17, wherein the BTK inhibitor is selected from the group consisting of: 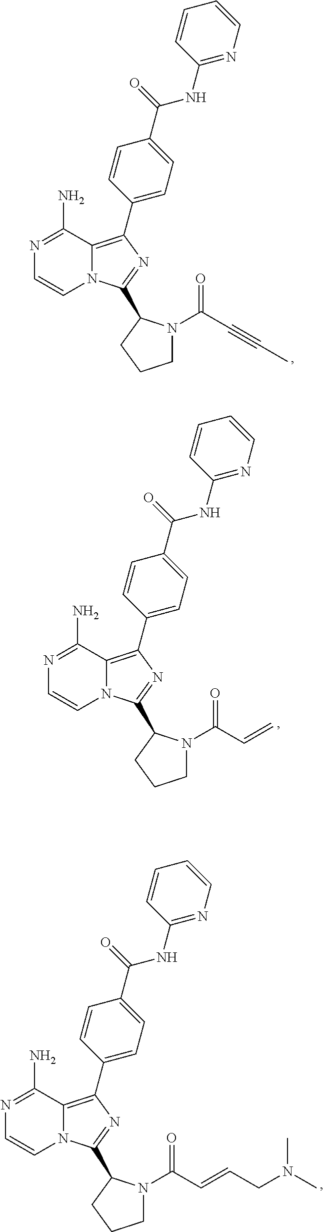 [SMILES: CC#CC(=O)N1CCC[C@H]1c1nc(-c2ccc(C(=O)Nc3ccccn3)cc2)c2c(N)nccn12 ; "," ; C=CC(=O)N1CCC[C@H]1c1nc(-c2ccc(C(=O)Nc3ccccn3)cc2)c2c(N)nccn12 ; "," ; CN(C)C/C=C/C(=O)N1CCC[C@H]1c1nc(-c2ccc(C(=O)Nc3ccccn3)cc2)c2c(N)nccn12 ; ","] 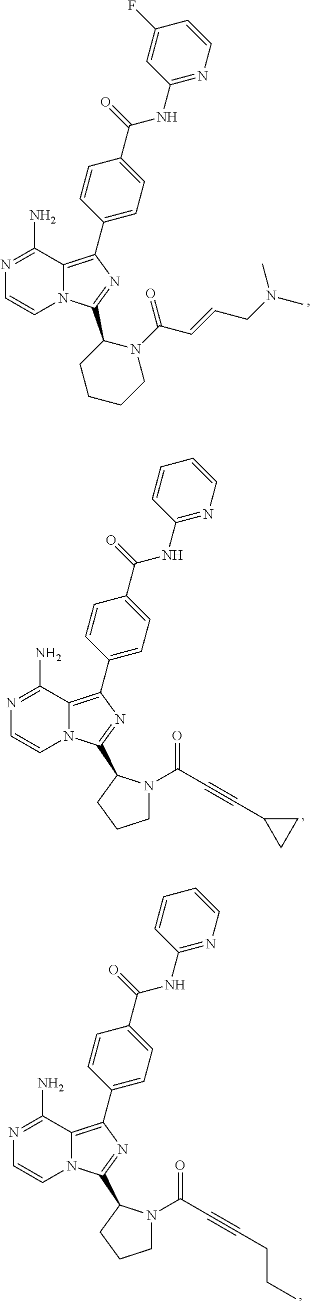 [SMILES: CN(C)C/C=C/C(=O)N1CCCC[C@H]1c1nc(-c2ccc(C(=O)Nc3cc(F)ccn3)cc2)c2c(N)nccn12 ; "," ; Nc1nccn2c([C@@H]3CCCN3C(=O)C#CC3CC3)nc(-c3ccc(C(=O)Nc4ccccn4)cc3)c12 ; "," ; CCCC#CC(=O)N1CCC[C@H]1c1nc(-c2ccc(C(=O)Nc3ccccn3)cc2)c2c(N)nccn12 ; ","]
and pharmaceutically-acceptable salts, cocrystals, hydrates, solvates, and prodrugs thereof.

19. The composition of claim 17, wherein the BTK inhibitor is selected from the group consisting of: 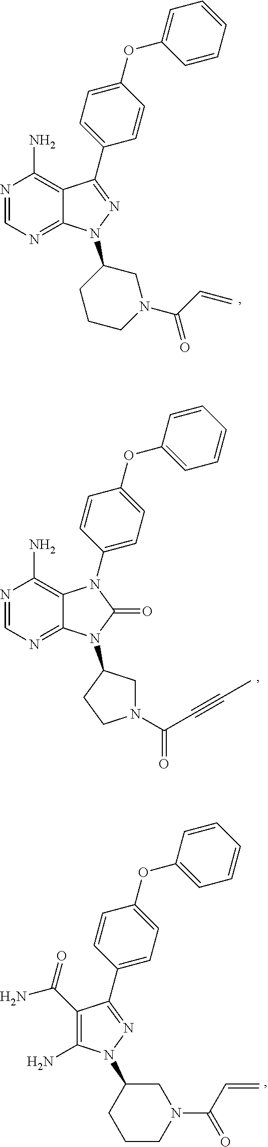 [SMILES: C=CC(=O)N1CCC[C@@H](n2nc(-c3ccc(Oc4ccccc4)cc3)c3c(N)ncnc32)C1 ; CC#CC(=O)N1CC[C@@H](n2c(=O)n(-c3ccc(Oc4ccccc4)cc3)c3c(N)ncnc32)C1 ; C=CC(=O)N1CCC[C@@H](n2nc(-c3ccc(Oc4ccccc4)cc3)c(C(N)=O)c2N)C1]
and pharmaceutically-acceptable salts, cocrystals, hydrates, solvates, and prodrugs thereof.

20. The composition of claim 17, wherein the IRAK4 inhibitor is selected from the group consisting of: 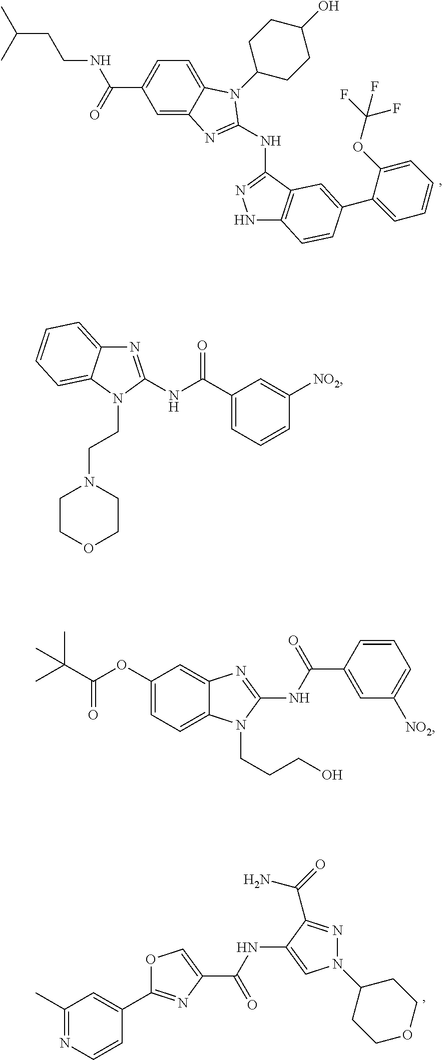 [SMILES: CC(C)CCNC(=O)c1ccc2c(c1)nc(Nc1n[nH]c3ccc(-c4ccccc4OC(F)(F)F)cc13)n2C1CCC(O)CC1 ; O=C(Nc1nc2ccccc2n1CCN1CCOCC1)c1cccc([N+](=O)[O-])c1 ; CC(C)(C)C(=O)Oc1ccc2c(c1)nc(NC(=O)c1cccc([N+](=O)[O-])c1)n2CCCO ; Cc1cc(-c2nc(C(=O)Nc3cn(C4CCOCC4)nc3C(N)=O)co2)ccn1]
and pharmaceutically-acceptable salts, cocrystals, hydrates, solvates, and prodrugs thereof.
